SEQUENCE LISTING (143 sequences)

SEQ ID NO: 1 - DNA, 4027 bp, Homo sapiens
agcgcccaatacgcaaaccgcccctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcaaattctatttcaaggagacagtcataatgaaatacctattgcctacggcagccgctggattgttattactcgcggcccagccggccatggccgactacaaggcaaagcaggtgcagctggtgcaatcagggggaggcgtggtccagcctgggaggtccctgagactctcctgtgcagcctctggattcaccttcagtagctatgctatgcactgggtccgccaggctccagggaaggggctggagtgggtctcagctattagtggtagtggtggtagcacatactacgcagactccgtgaagggccggttcaccatctccagagacaacgccaagaactcactgtatctgcaaatgaacagcctgagagccgaggacacggctgtgtattactgtgcgagagatacccgagggtacttcgatctctggggccgtggcaccctggtcaccgtctcctcaggtggaggcggttcaggcggaggtggctctggcggtggcggatcgtctgagctgactcaggaccctgctatgtctgtggccttgggacagacagtcagaatcacatgtcaaggggacagtctcagaaagtatcatgcaagctggtatcagcagaggccacggcaggcccctcgtcttgtcgtctatggtaagaatgaacgtccctcagggatcccagagcgattctctgggtccacctcaggagacacagcttccttgaccatcagtgggctccaggcggaagatgaggctgactattactgtcactcccgagactctaatgctgatcttgtggtgttcggcggagggaccaaggtcaccgtcctaggtgcggccgcagaacaaaaactcatctcagaagaggatctgaatggggccgcacatcaccatcatcaccattaataagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaatcccaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtccccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgcccacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgtccagcaacgcggcctttttacggttcctggccttttgctggccttttgctcacacgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 2 - protein, 290 aa, Homo sapiens
MKYLLPTAAAGLLLLAAQPAMADYKAKQVQLVQSGGGVVQPGRSLRLSCAASGFTFSSYAMHWVRQAPGKGLEWVSAISGSGGSTYYADSVKGRFTISRDNAKNSLYLQMNSLRAEDTAVYYCARDTRGYFDLWGRGTLVTVSSGGGGSGGGGSGGGGSSELTQDPAMSVALGQTVRITCQGDSLRKYHASWYQQRPRQAPRLVVYGKNERPSGIPERFSGSTSGDTASLTISGLQAEDEADYYCHSRDSNADLVVFGGGTKVTVLGAAAEQKLISEEDLNGAAHHHHHH

SEQ ID NO: 3 - DNA, 821 bp, Homo sapiens
aggatcccaaggcccaactccccgaaccactcagggtcctgtggacgctcacctagctgcaatggctacaggctcccggacgtccctgctcctggcttttggcctgctctgcctgccctggcttcaagagggcagtgccttcccaaccattcccttatccaggctttttgacaacgctagtctccgcgcccatcgtctgcaccagctggcctttgacacctaccaggagtttgaagaagcctatatcccaaaggaacagacgtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatcgcagcccccggactgggcagatcttcaagcagacctacagcaagttcgacacaaactcacacaacgatgacgcactactcaagaactacgggctgctctaatgcttcaggaaggacatggacaaggtcgagacattcctgcgcatcgtgcagtgccgctctgtggagggcagctgtggcttctagctgcccgggtggcatccctgtgacccctccccagtgcctctcctggccttggaagttgccactccagtgcccaccagccttgtcctaataaaattaagttgcatca

SEQ ID NO: 4 - protein, 217 aa, Homo sapiens [sequence omitted]

SEQ ID NO: 5 - DNA, 450 bp, Homo sapiens
gctgcatcagaagaggccatcaagcacatcactgtccttctgccatggccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaactactgcaactagacgcagcccgcaggcagccccccacccgccgcctcctgcaccgagagagatggaataaagcccttgaaccagc

SEQ ID NO: 6 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 7 - DNA, 724 bp, Homo sapiens
ggtgagccccgagattctggctcagagaggtgtcatgggcttccaaaagttgtcccccttcctggctctcagcatcttggtcctgttgcaggcaggcagcctccatgcagcaccattcaggtctgccctggagagcagcccagcagacccggccacgctcagtgaggacgaagcgcgcctcctgctggctgcactggtgcaggactatgtgcagatgaaggccagtgagctggagcaggagcaagagagagagggctccagcctggacagccccagatctaagcggtgcggtaatctgagtacttgcatgctgggcacatacacgcaggacttcaacaagtttcacacgttcccccaaactgcaattggggttggagcacctggaaagaaaagggatatgtccagcgacttggagagagaccatcgccctcatgttagcatgccccagaatgccaactaaactcctccctttccttcctaatttcccttcttgcatccttcctataacttgatgcatgtggtttggttcctctctggtggctctttgggctggtattggtggctttccttgtggcagaggatgtctcaaacttcagatgggaggaaagagagcaggactcacaggttggaagagaatcacctgggaaaataccagaaaatgagggccgctttgagtcccccagagatgtcatcagagctcctctgtcctgctttctgaatgtgc

SEQ ID NO: 8 - protein, 141 aa, Homo sapiens
MGFQKFSPFLALSILVLLQAGSLHAAPFRSALESSPADPATLSEDEARLLLAALVQDYVQMKASELEQEQEREGSSLDSPRSKRCGNLSTCMLGTYTQDFNKFHTFPQTAIGVGAPGKKRDMSSDLERDHRPHVSMPQNAN

SEQ ID NO: 9 - DNA, 804 bp, Salmo sp.
ctggttatgatgaagctctctgccctcctcattgcctatttcctggtcatttgtcagatgtacagctcacatgcagctccagccagaactggtttagagtccatgacagaccaagtcacgctaactgactatgaagcccgaaggctactcaacgccatcgtcaaggagtttgttcaaatgacttcagaggaactggagcaacaagccaatgaaggaaatagcctggatagacccatgtccaagcgttgctccaacctcagcacctgtgtgctgggcaaactgtcccaagagctgcacaaattgcagacgtacccccgcaccaacacgggaagtggcacgcctggcaagaaacgcagcctgcctgagagcaaccgctatgcaagctatggagactcatatgatggaatctgagcggtactcccctccatcaggccaagttaacctccctctgttccagcctagcctgatgattgctgatgcatgtggatcttgcttgcttgaccgactgcagacccaaccttgatgtcccgcaatgtccctcctctctttttcttttgttaaaataccctttttttgacagagaataaaatatataagtacaaagcagagtccaatcctttagatttagaaagtgaataatgatttagactaactcccctatcttaaggtagtatgatatccctatactatagacgatcattcacaatatataaaaaagtgttaatcaaaacaaaatcttaatcaactgcttcttctttcaaccatgactagggttcttgtttaataaacatagttgtttaaaaa

SEQ ID NO: 10 - protein, 136 aa, Salmo sp.
MVMMKLSALLIAYFLVICQMYSSHAAPARTGLESMTDQVTLTDYEARRLLNAIVKEFVQMTSEELEQQANEGNSLDRPMSKRCSNLSTCVLGKLSQELHKLQTYPRTNTGSGTPGKKRSLPESNRYASYGDSYDGI

SEQ ID NO: 11 - DNA, 462 bp, Homo sapiens
atgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactaacttga

SEQ ID NO: 12 - protein, 296 aa, Homo sapiens
MKYLLPTAAAGLLLLAAQPAMADYKAKQVQLVQSGGGVVQPGRSLRLSCAASGFTFSSYAMHWVRQAPGKGLEWVSAISGSGGSTYYADSVKGRFTISRDNAKNSLYLQMNSLRAEDTAVYYCARDTRGYFDLWGRGTLVTVSSGGGGSGGGGSGGGGSSELTQDPAMSVALGQTVRITCQGDSLRKYHASWYQQRPRQAPRLVVYGKNERPSGIPERFSGSTSGDTASLTISGLQAEDEADYYCHSRDSNADLVVFGGGTKVTVLGGGGGSCAAAEQKLISEEDLNGAAHHHHHH

SEQ ID NO: 13 - protein, 771 aa, Rattus sp.
MALFLLTCLLAVFSAATAQSSLLGPSSIFGPGEVNVLEGDSVSITCYYPTTSVTRHSRKFWCREEESGRCVTLASTGYTSQEYSGRGKLTDFPDKGEFVVTVDQLTQNDSGSYKCGVGVNGRGLDFGVNVLVSQKPEPDDVVYKQYESYTVTITCPFTYATRQLKKSFYKVEDGELVLIIDSSSKEAKDPRYKGRITLQIQSTTAKEFTVTIKHLQLNDAGQYVCQSGSDPTAEEQNVDLRLLTPGLLYGNLGGSVTFECALDSEDANAVASLRQVRGGNVVIDSQGTIDPAFEGRILFTKAENGHFSVVIAGLRKEDTGNYLCGVQSNGQSGDGPTQLRQLFVNEEIDVSRSPPVLKGFPGGSVTIRCPYNPKRSDSHLQLYLWEGSQTRHLLVDSGEGLVQKDYTGRLALFEEPGNGTFSVVLNQLTAEDEGFYWCVSDDDESLTTSVKLQIVDGEPSPTIDKFTAVQGEPVEITCHFPCKYFSSEKYWCKWNDHGCEDLPTKLSSSGDLVKCNNNLVLTLTLDSVSEDDEGWYWCGAKDGHEFEEVAAVRVELTEPAKVAVEPAKVPVDSPHINPTDANARAKDAPEEEAMESSVREDENKANLDPRLFADEREIQNAGDQAQENRASGNAGSAGGQSGSSKVLFSTLVPLGLVLAVGAVAVAIARARHRRNVDRVSIGSYRTDISMSDLENSREFGAIDNPSACPDARETALGGKDELATATESTVEIEEPKKAKRSSKEEADLAYSAFLLQSNTIAAEHQDGPKEA

SEQ ID NO: 14 - protein, 663 aa, Streptococcus pneumoniae
ENEGSTQAATSSNMAKTEHRKAAKQVVDEYIEKMLREIQLDRRKHTQNVALNIKLSAIKTKYLRELNVLEEKSKDELPSEIKAKLDAAFEKFKKDTLKPGEKVAEAKKKVEEAKKKAEDQKEEDRRNYPTNTYKTLELEIAEFDVKVKEAELELVKEEAKESRNEGTIKQAKEKVESKKAEATRLENIKTDRKKAEEEAKRKADAKLKEANVATSDQGKPKGRAKRGVPGELATPDKKENDAKSSDSSVGEETLPSSSLKSGKKVAEAEKKVEEAEKKAKDQKEEDRRNYPTNTYKTLDLEIAESDVKVKEAELELVKEEAKEPRDEEKIKQAKAKVESKKAEATRLENIKTDRKKAEEEAKRKAAEEDKVKEKPAEQPQPAPATQPEKPAPKPEKPAEQPKAEKTDDQQAEEDYARRSEEEYNRLTQQQPPKTEKPAQPSTPKTGWKQENGMWYFYNTDGSMATGWLQNNGSWYYLNANGAMATGWLQNNGSWYYLNANGSMATGWLQNNGSWYYLNANGAMATGWLQYNGSWYYLNSNGAMATGWLQYNGSWYYLNANGDMATGWLQNNGSWYYLNANGDMATGWLQYNGSWYYLNANGDMATGWVKDGDTWYYLEASGAMKASQWFKVSDKWYYVNGSGALAVNTTVDGYGVNANGEWVN

SEQ ID NO: 15 - DNA, 462 bp, Homo sapiens
atgggtctcacctcccaactgcttccccctctgttcttcctgctagcatgtgccggcaactttgtccacggacacaagtgcgatatcaccttacaggagatcatcaaaactttgaacagcctcacagagcagaagactctgtgcaccgagttgaccgtaacagacatctttgctgcctccaagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttgaattcctgtcctgtgaaggaagccaaccagagtacgttggaaaacttcttggaaaggctaaagacgatcatgagagagaaatattcaaagtgttcgagctga

SEQ ID NO: 16 - protein, 5 aa, Artificial Sequence
  amino acid sequence conserved in pIgR protein
LRKED

SEQ ID NO: 17 - protein, 7 aa, Artificial Sequence
  amino acid sequence conserved in pIgR protein
QLFVNEE

SEQ ID NO: 18 - protein, 5 aa, Artificial Sequence
  amino acid sequence conserved in pIgR protein
LNQLT

SEQ ID NO: 19 - protein, 5 aa, Artificial Sequence
  amino acid sequence conserved in pIgR protein
YWCKW

SEQ ID NO: 20 - protein, 5 aa, Artificial Sequence
  amino acid sequence conserved in pIgR protein
GWYWC

SEQ ID NO: 21 - protein, 6 aa, Artificial Sequence
  amino acid sequence conserved in pIgR protein
STLVPL

SEQ ID NO: 22 - protein, 5 aa, Artificial Sequence
  amino acid sequence conserved in pIgR protein
SYRTD

SEQ ID NO: 23 - protein, 5 aa, Artificial Sequence
  amino acid sequence conserved in pIgR protein
KRSSK

SEQ ID NO: 24 - protein, 15 aa, Artificial Sequence
  spacer sequence
GGGGSGGGGSGGGGS

SEQ ID NO: 25 - protein, 22 aa, Homo sapiens
MKYLLPTAAAGLLLLAAQPAMA

SEQ ID NO: 26 - protein, 23 aa, Homo sapiens
AAAEQKLISEEDLNGAAHHHHHH

SEQ ID NO: 27 - protein, 129 aa, Homo sapiens [sequence omitted]

SEQ ID NO: 28 - DNA, 28 bp, Artificial Sequence
  primer
gtatcgatctttttgccttcttgggytc

SEQ ID NO: 29 - DNA, 28 bp, Artificial Sequence
  primer
ggaattcgaraartaytggtgyaartgg

SEQ ID NO: 30 - DNA, 28 bp, Artificial Sequence
  primer
gtatcgatcnrttngcrttrttnggrtc

SEQ ID NO: 31 - DNA, 37 bp, Artificial Sequence
  primer
cgggaagatctggagtgaagcagggccacttctatgg

SEQ ID NO: 32 - DNA, 58 bp, Artificial Sequence
  primer
cggaattcctagtgatggtgatggtgatgtttggagctcccaccttgttcctcagagc

SEQ ID NO: 33 - DNA, 16 bp, Artificial Sequence
  primer
caggaaacagctagac

SEQ ID NO: 34 - DNA, 49 bp, Artificial Sequence
  primer
agttgcggccgcggcaggagccaccgccaccacctaggacggtgacctt

SEQ ID NO: 35 - DNA, 24 bp, Artificial Sequence
  primer
aaatacctattgcctacggcagcc

SEQ ID NO: 36 - DNA, 59 bp, Artificial Sequence
  primer
cggaattcctactagcagccaccgccacctgcggccgctaggacggtgaccttggtccc

SEQ ID NO: 37 - DNA, 60 bp, Artificial Sequence
  primer
ccggaattcgtcgactcatcagcagcctccaccgccacctaggacggtgaccttggtccc

SEQ ID NO: 38 - protein, 5 aa, Artificial Sequence
  linker sequence
GGGGS

SEQ ID NO: 39 - protein, 20 aa, Artificial Sequence
  linker sequence
GGGGSGGGGSGGGGSGGGGS

SEQ ID NO: 40 - protein, 25 aa, Artificial Sequence
  linker sequence
GGGGSGGGGSGGGGSGGGGSGGGGS

SEQ ID NO: 41 - DNA, 39 bp, Artificial Sequence
  primer
tgaccctccgccacctgaggagacggtgaccagggtgcc

SEQ ID NO: 42 - DNA, 45 bp, Artificial Sequence
  primer
ggaccctccgcctcctgaggagacggtgaccagggtgccacggcc

SEQ ID NO: 43 - DNA, 60 bp, Artificial Sequence
  primer
gctccctccgcctccggaccctccgcctcctgaggagacggtgaccagggtgccacggcc

SEQ ID NO: 44 - DNA, 86 bp, Artificial Sequence
  primer
cggaattcctcgagctactagtcgacctagtgatggtggtgatggtggtcgactgcggccgcacctaggacggtgaccttggtccc

SEQ ID NO: 45 - DNA, 39 bp, Artificial Sequence
  primer
ggtggcggagggtcatctgagctgactcaggaccctgct

SEQ ID NO: 46 - DNA, 80 bp, Artificial Sequence
  primer
ggaggcggagggtccggtggaggcggttcaggcggaggtggctctggcggtggcggatcgtctgagctgactcaggaccc

SEQ ID NO: 47 - DNA, 95 bp, Artificial Sequence
  primer
ggaggcggagggtccggaggcggagggagcggtggaggcggttcaggcggaggtggctctggcggtggcggatcgtctgagctgactcaggaccc

SEQ ID NO: 48 - DNA, 45 bp, Artificial Sequence
  primer
catgccatggccttcccaaccattcccttatccaggctttttgac

SEQ ID NO: 49 - DNA, 74 bp, Artificial Sequence
  primer
ccgcggccgctatggccgacgtcgactgaccctccgccaccgaagccacagctgccctccacagagcggcactg

SEQ ID NO: 50 - DNA, 59 bp, Artificial Sequence
  primer
ataagaatgcggccgccggtggaggcggttcaatggctacaggctcccggacgtccctg

SEQ ID NO: 51 - DNA, 68 bp, Artificial Sequence
  primer
cggaattcctactaatgatggtgatgatggtgtgcggccgcgaagccacagctgccctccacagagcg

SEQ ID NO: 52 - DNA, 65 bp, Artificial Sequence
  primer
ataagaatgcggccgcaggtggcggagggtcattcccaaccattcccttatccaggctttttgac

SEQ ID NO: 53 - DNA, 86 bp, Artificial Sequence
  primer
cggaattcctcgagctactagtcgacctagtgatggtggtgatggtggtcgacgaagccacagctgccctccacagagcggcactg

SEQ ID NO: 54 - DNA, 30 bp, Artificial Sequence
  primer
caccatgtacaggatgcaactgctgtcttg

SEQ ID NO: 55 - DNA, 51 bp, Artificial Sequence
  primer
gatttgccgctaccggaagtcgacccagttagtgttgagatgatgctttga

SEQ ID NO: 56 - protein, 13 aa, Artificial Sequence
  polypeptide spacer
GSTSGSGKSSEGK

SEQ ID NO: 57 - DNA, 53 bp, Artificial Sequence
  primer
gtagcggcaaatcctctgaaggcaaacaggtgcagctggtgcaatcaggggga

SEQ ID NO: 58 - DNA, 24 bp, Artificial Sequence
  primer
acctaggacggtgaccttggtccc

SEQ ID NO: 59 - DNA, 28 bp, Artificial Sequence
  primer
cacgatgggtctcacctcccaactgctt

SEQ ID NO: 60 - DNA, 51 bp, Artificial Sequence
  primer
gatttgccgctaccggaagtcgacccgctcgaacactttgagtatttctct

SEQ ID NO: 61 - DNA, 62 bp, Homo sapiens
ataagaatgcggccgcaggtggcggagggtcatttgtgaaccaacacctgtgcggctcacac

SEQ ID NO: 62 - DNA, 85 bp, Homo sapiens
cggaattcctcgagctactagtcgacctagtgatggtggtgatggtggtcgacgttgcagtagttctccagctggtagagggagc

SEQ ID NO: 63 - DNA, 36 bp, Homo sapiens
catgccatggccatgggcttccaaaagttctccccc

SEQ ID NO: 64 - DNA, 50 bp, Homo sapiens
catgccatggctgaaccgcctccaccgttggcattctggggcatgctaac

SEQ ID NO: 65 - DNA, 56 bp, Homo sapiens
ataagaatgcggccgccggtggaggcggttcaatgggcttccaaaagttctccccc

SEQ ID NO: 66 - DNA, 37 bp, Homo sapiens
cgaattctaataagttggcattctggggcatgctaac

SEQ ID NO: 67 - DNA, 120 bp, Salmo sp.
catggcctgctccaacctcagcacctgtgtgctgggcaaactgtcccaagagctgcacaaattgcagacgtacccccgcaccaacacgggaagtggcacgcctggtggaggcggttcagc

SEQ ID NO: 68 - DNA, 120 bp, Salmo sp.
catggctgaaccgcctccaccaggcgtgccacttcccgtgttggtgcgggggtacgtctgcaatttgtgcagctcttgggacagtttgcccagcacacaggtgctgaggttggagcaggc

SEQ ID NO: 69 - DNA, 122 bp, Salmo sp.
ggccggtggaggcggttcatgctccaacctcagcacctgtgtgctgggcaaactgtcccaagagctgcacaaattgcagacgtacccccgcaccaacacgggaagtggcacgccttaataag

SEQ ID NO: 70 - DNA, 122 bp, Salmo sp.
aattctaataaaggcgtgccacttcccgtgttggtgcgggggtacgtctgcaatttgtgcagctcttgggacagtttgcccagcacacaggtgctgaggttggagcatgaaccgcctccacc

SEQ ID NO: 71 - protein, 18 aa, Artificial Sequence
  synthetic oligopeptide
IENKAIQDPRLFAEEKAV

SEQ ID NO: 72 - protein, 23 aa, Artificial Sequence
  synthetic oligopeptide
CGGGGIENKAIQDPRLFAEEKAV

SEQ ID NO: 73 - protein, 24 aa, Artificial Sequence
  synthetic oligopeptide
IENKAIQDPRLFAEEKAVGGGGGC

SEQ ID NO: 74 - protein, 10 aa, Artificial Sequence
  synthetic oligopeptide
AIQDPRLFAE

SEQ ID NO: 75 - protein, 136 aa, Rattus sp.
MGFLKFSPFLVVSILLLYQACGLQAVPLRSTLESSPGMATLSEEEARLLAALVQNYMQMKVRELEQEEEQEAEGSSLDSPRSKRCGNLSTCMLGTYTQDLNKFHTFPQTSIGVGAPGKKRDMAKDLETNHHPYFGN

SEQ ID NO: 76 - protein, 136 aa, Murinae gen. sp.
MGFLKFSPFLVVSILLLYQACSLQAVPLRSILESSPGMATLSEEEVRLLAALVQDYMQMKARELEQEEEQEAEGSSLDSPRSKRCGNLSTCMLGTYTQDLNKFHTFPQTSIGVEAPGKKRDVAKDLETNHQSHFGN

SEQ ID NO: 77 - protein, 141 aa, Homo sapiens; identical to SEQ ID NO: 8

SEQ ID NO: 78 - protein, 143 aa, Ovis sp.
MGFGKSSPFLAFSILVLCQAGSLQATPLRSALETLPDPGALSEKEGRLLLAALVKAYVQRKTNELEQEEEQEETEDSSLDSSRAKRCSNLSTCVLSAYWKDLNNYHRYSGMGFGPETPGKKRDIANSLEKDLSSHFGVPTDAN

SEQ ID NO: 79 - protein, 138 aa, Gallus gallus
MVMLKISSFLAVYALVVCQMDSFQAAPVRPGLESITDRVTLSDYEARRLLNALVKDFIQMTAEELEQASEGNSLDRPISKRCASLSTCVLGKLSQELHKLQTYPRTDVGAGTPGKKRNVLNDLDHERYANYGETLGNN

SEQ ID NO: 80 - DNA, 39 bp, Murinae gen. sp.
gcccaagcttggccaatgaggctctacttgttcacgctc

SEQ ID NO: 81 - DNA, 37 bp, Murinae gen. sp.
tccccccggggggggctcaggcgctagcacctggagg

SEQ ID NO: 82 - DNA, 39 bp, Murinae gen. sp.
gcccaagcttggccacctccaggtgctagcgcctgagcc

SEQ ID NO: 83 - DNA, 37 bp, Murinae gen. sp.
tccccccgggggggttggcaaaaggcccgggatttgg

SEQ ID NO: 84 - DNA, 39 bp, Murinae gen. sp.
gcccaagcttggccaattccaaatcccgggccttttgcc

SEQ ID NO: 85 - DNA, 35 bp, Murinae gen. sp.
ctagtctagacacctaggcttcctggggaccatcg

SEQ ID NO: 86 - DNA, 37 bp, Murinae gen. sp.
tccccccgggggggcgttctgtggcgtcacctcaagg

SEQ ID NO: 87 - DNA, 39 bp, Murinae gen. sp.
gcccaagcttggccaccttgaggtgacgccacagaacgc

SEQ ID NO: 88 - DNA, 39 bp, Homo sapiens
gcccaagcttggacccaccagcaatgctgctcttcgtgc

SEQ ID NO: 89 - DNA, 24 bp, Homo sapiens
gtgacattccctggtaccttgagg

SEQ ID NO: 90 - DNA, 24 bp, Homo sapiens
cctcaaggtaccagggaatgtcac

SEQ ID NO: 91 - DNA, 26 bp, Homo sapiens
aaactcggtcgacgttcttcctgtgc

SEQ ID NO: 92 - DNA, 24 bp, Homo sapiens
ggcacaggaagaacgtcgaccgag

SEQ ID NO: 93 - DNA, 34 bp, Homo sapiens
ctagtctagaacaccgtctaggcttcctgggggc

SEQ ID NO: 94 - DNA, 34 bp, Homo sapiens
ctagtctagacctcctcatgccaccctcatcccc

SEQ ID NO: 95 - DNA, 39 bp, Rattus sp.
gcccaagcttggccacaagcgatgaggctctccttgttc

SEQ ID NO: 96 - DNA, 24 bp, Rattus sp.
gggttagcaggatcctgccttcaa

SEQ ID NO: 97 - DNA, 24 bp, Rattus sp.
gaaggcaggatcctgctaaccccc

SEQ ID NO: 98 - DNA, 24 bp, Rattus sp.
aggactttggagctcccgctttgt

SEQ ID NO: 99 - DNA, 24 bp, Rattus sp.
acaaagcgggagctccaaagtcct

SEQ ID NO: 100 - DNA, 34 bp, Rattus sp.
ctagtctagacagcactgcctaggcttcctgggg

SEQ ID NO: 101 - DNA, 27 bp, Rattus sp.
cttagcaacctgcagttctatcgtggt

SEQ ID NO: 102 - DNA, 27 bp, Rattus sp.
acgatagaactgcaggttgctgaagct

SEQ ID NO: 103 - protein, 102 aa, Simian
GSGVKQGHFYGETAAVYVAVEEKKVAGSRYVSPAKADAAPDEKVLDSGVREIENKAIQDPRLFAEEKVVADTGDQAGGSRASVDSSSSEEQGGSSKHHHHHH

SEQ ID NO: 104 - protein, 102 aa, Homo sapiens
GSGVKQGHFYGETAAVYVAVEERKAAGSRDVSLAKADAAPDEKVLDSGFREIENKAIQDPRLFAEEKAVADTRDQADGSRASVDSGSSEEQGGSSRHHHHHH

SEQ ID NO: 105 - protein, 117 aa, Rattus sp.
GSGVKEGQVYGETTAIYVAVEERTRGSPHINPTDANARAKDAPEEEAMESSVREDENKANLDPRLFADEREIQNAGDQAQENRASGNAGSAGGQSGSSKRIPNSPSPSPLEQFIVTD

SEQ ID NO: 106 - protein, 117 aa, Oryctolagus cuniculus
GSGVKDGHEFEEVAAVRVELTEPAKVAVEPAKVPVDPAKVAPAPAEEKAKAAVPSAQEKAVVPIVKEAENKVVQKPRLLAEEVAVQSAEDPASGSRASVDASSASGQSGSAKRIHRD

SEQ ID NO: 107 - protein, 94 aa, Artificial Sequence
  consensus sequence
GSGVKQGHFYGETAAVYVAVEERKKAGISRVAAKAKAAPDEKVLDSGVREIENKAIQDPRLFAEEKAVQDTGDQAGSRASVDASSAEGQSGSSK

SEQ ID NO: 108 - protein, 243 aa, Homo sapiens
EKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRALVSTLVPLGLVLAVGAVAVGVARARHRKNVDRVSIRSYRTDISMSDFENSREFGANDNMGASSITQETSLGGKEEFVATTESTTETKEPKKAKRS

SEQ ID NO: 109 - protein, 244 aa, Simian
EKYWCKWSNTGCQTLPSQDEGPSEAFVNCDENSRLVSLTLNPVTRADEGWYWCGVKQGHFYCETAAVYVAVEEKKVAGSRYVSPAKADAAPDEKVLDSGVREIENKAIQDPRLFAEEICVVADTGDQAGGSRASVDSSSSEFQGGSSKALVSTLVPLGLVLALGAVWCVARARFIRKNVDRVSIRSYRTDISMSDFENSREFGANDNMGASSITQETSLGGKDEFVATPESTTETKEPKKAKRS

SEQ ID NO: 110 - protein, 249 aa, Rattus sp.
EKYWCKWSNDGCIIILPSHDEGARQSSVSCDQSSQIVSMTLNPVKKEDEGWYWCGVKEGQVYGETTPAIYVAVEERTRGSPHINPTDANARAKDAPEEEAMESSVREDEIVKANLDPRLFADEREIQNAGDQAQENRASGIVAGSAGGQSGSSKVLFSTLVPLGLVLAVGAVAVWVARVRHRKNVDRMSISSYTDISMGDLENSREFGAIDNPSACPDARETALGGKDELATATESTVEIEEPKKAKRS

SEQ ID NO: 111 - protein, 258 aa, Oryctolagus cuniculus
EKYWCKWNDHGCEDLPTKLSSSGDLVKCNNNLVLTLTLDSVSEDDEGWYWCGAKDGHEFEEVAAVRVELTEPAICVAVEFAKVPVDPAKAAPAPAEEKAKAAVPSAQEKAVVPIVKEAENKVVQKPRLLAEEVAVQSAEDPASGSRASVDASSASGQSGSAKVLISTLVPLGLVLAAGAMAVAIARARHRRNVDRVSIGSYRTDISMSDLENSREFGAIDNPSACPDARETALGCKDELATATESTVEIEEPKKAKRS

SEQ ID NO: 112 - DNA, 649 bp, Homo sapiens
gagaaatactggtgcaagtggaataacacgggctgccaggccctgcccagccaagacgaaggccccagcaaggccttcgtgaactgtgacgagaacagccggcttgtctccctgaccctgaacctggtgaccagggctgatgagggctggtactggtgtggagtgaagcagggccacttctatggagagactgcagccgtctatgtggcagttgaagagaggaaggcagcggggtcccgcgatgtcagcctagcgaaggcagacgctgctcctgatgagaaggtgctagactctggttttcgggagattgagaacaaagccattcaggatcccaggctttttgcagaggaaaaggcggtggcagatacaagagatcaagccgatgggagcagagcatctgtggattccggcagctctcaggaacaaggtggaagctccagagcgctggtctccaccctggtgcccctgggcctggtgctggcagtgggagccgtggctgtgggggtagaactccagggaatttggagccaatgacaacatgggagcctcttcgatcactcaggagacatccctcggaggaaaagaagagtttgttgccaccactgagagcaccacagagaccaaagaacccaacaaggcaaaaaggtca

SEQ ID NO: 113 - DNA, 729 bp, Simian
gagaagtattggtgtaagtggagtaacacgggctgccagaccctgcccagccaagacgaaggccccagcgaggccttcgtaaactgtgacgagaacagccggcttgtctccctgaccctgaacccagtgaccagggcagacgaggcctggtactggtgtggagtgaagcagggccacttctatggagagactgcagctgtctatgtggcagttgaagagaagaaggtagcagggtcccgctatgtcagcccagcgaaggcagacgctgctcctgatgagaaggtgctagactctggtgtccgggagattgagaacaaagccattcaggatcccaggctttttgcagaggaaaaggtcgtggcagatacgggagatcaagctggtgggagcagagcatctgtggattccagcagctctgaggaacaaggtgggagctccaaagcgctggtctccactctggtgcccctgggcctggtgctggcactgggagccgtggttgtgggggtggccagagcccggcacaggaagaacgtcgaccgagtttcaatcagaagctacaggacagacattagcatgtcagacttcgagaactccagggaatttggagccaatgacaacatgggagcctcttcgatcactcaggagacatccctcggaggaaaagatgagtttgttgccacccctgagagcaccacggagaccaaagagcccaagaaggcaaaaagatcg

SEQ ID NO: 114 - DNA, 736 bp, Rattus sp.
gagaaatactggtgcaagtggagcaacgacggctgccacatcctgccgagccatgatgaaggtgcccgccagtcctctgtgagctgtgaccagagcagccagatcgtctccatgaccctgaacccggtcaaaaaggaagatgaaggctggtactggtgtggggtaaaagaaggtcaggtctatggagaaactacagccatctatgtagcagttgaagagaggaccagagggtcaccccacatcaacccgacagatgcaaacgcacgtgcaaaagatgctccagaggaagaggcaatggaatcctctgtcagggaggatgaaaacaaggccaatctggaccccaggctttttgcagacgaaagagagatacagaatgcgggagaccaagctcaggagaacagagcatctgcgaatgctggcagtgctggtggacaaagcgggagctccaaagtcctattctccaccctggtgcccctgggtttggtgctggcagtgggtgctgtggctgtgtgggtggccagagtccgacatcggaagaatgtagaccgcatgtcaatcagcagctacaggacagacattagctgggagacttcaggaactccagggatttgggaggcaatgacaacatgggcgccactccagacacacaagaaacagtcctcgaaggaaaagatgaaatagagactaccaccgagtgtaccaccgagccagaggaatccaagaaagcaaaaaggtc

SEQ ID NO: 115 - DNA, 771 bp, Oryctolagus cuniculus
gagaagtactggtgcaagtggaatgaccatggctgcgaggacctgcccactaagctcagctccagcggcgaccttgtgaaatgcaacaacaacctggtcctcaccctgaccttggactcggtcagcgaagatgacgagggctggtactggtgtggcgcgaaagacgggcacgagtttgaagaggttgcggccgtcagggtggagctgacagagccagccaaggtagctgtcgagccagccaaggtacctgtcgacccagccaaggcagcccccgcgcctcctgaggagaaggccaaggccgcggtgcccagtgcccaggagaaggcagtggtacccattgtcaaggaagctcagaacaaagttgtccagaaacctcgcctccttgcggaggaggtagcagtgcagagtgcggaagacccagccagtgggagcagagcgtctgtggatgccagcagtgcttcgggacaaagcgggagtgccaaagtactgatctccaccctggtgcccttggggctggtgctggcagcgggggccatggccgtggccatagccagagcccggcacaggaggaacgtggaccgagtttccatcggaagctacaggacagacattagcatgtcagacttggagaactccagggagttcggagccattgacaacccaagcgcctgccccgatgcccgggagacggccctcggaggaaaggatgagttagcgacggccaccgagagcaccgtggagattgaggagcccaagaaggcaaaacggtca

SEQ ID NO: 116 - protein, 6 aa, artificial
  description of artificial sequence histidine tag
HHHHHH

SEQ ID NO: 117 - protein, 7 aa, artificial
  description of artificial sequence amino- terminal border
EKYWCKW

SEQ ID NO: 118 - protein, 8 aa, artificial
  description of artificial sequence carboxy- terminal border
DEGWYWCG

SEQ ID NO: 119 - DNA, 21 bp, artificial
  description of artificial sequence illustrative oligonucleotide
atgcctaaagccgctccctca

SEQ ID NO: 120 - DNA, 15 bp, artificial
  description of artificial sequence illustrative oligonucleotide
atgaagccgctccct

SEQ ID NO: 121 - RNA, 15 nt, artificial
  description of artificial sequence illustrative oligonucleotide
augaagccgcucccu

SEQ ID NO: 122 - protein, 5 aa, artificial
  description of artificial sequence illustrative oligonucleotide
MPKAA

SEQ ID NO: 123 - DNA, 15 bp, artificial
  description of artificial sequence illustrative oligonucleotide
tgggttactcactca

SEQ ID NO: 124 - RNA, 15 nt, artificial
  description of artificial sequence illustrative oligonucleotide
uggguuacucacuca

SEQ ID NO: 125 - protein, 5 aa, artificial
  description of artificial sequence illustrative peptide
WVTHS

SEQ ID NO: 126 - DNA, 15 bp, artificial
  description of artificial sequence illustrative oligonucleotide
atgaagccgcactca

SEQ ID NO: 127 - RNA, 15 nt, artificial
  description of artificial sequence illustrative oligonucleotide
augaagccgcacuca

SEQ ID NO: 128 - protein, 5 aa, artificial
  description of artificial sequence illustrative peptide
MPKHS

SEQ ID NO: 129 - protein, 10 aa, unknown
  description of unknown organism cleaved peptide
QFQGVQFAGV

SEQ ID NO: 130 - protein, 10 aa, unknown
  description of unknown organism cleaved peptide
GFGSVQFAGF

SEQ ID NO: 131 - protein, 10 aa, unknown
  description of unknown organism cleaved peptide
GLVGGAGAGF

SEQ ID NO: 132 - protein, 10 aa, unknown
  description of unknown organism cleaved peptide
GGFLGLGAGF

SEQ ID NO: 133 - protein, 10 aa, unknown
  description of unknown organism cleaved peptide
GFGSTFFAGF

SEQ ID NO: 134 - protein, 5 aa, unknown
  description of unknown organism binding epitope
DPRLF

SEQ ID NO: 135 - protein, 6 aa, unknown
  description of unknown organism binding epitope
QDPRLF

SEQ ID NO: 136 - protein, 6 aa, unknown
  description of unknown organism binding epitope
LDPRLF

SEQ ID NO: 137 - protein, 8 aa, artificial
  description of artificial sequence synthetic peptide
EPKKAKRS

SEQ ID NO: 138 - protein, 6 aa, artificial
  description of artificial sequence synthetic peptide
EPKKAR

SEQ ID NO: 139 - protein, 107 aa, Streptococcus pneumoniae
LKPGEKVAEAKKKVEEAKKKAEDQKEEDRRNYPTNTYKTLELEIAEFDVKVKEAELELVKEEAKESRNEGTIKQAKEKVESKKAEATRLENIKTDRKKAEEEAKRKA

SEQ ID NO: 140 - protein, 107 aa, Streptococcus pneumoniae
LKSGKKVAEAEKKVEEAEKKAKDQKEEDRRNYPTNTYKTLDLEIAESDVKVKEAELELVKEEAKEPRDEEKIKQAKAKVESKKAEATRLENIKTDRKKAEEEAKRKA

SEQ ID NO: 141 - protein, 5 aa, artificial
  description of artificial sequence synthetic peptide
GGGSC

SEQ ID NO: 142 - protein, 5 aa, artificial
  description of artificial sequence synthetic peptide
GGGGC

SEQ ID NO: 143 - protein, 14 aa, artificial
  description of artificial sequence spacer peptide
EGKSSGSGSESKEF

(19) United States
(12) Patent Application Publication
(10) Pub. No.: US 2006/0099150 A1
(43) Pub. Date: May 11, 2006

(54) Compositions and methods for the transport of biologically active agents across cellular barriers

(76) Inventors: L. L. Houston, Del Mar, CA (US); Philip L. Sheridan, San Diego, CA (US); Stephen B. Hawley, San Diego, CA (US); Jacqueline M. Glynn, San Diego, CA (US); Steven Chapin, San Diego, CA (US)

Correspondence Address: JONES DAY, 222 EAST 41ST ST, NEW YORK, NY, 10017, US

(21) Appl. No.: 11/169,140

(22) Filed: Jun. 27, 2005

Related U.S. Application Data
(63) Continuation of application No. 09/969,748, filed on Oct. 2, 2001, now abandoned.
(63) Continuation-in-part of application No. 09/949,039, filed on Sep. 6, 2001, now abandoned.
(60) Provisional application No. 60/237,929, filed on Oct. 2, 2000.
(60) Provisional application No. 60/248,478, filed on Nov. 13, 2000.
(60) Provisional application No. 60/248,819, filed on Nov. 14, 2000.
(60) Provisional application No. 60/267,601, filed on Feb. 9, 2001.
(60) Provisional application No. 60/331,342, filed on Jul. 2, 2001.

(51) Int. Cl. A61K 39/395; A61K 9/14; A61L 9/04

(52) U.S. Cl. 424/46; 424/143.1

(57) ABSTRACT

Disclosed herein are complexes and compounds that pass through cellular barriers to deliver compounds into, through and out of cells, and methods of producing and using such complexes and compounds. The complexes and compounds of the invention comprise a biologically active portion and a targeting element directed to a ligand that confers transcellular, transcytotic or paracellular transporting properties to an agent specifically bound to the ligand. Also disclosed are complexes and compounds that comprise two or more targeting elements directed to a ligand that confers transcellular, transcytotic or paracellular transporting properties to an agent specifically bound to the ligand. Preferred ligands include but are not limited to the stalk of pIgR, a pIgR domain, an amino acid sequence that is conserved among pIgR's from different animals, and one of several regions of pIgR defined herein.

This application claims priority to each of:

(a) U.S. patent application Ser. No. 60/237,929 (attorney docket No. 057220.0301 {030854.0009.PRV1}) entitled “Genetic Fusions of pIgR Ligands and Biologically Active Polypeptides for the Delivery of Therapeutic and Diagnostic Proteins” by Houston, L. L., Glynn, Jacqueline M., and Sheridan, Philip L., filed Oct. 2, 2000, is drawn to fusion proteins comprising targeting elements and biologically active polypeptides.

(b) U.S. patent application Ser. Nos. 60/248,478 and 60/248,819 (attorney docket No. 057220.0601 {030854.0009.PRV2}, and 057220.0602 {030854.0009.PRV3}, respectively), both entitled “Protein Conjugates of pIgR Ligands for the Delivery of Therapeutic and Diagnostic Proteins” by Houston, L. L., and Hawley, Stephen, filed Nov. 13, 2000 and Nov. 14, 2000 respectively, are drawn to protein conjugates comprising targeting elements and biologically active polypeptides.

(c) U.S. patent application Ser. No. 60/267,601 (attorney docket No. 057220.0401) entitled “Polyspecific Binding Molecules Having a Polymeric Immunoglobulin Receptor Binding Region” by Houston, L. L., and Sheridan, Philip L., filed Feb. 9, 2001, is drawn to polyspecific compositions and compounds having (a) at least one ligand that specifically binds a pIgR molecule or the stalk molecule and (b) at least one ligand that (i) specifically binds a biologically active compound and/or (ii) is itself a biologically active compound.

(d) U.S. patent application Ser. No. 09/898,503 (attorney docket No. 057220.1401) entitled “Compositions, Compounds And Methods For The Delivery Of Monoclonal Antibodies” by Hawley, Stephen, Chapin, Steve, and Houston, L. L., filed Jul. 2, 2001, is drawn to the use of targeting elements and ligands to deliver monoclonal antibodies and related compounds and compositions.

(e) U.S. patent application Ser. No. ______ (attorney docket No. 057220.1301) entitled “Compounds and Molecular Complexes Comprising Multiple Binding Regions Directed to Transcytotic Ligands” by Hawley, Stephen, Chapin, Steve, Sheridan, Philip, and Houston, L. L., filed Sep. 6, 2001, is drawn to multivalent compounds having transcytotic properties.

Each of application (a)-(e) is hereby incorporated by reference in their entirety including drawings.

### FIELD OF THE INVENTION

The inventions disclosed herein relate to compositions that pass through cellular barriers to deliver compounds into, through and out of cells, and methods of producing and using such compositions.

### BACKGROUND OF THE INVENTION

The following description of the background of the invention is provided simply as an aid in understanding the invention and is not admitted to describe or constitute prior art to the invention.

Therapeutic drugs can be introduced into the body using a variety of formulations and by various of routes of administration. For many reasons, a preferred route of administration is one that is non-invasive, i.e, does not involve any physical damage to the body. Generally, physical damage of this type results from the use of a medical device, such as a needle, to penetrate or breach a dermal surface or other external surface of an animal. Invasive routes of administration include, for example, surgical implants and injections. Injections can be intravascular, intrathecal or subcutaneous, all of which have undesirable features. Non-invasive routes of administration include uptake from the gastrointestinal tract as well as non-invasive parenteral (i.e, other than gastrointestinal) routes such as, e.g., inhalation therapy.

Presently, there are few, if any, formulations for the administration of proteins, a relatively new type of therapeutic drug. This is especially true in the case of non-invasive routes of administration and formulations therefor. Despite the enormous potential of therapeutic proteins, the lack of compositions and methods for the non-invasive administration of proteins has, depending on the particular protein in question, limited or prevented the medical use thereof.

Compounds are trafficked into, out from and within a cell by various molecules. “Endocytosis” is a general term for the process of cellular internalization of molecules, i.e, processes in which cells takes in molecules from their environment, either passively or actively. “Exocytosis” is a general term for processes in which molecules are passively or actively moved from the interior of a cell into the medium surrounding the cell. “Transcytosis” is a general term for processes in which molecules are transported from one surface of a cell to another.

Active endocytosis, exocytosis and transcytosis typically involve or are mediated by receptors, molecules that are at least partially displayed on the surface of cells. Receptors have varying degrees of specificity; some are specific for a single molecule (e.g., a receptor specific for epidermal growth factor; or a receptor that specifically recognizes Ca⁺⁺); some are semi-specific (e.g., a receptor that mediates the cellular internalization of many members of a family of cellular growth factors, or a receptor that recognizes Ca⁺⁺, Mg⁺⁺ and Zn⁺⁺); or of limited specificity (e.g., a receptor that mediates the cellular internalization of any phosphorylated protein, or a receptor that recognizes any divalent cation). Other types of molecules that can cause or influence the entry of molecules into cells include, e.g., cellular pores, pumps, and coated pits. Pores such as gated channels and ionophores form a channel that extends through the cellular membrane and through which certain molecules can pass. Cellular pumps exchange one type of molecule within a cell for another type of molecule in the cell's environment. Coated pits are depressions in the cellular surface that are “coated” with bristlelike structures and which condense to surround external molecules; the condensed coated pits then “pinch off” to form membrane-bound, coated vesicles within the cell.

Molecules that cause, influence or undergo endocytosis, exocytosis and/or transcytosis can do so constitutively, i.e, at all times, or regulated, for example, only under certain conditions or at specific times. Some such molecules can only mediate and/or undergo endocytosis, whereas some mediate and/or undergo transcytosis as well as endocytosis. Moreover, some such molecules are present in all or most cells (i.e, are ubiquitous), or are present mostly or only in certain tissues (i.e, are tissue-specific) or particular cell types.

The lack of compositions and methods causing, enhancing, mediating or regulating the endocytosis of therapeutic, diagnostic or analytical compounds and compositions hinders or prevents various uses of such compounds. In particular, the full therapeutic potential of many compounds could be realized if they were taken up by cells lining the gastrointestinal tract, as one could then formulate pills or tablets for the administration of therapeutic agents to patients. Typically, pills and other formulations for the oral delivery, and suppositories for the rectal delivery, of therapeutic agents to the gastrointestinal tract result in better patient compliance, and less use of medical resources, as opposed to other delivery modalities such as, e.g., intravenous administration. Similarly, the therapeutic potential of many compounds could be realized if they were taken up by cells lining the respiratory tract, including the nasal cavity; cells lining the gastrointestinal tract; vaginal surfaces; on dermal surfaces; and ocular surfaces and buccal surfaces (see Sayani et al., Crit. Rev. Ther. Drug Carrier Systems 13:85-184, 1996). Attempts to develop oral delivery formulations for proteins are discussed by Wang (J. Drug Targeting 4:195-232, 1996), Sinko et al. (Pharm. Res. 16:527, 1999) and Stoll et al. (J. Controlled Release 64:217-228, 2000).

In addition to the need for compositions and methods for the entry of biologically active molecules into cells, there is a further need for compositions and methods for causing, enhancing, mediating or regulating, or that control the direction of, transcytosis. Transcytosis is the general term given for processes whereby molecules, including biologically active molecules, move from one side or surface of a cell to another.

Furthermore, degradation and inefficient absorption of compounds delivered by conventional means further reduces the efficacy of those compounds. The ability to utilize alternative delivery pathways, target particular cells and tissues for delivery, improve the retention and absorption of compounds to be delivered, and protect the effective compound during delivery would be of significant import to the pharmaceutical and biopharmaceutical industries.

The above limitations vis-à-vis cellular transport of molecules are present both in vitro (e.g., in cellular cultures) and in vivo (e.g., in animals). Such limitations prevent or limit the therapeutic, diagnostic and/or analytical uses as of various compounds and compositions in an animal, including a mammal which may be a human. Such uses are described herein.

One example of a molecule that undergoes or mediates endocytosis, exocytosis as well as forward and reverse transcytosis is the polymeric immunoglobulin receptor (pIgR). The following information regarding pIgR is provided to assist in understanding the background of the invention.

Typically, pIgR molecules are displayed on epithelial cells. Epithelial cells line the interior of organs that have enclosed, semi-enclosed or compartmentalized spaces. The interior (e.g., canals, ducts, cavities, etc.) of such organs is generically referred to as the lumen. The lumen of a particular organ may have a specific name, e.g., the gastrointestinal lumen, pulmonary lumen, nasal lumen, nasopharyngeal lumen, pharyngeal lumen, buccal (within the mouth) lumen, sublingual (under the tongue) lumen, vaginal lumen, urogenital lumen, ocular lumen, or tympanic lumen. See, for example, Fahey et al., Immunol. Invest. 27:167-180, 1998; Brandtzaeg, J. Reprod. Immunol. 36:23-50, 1997; Kaushic et al., Biol. Reprod. 57:958-966, 1997; Richardson et al., J. Reprod. Immunol. 33:95-112; Kaushic et al., Endocrinology 136:2836-2844, 1995. Some of these might also be characterized as surfaces, e.g., the ocular surface.

Adjacent epithelial cells are connected by tight junctions. Disruption of tight junctions allows agents within the lumen, which often has an opening to the external environment of an animal, to penetrate into the body. Although such agents might include therapeutic agents, entry into the body via a disrupted tight junction is not specific; undesirable agents (e.g., bacteria, viruses, toxins and the like) will also be taken into the body. Due to this lack of specificity, as well as other factors, disruption of tight junctions for drug delivery purposes is generally not feasible and would, in any event, have many potential undesirable side effects.

Epithelial cells have two distinct surfaces: the apical side, which faces the lumen and is exposed to the aqueous or gaseous medium present therein; and an opposing basolateral (a.k.a. basal lateral) side that rests upon and is supported by an underlying basement membrane. The tight junctions between adjacent epithelial cells separate the apical and basolateral sides of an individual epithelial cell.

Epithelial cells are said to have polarity, that is, they are capable of generating gradients between the compartments they separate (for reviews, see Knust, Curr. Op. Genet. Develop. 10:471-475, 2000; Matter, Curr. Op. Genet. Develop. 10:R39-R42, 2000; Yeaman et al., Physiol. Rev. 79:73-98, 1999). This polarity reflects that fact that the cell has distinct plasma membrane domains (apical and basolateral) having distinct transport and permeability characteristics. For example, the apical side often contains microvilli for the adsorption of substances from the lumen, and, in ciliated cells, cilia are found on the apical membrane. As another example, the Na⁺/K⁺-ATPase pump is characteristically found only on the basolateral membrane.

FIG. 1 shows the pathways of cellular transport involving the pIgR protein, which undergoes or mediates endocytosis, exocytosis as well as forward and reverse transcytosis, in epithelial cells. Molecules of pIgR are typically displayed on the surfaces of epithelial cells and direct the trafficking of immunoglobulin (IgA) molecules. Other classes and species of immunoglobulins may also be trafficked. The right side of FIG. 1 illustrates the “forward” (i.e, basolateral to apical) transcytosis of pIgR molecules, whereas “reverse” (apical to basolateral) transcytosis is shown on the left side of the Figure.

Forward transcytosis is the best characterized biological function of pIgR, and serves to convey protective antibodies (IgA and IgM immunoglobulins) from the circulatory system to the lumen of an organ. In forward transcytosis, pIgR molecules displayed on the basolateral side of the cell bind IgA molecules in the bloodstream, and pIgR:IgA complexes are then endocytosed, i.e, taken up into the cell and into a vesicle. The pIgR:IgA complexes are transported to the apical side of the cell, where they are displayed on the cell surface. Delivery of IgA into the lumen occurs when the pIgR portion of a pIgR:IgA complex is cleaved, i.e, undergo proteolysis. This event separates the pIgR molecule into two components: the “secretory component” (SC), which is released into the lumen, and which remains bound to IgA in order to protect IgA from degradation, and the “stalk,” which remains displayed, at least temporarily, on the apical surface of the cell.

Surprisingly, ligands bound to stalks displayed on the apical side of a cell can undergo reverse transcytosis, i.e, transcytosis in the opposite direction of forward transcytosis, i.e, from the apical side of a cell to its basolateral side. In reverse transcytosis, pIgR molecules or portions thereof move from the apical surfaces of cells that line the lumen of an organ to the basolateral surfaces of these cells. In theory, pIgR-mediated reverse transcytosis could be used to deliver agents from a lumen (e.g., the interior of the gut or the airways of the lung) to the circulatory system or some other interior system, organ, tissue, portion or fluid of the body including by way of non-limiting example the lymphatic system, the vitreous humor, etc. For example, as is shown in FIG. 1, a compound having an element that binds to a portion of pIgR that undergoes reverse transcytosis could, due to its association with the pIgR stalk, be carried to the basolateral side of a cell, where it would be contacted with and/or released into the bloodstream.

Evidence has been presented that forward transcytosis is mediated by a vesicular process (Apodaca et al., J. Cell Biol. 125:67-86, 1994; Mostov, Annu. Rev. Immunol. 12:63-84, 1994), although the process may vary between different cell types (Sarnataro et al., Detergent insoluble microdomains are not involved in transcytosis of polymeric Ig receptor in FRT and MDCK cells, Traffic 2000 October; 1(10):794-802). Although not wishing to be bound by any particular theory, FIG. 1 shows a similar vesicular mediated transport mechanism for reverse transcytosis. FIG. 1 is not intended to imply that such a mechanism actually exists because evidence to this fact is not available; the vesicular nature of reverse transcytosis is only a hypothesis based on what is known about forward transcytosis.

The polyimmunoglobulin receptor (pIgR) is reviewed by Mostov and Kaetzel, Chapter 12 in: Mucosal Immunology, Academic Press, 1999, pages 181-211 (1999). Other reviews of pIgR, transcytosis and mucosal immunity include Apodaca et al., The polymeric immunoglobulin receptor. A model protein to study transcytosis, J Clin Invest 87:1877-82, 1991; Kaetzel, Polymeric Ig receptor: defender of the fort or Trojan horse? Curr Biol 11:R35-8, 2001; Mostov, Regulation of protein traffic in polarized epithelial cells Histol Histopathol 10:423-31, 1995; Mostov et al., Regulation of protein traffic in polarized epithelial cells, Bioessays 17:129-38, 1995; Mostov, Transepithelial transport of immunoglobulins, Annu Rev Immunol 12:63-84, 1994; Brandtzaeg et al., The B-cell system of human mucosae and exocrine glands, Immunol Rev 1999 October; 171:45-87; and Norderhaug et al., Regulation of the formation and external transport of secretory immunoglobulins (Review), Crit Rev Immunol 1999; 19(5-6):481-508.

Transgenic animals that have alterations in the structure or expression of pIgR have been described Shimada et al., Generation of polymeric immunoglobulin receptor-deficient mouse with marked reduction of secretory IgA, J Immunol 1999 November 15:163(10):5367-73; Johansen et al., Absence of epithelial immunoglobulin A transport, with increased mucosal leakiness, in polymeric immunoglobulin receptor/secretory component-deficient mice, J Exp Med 1999 October 4:190(7):915-22; and de Groot et al., Over-expression of the murine polymeric immunoglobulin receptor gene in the mammary gland of transgenic mice, Transgenic Res 1999 April; 8(2):125-35, Erratum in: Transgenic Res 1999 August; 8(4):319.

Phillips-Quagliata et al., The IgA/IgM receptor expressed on a murine B cell lymphoma is poly-Ig receptor, J Immunol 2000 Sep. 1; 165(5):2544-55, is stated to demonstrate that T560, a mouse B lymphoma that originated in gut-associated lymphoid tissue, expresses pIgR.

The structures of pIgR and its Ig ligands have been investigated using molecular genetic techniques. Norderhaug et al., Domain deletions in the human polymeric Ig receptor disclose differences between its dimeric IgA and pentameric IgM interaction, Eur J Immunol 1999 October; 29(10):3401-9; Crottet et al., Covalent homodimers of murine secretory component induced by epitope substitution unravel the capacity of the polymeric Ig receptor to dimerize noncovalently in the absence of IgA ligand, J Biol Chem 1999 Oct. 29; 274(44):31445-55; Breitfeld et al., Deletions in the cytoplasmic domain of the polymeric immunoglobulin receptor differentially affect endocytotic rate and postendocytotic traffic, J Biol: Chem 1990 Aug. 15; 265(23):13750-7; Casanova et al., Phosphorylation of the polymeric immunoglobulin receptor required for its efficient transcytosis, Science 1990 May 11; 248(4956):742-5

Singer et al., Dimerization of the polymeric immunolgobulin receptor controls its transcytotic trafficking, Mol Biol Cell 1998 April; 9(4):901-15, is stated to demonstrate that binding of dimeric IgA to chimeric pIgR/TCR molecules induces its dimerization (TCR is an abbreviation for the T cell receptor). The cytoplasmic domain of the T cell receptor-zeta chain was used as an indicator of receptor oligomerization to show that a pIgR:zeta chimeric receptor expressed in Jurkat cells initiates a zeta-specific signal transduction cascade when exposed to dimeric or tetrameric IgA, but not when exposed to monomeric IgA.

Eckman et al., Am J Respir Cell Mol Biol 1999 August; 21(2):246-52, is stated to disclose a fusion protein consisting of a sFv directed to the secretory component (SC) of human pIgR and an human alpha-(1)-antitrypsin. Ferkol et al., Am. J. Respir. Crit. Care Med. 161:944-951, 2000, is stated to describe the basolateral-to-apical transport of the fusion protein of Eckman et al. across in vitro model systems of polarized respiratory epithelial cells.

Gupta et al., Gene Ther 8:586-92, 2001, is stated to disclose the use of a single-chain antibody directed to the secretory component (SC) of human pIgR to deliver reporter genes to epithelial cells in vitro. The sFv is stated to be conjugated to polylysine using the cross-linker SPDP.

Zhang et al., Cell 102:827-837, 2000, states that pIgR translocates, Streptococcus pneumoniae across nasopharyngeal epithelial cells. The bacterial translocation is reported to occur in the apical to basolateral (reverse) direction.

Pilett et al., Reduced epithelial expression of secretory component in small airways correlates with airflow obstruction in chronic obstructive pulmonary disease, Am J Respir Crit Care Med 2001 January; 163(1):185-94, is stated demonstrate that reduced expression of SC in airway epithelium is associated with airflow obstruction and neutrophil infiltration in severe chronic obstructive pulmonary disease.

U.S. Pat. No. 5,484,707 to Goldblum et al. is drawn to methods for monitoring organ rejection in an animal based on the concentration of the free secretory component of (SC) pIgR.

PCT patent applications WO 98/30592 and WO 99/20310, both to Hein et al., and U.S. Pat. No. 6,045,774 to Hiatt et al., are drawn to synthetic proteins that mimic IgA molecules and are thus associated with the proteolytically generated secretory component (SC) of pIgR.

U.S. Pat. No. 6,072,041 to Davis et al. is drawn to fusion proteins that comprise single-chain antibodies directed to the secretory component of pIgR. The compositions of Davis et al. are stated to be transported specifically from the basolateral surface of epithelial cells to the apical surface.

U.S. Pat. No. 6,261,787 B1 to Davis et al. is drawn to bifunctional molecules comprising (1) a ligand directed to the secretory component of pIgR and (2) a non-protein therapeutic molecule. The bifunctional molecules are said to be transported specifically from the basolateral surface of an epithelial cell to the apical surface thereof.

U.S. Pat. No. 6,287,817 B1 to Davis et al is drawn to a method of delivering a therapeutic protein to an epithelial cell by using a fusion protein that comprises a single-chain antibody directed to the secretory component of pIgR. The proteins are said to be transported specifically from the basolateral surface of an epithelial cell to the apical surface thereof.

PCT application No. WO 00/53623, published Sep. 14, 2000, entitled “Bifunctional Molecules for Delivery of Therapeutics” by Davis, Pamela B., Ferkol Jr., Thomas W., and Eckman, Elizabeth, is stated to disclose bifunctional molecules that specifically bind secretory component (SC) of pIgR. The bifunctional molecules are said to be transported specifically from the basolateral surface of an epithelial cell to the apical surface thereof.

PCT application No. WO 00/53623, by Ziady, Assem, Davis, Pamela B., Ferkol Jr., Thomas W., and Malouf, Alfred, was published Sep. 14, 2000 and is entitled “Enhanced Delivery Via Serpin Enzyme Complex Receptor”.

U.S. Pat. No. 6,042,833 to Mostov et al. is drawn to a method by which a ligand that binds to a portion of a pIgR molecule is thereby internalized into, or transported across, a cell expressing or displaying pIgR, Ser. No. 09/475,088 (attorney reference Nos. 2307E-067911US and 057220-0908) is a Divisional application of U.S. Pat. No. 6,042,833, that was filed Dec. 30, 1999. The corresponding PCT application was published as WO 97/46588, entitled “Cellular Internalization of pIgR Stalk and Associated Ligands” on Dec. 11, 1997.

U.S. patent application Ser. No. ______ (attorney docket No. 18062E-000900US) is entitled “Ligands Directed To The Non-Secretory Component, Non-Stalk Region of pIgR and Methods of Use Thereof” and was filed Mar. 26, 2001, by Mostov et al.

U.S. patent application Ser. No. 09/839,746 (attorney docket No. 057220.0202), filed Apr. 19, 2001, entitled “Compositions Comprising Carriers and Transportable Complexes” by Houston, L. L., disclose various pharmaceutical compositions that may be applied to compositions and methods of the present invention.

U.S. patent application Ser. No. 60/237,929 (attorney docket No. 057220.0301 {030854.0009.PRV1}) entitled “Genetic Fusions of pIgR Ligands and Biologically Active Polypeptides for the Delivery of Therapeutic and Diagnostic Proteins” by Houston, L. L., Glynn, Jacqueline M., and Sheridan, Philip L., filed Oct. 2, 2000, is drawn to fusion proteins comprising targeting elements and biologically active polypeptides.

U.S. patent application Ser. Nos. 60/248,478 and 60/248,819 (attorney docket No. 057220.0601 {030854.0009.PRV2}, and 057220.0602 {030854.0009.PRV3}, respectively), both entitled “Protein Conjugates of pIgR Ligands for the Delivery of Therapeutic and Diagnostic Proteins” by Houston, L. L., and Hawley, Stephen, filed Nov. 13, 2000 and Nov. 14, 2000 respectively, are drawn to protein conjugates comprising targeting elements and biologically active polypeptides.

U.S. patent application Ser. No. 60/266,182 (attorney docket No. 057220.0701) entitled “Compositions and Methods for Identifying, Characterizing, Optimizing and Using Ligands to Transcytotic Molecules” by Houston, L. L., and Sheridan, Philip L., filed Feb. 2, 2001, is drawn to the identification and use of ligands and targeting elements directed to transcytotic and transepithelial molecules.

U.S. patent application Ser. No. 60/267,601 (attorney docket No. 057220.0401) entitled “Polyspecific Binding Molecules Having a Polymeric Immunoglobulin Receptor Binding Region” by Houston, L. L., and Sheridan, Philip L., filed Feb. 9, 2001, is drawn to polyspecific compositions and compounds having (a) at least one ligand that specifically binds a pIgR molecule or the stalk molecule and (b) at least one ligand that (i) specifically binds a biologically active compound and/or (ii) is itself a biologically active compound.

U.S. patent application Ser. No. 60/281,275 (attorney docket No. 057220.0501) entitled “Compositions and Methods for Transepithelial Transport of Membrane-Bounded Vesicles and Virions” by Sheridan, Philip L., and Houston, L. L., filed Apr. 3, 2001, is drawn to the use of targeting elements and ligands to deliver bounded compositions such as liposomes, virions, and the like.

U.S. patent application Ser. No. 09/898,503 (attorney docket No. 057220.1401) entitled “Compositions, Compounds And Methods For The Delivery Of Monoclonal Antibodies” by Hawley, Stephen, Chapin, Steve, and Houston, L. L., filed Jul. 2, 2001, is drawn to the use of targeting elements and ligands to deliver monoclonal antibodies and related compounds and compositions.

U.S. patent application Ser. No. ______ (attorney docket No. 057220.1301) entitled “Compounds and Molecular Complexes Comprising Multiple Binding Regions Directed to Transcytotic Ligands” by Hawley, Stephen, Chapin, Steve, Sheridan, Philip, and Houston, L. L., filed Sep. 6, 2001, is drawn to multivalent compounds having transcytotic properties.

### SUMMARY OF THE INVENTION

In one aspect, the invention provides a complex or compound comprising a biologically active portion and a targeting element directed to a ligand that confers transcellular, transcytotic or paracellular transporting properties to an agent specifically bound to the ligand, wherein the targeting element is not an antibody.

The term “compound” is used herein as in the field of chemistry, i.e., a substance of two or more elements, where the elements are present in fixed proportions, having a defined chemical structure. A “molecular complex” or “complex” comprises at least two distinct molecules that are associated with each other by noncovalent interactions. By “associated” it is meant that the molecules in a complex, or moieties in a compound, are specifically bound to each other. In a compound of the invention, a targeting element and biologically active portion have a covalent association with each other whereas, in a molecular complex of the invention, the targeting element and biological active portion have a non-covalent association.

A “target molecule” or “molecular target” is a compound, a molecular complex of two or more compounds, a moiety (a portion of a compound), or an interface formed between two or more compounds, to which a targeting element or ligand is directed. The term “ligand” encompasses any type of composition or compound that is capable of specifically binding to a molecular target. The term “targeting element” encompasses any moiety or compound that is included within, respectively, a compound or a molecular complex, that is capable of specifically binding to a molecular target. A ligand is a targeting element when it is either covalently or non-covalently associated with another compound. A compound or composition comprising a targeting element directed to (capable of specifically binding) a molecular target is a ligand of that target. When a ligand is incorporated into a compound or complex, it becomes a targeting element so long as retains the ability to specifically bind to the target. Conversely, when a targeting element is separated from the remainder of a compound, it becomes a ligand so long as it retains the ability to specifically bind a molecular target. Any portion or derivative of a ligand that is capable of specifically binding a molecular target is a ligand.

The term “biologically active” (synonymous with “bioactive”) indicates that a composition or compound itself has a biological effect, or that it modifies, causes, promotes, enhances, blocks, reduces, limits the production or activity of, or reacts with or binds to an endogenous molecule that has a biological effect. A “biological effect” may be but is not limited to one that stimulates or causes an immunoreactive response; one that impacts a biological process in an animal; one that impacts a biological process in a pathogen or parasite; one that generates or causes to be generated a detectable signal; and the like. Biologically active compositions, complexes or compounds may be used in therapeutic, prophylactic and diagnostic methods and compositions. Biologically active compositions, complexes or compounds act to cause or stimulate a desired effect upon an animal. Non-limiting examples of desired effects include, for example, preventing, treating or curing a disease or condition in an animal suffering therefrom; limiting the growth of or killing a pathogen in an animal infected thereby; augmenting the phenotype or genotype of an animal; stimulating a prophylactic immunoreactive response in an animal; or diagnosing a disease or disorder in an animal.

In the context of therapeutic applications of the invention, the term “biologically active” indicates that the composition, complex or compound has an activity that impacts an animal suffering from a disease or disorder in a positive sense and/or impacts a pathogen or parasite in a negative sense. Thus, a biologically active composition, complex or compound may cause or promote a biological or biochemical activity within an animal that is detrimental to the growth and/or maintenance of a pathogen or parasite; or of cells, tissues or organs of an animal that have abnormal growth or biochemical characteristics, such as cancer cells.

In the context of diagnostic applications of the invention, the term “biologically active” indicates that the composition, complex or compound can be used for in vivo or ex vivo diagnostic methods and in diagnostic compositions and kits. For diagnostic purposes, a preferred biologically active composition or compound is one that can be detected, typically (but not necessarily) by virtue of comprising a detectable polypeptide. Antibodies to an epitope found on composition or compound may also be used for its detection.

In the context of prophylactic applications of the invention, the term “biologically active” indicates that the composition or compound induces or stimluates an immunoreactive response. In some preferred embodiments, the immunoreactive response is designed to be prophylactic, i.e, prevents infection by a pathogen. In other preferred embodiments, the immunoreactive response is designed to cause the immune system of an animal to react to the detriment of cells of an animal, such as cancer cells, that have abnormal growth or biochemical characteristics. In this application of the invention, compositions, complexes or compounds comprising antigens are formulated as a vaccine.

It will be understood by those skilled in the art that a given composition, complex or compound may be biologically active in therapeutic, diagnostic and prophylactic applications. A composition, complex or compound that is described as being “biologically active in a cell” is one that has biological activity in vitro (i.e, in a cell culture) or in vivo (i.e, in the cells of an animal). A “biologically active component” of a composition or compound is a portion thereof that is biologically active once it is liberated from the composition or compound. It should be noted, however, that such a component may also be biologically active in the context of the composition or compound.

Specific examples of compositions, complexes and compounds that are not biologically active include elements that have no effect on biological functions but which are incorporated for ease of manipulation of the conjugate or member thereof such as, e.g., poly-(L)-lysine for the in vitro chemical conjugation of the composition or compound to another molecule; a polypeptide derived from a phage surface protein intended for compositions or compounds to be used in vitro in phage display libraries; or a composition or compound that serves as a carrier for another composition or compound such as, e.g., KLH (keyhole limpet hemocyanin), which serves as a carrier for immunogenic compositions or compounds; or the herein-disclosed “optional fusion protein elements.”

A “transcellular property” is an attribute that causes, promotes or enhances any type of process that results in the movement of a molecule from side of a cell to another, regardless of the mechanism of the movement.

A “transcytotic property” is an attribute that causes, promotes or enhances endocytosis, exocytosis, transcytosis and/or intracellular delivery. Transcytotic properties include, by way of non-limiting example, the ability to undergo a least one process selected from the group consisting of apical endocytosis, apical exocytosis, apical to basolateral transcytosis, basolateral endocytosis, basolateral exocytosis, basolateral to apical transcytosis, and intracellular delivery.

By “intracellular delivery,” it is meant that a complex or compound is delivered into, and remains inside, the interior of a cell, whether within the cytosol or an organelle. In a related aspect, the compositions and compounds of the invention include an organelle-targeting sequence for transport to selected organelles. An “organelle” is a subcellular component that carries out one or more specific biological and/or biochemical functions. An “organelle-targeting sequence” is an amino acid sequence that mediates the delivery of a complex or compound having the organelle targeting sequence to an organelle of interest such as, e.g., a mitochondrion, the endoplasmic reticulum, the Golgi apparatus, lysosomes, peroxisomes, endosomes, the cell membrane or any membrane contained within a cell, the nucleus, or the nucleolus.

A “paracellular transporting property” is an attribute that causes, promotes paracellular transport including, by way of non-limiting example, transport through the tight junctions found in epithelial or mucosal cell layers. Tight junctions seal adjacent epithelial cells in a narrow band just near their apical surface and, as a result, agents on one side of an epithelial layer must move through epithelial cells in order to reach the other side of the barrier. Such movement may result from simple diffusion (passive transport), or as a result of cellular, usually ATP-dependent, activity (active transport).

As is explained in more detail herein, the term “antibody” includes polyclonal, monospecific, monoclonal, camelized, humanized and single-chain antibodies; Fab, Fab′ (Fab′)₂ fragments; CDRs; and the like.

The targeting element that is not an antibody can be any type of molecule or moeity, regardless of chemical structure, that functions as a targeting element for the ligand of choice. Thus, the targeting element can be a lipid, a carbohydrate, a small molecule, or a nucleic acid. Nucleic acids, such as aptamers, that bind specifically to a preselected molecular target are used as targeting elements in the complexes and compounds of the invention. The targeting element can also be a polypeptide that is not an antibody. When both the bioactive portion of a compound are polypeptides, the compound is a fusion protein or a protein conjugate, as those terms are used herein.

In this and other aspects of the invention, the chosen ligand that confers transcellular, transcytotic or paracellular transporting properties to an agent specifically bound to the ligand, is the pIgR stalk or a domain, conserved sequence or region thereof.

In a related aspect, the ligand may be a polypeptide that corresponds to an amino acid sequence that is conserved in pIgR proteins from a variety of species, e.g., the ligand is a polypeptide having an amino acid sequence selected from the group consisting of LRKED, QLFVNEE, LNQLT, YWCKW, GWYWC, STLVPL, SYRTD, and KRSSK.

In a related aspect, the ligand may be a polypeptide that corresponds to an amino acid sequence present in a defined region selected from the group consisting of:
R1 From KRSSK to the carboxy terminus of pIgR,
R2a From SYRTD to the carboxy terminus of pIgR,
R2b From SYRTD to KRSSK,
R3a From STLVPL to the carboxy terminus of pIgR,
R3b From STLVPL to KRSSK,
R3c From STLVPL to SYRTD,
R4a From GWYWC to the carboxy terminus of pIgR,
R4b From GWYWC to KRSSK,
R4c From GWYWC to SYRTD,
R4d From GWYWC to STLVPL,
R5a From YWCKW to the carboxy terminus of pIgR,
R5b From YWCKW to KRSSK,
R5c From YWCKW to SYRTD,
R5d From YWCKW to STLVPL,
R5e From YWCKW to GWYWC,
R6a From LNQLT to the carboxy terminus of pIgR,
R6b From LNQLT to KRSSK,
R6c From LNQLT to SYRTD,
R6d From LNQLT to STLVPL,
R6e From LNQLT to GWYWC,
R6f From LNQLT to YWCKW,
R7a From QLFVNEE to the carboxy terminus of pIgR,
R7b From QLFVNEE to KRSSK,
R7c From QLFVNEE to SYRTD,
R7d From LNQLT to STLVPL,
R7e From QLFVNEE to GWYWC,
R7f From QLFVNEE to YWCKW,
R7g From QLFVNEE to LNQLT,
R8a From LRKED to the carboxy terminus of pIgR,
R8b From LRKED to KRSSK,
R8c From LRKED to SYRTD,
R8d From LRKED to STLVPL,
R8e From LRKED to GWYWC,
R8f From LRKED to YWCKW,
R8g From LRKED to LNQLT, and
R8h From LRKED to QLFVNEE.

When the ligand is a pIgR stalk or a portion thereof, a targeting element may, by way of non-limiting example, be a polypeptide derived from a protein that binds the pIgR stalk or portion thereof.

In particular, a polypeptide that functions as a targeting element directed to the pIgR stalk may be derived from a polypeptide derived from a calmodulin, an AP-1 Golgi adaptor or a bacterial polypeptide.

Non limiting examples of polypeptides from bacterial proteins that may be used as pIgR-stalk-directed targeting elements are those amino acid sequences from CbpA that are underlined in FIG. 17.

In a related aspect, the complexes and compounds of the invention further comprises a PTD or MTS.

“Protein transduction domains” (PTD) and “membrane transport signals” (MTS) are polypeptides, typically about 10-35 amino acids long, that facilitate, promote or induce the uptake of proteins and other polypeptides by cells. The PTD are derived from HIV-TAT, HSV-VP22 and Antenapedia (the source of Penetratin), and are characterized by having a high content of positively charged arginine (Arg) and lysine (Lys) residues. The MTS are very hydrophobic peptides derived from secretory signal sequences, which partition into the hydrophobic layer of a membrane lipid bilayers.

The biologically active portion can be any type of molecule or moeity, regardless of chemical structure, capable of achieving the desired biological effect. Thus, the biologically active portion is a polypeptide including a peptidomimetic, a nucleic acid, a lipid, a carbohydrate, a compound or complex comprising a metal, a small molecule, or a functional derivative of any of the preceding.

The term “functional derivative” indicates a chemically modified version, an analog, or a homolog of a compound that retains a biological function of interest of that compound for any given application. In the case of polypeptides, chemical modification may include, by way of non-limiting example, adding chemical groups to a compound (e.g., glycosylation, phosphorylation, thiolation, etc.), eliminating parts of a compound that do not impact the function of interest (preparing a truncated form of a protein that retains an activity of interest, e.g., Klenow fragment), changing sets of one or more amino acids in the polypeptide (preparing muteins); analogs are exemplified by peptidomimetics; and homologs are polypeptides from other species of animals that retain biological activity (e.g., human and porcine insulin, human and salmon calcitonin, etc.) or intraspecies isomers of a polypeptide (protein “families” such as the cytochrome P450 family).

The bioactive portion of the complex or compound can be a complex or compound comprising a metal. Such metals include, by way of non-limiting example, platinum(II), palladium(II), zinc, cobalt(III). Metal-based or comprising drugs include, but are not limited to, Cisplatin.

The biologically active portion is a nucleic acid. Bioactive nucleic acids include, by way of non-limiting example, aptamers, antisense molecules including ribozymes, nucleic acids that encode therapeutic polypeptides and nucleic acids that serve as a template for the production of a biologically active nucleic acid.

The biologically active portion of the complex or compound can be a polypeptide. By way of non-limiting example, a bioactive polypeptide may be a growth factor, an interleukin, an immunogen, a hormone, an enzyme, an enzyme inhibitor, an antibody, a clotting factor, a receptor, a ligand for a receptor, a kinase, a phosphoptase, a scaffold protein, an adaptor protein, a dominant negative mutant, a protease, a signaling molecule, a regulatory molecule, transporter, a transcriptional regulator, a nucleic acid binding protein, and a functional derivative of any of the preceding. Bioactive polypeptides of particular interest include insulin, IL-2, IL-4, hGH, sCT and hCT.

In a related aspect, the biologically active portion is a second targeting element that is directed to a molecular target other than said ligand. The targeting element, directed to a molecular target other than the ligand that confers transcellular, transcytotic or paracellular transporting properties to an agent specifically bound to said ligand, is one type of bioactive portion. Such compounds are bispecific (polyspecific) in that they bind more than 1 molecular target. Polyspecific complexes or compounds may be formulated with the target of the second targeting element and then administered, in order to deliver an exogenous target molecule into the body. Alternatively, polyspecific complexes or compounds may be formulated separately, i.e., without the target of the second targeting element. When administered, the latter type complex or compound binds to an endogenous molecular target. Thus, this aspect of the invention provides for the delivery of exogenous drugs and “molecular sponges” that bind, neutralize and/or sequester, endogenous molecules.

In a related aspect, the invention provides a polyspecific complex or compound that further comprise a biologically active portion that is not a targeting element. Thus, for example, a complex or compound of the invention could comprise (1) a targeting element directed to a ligand that confers transcellular, transcytotic or paracellular transporting properties to an agent specifically bound to said ligand, which functions to deliver the complex or compound into the body; (2) a second targeting element that is directed to a molecular target that is found only or predominantly on a preselected cell or tissue, which functions to target the complex or compound to the cell or tissue; and (3) a bioactive portion that is preferentially delivered to the cell or tissue of interest. Portion (3) may, by way of non-limiting example, be a cytotoxin that is preferably delivered to diseased cells (e.g., cancer cells or virally infected cells), or a therapeutically beneficial agent that is preferable delivered to cells in need of such treatment.

The second targeting element may be an antibody or an antibody derivative. Antibodies per se include, but are not limited to, polyclonal, monospecific, and monoclonal antibodies. Antibody derivatives include those prepared by recombinant DNA technology, e.g., single-chain (sFv) antibodies, and those prepared from whole antibodies by chemical manipulation, e.g., Fab, Fab′ and (Fab)₂ fragments.

Another aspect of the invention provides a multivalent complex or compound, i.e., a complex or compound comprising 2 or more targeting elements directed to one or more ligands that confer transcellular, transcytotic or paracellular transporting properties to an agent specifically bound to said ligand.

By way of non-limiting example, multivalent complexes or compounds of the invention comprise two, three, or four targeting elements directed to the ligand (dimers, trimers, tetramers, respectively). In any given multivalent compound or complex, the targeting elements (T1, T2) may be identical (T1=T2), substantially the same (T1˜T2) or different from each other, as these terms are used herein. Thus, in related aspects, the invention provides complexes or compounds of the invention, wherein at least one of the targeting elements in the complex or compound is identical or substantially identical to at least one other targeting element in the complex or compound; as well as complexes or compounds wherein at least one of said targeting elements in the complex or compound is different from at least one other targeting element of the second complex or compound.

In a related aspect, the ligand that confers transcellular, transcytotic or paracellular transporting properties to a multivalent agent specifically bound thereto is the pIgR stalk or a domain, conserved sequence or region thereof.

In a related aspect, the ligand may be a polypeptide that corresponds to an amino acid sequence that is conserved in pIgR proteins from a variety of species, e.g., a polypeptide having an amino acid sequence selected from the group consisting of LRKED, QLFVNEE, LNQLT, YWCKW, GWYWC, STLVPL, SYRTD, and KRSSK.

In a related aspect, the ligand may be a polypeptide that corresponds to an amino acid sequence present in a defined region, e.g., a region of a pIgR, wherein said pIgR can be from any animal, and wherein said region is selected from the group consisting of:
R1 From KRSSK to the carboxy terminus of pIgR,
R2a From SYRTD to the carboxy terminus of pIgR,
R2b From SYRTD to KRSSK,
R3a From STLVPL to the carboxy terminus of pIgR,
R3b From STLVPL to KRSSK,
R3c From STLVPL to SYRTD,
R5e From YWCKW to GWYWC,
R6e From LNQLT to GWYWC,
R6f From LNQLT to YWCKW,
R7e From QLFVNEE to GWYWC,
R7f From QLFVNEE to YWCKW,
R7g From QLFVNEE to LNQLT,
R8e From LRKED to GWYWC,
R8f From LRKED to YWCKW,
R8g From LRKED to LNQLT, and
R8h From LRKED to QLFVNEE.

In a related aspect, a complex or compound directed to the pIgR stalk or a portion, domain or region thereof, is a cytotoxic agent, and is delivered to a cancerous or otherwise diseased cell that displays pIgR or the pIgR stalk.

In another aspect, the invention provides a compound comprising n targeting elements directed to a ligand that confers transcellular, transcytotic or paracellular transporting properties to a compound bound to said ligand, wherein one or more of desirable attributes of said compound is enhanced as compared to a second compound having m targeting elements, wherein n and m are both whole integers, and n>m.

By “enhanced” it is meant that one or more desirable attributes, including but not limited to transcytotic and paracellular transportation properties, is augmented, improved or introduced into a complex or compound. By way of non-limiting example, enhanced transcytotic properties include an increase in the relative rate of one or more processes such as of endocytosis, transcytosis or exocytosis; an increased range of recognition, or a higher degree of specificity, for particular types and species of pIgR and stalk molecules; or the ability to transcytose compounds of a larger molecular weight and/or a different composition. Similarly, enhanced properties of paracellular transport include but are not limited to an increase in the relative rate of transport; or the ability to transport compounds of a larger molecular weight.

The “relative rate” of a multimeric compound or complex of the invention refers to the number of molecules of a multimer undergoing a given process (endocytosis, transcytosis, paracellular transport, etc.) over a set period of time compared to the number of molecules of a comparable monomer undergoing the same process over the same period of time. Rates may also be expressed in absolute terms, e.g., x moles of molecules per nanosecond. Similarly, other properties of complexes and compounds may be measured in absolute or relative terms.

An enhanced property may also be a preference for reverse transcytosis (apical to basolateral transcytosis) as compared to forward (basolateral to apical) transcytosis. A preference for reverse trancytosis is desirable in aspects of the invention where delivery of complex and compounds from the lumen of an organ to the circulatory system is the desired goal.

Other properties that may be enhanced in the complexes and compounds of the invention include, by way of non-limiting example, increased stability of complexes and compounds in vitro or in vivo; increased yield or improved purity of complexes and compounds, particularly as produced by recombinant DNA expression systems; removal or reduction of one or more undesirable properties, e.g., undesired side effects; and the like.

Desirable attributes include, but are not limited to, those related to the of transport complexes and compounds through cells, particularly epithelial cells, and epithelial or mucosal barriers, i.e., transcellular properties, endocytotic properties, transcytotic properties, exocytotic properties, and paracellular transporting properties. Thus, for example, desirable attributes include, but are not limited to, an increase in the relative rate of a process such as endocytosis, transcytosis, exocytosis, transcellular and paracellular transport, or a preference for transcytosis in one direction or another, apical to basolateral transcytosis and transcellular movement being preferred.

Another type of desirable attribute involves the binding properties of the complex or compound, including, by way of non-limiting example, a change in affinity or avidity for a ligand. Another type of desirable attribute involves pharmacological properties such as half-life, decreased secretion, efficacy, selectivity, and the like.

In another aspect, the invention provides a complex or compound comprising 2 or more targeting elements directed to one or more ligands that confer transcellular, transcytotic or paracellular transporting properties to an agent specifically bound to the ligand, and at least one biologically active portion.

Such multivalent bioactive complexes and compounds preferably have enhancements in one or more desirable attributes as compared to similar complexes and compounds that have only 1 targeting element directed to the ligand.

In another aspect, the invention provides a complex or compound comprising a biologically active portion and a targeting element directed to a ligand that confers transcellular, transcytotic or paracellular transporting properties to an agent specifically bound to said ligand, wherein said targeting element is not an antibody, wherein said complex or compound, or a biologically active portion or metabolite thereof, is absorbed from the lumen of an organ into the body of an animal.

In another aspect, the invention provides a complex or compound comprising a biologically active portion and a targeting element directed to a ligand that confers transcellular, transcytotic or paracellular transporting properties to an agent specifically bound to said ligand, wherein said targeting element is not an antibody, and at least one biologically active portion, wherein said complex or compound, or a biologically active portion or metabolite thereof, is absorbed from the lumen of an organ into the body of an animal.

Another aspect of the invention is a complex or compound comprising 2 or more targeting elements directed to one or more ligands that confer transcellular, transcytotic or paracellular transporting properties to an agent specifically bound to said ligand, and at least one biologically active portion, wherein said complex or compound, or a biologically active portion or metabolite thereof, is absorbed from the lumen of an organ into the body of an animal.

Epithelial cells, representing a cellular barrier, line the interior of said lumen. Lumen of particular interest include, by way of non-limiting example, gastrointestinal lumen, the pulmonary lumen, the nasal lumen, a nasopharyngeal lumen, a pharyngeal lumen, a buccal lumen, a sublingual lumen, a vaginal lumen, a urogenital lumen, an ocular lumen, a tympanic lumen, an ocular surface, uterine, urethral, bladder, mammary, salivary, lacrimal, respiratory sinus, biliary, sweat gland.

The complex or compound may be delivered to a fluid portion of the body, e.g., to the blood, lymph, interstitial fluid or amniotic fluid of the animal.

The complex or compound is preferably delivered into the body with a pharmacokinetic profile that results in the delivery of an effective dose of said compound or a biologically active portion thereof.

In another aspect of the invention, the complexes and compounds of the invention are capable of undergoing one or more of a variety of processes relating to molecular transport. Thus, a complex or compound will be capable of undergoing transcellular movement, and/or apical to basolateral transcytosis; apical endocytosis, basolateral exocytosis, intracellular transport can lead to delivery to an intracellular compartment, i.e., an organelle. The complex or compound is, preferably if need be, transported across a cellular barrier. The cellular barrier may be an epithelial or mucosal barrier.

The invention further provides pharmaceutical composition comprising the complexes and compounds. The pharmaceutical compositions can further compromise one or more antiproteases or carrier polypeptides.

Representative antiproteases include, but are not limited to, leupeptin, aprotinin, and chymostatin. Representative carrier proteins include, but are not limited to, albumin, serum albumin, ovalbumin, casein, whey, soy bean protein, hemoglobin, and gluten.

The invention further provides a method of delivering a biologically active agent to an animal in need thereof, comprising contacting said animal with the complex or compound of the invention. Thus, the invention provides a method for transporting a biologically active agent through an epithelial or mucosal barrier, comprising contacting said epithelial or mucosal barrier with a complex or compound of the invention.

The invention further provides a method of treating a disease in an animal, comprising contacting said animal with a complex or compound of the invention. The invention further provides medical devices and kits comprising the pharmaceutical compositions of the invention.

The complexes and compounds of the invention may further comprises a detectable moiety These complexes and compounds are used in methods of identifying a disease in an animal, comprising contacting said animal with the complex or compound. The invention further provides a diagnostic composition comprising the detectably labeled compound or composition, and diagnostic kits comprising the diagnostic composition.

### BRIEF DESCRIPTION OF THE DRAWINGS

FIG. 1 shows forward and reverse transcytotic pathways of the polyimmunoglobulin receptor (pIgR) in epithelial cells.

FIG. 2 shows alignments of the amino acid sequences of pIgR homologs. Panel 2A, alignment of human, pig, cow, mouse, rat, possum and rabbit pIgR molecules, showing the relative positions of the leader sequence, the region of pIgR the secretory component that binds immunoglobulin (Ig), conserved sequences (boxed with thick upper border), domains 1-6, and the transmembrane domain (arrows, border between domains); Panel 2B, alignment of human, simian (CynMonk), rabbit and rat stalk amino acid sequences; Panel 2C, alignment of nucleotide sequences.

FIG. 3 shows the nucleotide sequence of pSyn5AF (SEQ ID NO:1), a plasmid that encodes single chain antibody sFv5AF. The emboldened nucleotide sequence indicates the reading frame (ATG, start codon; TAA, stop codon); boxed sequences indicate restriction enzyme sites (aagctt, Hind III site; gaattc, EcoRI site).

FIG. 4 shows the amino acid sequence (SEQ ID NO:2) of the secreted form of the sFv5AF encoded by pSyn5AF. Symbols: Pelb leader, a leader sequence that directs secretion from E. coli; FLAG, FLAG epitope; linker, amino acid sequence (GGGGS)₃; myc, c-myc epitope; 6 HIS, 6×His tag; CDR, complementarity-determining region; FR, framework element; and the heavy and light chains of the sFv are indicated. The sequence of sFv5AF is similar to that of sFv5A, but a FLAG tag is present in sFv5AF, and the 5th residue in the sFv sequence is glutamine (Q) in 5A and valine (V) in 5AF. The amino-terminal Pelb leader sequence is MKYLLPTAAAGLLLLAAQPAMA, and the carboxy terminal sequence is AAAEQKLISEEDLNGAAHHHHHH.

FIG. 5 shows the amino acid sequence of the secreted form of the sFv5AF-Cys (SEQ ID NO:12). The sFv5AF-Cys protein consists of, from an amino- to carboxy-terminal direcetion, a pelb leader (for secretion in E. coli), a FLAG epitope tag, a heavy chain variable region, a spacer sequence [GGGGS repeated three times, i.e., (G4S)3], a light chain variable region, another (G4S)₃ linker, a cysteine residue (emboldened “C”) that has been introduced into the sFv relative to sFv5AF, a c-myc epitope tag, and a 6×His tag (for purification by Immobilized Metal-ion Affinity Chromatography, IMAC). The framework (FR) and complementarity-determining regions (CDR) of the heavy chain and light chain are indicated. The non-immunoglobulin regions (Pelb leader, FLAG epitope tag, linker (G4S)₃, c-myc tag and 6×His tag) are shaded. In addition to the FLAG tag, the amino acid sequence of sFv5AF differs from sFv5A in that the 5th residue in the sFv sequence is changed from a glutamine (Q) to a valine (V) amino acid residue.

FIGS. 6, 7 and 8 illustrate reaction schemes for of forming a disulfide bond between two different binding regions (e.g., between 2 sFv molecules, 2 Fab molecules, or a sFv and a Fab molecule). FIG. 7 illustrates a bispecific binding molecule prepared using one sFv that has been derivatized with 2-iminothiolane, and one sFv that has been derivatized with SPDP (N-succinimidyl-3-(2-pyridyldithio)-propionate.

FIG. 9 shows the nucleotide sequence of a cDNA that encodes, and the amino acid sequence of, human calcitonin (GenBank Accession No. M26095; SEQ ID NOS:7-8). The start (ATG) and stop (TAA) codons are underlined.

FIG. 10 shows the nucleotide sequence of a cDNA that encodes, and the amino acid sequence of, salmon calcitonin (GenBank Accession No. 64312; SEQ ID NOS:9-10). The start (ATG) and stop (TGA) codons are underlined.

FIG. 11 shows an amino acid sequence alignment for several representative calcitonin proteins from different species.

FIG. 12 shows the strategy and sequences used to clone mouse pIgR sequences.

FIG. 13 shows the strategy and sequences used to clone human pIgR sequences.

FIG. 14 shows a strategy and sequences for cloning rat pIgR sequences.

FIG. 15 shows the chimeric rabbit/rat pIgR molecule. Panel 15A shows the structure of the chimeric pIgR. Panel 15B shows the amino acid sequence of the chimeric pIgR (SEQ ID NO:13). The transmembrane domain of the chimera is underlined. The rat portion of the molecule is emboldened. This segment consists of half of domain 5, domain 6, and most of the transmembrane domain. The cleavage site of the signal sequence is indicated by a filled circle.

FIG. 16 shows the amino acid sequences for various pIgR species encoded within GST-pIgR fusion proteins. Amino acids not contained within the pIgR protein are shown in bold and underlined. The most amino terminal amino acids in the sequences (GS) denote the amino acid residues glycine and serine residues contained at the carboxy terminus of the GST portion of the fusion protein. The carboxy termini of the fusion proteins contain additional amino acids not contained within the pIgR protein; in some cases these additional residues include a “His epitope tag” (HHHHHH). A consensus amino acid sequence for this part of the pIgR protein is shown below the sequences for cynomolgus, human, rat and rabbit sequences.

FIG. 17 shows the partial amino acid sequence of a bacterial adhesion protein, CbpA (SEQ ID NO:14). Emboldened and underlined amino acid sequences indicate amino acid sequences that bind, or contain an element that binds, pIgR.

FIG. 18 shows the transwell transcytosis assay system.

FIG. 19 shows the results of assays that compare the transcytosis of sFv5AF-Cys monomers and dimers.

FIG. 20 shows the results of assays that demonstrate sFv5AF-mediated M1 antibody transcytosis.

FIG. 21 shows the time course of transcytosis of monovalent (monomers) and multivalent (dimers)ₛFv5 molecules.

FIG. 22 shows the nucleotide sequence of a cDNA that encodes, and the amino acid sequence of, human growth hormone (hGH; GenBank Accession No. 4503988; SEQ ID NOS:3-4). The reading frame for hGH is emboldened and the start (ATG) and stop (TAG) codons thereof are underlined.

FIG. 23 shows the pharmacokinetic profile of a sFv5AF-human growth hormone fusion protein (5AF-hGH) in rats. Panel (A) shows the response following intravenous (IV) administration of 0.33 mg/kg of 5AF-hGH. Panel (B) shows the response following intrajejunal (U) of 2.0 mg/kg of 5AF-hGH. The data represent mean of two animals per dosing group.

FIG. 24 shows the sequence of a cDNA encoding human interleukin-2 (IL-2; SEQ ID NO:11).

FIG. 25 shows the sequence of a cDNA encoding human interleukin-4 (IL-4; GenBank Accession No. M13982; SEQ ID NO:15). The start (ATG) and stop codons (TGA) of the IL-4 reading frame are underlined.

FIG. 26 shows the nucleotide sequence of a cDNA that encodes, and the amino acid sequence of, human insulin (GenBank Accession No. 4557670; SEQ ID NOS:5-6). The start (ATG) and stop (TAG) codons are underlined. After removal of the precursor signal peptide, proinsulin is post-translationally cleaved into two chains (peptide A and peptide B) that are covalently linked via two disulfide bonds. Binding of this mature form of insulin to the insulin receptor (INSR) stimulates glucose uptake. A variety of mutant alleles with changes in the coding region have been identified.

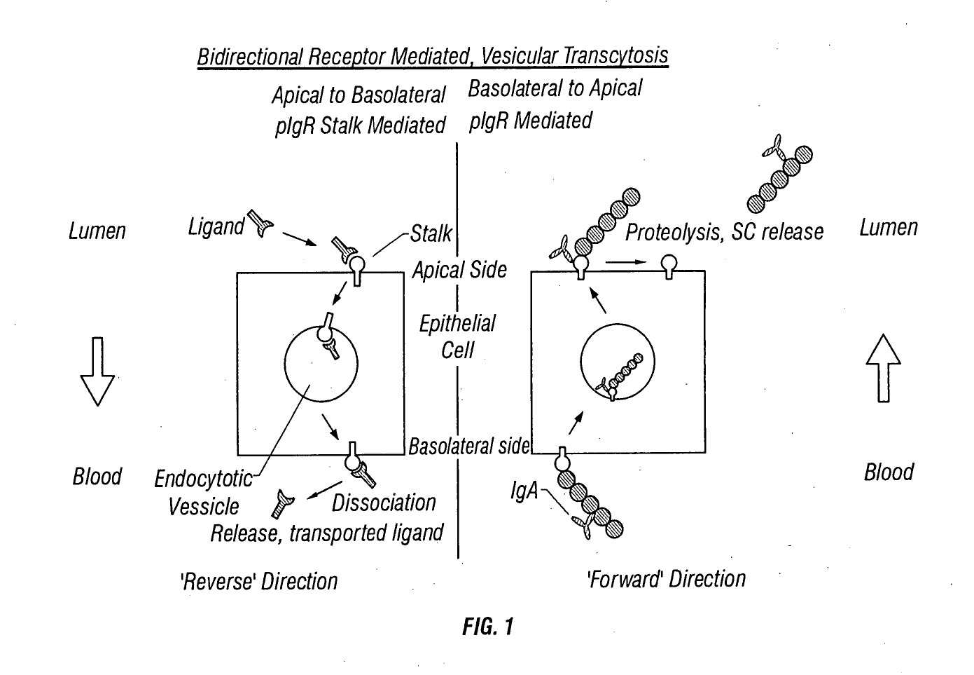

### LISTING OF TABLES

Table 1: Amino Acid Sequences that are Conserved in pIgR Homologs

Table 2: Abbreviations for Amino Acids

Table 3: The Genetic Code

Table 4: Regions of pIgR and Stalk Molecules

Table 5: pIgR and pIgR-Like Proteins from Non-Human Species

Table 6: Conservative Amino Acid Substitutions

Table 7: Classes of Chemical Reactivity of Cross-Linkers

Table 8: Chemical Cross-Linkers and Some of their Properties

Table 9: Calcitonin Homologs and Analogs

Table 10: Sites In IL-4 For Cysteine Substitutions or Insertions

Table 11: Monoclonal Antibodies and Applications Thereof

Table 12: GST-Stalk Fusion Proteins

Table 13: Crosslinkers Used to Derivatize Salmon Calcitonin

Table 14: Comparison of sFv5AF-Cys Binding to Purified Monomer or Dimer sFvAF-Cys Binding

Table 15: Results of Biacore Assay of Scalcitonin-sFv Conjugates

Table 16: Reactants and Order of Addition for SPDP Reactions

Table 17: Biacore Results With Conjugate Preparations AZ008 and AZ009

Table 18: Biacore Results With conjugate preparations AZ018a, AZ018b and AZ019

### DETAILED DESCRIPTION OF THE INVENTION

The inventions disclosed herein relate to complexes and compounds that pass through cellular barriers to deliver compounds into, through and out of cells, and methods of producing and using such complexes and compounds. The complexes and compounds of the invention comprise a biologically active portion and a targeting element directed to a ligand that confers transcellular, transcytotic or paracellular transporting properties to an agent specifically bound to the ligand, with the proviso that the targeting element is not an antibody. In a separate embodiment, a complex or compound of the invention comprises 2 or more targeting elements directed to a ligand that confers transcellular, transcytotic or paracellular transporting properties to an agent specifically bound to the ligand. Preferred ligands include but are not limited to the stalk of pIgR, a pIgR domain, an amino acid sequence that is conserved among pIgR's from different animals, and one of several regions of pIgR defined herein.

I. Structure and Function of pIgR

I.A. Structure of pIgR

A polyimmunoglobulin receptor (pIgR) molecule has several structurally and functionally distinct regions that are defined as follows. In the art, a pIgR molecule is generally described as consisting of two different, loosely defined regions called the “stalk” and the “secretory component” (SC). A pIgR molecule binds polymeric immunoglobulins (IgA or IgM) on the basolateral side, and then transports the immunoglobulin to the apical side. Proteolyic cleavage of pIgR takes place on the apical side of an epithelial cell between the SC and the stalk. The SC molecule is released from the cellular membrane and remains bound to and protects the immunoglobulins, whereas the stalk molecule remains bound to the cellular membrane (see “Mucosal Immunoglobulins” by Mestecky et al. in: Mucosoal Immunology, edited by P. L. Ogra, M. E. Lamm, J. Bienenstock, and J. R. McGhee, Academic Press, 1999).

Particularly preferred pIgR molecules are those described in U.S. Pat. No. 6,042,833, and the simian pIgR described in U.S. patent application Ser. No. 60/266,182 (attorney docket No. 057220.0701) entitled “Compositions and Methods for Identifying, Characterizing, Optimizing and Using Ligands to Transcytotic Molecules” by Houston, L. L., and Sheridan, Philip L., which was filed on Feb. 2, 2001. However, it is understood that, in the context of this invention, pIgR also refers to any of that receptor's family or superfamily members, any homolog of those receptors identified in other organisms, any isoforms of these receptors, any pIgR-like molecule, as well as any fragments, derivatives, mutations, or other modifications expressed on or by cells such as those located in the respiratory tract, the gastrointestinal tract, the urinary and reproductive tracts, the nasal cavity, buccal cavity, ocular surfaces, dermal surfaces and any other mucosal epithelial cells. Preferred pIgR and pIgR-like proteins are those that direct the endocytosis or transcytosis of proteins into or across epithelial cells.

As used herein, the terms “secretory component” and “SC” refers to the smallest (shortest amino acid sequence) portion of an apical proteolyzed pIgR molecule that retains the ability to bind immunoglobulins (IgA and IgM). After proteolytic cleavage of pIgR, some amino acid residues remain associated with SC:immunoglobulin complexes but are eventually degaraded and/or removed from such complexes (Ahnen et al., J. Clin. Invest. 77:1841-1848, 1986). According to the definiton of the secretory component used herein, such amino acids are not part of the SC. In certain embodiments of the invention, pIgR-targeting elements that do not recognize or bind to the SC are preferred.

As used herein, the term “stalk” refers to a molecule having an amino acid sequence derived from a pIgR, wherein the stalk sequence does not comprise amino acid sequences derived from the SC. A stalk molecule comprises amino acid sequences that remain bound to the apical membrane following the apical proteolytic cleavage when such cleavage occurs and amino acid sequences required for such cleavage. Preferred stalk molecules confer one or more transcytotic properties to a ligand bound thereto. Most preferred are stalk molecules that confer the ability to undergo apical to basolateral transcytosis to a ligand bound thereto.

I.A.1. Protein Domains

Another way in which different portions of a pIgR molecule, and SC and stalk molecules derived therefrom, can be delineated is by reference to the domains thereof. A protein “domain” is a relatively small (i.e., <about 150 amino acids) globular unit that is part of a protein. A protein may comprise two or more domains that are linked by relatively flexible stretches of amino acids. In addition to having a semi-independent structure, a given domain may be largely or wholly responsible for carrying out functions that are normally carried out by the intact protein. In addition to domains that have been determined by in vitro manipulations of protein molecules, it is understood in the art that a “domain” may also have been identified in silico, i.e, by software designed to analyze the amino acid sequences encoded by a nucleic acid in order to predict the limits of domains. The latter type of domain is more accurately called a “predicted” or “putative” domain but, in the present disclosure, the term domain encompasses both known and predicted domains unless stated otherwise.

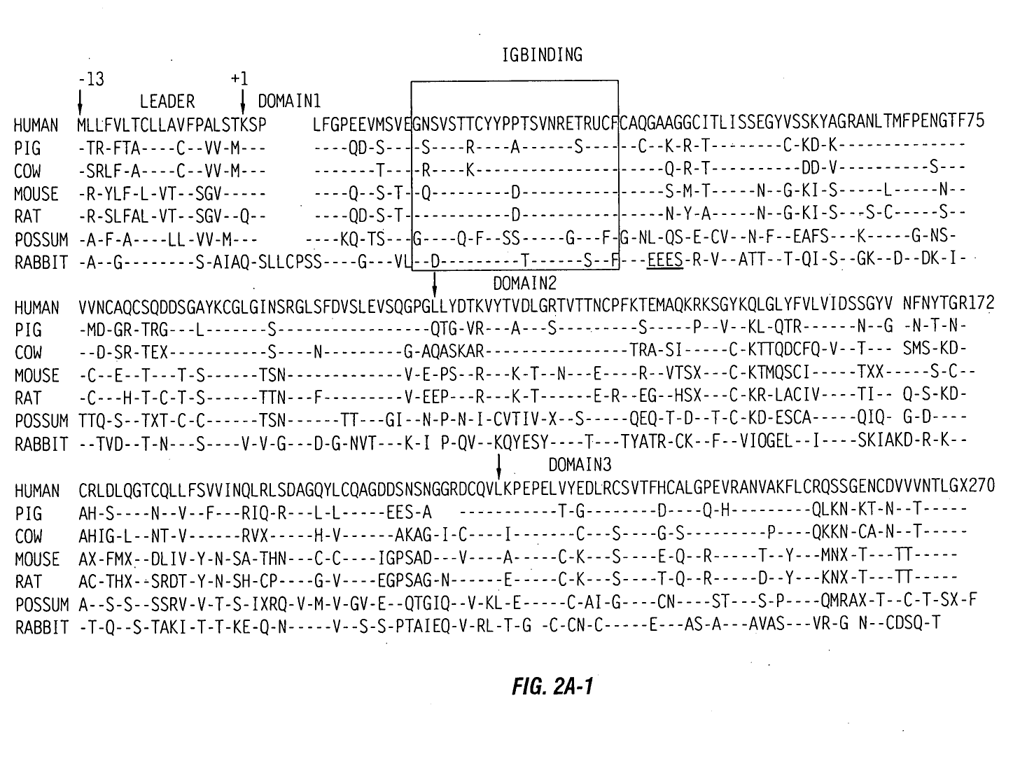

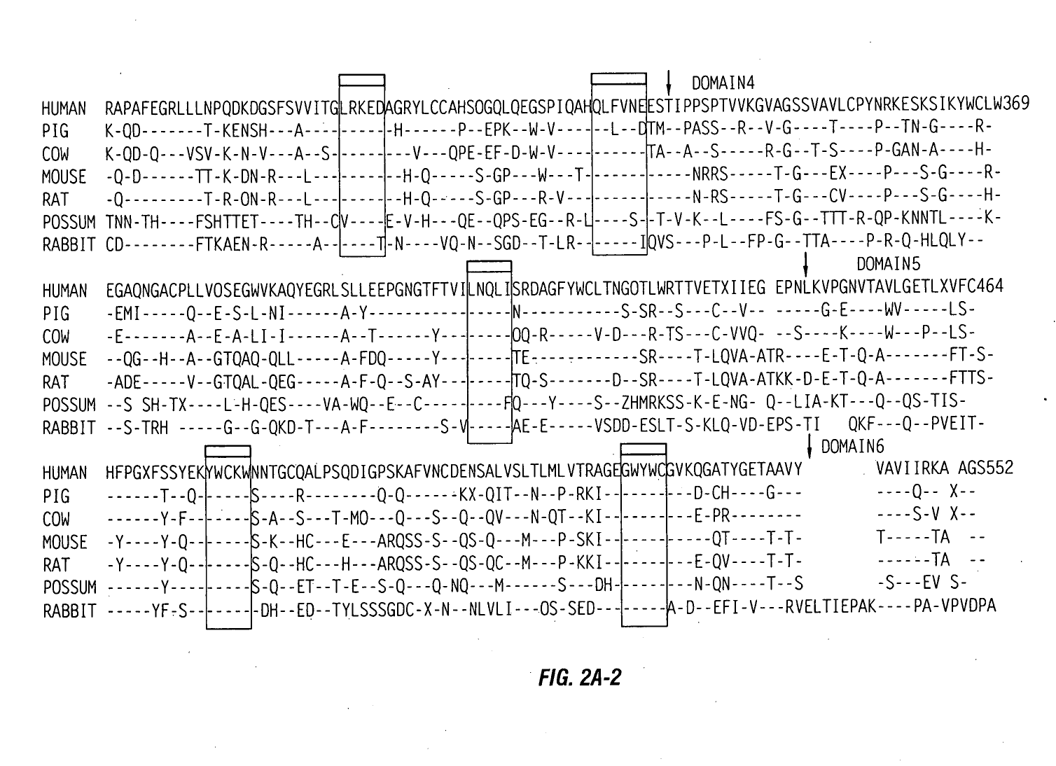

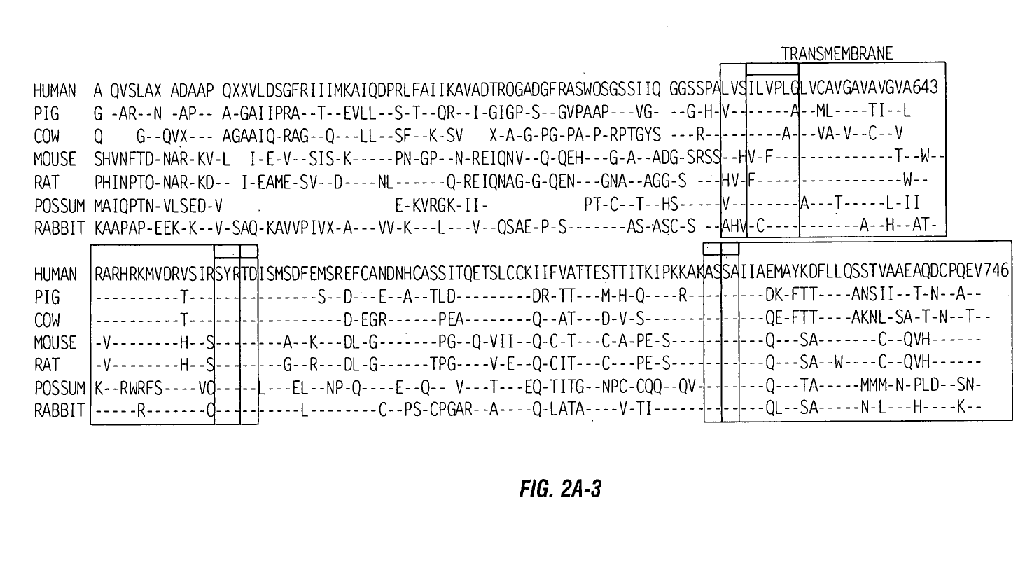

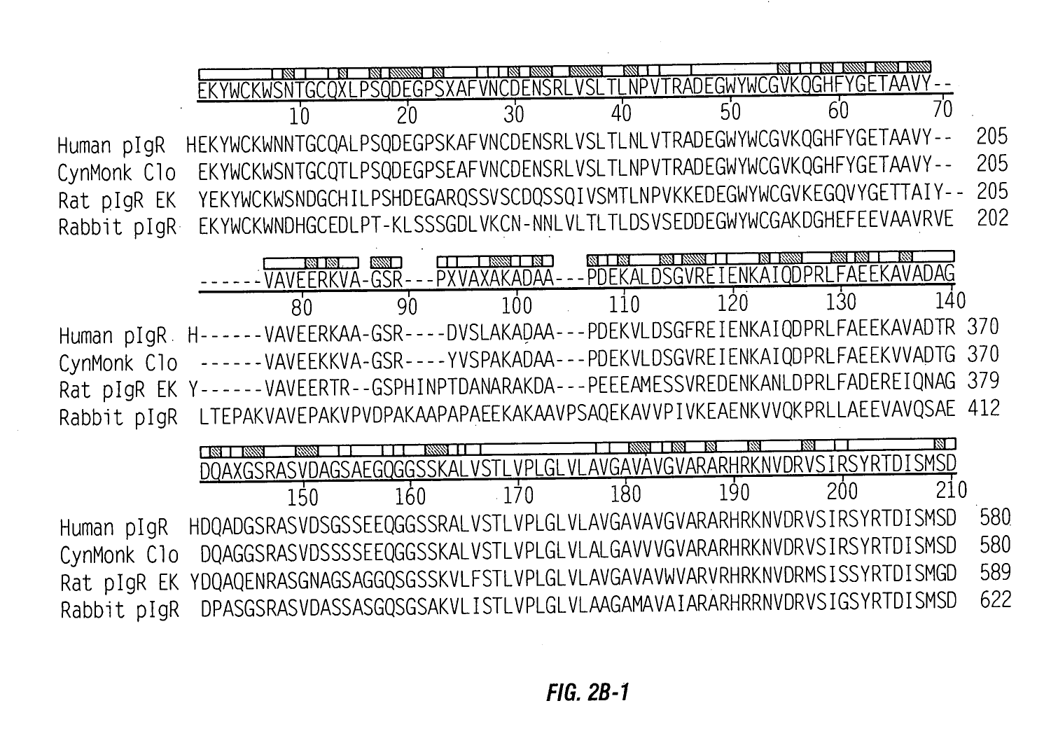

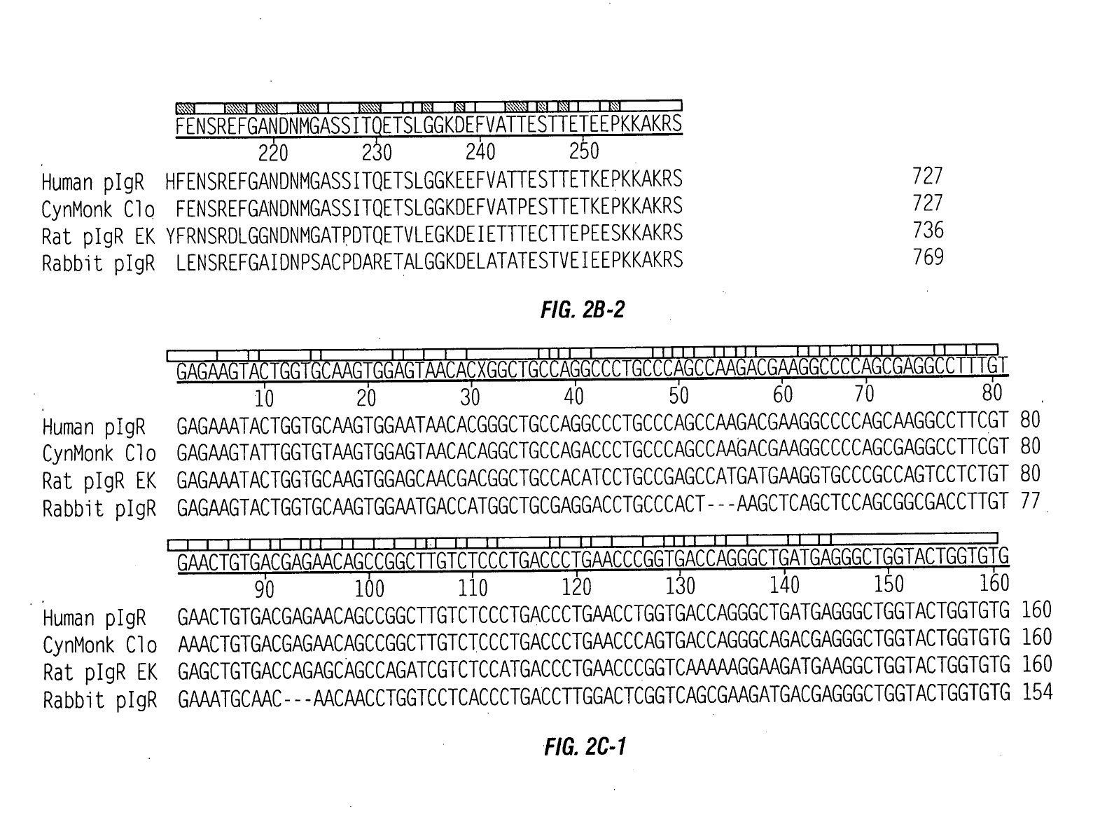

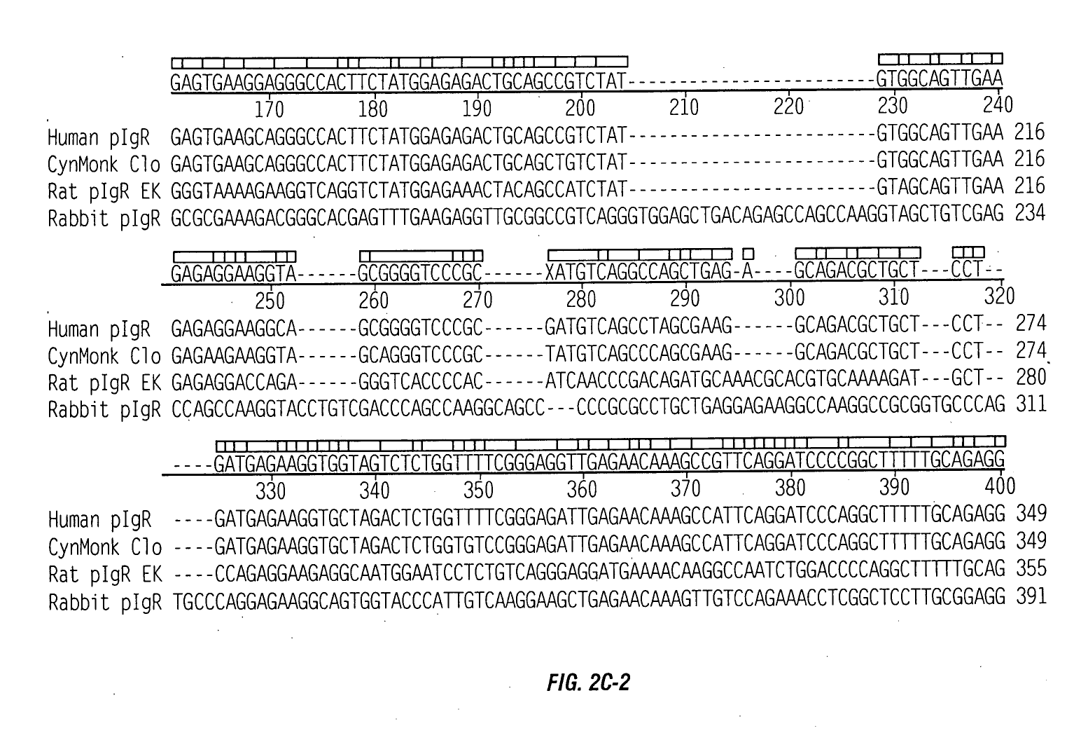

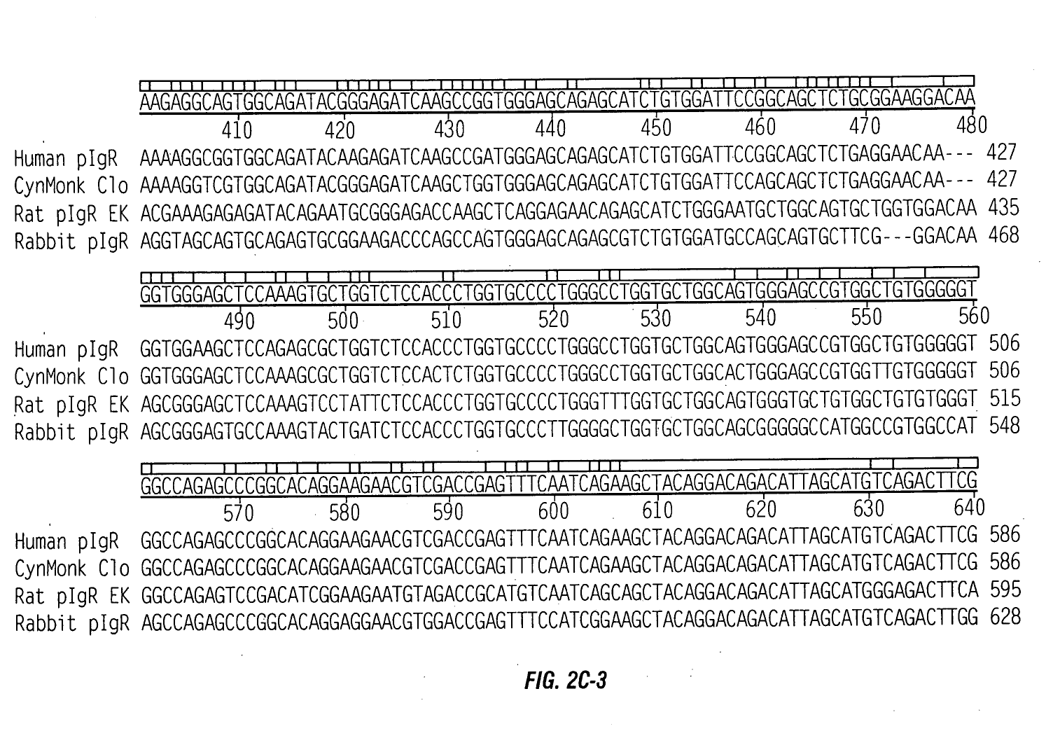

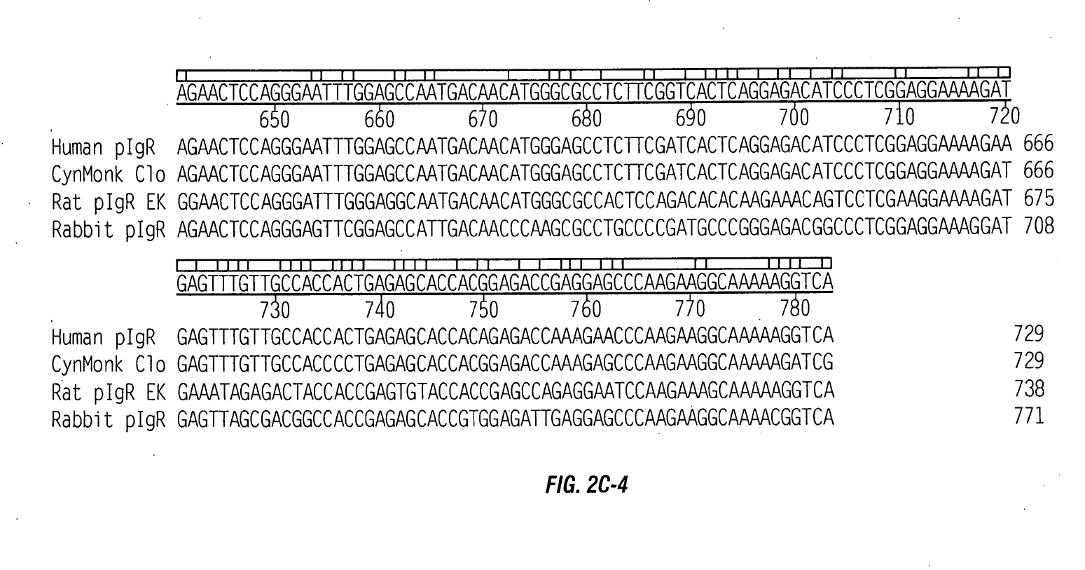

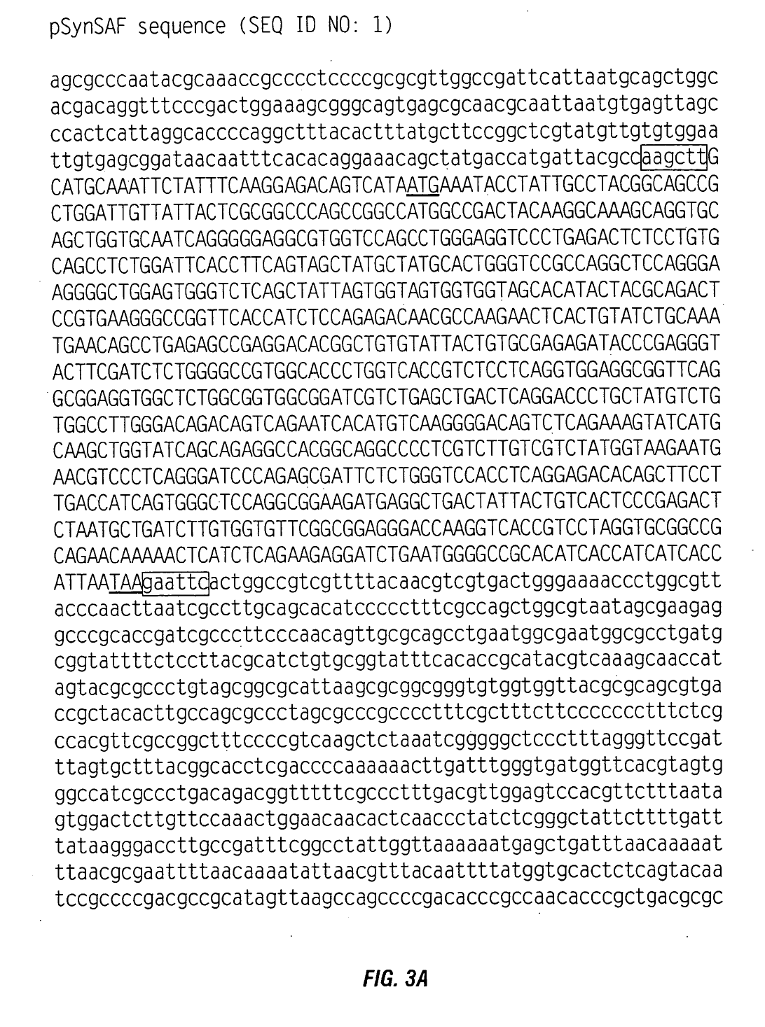

Domains of pIgR molecules include a leader sequence, extracellular domains 1 through 6, a transmembrane domain and an intracellular domain (see FIG. 2 herein and FIG. 3 of Piskurich et al., J. Immunol. 154:1735-1747, 1995). The intracellular domain contains signals for transcytosis and endocytosis. Domains of a pIgR molecule that are of particular interest in the present disclosure include but are not limited to domain 5, domain 6, the transmembrane domain and the intracellular domain. Preferred domains confer the ability to undergo apical to basolateral transcytosis to a ligand bound thereto.

I.A.2. Regions Defined by Conserved Sequences

Another way in which different portions of a pIgR molecule can be defined is by reference to amino acid sequences that are conserved between pIgR homologs (i.e., pIgR molecules isolated from non-human species; see below). Non-limiting examples of conserved amino acid sequences include those found in Table 1; see also FIG. 2. (For brevity's sake, the one letter abbreviations for amino acids is used in Table 1, but versions of sequences that employ the three letter amino acid designations may be found in the Sequence Listing; see also Table 2.)

TABLE 1
AMINO ACID SEQUENCES THAT ARE
CONSERVED IN PIGR HOMOLOGS

| Amino Acid Sequence | Position of Amino Acid Residues | |
|---|---|---|
| Conserved among pIgR | in Human pIgR Relative to | SEQ |
| Homologs | Amino Terminal Methionine* | ID NO: |
| LRKED | 297-301, inclusive | |
| QLFVNEE | 325-331, inclusive | |
| LNQLT | 410-414, inclusive | |
| YWCKW | 476-480, inclusive | |
| GWYWC | 522-526, inclusive | |
| STLVPL | 624-629, inclusive | |
| SYRTD | 658-662, inclusive | |
| KRSSK | 732-737, inclusive | |

*As described in FIG. 3 of Mostov and Kaetzel, Chapter 12 in: Mucosal Immunology, Academic Press, 1999, pages 181-211.

TABLE 2
ABBREVIATIONS FOR AMINO ACIDS

| Amino acid | Three-letter Abbreviation | One letter symbol |
|---|---|---|
| Alanine | Ala | A |
| Arginine | Arg | R |
| Asparagine | Asn | N |
| Aspartic Acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

TABLE 3
THE GENETIC CODE

| First | | | | | Third |
|---|---|---|---|---|---|
| position (5′ | Second Position | | | | position (3′ |
| end) | U | C | A | G | end) |
| U | Phe | Ser | Tyr | Cys | U |
| | Phe | Ser | Tyr | Cys | C |
| | Leu | Ser | Stop | Stop | A |
| | Leu | Ser | Stop | Trp | G |
| C | Leu | Pro | His | Arg | U |
| | Leu | Pro | His | Arg | C |
| | Leu | Pro | GIn | Arg | A |
| | Leu | Pro | GIn | Arg | G |
| A | Ile | Thr | Asn | Ser | U |
| | Ile | Thr | Asn | Ser | C |
| | Ile | Thr | Lys | Arg | A |
| | Met | Thr | Lys | Arg | G |
| G | Val | Ala | Asp | Gly | U |
| | Val | Ala | Asp | Gly | C |
| | Val | Ala | Glu | Gly | A |
| | Val | Ala | Glu | Gly | G |

Thus, for example, a specific internal portion of a given pIgR molecule might be defined as a region that has an amino-terminal border that has the amino acid sequence EKYWCKW and a carboxy-terminal border having the amino acid sequence side having the amino acid sequence DEGWYWCG. In human pIgR, the region so defined would be the amino acid sequence of residues 474 through 529. In the present invention, regions of any given pIgR molecule that are of particular interest include but are not limited to the regions described in Table 4 that are not conserved between pIgR homologs from different species:

TABLE 4
REGIONS OF PIGR AND STALK MOLECULES

| Region | Description |
|---|---|
| R1 | From KRSSK to the carboxy terminus, |
| R2a | From SYRTD to the carboxy terminus, |
| R2b | From SYRTD to KRSSK, |
| R3a | From STLVPL to the carboxy terminus, |
| R3b | From STLVPL to KRSSK, |
| R3c | From STLVPL to SYRTD, |
| R4a | From GWYWC to the carboxy terminus, |
| R4b | From GWYWC to KRSSK, |
| R4c | From GWYWC to SYRTD, |
| R4d | From GWYWC to STLVPL, |
| R5a | From YWCKW to the carboxy terminus, |
| R5b | From YWCKW to KRSSK, |
| R5c | From YWCKW to SYRTD, |
| R5d | From YWCKW to STLVPL, |
| R5e | From YWCKW to GWYWC, |
| R6a | From LNQLT to the carboxy terminus, |
| R6b | From LNQLT to KRSSK, |
| R6c | From LNQLT to SYRTD |
| R6d | From LNQLT to STLVPL, |
| R6e | From LNQLT to GWYWC, |
| R6f | From LNQLT to YWCKW, |
| R7a | From QLFVNEE to the carboxy terminus, |
| R7b | From QLFVNEE to KRSSK, |
| R7c | From QLFVNEE to SYRTD, |
| R7d | From LNQLT to STLVPL, |
| R7e | From QLFVNEE to GWYWC, |
| R7f | From QLFVNEE to YWCKW, |
| R7g | From QLFVNEE to LNQLT, |
| R8a | From LRKED to the carboxy terminus, |
| R8b | From LRKED to KRSSK, |
| R8c | From LRKED to SYRTD, |
| R8d | From LRKED to STLVPL, |
| R8e | From LRKED to GWYWC, |
| R8f | From LRKED to YWCKW, |
| R8g | From LRKED to LNQLT, and |
| R8h | From LRKED to QLFVNEE. |

Preferred regions confer the ability to undergo apical to basolateral transcytosis to a ligand bound thereto.

I.A.3. Target Molecules

Target molecules derived from a pIgR molecule, a secretory component (SC) molecule, or a stalk molecule, or to domains, conserved sequences, and defined regions thereof, are prepared as described herein and used as target molecules for the preparation of ligands and targeting elements of the invention. Preferred target molecules do not comprise amino acid sequences derived from the SC.

Target molecules may be chimeric, i.e., hybrid molecules derived from molecules from at least two different species. An example of a chimeric stalk target molecule is the rat/rabbit hybrid stalk molecule described herein. A target molecule may also be a fusion protein, such as the domain 6-GST fusion proteins described in the Examples.

Preferred target molecules confer the ability to undergo apical to basolateral transcytosis to a ligand bound to a pIgR molecule or a stalk molecule, wherein the ligand does not bind specifically to an SC molecule. Other preferred target molecules comprise sequences from a stalk molecule. Target molecules may be produced using suitable techniques such as recombinant gene expression systems, chemical or enzymatic digestion of pIgR, SC or stalk molecules, or by in vitro synthesis of oligopeptides. Additionally or alternatively, target molecules may be genetically expressed in cells for techniques and experiments designed to assess transcytotic properties.

I.B. Proteins Related to pIgR

I.B.1. Homologs of pIgR

Homologs of pIgR are also within the scope of the invention. Homologs of pIgR are pIgR proteins from species other than Homo sapiens. By way of non-limiting example, pIgR proteins from various species include those from humans, the rat, mouse, rabbit, cow and possum (Table 5). See also FIG. 3 in Mostov and Kaetzel, Chapter 12, “Immunoglobulin Transport and the Polymeric Immunoglobulin Receptor” in Mucosal Immunity, Academic Press, 1999, pages 181-211; and Piskurich et al., J. Immunol. 154:1735-1747, 1995).

TABLE 5
PIGR AND PIGR-LIKE PROTEINS FROM NON-HUMAN SPECIES

| ORGANISM | ACCESSION NUMBER(S) |
|---|---|
| Zebrafish | 9863256, 8713834, 8282255, & 7282118 |
| (Brachydanio rerio) | |
| Mouse | 8099664, 2804245, 6997240, 4585867, 4585866, |
| (Mus musculus) | 2688814, 2688813, 2688812, 2688811, 2688810, |
| | 2688809, 2688808, 2688807, 3097245, 3046754, |
| | 3046752, 3046751, 3046756, 3046755, 3046750, |
| | 3046748, 3046747 and 2247711 |
| Rat | 2222806, 475572, 475571, 473408, 603168 and |
| (Rattas norvegicus) | 603167 |
| Cow (Bos taurus) | 388279 |
| Possum | 5305520, 5305518, 5305514 and 5305512 |
| (Trichosuras | |
| vulpecula) | |

I.B.2. pIgR-Like Proteins

Also within the scope of the invention are pIgR-like proteins. A “pIgR-like protein” is a protein that has an amino acid sequence having homolgy to a known pIgR protein. In many instances, the amino acid sequences of such pIgR-like molecules have been generated by the in silico translation of a nucleic acid, wherein the nucleotide sequence of the nucleic acid has been determined but is not known to encode a protein. By way of non-limiting example, pIgR-like proteins include PIGRL1 (U.S. Pat. No. 6,114,515); PIGR-1 (U.S. Pat. No. 6,232,441); a mouse gene having an exon similar to one of pIgR's (GenBank Accession No. 6826652); human proteins translated in silico that have homology to pIgR proteins (GenBank Accession Nos. 1062747 and 1062741); and Digr1 (Luo et al., Digr1, a novel membrane receptor of the immunoglobulin gene superfamily, is preferentially expressed by antigen-presenting cells, Biochem Biophys Res Commun 287(1):35-41, 2001)

I.B.3. Substantially Identical and Homologous pIgR Molecules

As used herein, a “homolog” of a pIgR protein or a pIgR-like protein is an isoform or mutant of human pIgR, or a protein in a non-human species that either (i) is “identical” with or is “substantially identical” (determined as described below) to an amino acid sequence in human pIgR, or (ii) is encoded by a gene that is identical or substantially identical to the gene encoding human pIgR. Non-limiting examples of types of pIgR isoforms include isoforms of differing molecular weight that result from, e.g., alternate RNA splicing or proteolytic cleavage; and isoforms having different post-translational modifications, such as glycosylation; and the like.

Two amino acid sequences are said to be “identical” if the two sequences, when aligned with each other, are exactly the same with no gaps, substitutions, insertions or deletions. Two amino acid sequences are defined as being “substantially identical” if, when aligned with each other, (i) no more than 30%, preferably 20%, most preferably 15% or 10%, of the identities of the amino acid residues vary between the two sequences; (ii) the number of gaps between or insertions in, deletions of and subsitutions of, is no more than 10%, preferably 5%, of the number of amino acid residues that occur over the length of the shortest of two aligned sequences; or (iii) have only conservative amino acid substitutions (in one polypeptide as compared to another) that do not significantly affect the folding or activity of the polypeptide. Conservative amino acid substitutions are as described in Table 6). The entire amino acid sequence of two proteins may be substantially identical to one another, or sequences within proteins may demonstrate identity or substantial identity with sequences of similar length in other proteins. In either case, such proteins are substantially identical to each other. Typically, stretches of identical or substantially identical sequences occur over 5 to 25, preferably 6 to 15, and most preferably 7 to 10, nucleotides or amino acids.

TABLE 6
CONSERVATIVE AMINO ACID SUBSTITUTIONS

| Type of Amino | Groups of Amino Acids that Are Conservative |
|---|---|
| Acid Side Chain | Substitutions Relative to Each Other |
| Short side chain | Glycine, Alanine, Serine, Threonine and Methionine |
| Hydrophobic | Leucine, Isoleucine and Valine |
| Polar | Glutamine and Asparagine |
| Acidic | Glutamic Acid and Aspartic Acid |
| Basic | Arginine, Lysine and Histidine |
| Aromatic | Phenylalanine, Tryptophan and Tyrosine |

One indication that nucleotide sequences encoding pIgR proteins are substantially identical is if two nucleic acid molecules hybridize to each other under stringent conditions. Stringent conditions are sequence dependent and will be different in different circumstances. Generally, stringent conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Typically, stringent conditions will be those in which the salt concentration is about 0.02 M at pH 7 and the temperature is at least about 60° C.

Another way by which it can be determined if two sequences are substantially identical is by using an appropriate algorithm to determine if the above-described critera for substantially identical sequences are met. Sequence comparisons between two (or more) polynucleotides or polypeptides are typically performed by algorithms such as, for example, the local homology algorithm of Smith and Waterman (Adv. Appl. Math. 2:482, 1981), by the homology alignment algorithm of Needleman and Wunsch (J. Mol. Biol. 48:443, 1970), by the search for similarity method of Pearson and Lipman (Proc. Natl. Acad. Sci. U.S.A. 85:2444, 1988), by computerized implementations of these algorithms (GAP, BESTFIT, FASTA, and TFASTA in the Wisconsin Genetics Software Package, Genetics Computer Group (GCG), 575 Science Dr., Madison, Wis.), or by visual inspection.

I.C. Binding and Transcytotic Assays

The ability of a pIgR ligand of the invention to bind different pIgR molecules, fragments and derivatives thereof, and to undergo endocytosis, transcytosis, and/or exocytosis is a desirable attribute of these proteins. The pIgR-binding capacity of fusion proteins are examined using the following techniques. Non-limiting examples of such assays include the following.

Cell lines that may be used in such assays are generally epithelial cells, particularly polarized cells having apical and basolateral surfaces. Such cells include those that naturally express pIgR or the pIgR stalk, preferably in response to factors and conditions that can be altered or manipulated, and cells that are transfected with nucleic acids encoding pIgR molecules, stalk molecules or target molecules prepared therefrom.

A non-limiting example of the former type of cells are epithelial cells isolated from human trachea, nasopharynx or bronchi. When grown on plastic, these primary cultures down-regulate expression of pIgR whereas, when grown on collagen-coated porous filters, the cultures produce pIgR (U.S. Pat. No. 6,261,787 B1 and Ferkol et al., Am. J. Respir. Crit. Care Med. 161:944-951, 2000).

Other non-limiting examples are T560, a mouse B lymphoma that originated in gut-associated lymphoid tissue and which expresses pIgR (Phillips-Quagliata et al., The IgA/IgM receptor expressed on a murine B cell lymphoma is poly-Ig receptor, J Immunol 2000 Sep. 1; 165(5):2544-55); and Fischer rat thyroid (FRT) cells (Sarnataro et al., Detergent insoluble microdomains are not involved in transcytosis of polymeric Ig receptor in FRT and MDCK cells, Traffic 2000 October; 1(10):794-802; Aging effects on hepatic NADPH cytochrome P450 reductase, CYP2B1&2, and polymeric immunoglobulin receptor mRNAs in male Fischer 344 rats).

Cell lines that do not normally express the pIgR or the stalk, but which can be genetically transfected or transfected to express the pIgR, the stalk or target molecules include Madin-Darby canine kidney (MDCK) cells (as described throughout the specification and in Giffroy et al., Scand. J. immunol. 53:56-64, 2001); chinese hamster ovary (CHO) cells (Asano et al., Molecular maturation and functional expression of mouse polymeric immunoglobulin receptor, J Immunol Methods 1998 May 1; 214(1-2):131-9); endothelial cell lines such as ECV 304 (Su et al., Opposite sorting and transcytosis of the polymeric immunoglobulin receptor in transfected endothelial and epithelial cells, J Cell Sci 1998 May; 111 (Pt 9):1197-206); and, particularly in instances where inhalation delivery of compounds is being tested, in cells from the 16HBEo bronchial cell line (Ferkol et al., Am. J. Crit. Care 16:944-951, 2000). Methods of transfecting cells in order to direct the expression of pIgR molecules therein are known in the art (Breitfeld et al., Methods in Cell Biology 32:329-337, 1989).

I.C.1. Ex Vivo Testing of Ligand Binding

The ex vivo pIgR binding capacity of a pIgR-targeted protein is assessed by measuring endocytosis or transcytosis of bound ligand in mammalian epithelial cells. Receptor-mediated endocytosis provides an efficient means of causing a cell to ingest material which binds to a cell surface receptor. (See Wu et al., J. Biol. Chem. 262:4429-4432, 1987; Wagner et al., Proc. Natl. Acad. Sci. USA 87:3410-3414, 1990, and published EPO patent application EP-A1 0388758). Any number of well known methods for assaying endocytosis may be used to assess binding. For example, binding, transcytosis, and internalization assays are described at length in Breiftfeld et al. (J. Cell Biol. 109:475-486, 1989).

Ligand-pIgR binding is measured by a variety of techniques known in the art, e.g., immunoassays and immunoprecipitation. By way of example, antibodies to the biologically active portion of a protein conjugate can be used to bind and precipitate detectably labeled pIgR or stalk molecules; the amount of labeled material thus precipitated corresponds to the degree of pIgR binding to a ligand such as, e.g., a protein conjugate having a pIgR-targeting element (see Tajima, J. Oral Sci. 42:27-31, 2000).

I.C.2. Apical Endocytosis

Apical endocytosis is conveniently measured by binding a ligand, such as sFv5A or a derivative thereof (see FIGS. 3 to 5), to a stalk molecule at the apical surface of transfected Madin-Darby canine kidney (MDCK) cells at 4° C., warming to 37° C. for brief periods (0-10 min), and cooling the cells back down to 4° C. Ligand molecules remaining on the surface are removed by stripping at pH 2.3. Intracellular ligand molecules are those that remain cell-associated after the stripping, while surface-bound ligand molecules are those removed by the acid wash. Controls for non-specific sticking include using molecules that are structurally related to the ligand but which do not bind to a pIgR or stalk molecule (e.g., an unrelated sFv in the case of sF5), and/or MDCK cells that are not transfected with genetic sequences encoding a pIgR molecule or a stalk molecule.

I.C.3. Apical to Basolateral Transcytosis

Apical to basolateral (“reverse”) transcytosis is assessed by allowing MDCK cells to bind the ligand at the apical surface at 4° C., followed by incubation at 37° C. for 0 to 240 min, and then measuring the amount of ligand delivered into the basolateral medium. This basolaterally-delivered ligand is compared to the sum of ligand that remains associated with the cells (intracellular or acid-stripped) and the ligand released back into the apical medium.

Alternatively, transcystosis is assessed as follows. Typically, and unless noted otherwise herein, the general protocol for apical to basolateral transcytosis assays was as follows.

Non-transfected (or wild type) MDCK cells, and MDCK cells that have been transfected with a gene encoding a pIgR from a variety of species, or hybrid pIgR molecules such as the rat/rabbit hybrid pIgR described herein, are grown on the surface of a porous membrane in a transwell plate (Corning Costar, #3401, 12 mm diameter, 0.4 micrometer pore size polycarbonate membrane). The cells are grown until they are confluent and form tight junctions that do not allow leakage of substances through the cell layer. MDCK cells are polarized when grown in this manner in a transwell chamber. Cells in transwells are washed 3× with MEM/BSA (Sigma No. M4642, with 20 mM Hepes, pH 7.4, 0.6% BSA, containing penicillin and streptomycin), and test or control articles are placed in the upper chamber (apical surface) of the transwell compartment in a volume of 300 μl MEM/BSA. The transwells are placed in a 12 well plate with 800 ul of MEM/BSA in the basolateral compartments. After a period of time, usually 8 to 16 hours, samples of the upper and lower (basolateral) chamber are removed and analyzed for the presence of the test and control articles.

The apical and basolateral media are adjusted to a volume of 1 ml with MEM/BSA. One hundred (100) μl of the apical media is then added to 900 l of MEM/BSA to give a 1/10 dilution. The entire μvolume of the basolateral media (100%), and the 1/10 dilution of the apical media (10%), are prepared and incubated with an appropriate affinity matrix. (Alternatively, 500 μl (50%) of the adjusted basolateral media and 50 ul (5%) of the adjusted apical media can be assayed.) For complexes or compounds comprising the sFv5A polypeptide, protein A sepharose (Pharmacia) is the affinity matrix of choice. Typically 100 μl of a 10% slurry of protein A sepharose is added to the samples. After overnight incubation rotating at 4 degrees, beads are pelleted by centrifugation in a Beckman Microfuge for 2 minutes at full speed. The supernatant is removed, and 1 ml PBS is added, followed by centrifugation and removal of the supernatant. The wash is repeated 2 more times. Beads are dried by removing the excess liquid with a Hamilton syringe. Fifty (50) μl of SDS-PAGE sample buffer is added, samples are boiled for 5 minutes, centrifuged briefly, and 20 μl is analyzed by SDS-PAGE (typically on an 8-16% Tris-HCl, 1.0 mm Criterion Precast Gel (Bio-Rad 345-0038) and run at 150 mamps for 60-80 minutes) followed by transfer to PVDF filters for Western blotting. The filters are probed with antibodies to sFv5A, the FLAG or other epitope tag, or to the biologically active portion of the complex or compound of interest. Lanes on the Western blot correspond to samples taken from the apical chamber or the basolateral chamber of the transwell compartment. The relative intensity of the staining between the apical and basolateral lanes is an indication of the efficiency of transcytosis. Equal intensity of these bands represents approximately 10% transcytosis, since the volume of the apical media that is present on the gel is tenfold less than that of the basolateral media.

I.C.4. Basolateral Endocytosis

Basolateral endocytosis is assessed by methods such as those described by Tajima (J. Oral Sci. 42:27-31, 2000). Non-specific transport (e.g., fluid phase endocytosis and transcytosis, or paracellular leakage between cells) can be assessed as a control by using MDCK cells that are not transfected with a pIgR or stalk protein, and/or by the addition of antibody not directed to the pIgR or stalk molecule.

I.C.5. In Vivo Assays

In vivo transcytosis is assessed using pathogen-free experimental animals such as Sprague-Dawley rats. Detectably labeled ligand (e.g., a radioiodinated antibody) is administered into, e.g., the nares (the pair of openings of the nose or nasal cavity of a vertebrate) or the intestine (more details of these types of assays are provided herein in the Examples). As will be understood by those of skill in the art, a “detectable label” is a composition or moiety that is detectable by spectroscopic, photochemical, biochemical, immunochemical, electromagnetic, radiochemical, or chemical means such as fluorescence, chemifluoresence, or chemiluminescence, or any other appropriate method.

In vivo apical to basolateral (“reverse”) transcytosis is assessed by measuring the delivery of a pIgR-targeting ligand into the circulation as measured by the presence of a detectable label that has been incorporated into the protein that is being tested. The integrity of the ligand recovered from the circulation can be assessed by analyzing the ligand on SDS polyacrylamide gel electrophoresis. Such assays are described in more detail in the Examples.

In vivo basolateral to apical (“forward”) transcytosis is assessed according to methods described in U.S. Pat. No. 6,072,041, which issued Jun. 6, 2000 to Davis et al.; U.S. Pat. No. 6,261,787 B1, which issued Jul. 17, 2001 to Davis et al.; published PCT application No. WO 00/53623, published Sep. 14, 2000, by Davis et al.; Eckman et al., Am J Respir Cell Mol Biol 1999 August; 21(2):246-52; and Ferkol et al., Am. J. Respir. Crit. Care Med. 161:944-951, 2000.

I.C.6. Specificity of Binding

The binding of a ligand is target-specific in the sense that, although other molecules may be present in a mixture in which ligands and target molecules are contacted with each other, the ligand does not appreciably bind to other (non-target) molecules. For example, in the case of pIgR, it is recognized that the strength of binding between pIgR and a pIgR ligand, i.e., the affinity of a pIgR ligand for pIgR, is a matter of degree. As used herein, “target-specific” means that the pIgR ligand has a stronger affinity for its target molecule (pIgR) than for contaminating molecules, and this difference in affinity is sufficient for a given aspect of the invention. In general, the target specificity of a pIgR ligand for pIgR is comparable to the specificity of antibodies for their antigens. Thus, by way of non-limiting example, the specificity for a ligand for pIgR should be at least approximately that of a single chain antibody (sFv) for pIgR. Examples of sFv's that can be used to evaluate the target specificity of a pIgR ligand include but are not limited to sFv5A and derivatives thereof, such as sFv5AF, which bind to the stalk of pIgR and are described herein; and sFv's that bind to the secretory component (SC) such as, e.g., those described in U.S. Pat. No. 6,072,041.

The specificity of the binding is defined in terms of the values of absolute and relative binding parameters, such as the comparative dissociation constants (Kd) of a ligand for its target molecule as compared to the dissociation constant with respect to the ligand and unrelated molecules and compositions. Typically, the Kd of a ligand with respect to its target molecule will be 2-fold, preferably 5-fold, more preferably 10-fold less, than the Kd of the ligand for unrelated molecules and compositions. Even more preferably the Kd will be 50-fold less, more preferably 100-fold less, and more preferably 200-fold less.

The binding affinity of the ligands with respect to target molecules is defined in terms of the dissociation constant (Kd). The value of Kd can be determined directly by well-known methods, and can be computed even for complex mixtures by methods such as those, for example, set forth in Caceci, M., et al., Byte (1984) 9:340-362. In some situations, direct determination of Kd is problematic and can lead to misleading results. Under such circumstances, a competitive binding assay can be conducted to compare the affinity of a ligand for its target molecule with the affinity of molecules known to bind the target molecule. The value of the concentration at which 50% inhibition occurs (Ki) is, under ideal conditions, roughly equivalent to Kd. Moreover, Ki cannot be less than Kd; determination of Ki sets a maximal value for the value of Kd. Under circumstances where technical difficulties preclude accurate measurement of Kd, measurement of Ki can conveniently be substituted to provide, at the very least, an upper limit for Kd.

Kd may be measured in solution using techniques and compositions described in the following publications. Blake, D. A.; Blake, R. C.; Khosraviani, M.; Pavlov, A. R. “Immunoassays for Metal Ions.” Analytica Chimica Acta 1998, 376, 13-19. Blake, D. A.; Chakrabarti, P.; Khosraviani, M.; Hatcher, F. M.; Westhoff, C. M.; Goebel, P.; Wylie, D. E.; Blake, R. C. “Metal Binding Properties of a Monoclonal Antibody Directed toward Metal-Chelate Complexes.” Journal of Biological Chemistry 1996, 271(44), 27677-27685. Blake, D. A.; Khosraviani, M.; Pavlov, A. R.; Blake, R. C. “Characterization of a Metal-Specific Monoclonal Antibody.” Aga, D. S.; Thurman, E. M., Eds.; ACS Symposium Series 657; American Chemical Society: Washington, D.C., 1997; pp 49-60.

Binding constants and kinetic constants are estimated using calorimetry, equilibrium dialysis, and stopped flow methods using absorbance, fluorsescence, light scattering, turbidity, fluorescence anisotropy, and the like. Additionally or alternatively, Kd is measured using immobilized binding components on a chip, for example, on a BIAcore chip using surface plasmon resonance.

I.C.7. Surface Plasmon Resonance

Binding parameters are measured using surface plasmon resonance, for example, with a BIAcore® chip coated with immobilized binding components. Surface plasmon resonance is used to characterize the microscopic association and dissociation constants of reaction between an sFv or other ligand directed against pIgG associated molecules and pIgR and pIgR fragments. Such methods are generally described in the following references which are incorporated herein by reference. Vely F. et al., BIAcore analysis to test phosphopeptide-SH2 domain interactions, Methods in Molecular Biology. 121:313-21, 2000; Liparoto et al., Biosensor analysis of the interleukin-2 receptor complex, Journal of Molecular Recognition. 12:316-21, 1999; Lipschultz et al., Experimental design for analysis of complex kinetics using surface plasmon resonance, Methods. 20):310-8, 2000; Malmqvist., BIACORE: an affinity biosensor system for characterization of biomolecular interactions, Biochemical Society Transactions 27:335-40, 1999; Alfthan, Surface plasmon resonance biosensors as a tool in antibody engineering, Biosensors & Bioelectronics. 13:653-63, 1998; Fivash et al., BIAcore for macromolecular interaction, Current Opinion in Biotechnology. 9:97-101, 1998; Price et al.; Summary report on the ISOBM TD-4 Workshop: analysis of 56 monoclonal antibodies against the MUC1 mucin. Tumour Biology 19 Suppl 1:1-20, 1998; Malmqvist et al, Biomolecular interaction analysis: affinity biosensor technologies for functional analysis of proteins, Current Opinion in Chemical Biology. 1:378-83, 1997; O'Shannessy et al., Interpretation of deviations from pseudo-first-order kinetic behavior in the characterization of ligand binding by biosensor technology, Analytical Biochemistry. 236:275-83, 1996; Malmborg et al., BIAcore as a tool in antibody engineering, Journal of Immunological Methods. 183:7-13, 1995; Van Regenmortel, Use of biosensors to characterize recombinant proteins, Developments in Biological Standardization. 83:143-51, 1994; and O'Shannessy, Determination of kinetic rate and equilibrium binding constants for macromolecular interactions: a critique of the surface plasmon resonance literature, Current Opinions in Biotechnology. 5:65-71, 1994.

BIAcore® uses the optical properties of surface plasmon resonance (SPR) to detect alterations in protein concentration bound within to a dextran matrix lying on the surface of a gold/glass sensor chip interface, a dextran biosensor matrix. In brief, proteins are covalently bound to the dextran matrix at a known concentration and a ligand for the protein (e.g., antibody) is injected through the dextran matrix. Near infrared light, directed onto the opposite side of the sensor chip surface is reflected and also induces an evanescent wave in the gold film, which in turn, causes an intensity dip in the reflected light at a particular angle known as the resonance angle. If the refractive index of the sensor chip surface is altered (e.g., by ligand binding to the bound protein) a shift occurs in the resonance angle. This angle shift can be measured and is expressed as resonance units (RUs) such that 1000 RUs is equivalent to a change in surface protein concentration of 1 ng/mm². These changes are displayed with respect to time along the y-axis of a sensorgram, which depicts the association and dissociation of any biological reaction.

II. Chemical Structures of Ligands and Targeting Elements

In complexes and compound of the invention, targeting elements and biologically active molecules are independently small molecules, nucleic acids or polypeptides.

Examples of compounds and moities that may be used as targeting elements in the compositions and compounds of the invention are as follows.

II.A. Small Molecules & Derivatives

The term “small molecule” includes any chemical or other moiety that can act to affect biological processes. Small molecules can include any number of therapeutic agents presently known and used, or can be small molecules synthesized in a library of such molecules for the purpose of screening for biological function(s). Small molecules are distinguished from macromolecules by size. The small molecules of this invention usually have molecular weight less than about 5,000 daltons (Da), preferably less than about 2,500 Da, more preferably less than 1,000 Da, most preferably less than about 500 Da.

Small molecules include without limitation organic compounds, peptidomimetics and conjugates thereof. As used herein, the term “organic compound” refers to any carbon-based compound other than macromolecules such nucleic acids and polypeptides. In addition to carbon, organic compounds may contain calcium, chlorine, fluorine, copper, hydrogen, iron, potassium, nitrogen, oxygen, sulfur and other elements. An organic compound may be in an aromatic or aliphatic form. Non-limiting examples of organic compounds include acetones, alcohols, anilines, carbohydrates, monosaccharides, oligosaccharides, polysaccharides, amino acids, nucleosides, nucleotides, lipids, retinoids, steroids, proteoglycans, ketones, aldehydes, saturated, unsaturated and polyunsaturated fats, oils and waxes, alkenes, esters, ethers, thiols, sulfides, cyclic compounds, heterocylcic compounds, imidizoles and phenols. An organic compound as used herein also includes nitrated organic compounds and halogenated (e.g., chlorinated) organic compounds. Methods for preparing peptidomimetics are described below. Collections of small molecules, and small molecules identified according to the invention are characterized by techniques such as accelerator mass spectrometry (AMS; see Turteltaub et al., Curr Pharm Des 2000 6(10):991-1007, Bioanalytical applications of accelerator mass spectrometry for pharmaceutical research; and Enjalbal et al., Mass Spectrom Rev 2000 19(3): 139-61, Mass spectrometry in combinatorial chemistry.)

Preferred small molecules are relatively easier and less expensively manufactured, formulated or otherwise prepared. Preferred small molecules are stable under a variety of storage conditions. Preferred small molecules may be placed in tight association with macromolecules to form molecules that are biologically active and that have improved pharmaceutical properties. Improved pharmaceutical properties include changes in circulation time, distribution, metabolism, modification, excretion, secretion, elimination, and stability that are favorable to the desired biological activity. Improved pharmaceutical properties include changes in the toxicological and efficacy characteristics of the chemical entity.

II.B. Nucleic Acids

Traditionally, techniques for detecting and purifying target molecules have used polypeptides, such as antibodies, that specifically bind such targets. Nucleic acids have long been known to specifically bind other nucleic acids (e.g., ones having complementary sequences). However, aptamers, nucleic acids that bind non-nucleic target molecules have been disclosed. See, e.g., Blackwell et al., Science (1990) 250:1104-1110; Blackwell et al., Science (1990) 250:1149-1152; Tuerk et al., Science (1990) 249:505-510; Joyce, Gene (1989) 82:83-87; and U.S. Pat. No. 5,840,867 entitled “Aptamer analogs specific for biomolecules”.

As applied to aptamers, the term “binding” specifically excludes the “Watson-Crick”-type binding interactions (i.e., A:T and G:C base-pairing) traditionally associated with the DNA double helix. The term “aptamer” thus refers to a nucleic acid or a nucleic acid derivative that specifically binds to a target molecule, wherein the target molecule is either (i) not a nucleic acid, or (ii) a nucleic acid or structural element thereof that is bound through mechanisms other than duplex- or triplex-type base pairing. Such a molecule is called a “non-nucleic molecule” herein.

II.B.1. Structures of Nucleic Acids

“Nucleic acids,” as used herein, refers to nucleic acids that are isolated a natural source; prepared in vitro, using techniques such as PCR amplification or chemical synthesis; prepared in vivo, e.g., via recombinant DNA technology; or by any appropriate method. Nucleic acids may be of any shape (linear, circular, etc.) or topology (single-stranded, double-stranded, supercoiled, etc.). The term “nucleic acids” also includes without limitation nucleic acid derivatives such as peptide nucleic acids (PNA's) and polypeptide-nucleic acid conjugates; nucleic acids having at least one chemically modified sugar residue, backbone, internucleotide linkage, base, nucleoside, or nucleotide analog; as well as nucleic acids having chemically modified 5′ or 3′ ends; and nucleic acids having two or more of such modifications. Not all linkages in a nucleic acid need to be identical.

Nucleic acids that are aptamers are often, but need not be, prepared as oligonucleotides. Oligonucleotides include without limitation RNA, DNA and mixed RNA-DNA molecules having sequences of lengths that have minimum lengths of 2, 4, 6, 8, 10, 11, 12, 13, 14 or 15 nucleotides, and maximum lengths of about 100, 75, 50, 40, 25, 20 or 15 or more nucleotides, irrespectively. In general, a minimum of approximately 6 nucleotides, preferably 10, and more preferably 14 or 15 nucleotides, is necessary to effect specific binding.

In general, the oligonucleotides may be single-stranded (ss) or double-stranded (ds) DNA or RNA, or conjugates (e.g., RNA molecules having 5′ and 3′ DNA “clamps”) or hybrids (e.g., RNA:DNA paired molecules), or derivatives (chemically modified forms thereof). However, single-stranded DNA is preferred, as DNA is less susceptible to nuclease degradation than RNA. Similarly, chemical modifications that enhance an aptamer's specificity or stability are preferred.

II.B.2. Chemical Modifications of Nucleic Acids

Chemical modifications that may be incorporated into aptamers and other nucleic acids include with neither limitation nor exclusivity base modifications, sugar modifications, and backbone modifications.

II.B.2.a. Base Modifications

The base residues in aptamers may be other than naturally occurring bases (e.g., A, G, C, T, U, 5MC, and the like). Derivatives of purines and pyrimidines are known in the art; an exemplary but not exhaustive list includes aziridinylcytosine, 4-acetylcytosine, 5-fluorouracil, 5-bromouracil, 5-carboxymethylaminomethyl-2-thiouracil, 5-carboxymethylaminomethyluracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine (5MC), N6-methyladenine, 7-methylguanine, 5-methylaminomethyluracil, 5-methoxyaminomethyl-2-thiouracil, beta-D-mannosylqueosine, 5-methoxyuracil, 2-methylthio-N-6-isopentenyladenine, uracil-5-oxyacetic acid methylester, pseudouracil, queosine, 2-thiocytosine, 5-methyl-2-thiouracil, 2-thiouracil, 4-thiouracil, 5-methyluracil, uracil-5oxyacetic acid, and 2,6-diaminopurine. In addition to nucleic acids that incorporate one or more of such base derivatives, nucleic acids having nucleotide residues that are devoid of a purine or a pyrimidine base may also be included in aptamers.

II.B.2.b. Sugar Modifications

The sugar residues in aptamers may be other than conventional ribose and deoxyribose residues. By way of non-limiting example, substitution at the 2′-position of the furanose residue enhances nuclease stability. An exemplary, but not exhaustive list, of modified sugar residues includes 2′ substituted sugars such as 2′-O-methyl-, 2′-O-alkyl, 2′-O-allyl, 2′-S-alkyl, 2′-S-allyl, 2′-fluoro-, 2′-halo, or 2′-azido-ribose, carbocyclic sugar analogs, alpha-anomeric sugars, epimeric sugars such as arabinose, xyloses or lyxoses, pyranose sugars, furanose sugars, sedoheptuloses, acyclic analogs and abasic nucleoside analogs such as methyl riboside, ethyl riboside or propylriboside.

II.B.2.c Backbone Modifications

Chemically modified backbones include, by way of non-limiting example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Chemically modified backbones that do not contain a phosphorus atom have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages, including without limitation morpholino linkages; siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; and amide backbones.

II.B.3. Nucleic Acid Targeting Elements

One type of nucleic acid targeting element is an aptamer. In general, techniques for identifying aptamers involve incubating a preselected non-nucleic target molecule with mixtures (2 to 50 members), pools (50 to 5,000 members) or libraries (50 or more members) of different nucleic acids that are potential aptamers under conditions that allow complexes of target molecules and aptamers to form. By “different nucleic acids” it is meant that the nucleotide sequence of each potential aptamer may be different from that of any other member, that is, the sequences of the potential aptamers are random with respect to each other. Randomness can be introduced in a variety of manners such as, e.g., mutagenesis, which can be carried out in vivo by exposing cells harboring a nucleic acid with mutagenic agents, in vitro by chemical treatment of a nucleic acid, or in vitro by biochemical replication (e.g., PCR) that is deliberately allowed to proceed under conditions that reduce fidelity of replication process; randomized chemical synthesis, i.e., by synthesizing a plurality of nucleic acids having a preselected sequence that, with regards to at least one position in the sequence, is random. By “random at a position in a preselected sequence” it is meant that a position in a sequence that is normally synthesized as, e.g., as close to 100% A as possible (e.g., 5′-C-T-T-A-G-T-3′) is allowed to be randomly synthesized at that position (C-T-T-N-G-T, wherein N indicates a randomized position where, for example, the synthesizing reaction contains 25% each of A, T, C and G; or x % A, w % T, y % C and z % G, wherein x+w+y+z=100. In later stages of the process, the sequences are increasingly less randomized and consensus sequences may appear; in any event, it is preferred to ultimately obtain an aptamer having a unique nucleotide sequence.

Aptamers and pools of aptamers are prepared, identified, characterized and/or purified by any appropriate technique, including those utilizing in vitro synthesis, recombinant DNA techniques, PCR amplification, and the like. After their formation, target:aptamer complexes are then separated from the uncomplexed members of the nucleic acid mixture, and the nucleic acids that can be prepared from the complexes are candidate aptamers (at early stages of the technique, the aptamers generally being a population of a multiplicity of nucleotide sequences having varying degrees of specificity for the target). The resulting aptamer (mixture or pool) is then substituted for the starting apatamer (library or pool) in repeated iterations of this series of steps. When a limited number (e.g., a pool or mixture, preferably a mixture with less than 10 members, most preferably 1) of nucleic acids having satisfactory specificity is obtained, the aptamer is sequenced and characterized. Pure preparations of a given aptamer are generated by any appropriate technique (e.g., PCR amplification, in vitro chemical synthesis, and the like).

For example, Tuerk and Gold (Science (1990) 249:505-510) disclose the use of a procedure termed “systematic evolution of ligands by exponential enrichment” (SELEX). In this method, pools of nucleic acid molecules that are randomized at specific positions are subjected to selection for binding to a nucleic acid-binding protein (see, e.g., PCT International Publication No. WO 91/19813 and U.S. Pat. No. 5,270,163). The oligonucleotides so obtained are sequenced and otherwise characterized. Kinzler, K. W., et al. (Nucleic Acids Res. (1989) 17:3645-3653) used a similar technique to identify synthetic double-stranded DNA molecules that are specifically bound by DNA-binding polypeptides. Ellington, A. D., et al. (Nature (1990) 346: 818-822) disclose the production of a large number of random sequence RNA molecules and the selection and identification of those that bind specifically to specific dyes such as Cibacron blue.

Another technique for identifying nucleic acids that bind non-nucleic target molecules is the oligonucleotide combinatorial technique disclosed by Ecker, D. J. et al. (Nuc. Acids Res. 21, 1853 (1993)) known as “synthetic unrandomization of randomized fragments” (SURF), which is based on repetitive synthesis and screening of increasingly simplified sets of oligonucleotide analogue libraries, pools and mixtures (Tuerk, C. and Gold, L. (Science 249, 505 (1990)). The starting library consists of oligonucleotide analogues of defined length with one position in each pool containing a known analogue and the remaining positions containing equimolar mixtures of all other analogues. With each round of synthesis and selection, the identity of at least one position of the oligomer is determined until the sequences of optimized nucleic acid ligand aptamers are discovered.

Once a particular candidate aptamer has been identified through a SURF, SELEX or any other technique, its nucleotide sequence can be determined (as is known in the art), and its three-dimensional molecular structure can be examined by nuclear magnetic resonance (NMR). These techniques are explained in relation to the determination of the three-dimensional structure of a nucleic acid ligand that binds thrombin in Padmanabhan, K. et al., J. Biol. Chem. 24, 17651 (1993); Wang, K. Y. et al., Biochemistry 32, 1899 (1993); and Macaya, R. F. et al., Proc. Nat'l. Acad. Sci. USA 90, 3745 (1993). Selected aptamers may be resynthesized using one or more modified bases, sugars or backbone linkages. Aptamers consist essentially of the minimum sequence of nucleic acid needed to confer binding specificity, but may be extended on the 5′ end, the 3′ end, or both, or may be otherwise derivatized or conjugated.

II.B.4. Biologically Active Nucleic Acids

Bioactive nucleic acids, and/or templates therefor, can be a bioactive portion of a complex or compound of the invention. By way of non-limiting example, a bioactive nucleic acid may be an antisense oligonucleotide, an aptamer, an antisense transcript, an enzymatic nucleic acid such as a ribozyme, a ribosomal RNA (rRNA), a transfer RNA (tRNA), or a molecular decoy.

A variety of chemical types and structural forms of nucleic acids can be biologically active. These include, by way of non-limiting example, DNA, including single-stranded (ssDNA) and double-stranded (dsRNA); RNA, including but not limited to ssRNA, dsRNA, tRNA, mRNA, rRNA, enzymatic RNA; RNA:DNA hybrids; triplexed DNA (e.g., dsDNA in association with a short oligonucleotide); and the like.

The sequence of a nucleic acid may be a template for a bioactive nucleic acid such as an antisense transcript or a ribozyme. The nucleic acid sequence may be an ORF (open reading frame) that encodes a polypeptide. ORFs of particular interest in this aspect of the invention include but are not limited to ones that encode a polypeptide that is absent or deficient in a cell; a polypeptide activity or expression of which increased or decreased for therapeutic benefit or diagnostic use; a dominant negative mutant of a polypeptide the activity of which is increased or decreased for therapeutic benefit or diagnostic use; and a detectable polypeptide, which may be used in diagnostic applications.

II.C. Polypeptides and Derivatives

As used herein, the term “polypeptide” includes proteins, fusion proteins, oligopeptides and polypeptide derivatives, with the exception that peptidomimetics are considered to be small molecules herein. Antibodies and antibody derivatives are disclosed in a separate section, but antibodies and antibody derivatives are, for purposes of the invention, treated as a subclass of the polypeptides and derivatives.

A “protein” is a molecule having a sequence of amino acids that are linked to each other in a linear molecule by peptide bonds. The term protein refers to a polypeptide that is isolated from a natural source, or produced from an isolated cDNA using recombinant DNA technology; and has a sequence of amino acids having a length of at least about 200 amino acids.

A “fusion protein” is a type of protein that has an amino acid sequence that results from the linkage of the amino acid sequences of two or more normally separate polypeptides and which is encoded by a chimeric reading frame. Methods of preparing and using fusion proteins are disclosed in U.S. patent application Ser. No. 60/237,929 (attorney docket No. 030854.0009 entitled “Genetic Fusions of pIgR Ligands and Biologically Active Polypeptides for the Delivery of Therapeutic and Diagnostic Proteins” by Houston, L. L., Glynn, Jacqueline M., and Sheridan, Philip L.), filed Oct. 2, 2000, which is incorporated in its entirety herein.

A “protein fragment” is a proteolytic fragment of a larger polypeptide, which may be a protein or a fusion protein. A proteolytic fragment may be prepared by in vivo or in vitro proteolytic cleavage of a larger polypeptide, and is generally too large to be prepared by chemical synthesis. Proteolytic fragments have amino acid sequences having a length from about 200 to about 1,000 amino acids.

An “oligopeptide” is a polypeptide having a short amino acid sequence (i.e., 2 to about 200 amino acids). An oligopeptide is generally prepared by chemical synthesis.

Although oligopeptides and protein fragments may be otherwise prepared, it is possible to use recombinant DNA technology and/or in vitro biochemical manipulations. For example, a nucleic acid encoding an amino acid sequence may be prepared and used as a template for in vitro transcription/translation reactions. In such reactions, an exogenous nucleic acid encoding a preselected polypeptide is introduced into a mixture that is essentially depleted of exogenous nucleic acids that contains all of the cellular components required for transcription and translation. One or more radiolabeled amino acids are added before or with the exogenous DNA, and transcription and translation are allowed to proceed. Because the only nucleic acid present in the reaction mix is the exogenous nucleic acid added to the reaction, only polypeptides encoded thereby are produced, and incorporate the radiolabelled amino acid(s). In this manner, polypeptides encoded by a preselected exogenous nucleic acid are radiolabeled. Although other proteins are present in the reaction mix, the preselected polypeptide is the only one that is produced in the presence of the radiolabeled amino acids and is thus uniquely labeled.

As is explained in detail below, “polypeptide derivatives” include without limitation mutant polypeptides, chemically modified polypeptides, and peptidomimetics.

The polypeptides of this invention, including the analogs and other modified variants, may generally be prepared following known techniques. Preferably, synthetic production of the polypeptide of the invention may be according to the solid phase synthetic method. For example, the solid phase synthesis is well understood and is a common method for preparation of polypeptides, as are a variety of modifications of that technique [Merrifield (1964), J. Am. Chem. Soc., 85: 2149; Stewart and Young (1984), Solid Phase polypeptide Synthesis, Pierce Chemical Company, Rockford, Ill.; Bodansky and Bodanszky (1984), The Practice of polypeptide Synthesis, Springer-Verlag, New York; Atherton and Sheppard (1989), Solid Phase polypeptide Synthesis: A Practical Approach, IRL Press, New York]. See, also, the specific method disclosed in Example 1 below.

Alternatively, polypeptides of this invention may be prepared in recombinant systems using polynucleotide sequences encoding the polypeptides. For example, fusion proteins are typically prepared using recombinant DNA technology.

II.C.1. Polypeptide Derivatives

A “derivative” of a polypeptide is a compound that is not, by definition, a polypeptide, i.e., it contains at least one chemical linkage that is not a peptide bond. Thus, polypeptide derivatives include without limitation proteins that naturally undergo post-translational modifications such as, e.g., glycosylation. It is understood that a polypeptide of the invention may contain more than one of the following modifications within the same polypeptide. Preferred polypeptide derivatives retain a desirable attribute, which may be biological activity; more preferably, a polypeptide derivative is enhanced with regard to one or more desirable attributes, or has one or more desirable attributes not found in the parent polypeptide. Although they are described in this section, peptidomimetics are taken as small molecules in the present disclosure.

II.C.1.a. Mutant Polypeptides

A polypeptide having an amino acid sequence identical to that found in a protein prepared from a natural source is a “wildtype” polypeptide. Mutant oligopeptides can be prepared by chemical synthesis, including without limitation combinatorial synthesis.

Mutant polypeptides larger than oligopeptides can be prepared using recombinant DNA technology by altering the nucleotide sequence of a nucleic acid encoding a polypeptide. Although some alterations in the nucleotide sequence will not alter the amino acid sequence of the polypeptide encoded thereby (“silent” mutations), many will result in a polypeptide having an altered amino acid sequence that is altered relative to the parent sequence. Such altered amino acid sequences may comprise substitutions, deletions and additions of amino acids, with the proviso that such amino acids are naturally occurring amino acids.

Thus, subjecting a nucleic acid that encodes a polypeptide to mutagenesis is one technique that can be used to prepare mutant polypeptides, particularly ones having substitutions of amino acids but no deletions or insertions thereof. A variety of mutagenic techniques are known that can be used in vitro or in vivo including without limitation chemical mutagenesis and PCR-mediated mutagenesis. Such mutagenesis may be randomly targeted (i.e., mutations may occur anywhere within the nucleic acid) or directed to a section of the nucleic acid that encodes a stretch of amino acids of particular interest. Using such techniques, it is possible to prepare randomized, combinatorial or focused compound libraries, pools and mixtures.

Polypeptides having deletions or insertions of naturally occurring amino acids may be synthetic oligopeptides that result from the chemical synthesis of amino acid sequences that are based on the amino acid sequence of a parent polypeptide but which have one or more amino acids inserted or deleted relative to the sequence of the parent polypeptide. Insertions and deletions of amino acid residues in polypeptides having longer amino acid sequences may be prepared by directed mutagenesis.

II.C.1.b. Chemically Modified Polypeptides

As contemplated by this invention, the term “polypeptide” includes those having one or more chemical modification relative to another polypeptide, i.e., chemically modified polypeptides. The polypeptide from which a chemically modified polypeptide is derived may be a wildtype protein, a mutant protein or a mutant polypeptide, or polypeptide fragments thereof; an antibody or other polypeptide ligand according to the invention including without limitation single-chain antibodies, bacterial proteins and polypeptide derivatives thereof; or polypeptide ligands prepared according to the disclosure. Preferably, the chemical modification(s) confer(s) or improve(s) desirable attributes of the polypeptide but does not substantially alter or compromise the biological activity thereof. Desirable attributes include but are limited to increased shelf-life; enhanced serum or other in vivo stability; resistance to proteases; and the like. Such modifications include by way of non-limiting example N-terminal acetylation, glycosylation, and biotinylation.

II.C.1.b.1. Polypeptides with N-Terminal or C-Terminal Chemical Groups

An effective approach to confer resistance to peptidases acting on the N-terminal or C-terminal residues of a polypeptide is to add chemical groups at to one or both of the polypeptide termini, such that the modified polypeptide is no longer a substrate for the peptidase. One such chemical modification is glycosylation of the polypeptides at either or both termini. Certain chemical modifications, in particular N-terminal glycosylation, have been shown to increase the stability of polypeptides in human serum (Powell et al. (1993), Pharma. Res. 10: 1268-1273). Other chemical modifications which enhance serum stability include, but are not limited to, the addition of an N-terminal alkyl group, consisting of a lower alkyl of from 1 to 20 carbons, such as an acetyl group, and/or the addition of a C-terminal amide or substituted amide group.

II.C.1.b.2. Polypeptides with a Terminal D-Amino Acid

The presence of an N-terminal D-amino acid increases the serum stability of a polypeptide that otherwise contains L-amino acids, because exopeptidases acting on the N-terminal residue cannot utilize a D-amino acid as a substrate. Similarly, the presence of a C-terminal D-amino acid also stabilizes a polypeptide, because serum exopeptidases acting on the C-terminal residue cannot utilize a D-amino acid as a substrate. With the exception of these terminal modifications, the amino acid sequences of polypeptides with N-terminal and/or C-terminal D-amino acids are usually identical to the sequences of the parent L-amino acid polypeptide.

II.C.1.b.3. Polypeptides with Substitution of Natural Amino Acids by Unnatural Amino Acids

Substitution of unnatural amino acids for natural amino acids in a subsequence of a polypeptide can confer or enhance desirable attributes including biological activity. Such a substitution can, for example, confer resistance to proteolysis by exopeptidases acting on the N-terminus. The synthesis of polypeptides with unnatural amino acids is routine and known in the art (see, for example, Coller, et al. (1993), cited above).

II.C.1.b.4. Post-Translational Chemical Modifications

Different host cells will contain different post-translational modification mechanisms that may provide particular types of post-translational modification of a fusion protein if the amino acid sequences required for such modifications is present in the fusion protein. A large number (˜100) of post-translational modifications have been described, a few of which are discussed herein. One skilled in the art will be able to choose appropriate host cells, and design chimeric genes that encode protein members comprising the amino acid sequence needed for a particular type of modification.

Glycosylation is one type of post-translational chemical modification that occurs in many eukaryotic systems, and may influence the activity, stability, pharmacogenetics, immunogenicity and/or antigenicity of proteins. However, specific amino acids must be present at such sites to recruit the appropriate glycosylation machinery, and not all host cells have the appropriate molecular machinery. Saccharomyces cerevisieae and Pichia pastoris provide for the production of glycosylated proteins, as do expression systems that utilize insect cells, although the pattern of glyscoylation may vary depending on which host cells are used to produce the fusion protein.

Another type of post-translation modification is the phosphorylation of a free hydroxyl group of the side chain of one or more Ser, Thr or Tyr residues. Protein kinases catalyze such reactions. Phosphorylation is often reversible due to the action of a protein phosphatase, an enzyme that catalyzes the dephosphorylation of amino acid residues.

Differences in the chemical structure of amino terminal residues result from different host cells, each of which may have a different chemical version of the methionine residue encoded by a start codon, and these will result in amino termini with different chemical modifications.

For example, many or most bacterial proteins are synthesized with an amino terminal amino acid that is a modified form of methionine, i.e, N-formyl-methionine (fMet). Although the statement is often made that all bacterial proteins are synthesized with an fMet initiator amino acid; although this may be true for E. coli, recent studies have shown that it is not true in the case of other bacteria such as Pseudomonas aeruginosa (Newton et al., J. Biol. Chem. 274:22143-22146, 1999). In any event, in E. coli, the formyl group of fMet is usually enzymatically removed after translation to yield an amino terminal methionine residue, although the entire fMet residue is sometimes removed (see Hershey, Chapter 40, “Protein Synthesis” in: Escherichia Coli and Salmonella Typhimurium: Cellular and Molecular Biology, Neidhardt, Frederick C., Editor in Chief, American Society for Microbiology, Washington, D.C., 1987, Volume 1, pages 613-647, and references cited therein.) E. coli mutants that lack the enzymes (such as, e.g., formylase) that catalyze such post-translational modifications will produce proteins having an amino terminal fMet residue (Guillon et al., J. Bacteriol. 174:4294-4301, 1992).

In eukaryotes, acetylation of the initiator methionine residue, or the penultimate residue if the initiator methionine has been removed, typically occurs co- or post-translationally. The acetylation reactions are catalyzed by N-terminal acetyltransferases (NATs, a.k.a. N-alpha-acetyltransferases), whereas removal of the initiator methionine residue is catalyzed by methionine aminopeptidases (for reviews, see Bradshaw et al., Trends Biochem. Sci. 23:263-267, 1998; and Driessen et al., CRC Crit. Rev. Biochem. 18:281-325, 1985). Amino terminally acetylated proteins are said to be “N-acetylated,” “N alpha acetylated” or simply “acetylated.”

Another post-translational process that occurs in eukaryotes is the alpha-amidation of the carboxy terminus. For reviews, see Eipper et al. Annu. Rev. Physiol. 50:333-344, 1988, and Bradbury et al. Lung Cancer 14:239-251, 1996. About 50% of known endocrine and neuroendocrine peptide hormones are alpha-amidated (Treston et al., Cell Growth Differ. 4:911-920, 1993). In most cases, carboxy alpha-amidation is required to activate these peptide hormones.

II.D. Peptidomimetics

In general, a polypeptide mimetic (“peptidomimetic”) is a molecule that mimics the biological activity of a polypeptide but is no longer peptidic in chemical nature. By strict definition, a peptidomimetic is a molecule that contains no peptide bonds (that is, amide bonds between amino acids). However, the term peptidomimetic is sometimes used to describe molecules that are no longer completely peptidic in nature, such as pseudo-peptides, semi-peptides and peptoids. Examples of some peptidomimetics by the broader definition (where part of a polypeptide is replaced by a structure lacking peptide bonds) are described below. Whether completely or partially non-peptide, peptidomimetics according to this invention provide a spatial arrangement of reactive chemical moieties that closely resembles the three-dimensional arrangement of active groups in the polypeptide on which the peptidomimetic is based. As a result of this similar active-site geometry, the peptidomimetic has effects on biological systems that are similar to the biological activity of the polypeptide.

There are several potential advantages for using a mimetic of a given polypeptide rather than the polypeptide itself. For example, polypeptides may exhibit two undesirable attributes, i.e., poor bioavailability and short duration of action. Peptidomimetics are often small enough to be both orally active and to have a long duration of action. There are also problems associated with stability, storage and immunoreactivity for polypeptides that are not experienced with peptidomimetics.

Candidate, lead and other polypeptides having a desired biological activity can be used in the development of peptidomimetics with similar biological activities. Techniques of developing peptidomimetics from polypeptides are known. Peptide bonds can be replaced by non-peptide bonds that allow the peptidomimetic to adopt a similar structure, and therefore biological activity, to the original polypeptide. Further modifications can also be made by replacing chemical groups of the amino acids with other chemical groups of similar structure. The development of peptidomimetics can be aided by determining the tertiary structure of the original polypeptide, either free or bound to a ligand, by NMR spectroscopy, crystallography and/or computer-aided molecular modeling. These techniques aid in the development of novel compositions of higher potency and/or greater bioavailability and/or greater stability than the original polypeptide (Dean (1994), BioEssays, 16: 683-687; Cohen and Shatzmiller (1993), J. Mol. Graph., 11: 166-173; Wiley and Rich (1993), Med. Res. Rev., 13: 327-384; Moore (1994), Trends Pharmacol. Sci., 15: 124-129; Hruby (1993), Biopolymers, 33: 1073-1082; Bugg et al. (1993), Sci. Am., 269: 92-98, all incorporated herein by reference].

Thus, through use of the methods described above, the present invention provides compounds exhibiting enhanced therapeutic activity in comparison to the polypeptides described above. The peptidomimetic compounds obtained by the above methods, having the biological activity of the above named polypeptides and similar three-dimensional structure, are encompassed by this invention. It will be readily apparent to one skilled in the art that a peptidomimetic can be generated from any of the modified polypeptides described in the previous section or from a polypeptide bearing more than one of the modifications described from the previous section. It will furthermore be apparent that the peptidomimetics of this invention can be further used for the development of even more potent non-peptidic compounds, in addition to their utility as therapeutic compounds.

Specific examples of peptidomimetics derived from the polypeptides described in the previous section are presented below. These examples are illustrative and not limiting in terms of the other or additional modifications.

II.D.1. Peptides with a Reduced Isostere Pseudopeptide Bond

Proteases act on peptide bonds. It therefore follows that substitution of peptide bonds by pseudopeptide bonds confers resistance to proteolysis. A number of pseudopeptide bonds have been described that in general do not affect polypeptide structure and biological activity. The reduced isostere pseudopeptide bond is a suitable pseudopeptide bond that is known to enhance stability to enzymatic cleavage with no or little loss of biological activity (Couder, et al. (1993), Int. J. Polypeptide Protein Res. 41:181-184, incorporated herein by reference). Thus, the amino acid sequences of these compounds may be identical to the sequences of their parent L-amino acid polypeptides, except that one or more of the peptide bonds are replaced by an isostere pseudopeptide bond. Preferably the most N-terminal peptide bond is substituted, since such a substitution would confer resistance to proteolysis by exopeptidases acting on the N-terminus.

II.D.2. Peptides with a Retro-Inverso Pseudopeptide Bond

To confer resistance to proteolysis, peptide bonds may also be substituted by retro-inverso pseudopeptide bonds (Dalpozzo, et al. (1993), Int. J. Polypeptide Protein Res. 41:561-566, incorporated herein by reference). According to this modification, the amino acid sequences of the compounds may be identical to the sequences of their L-amino acid parent polypeptides, except that one or more of the peptide bonds are replaced by a retro-inverso pseudopeptide bond. Preferably the most N-terminal peptide bond is substituted, since such a substitution will confer resistance to proteolysis by exopeptidases acting on the N-terminus.

II.D.3. Peptoid Derivatives

Peptoid derivatives of polypeptides represent another form of modified polypeptides that retain the important structural determinants for biological activity, yet eliminate the peptide bonds, thereby conferring resistance to proteolysis (Simon, et al., 1992, Proc. Natl. Acad. Sci. USA, 89:9367-9371 and incorporated herein by reference). Peptoids are oligomers of N-substituted glycines. A number of N-alkyl groups have been described, each corresponding to the side chain of a natural amino acid.

III. Antibodies, Including Monoclonal Antibodies

The term “antibody” is meant to encompass an immunoglobulin molecule obtained by in vitro or in vivo generation of an immunogenic response, and includes both polyclonal, monospecific and monoclonal antibodies. An “immunogenic response” is one that results in the production of antibodies directed to one or more proteins after the appropriate cells have been contacted with such proteins, or polypeptide derivatives thereof, in a manner such that one or more portions of the protein function as epitopes. An epitope is a single antigenic determinant in a molecule. In proteins, particularly denatured proteins, an epitope is typically defined and represented by a contiguous amino acid sequence. However, in the case of nondenatured proteins, epitopes also include structures, such as active sites, that are formed by the three-dimensional folding of a protein in a manner such that amino acids from separate portions of the amino acid sequence of the protein are brought into close physical contact with each other.

Wildtype antibodies have four polypeptide chains, two identical heavy chains and two identical light chains. Both types of polypeptide chains have constant regions, which do not vary or vary minimally among antibodies of the same class (i.e, IgA, IgM, etc.), and variable regions. As is explained below, variable regions are unique to a particular antibody and comprise a recognition element for an epitope.

Each light chain of an antibody is associated with one heavy chain, and the two chains are linked by a disulfide bridge formed between cysteine residues in the carboxy-terminal region of each chain, which is distal from the amino terminal region of each chain that constitutes its portion of the antigen binding domain. Antibody molecules are further stabilized by disulfide bridges between the two heavy chains in an area known as the hinge region, at locations nearer the carboxy terminus of the heavy chains than the locations where the disulfide bridges between the heavy and light chains are made. The hinge region also provides flexibility for the antigen-binding portions of an antibody.

An antibody's specificity is determined by the variable regions located in the amino terminal regions of the light and heavy chains. The variable regions of a light chain and associated heavy chain form an “antigen binding domain” that recognizes a specific epitope; an antibody thus has two antigen binding domains. The antigen binding domains in a wildtype antibody are directed to the same epitope of an immunogenic protein, and a single wildtype antibody is thus capable of binding two molecules of the immunogenic protein at the same time.

III.A. Types of Antibodies

Compositions of antibodies have, depending on the manner in which they are prepared, different types of antibodies. Types of antibodies of particular interest include polyclonal, monospecific and monoclonal antibodies.

Polyclonal antibodies are generated in an immunogenic response to a protein having many epitopes. A composition of polyclonal antibodies thus includes a variety of different antibodies directed to the same and to different epitopes within the protein. Methods for producing polyclonal antibodies are known in the art (see, e.g., Cooper et al., Section III of Chapter 11 in: Short Protocols in Molecular Biology, 2nd Ed., Ausubel et al., eds., John Wiley and Sons, New York, 1992, pages 11-37 to 11-41).

Monospecific antibodies (a.k.a. antipeptide antibodies) are generated in a humoral response to a short (typically, 5 to 20 amino acids) immunogenic polypeptide that corresponds to a few (preferably one) isolated epitopes of the protein from which it is derived. A plurality of monospecific antibodies includes a variety of different antibodies directed to a specific portion of the protein, i.e, to an amino acid sequence that contains at least one, preferably only one, epitope. Methods for producing monospecific antibodies are known in the art (see, e.g., Cooper et al., Section III of Chapter 11 in: Short Protocols in Molecular Biology, 2nd Ed., Ausubel et al., eds., John Wiley and Sons, New York, 1992, pages 11-42 to 11-46).

A monoclonal antibody is a specific antibody that recognizes a single specific epitope of an immunogenic protein. In a plurality of a monoclonal antibody, each antibody molecule is identical to the others in the plurality. In order to isolate a monoclonal antibody, a clonal cell line that expresses, displays and/or secretes a particular monoclonal antibody is first identified; this clonal cell line can be used in one method of producing the antibodies of the invention. Methods for the preparation of clonal cell lines and of monoclonal antibodies expressed thereby are known in the art (see, for example, Fuller et al., Section II of Chapter 11 in: Short Protocols in Molecular Biology, 2nd Ed., Ausubel et al., eds., John Wiley and Sons, New York, 1992, pages 11-22 to 11-11-36).

Variants and derivatives of antibodies include antibody and T-cell receptor fragments that retain the ability to specifically bind to antigenic determinants. Preferred fragments include Fab fragments (i.e, an antibody fragment that contains the antigen-binding domain and comprises a light chain and part of a heavy chain bridged by a disulfide bond); Fab′ (an antibody fragment containing a single anti-binding domain comprising an Fab and an additional portion of the heavy chain through the hinge region); F(ab′)2 (two Fab′ molecules joined by interchain disulfide bonds in the hinge regions of the heavy chains; the Fab′ molecules may be directed toward the same or different epitopes); a bispecific Fab (an Fab molecule having two antigen binding domains, each of which may be directed to a different epitope); a single chain Fab chain comprising a variable region, a.k.a., a sFv (the variable, antigen-binding determinative region of a single light and heavy chain of an antibody linked together by a chain of 10-25 amino acids); a disulfide-linked Fv, or dsFv (the variable, antigen-binding determinative region of a single light and heavy chain of an antibody linked together by a disulfide bond); a camelized VH (the variable, antigen-binding determinative region of a single heavy chain of an antibody in which some amino acids at the VH interface are those found in the heavy chain of naturally occurring camel antibodies); a bispecific sFv (a sFv or a dsFv molecule having two antigen-binding domains, each of which may be directed to a different epitope); a diabody (a dimerized sFv formed when the VH domain of a first sFv assembles with the VL domain of a second sFv and the VL domain of the first sFv assembles with the VH domain of the second sFv; the two antigen-binding regions of the diabody may be directed towards the same or different epitopes); and a triabody (a trimerized sFv, formed in a manner similar to a diabody, but in which three antigen-binding domains are created in a single complex; the three antigen binding domains may be directed towards the same or different epitopes). Derivatives of antibodies also include one or more CDR sequences of an antibody combining site. The CDR sequences may be linked together on a scaffold when two or more CDR sequences are present.

The term “antibody” also includes genetically engineered antibodies and/or antibodies produced by recombinant DNA techniques and “humanized” antibodies. Humanized antibodies have been modified, by genetic manipulation and/or in vitro treatment to be more human, in terms of amino acid sequence, glycosylation pattern, etc., in order to reduce the antigenicity of the antibody or antibody fragment in an animal to which the antibody is intended to be administered (Gussow et al., Methods Enz. 203:99-121, 1991).

III.B. Methods of Preparing Antibodies and Antibody Variants

The antibodies and antibody fragments of the invention may be produced by any suitable method, for example, in vivo (in the case of polyclonal and monospecific antibodies), in cell culture (as is typically the case for monoclonal antibodies, wherein hybridoma cells expressing the desired antibody are cultured under appropriate conditions), in in vitro translation reactions, and in recombinant DNA expression systems (the latter method of producing proteins is disclosed in more detail herein in the section entitled “Methods of Producing Fusion Proteins”). Antibodies and antibody variants can be produced from a variety of animal cells, preferably from mammalian cells, with murine and human cells being particularly preferred. Antibodies that include non-naturally occurring antibody and T-cell receptor variants that retain only the desired antigen targeting capability conferred by an antigen binding site(s) of an antibody can be produced by known cell culture techniques and recombinant DNA expression systems (see, e.g., Johnson et al., Methods in Enzymol. 203:88-98, 1991; Molloy et al., Mol. Immunol. 32:73-81, 1998; Schodin et al., J. Immunol. Methods 200:69-77, 1997). Recombinant DNA expression systems are typically used in the production of antibody variants such as, e.g., bispecific antibodies and sFv molecules. Preferred recombinant DNA expression systems include those that utilize host cells and expression constructs that have been engineered to produce high levels of a particular protein. Preferred host cells and expression constructs include Escherichia coli; harboring expression constructs derived from plasmids or viruses (bacteriophage); yeast such as Saccharomyces cerevisiae or Pichia pastoris harboring episomal or chromosomally integrated expression constructs; insect cells and viruses such as Sf9 cells and baculovirus; and mammalian cells harboring episomal or chromosomally integrated (e.g., retroviral) expression constructs (for a review, see Verna et al., J. Immunol. Methods 216:165-181, 1998). Antibodies can also be produced in plants (U.S. Pat. No. 6,046,037; Ma et al., Science 268:716-719, 1995) or by phage display technology (Winter et al., Annu. Rev. Immunol. 12:433-455, 1994).

XenoMouse strains are genetically engineered mice in which the murine IgH and Igk loci have been functionally replaced by their Ig counterparts on yeast artificial YAC transgenes. These human Ig transgenes can carry the majority of the human variable repertoire and can undergo class switching from IgM to IgG isotypes. The immune system of the xenomouse recognizes administered human antigens as foreign and produces a strong humoral response. The use of XenoMouse in conjunction with well-established hybridomas techniques, results in fully human IgG mAbs with sub-nanomolar affinities for human antigens (see U.S. Pat. No. 5,770,429, entitled “Transgenic non-human animals capable of producing heterologous antibodies”; U.S. Pat. No. 6,162,963, entitled “Generation of Xenogenetic antibodies”; U.S. Pat. No. 6,150,584, entitled “Human antibodies derived from immunized xenomice”; U.S. Pat. No. 6,114,598, entitled Generation of xenogeneic antibodies; and U.S. Pat. No. 6,075,181, entitled “Human antibodies derived from immunized xenomice”; for reviews, see Green, Antibody engineering via genetic engineering of the mouse: XenoMouse strains are a vehicle for the facile generation of therapeutic human monoclonal antibodies, J. Immunol. Methods 231:11-23, 1999; Wells, Eek, a XenoMouse: Abgenix, Inc., Chem Biol 2000 August; 7(8):R185-6; and Davis et al., Transgenic mice as a source of fully human antibodies for the treatment of cancer, Cancer Metastasis Rev 1999; 18(4):421-5).

IV. Fusion Proteins

One type of compound of the invention is a fusion protein. A “fusion protein” is a single protein having amino acid sequences derived from two or more normally separate proteins, and which is encoded by a chimeric reading frame.

U.S. patent application Ser. No. 60/237,929 (attorney docket Nos. 030854.0009 and 057220.0301) entitled “Genetic Fusions of pIgR Ligands and Biologically Active Polypeptides for the Delivery of Therapeutic and Diagnostic Proteins” by Houston, L. L., Glynn, Jacqueline M., and Sheridan, Philip L., filed Oct. 2, 2000, is drawn to fusion proteins comprising pIgR ligands and biologically active polypeptides.

IV.A. Structure of Fusion Proteins and Chimeric Reading Frames

Polypeptides, which are polymers of amino acids, are encoded by another class of molecules, known as nucleic acids, which are polymers of structural units known as nucleotides. In particular, proteins are encoded by nucleic acids known as DNA and RNA (deoxyribonucleic acid and ribonucleic acid, respectively).

The nucleotide sequence of a nucleic acid contains the “blueprints” for a protein. Nucleic acids are polymers of nucleotides, four types of which are present in a given nucleic acid. The nucleotides in DNA are adenine, cytosine and guanine and thymine, (represented by A, C, G, and T respectively); in RNA, thymine (T) is replaced by uracil (U). The structures of nucleic acids are represented by the sequence of its nucleotides arranged in a 5′ (“5 prime”) to 3′ (“3 prime”) direction, e.g.,

5′-A-T-G-C-C-T-A-A-A-G-C-C-G-C-T-C-C-C-T-C-A-3′

In biological systems, proteins are typically produced in the following manner. A DNA molecule that has a nucleotide sequence that encodes the amino acid sequence of a protein is used as a template to guide the production of a messenger RNA (mRNA) that also encodes the protein; this process is known as transcription. In a subsequent process called translation, the mRNA is “read” and directs the synthesis of a protein having a particular amino acid sequence.

Each amino acid in a protein is encoded by a series of three contiguous nucleotides, each of which is known as a codon. In the “genetic code,” some amino acids are encoded by several codons, each codon having a different sequence; whereas other amino acids are encoded by only one codon sequence. An entire protein (i.e., a complete amino acid sequence) is encoded by a nucleic acid sequence called a reading frame. A reading frame is a continuous nucleotide sequence that encodes the amino acid sequence of a protein; the boundaries of a reading frame are defined by its initiation (start) and termination (stop) codons.

The process by which a protein is produced from a nucleic acid can be diagrammed as follows:

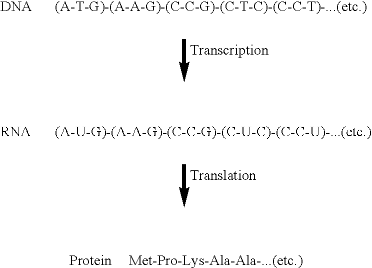

A chimeric reading frame encoding a fusion protein is prepared as follows. A “chimeric reading frame” is a genetically engineered reading frame that results from the fusion of two or more normally distinct reading frames, or fragments thereof, each of which normally encodes a separate polypeptide. Using recombinant DNA techniques, a first reading frame that encodes a first amino acid sequence is linked to a second reading frame that encodes a second amino acid sequence in order to generate a chimeric reading frame. Chimeric reading frames may also include nucleotide sequences that encode optional fusion protein elements (see below). A hypothetical example of a chimeric reading frame created from two normally separate reading frames is depicted in the following flowchart.

A first Reading Frame and “Protein-1”:

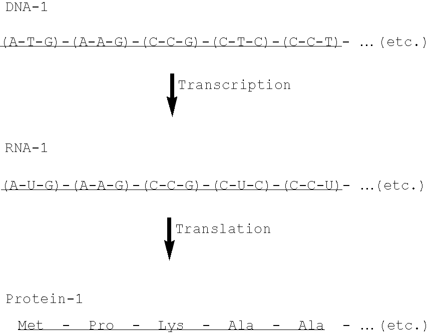

A second Reading Frame and “Protein-2”:

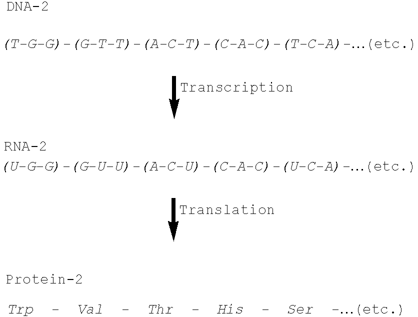

Chimeric Reading Frame that encodes a Fusion Protein that has sequences from Protein-1 and Protein-2:

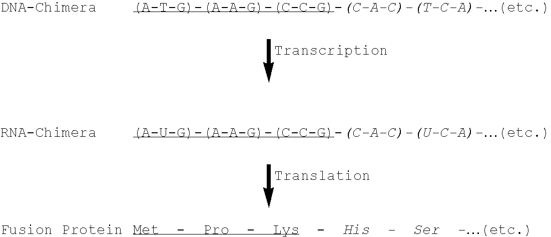

In order for a chimeric reading frame to be functional, each normally distinct reading frame therein must be fused to all of the other normally distinct reading frames in a manner such that all of the reading frames are in frame with each other. By “in frame with each other” it is meant that, in a chimeric reading frame, a first nucleic acid having a first reading frame is covalently linked to a second nucleic acid having a second reading frame in such a manner that the two reading frames are “read” (translated) in register with each other. As a result, the chimeric reading frame encodes one extended amino acid sequence that includes the amino acid sequences encoded by each of the normally separate reading frames.

A fusion protein of the invention comprises a polypeptide having the amino acid sequence of a monoclonal antibody and a polypeptide that is a targeting element. The targeting element may be an antibody derivative, such as a single-chain antibody, or some other polypeptide capable of binding the molecular target. Non-limiting examples of polypeptides that are pIgR-targeting elements are described in Example 1.

Methods of preparing fusion proteins are known in the art. White et al. (Protein Expr Purif 21:446-455, 2001) describe cloning vectors that allow for the creation of fusion proteins having the framework (part of the constant region) of an IgG molecule linked to an amino-terminal domain that is introduced thereinto via genetic manipulation. One method of generating the fusion proteins of the invention is to use PCR and other cloning techniques to introduce the variable regions of a monoclonal antibody into such vectors, and adding to the amino terminus an amino acid sequence of a polypeptide that is a targeting element.

IV.B. Optional Fusion Protein Elements

In addition to pIgR targeting elements and biologically active polypeptides, the fusion proteins of the invention may further comprise one or more non-biologically active amino acid sequences, i.e., optional fusion protein elements. Such non biologically active elements include, but are not limited to, the following optional fusion protein elements. It is understood that a chimeric reading frame will include nucleotide sequences that encode such optional elements, and that these nucleotide sequences will be positioned so as to be in frame with the reading frame encoding the fusion protein. Optional fusion protein elements may be inserted between the pIgR-targeting element and the biologically active polypeptide, upstream or downstream (amino proximal and carboxy proximal, respectively) of these and other elements, or within the pIgR-targeting element and the biologically active polypeptide. A person skilled in the art will be able to determine which optional element(s) should be included in a fusion protein of the invention, and in what order, based on the desired method of production or intended use of the fusion protein.

Protein delivery elements are optional fusion protein elements that facilitate the uptake of a protein into cells but which are not pIgR targeting elements. The ETA (detoxified exotoxin A) protein delivery element is described in U.S. Pat. No. 6,086,900 to Draper. The VP22 protein delivery element is derived from herpes simplex virus-1 and vectors containing sequences encoding the VP22 protein delivery element are commercially available from Invitrogen (Carlsbad, Calif.; see also U.S. Pat. No. 6,017,735 to Ohare et al.). The Tat protein delivery element is derived from the amino acid sequence of the Tat protein of human immunodeficiency virus (HIV). See U.S. Pat. Nos. 5,804,604; 5,747,641; and 5,674,980.

Organellar delivery elements are optional fusion protein elements that direct a fusion protein into or out of a specific organelle or organelles. For example, the ricin A chain can be included in a fusion protein to mediate its delivery from the endosome into the cytosol. Additionally or alternatively, delivery elements for other organelles or subcellular spaces such as the nucleus, nucleolus, mitochondria, the Golgi apparatus, the endoplasmic reticulum (ER), the cytoplasm, etc. Mammalian expression constructs that incorporate organellar delivery elements are commercially available from Invitrogen (Carlsbad, Calif.: pShooter™ vectors). An H/KDEL (i.e, His/Lys-Asp-Glu-Leu sequence) may be incorporated into a fusion protein of the invention, preferably at the carboxy-terminus, in order to direct a fusion protein to the ER (see Andres et al., J. Biol. Chem. 266:14277-142782, 1991; and Pelham, Trends Bio. Sci. 15:483-486, 1990).

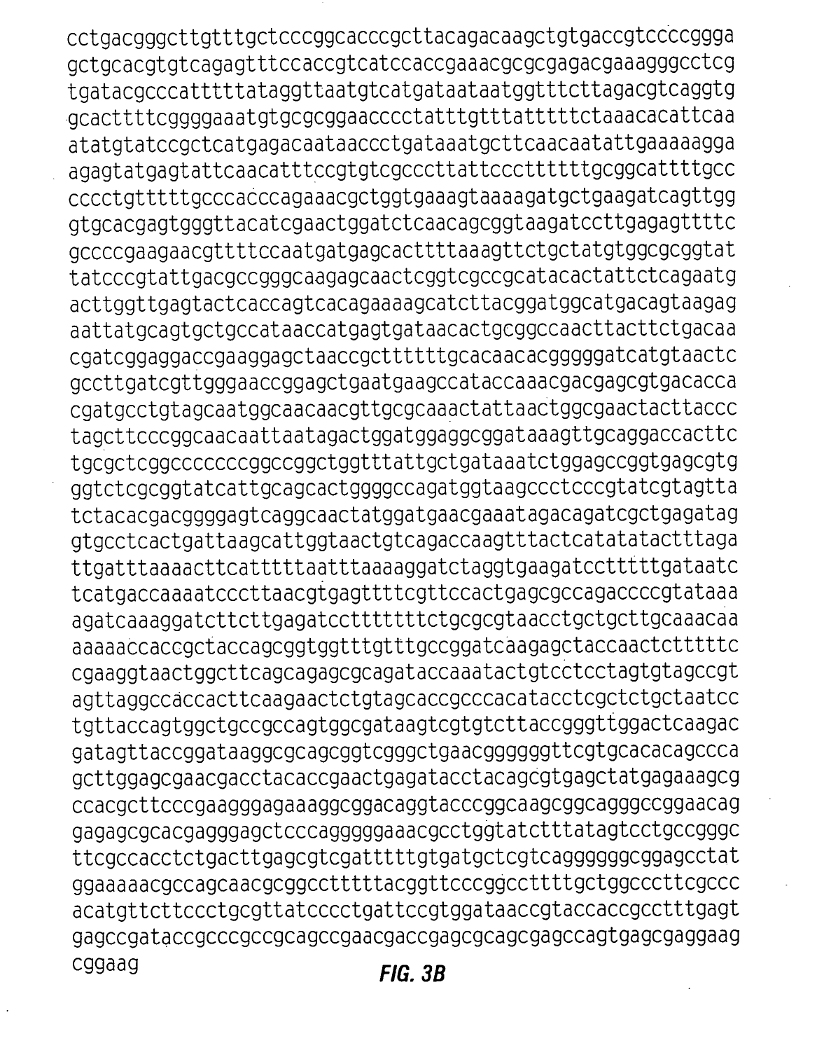

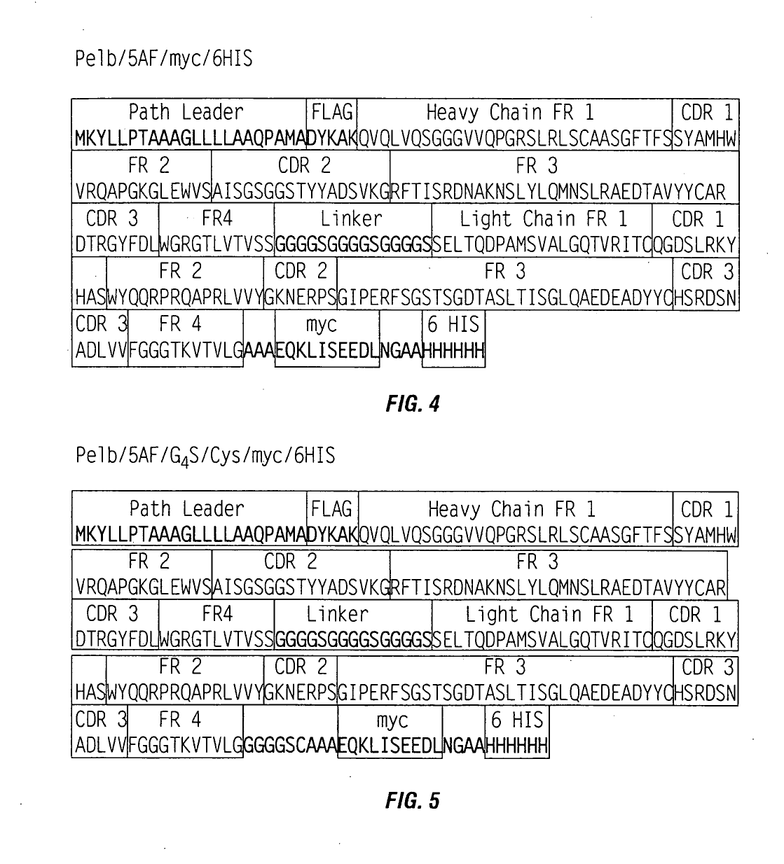

Another type of organellar delivery element is one which directs the fusion protein to the cell membrane and which may include a membrane anchoring element. Depending on the nature of the anchoring element, it can be cleaved on the internal or external leaflet of the membrane, thereby delivering the fusion protein to the intracellular or extracellular compartment, respectively. For example, it has been demonstrated that mammalian proteins can be linked to i) myristic acid by an amide-linkage to an N-terminal glycine residue, to ii) a fatty acid or diacylglycerol through an amide- or thioether-linkage of an N-terminal cysteine, respectively, or covalently to iii) a phophotidylinositol (PI) molecule through a C-terminal amino acid of a protein (for review, see Low, Biochem. J. 244:1-13, 1987). In the latter case, the PI molecule is linked to the C-terminus of the protein through an intervening glycan structure, and the PI then embeds itself into the phopholipid bilayer; hence the term “GPI” anchor. Specific examples of proteins know to have GPI anchors and their C-terminal amino acid sequences have been reported (see Table 1 and FIG. 4 in Low, Biochemica et Biophysica Acta, 988:427-454, 1989; and Table 3 in Ferguson, Ann. Rev. Biochem., 57:285-320, 1988). Incorporation of GPI anchors and other membrane-targeting elements into the amino- or carboxy-terminus of a fusion protein can direct the chimeric molecule to the cell surface.

Detectable polypeptides are optional fusion protein elements that either generate a detectable signal or are specifically recognized by a detectably labeled agent. An example of the former class of detectable polypeptide is green fluorescent protein (GFP). Examples of the latter class include epitopes such the “FLAG tag” and the c-myc epitope. These and other epitopes can be detected using labeled antibodies that are specific for the epitope; several such antibodies are commercially available.

Protein purification elements (a.k.a. protein isolation elements) are amino acid sequences that can be incorporated into a fusion protein in order to facilitate the purification or isolation of a fusion protein from a mixture containing other molecules.

Protein purification elements also include secretion sequences that direct recombinantly produced proteins out of the host cell and into the cellular media. Secreted proteins can then be separated from the host cells that produce them by simply collecting the media. Examples of secretion elements include those described in U.S. Pat. Nos. 5,846,818; 5,212,070; 5,631,144; 5,629,172; and 6,103,495; and Hardig et al., J. Biol. Chem. 268:3033-3036, 1993; Sizmann et al., Year Immunol. 7:119-130, 1993; and Power et al., Gene 113:95-99, 1992). Protein purification elements also include sequences that direct a recombinant protein to the periplasmic space of bacteria (Battistoni et al., Protein Expr. Purif. 6:579-587, 1995). Those skilled in the art will be able to determine which purification elements are desired, appropriate or necessary for a given fusion protein and/or expression system.

Of particular interest are purification elements that can be used to isolate a fusion protein from the host cells or media of an expression system. Examples of purification elements include a “His tag” (6 contiguous His residues, a.k.a. 6×His), which binds to surfaces that have been coated with nickel; streptavidin or avidin, which bind to surfaces that have been coated with biotin or “biotinylated” (see U.S. Pat. No. 4,839,293 and Airenne et al., Protein Expr. Purif. 17:139-145, 1999); and glutathione-s-transferase (GST), which binds glutathione (Kaplan et al., Protein Sci. 6:399-406, 1997; U.S. Pat. No. 5,654,176). Polypeptides that bind to lead ions have also been described (U.S. Pat. No. 6,111,079). “Epitope tags” such as the c-myc epitope or FLAG-tag can be used to purify recombinant proteins via affinity chromatography using antibodies to such epitope tags.

As used herein, the term “protein purification element” also includes elements designed to enhance the solubility and or assist in the proper folding of a protein. Such elements include GST and members of the 14-3-3 family of proteins (U.S. Pat. No. 6,077,689).

IV.C. Spacers

Spacers (a.k.a. linkers) are amino acid sequences that can be included in a fusion protein in between other portions of a fusion protein (e.g., between the biologically active polypeptide and the pIgR-targeting element, or between an optional fusion protein element and the remainder of the fusion protein). Spacers can be included for a variety of reasons. For example, a spacer can provide some physical separation between two parts of a protein that might otherwise interfere with each other via, e.g., steric hinderance. One example of a spacer of this type is the repeating amino acid sequence (Gly4-Ser)ₓ, wherein x is 1 to 10, and preferably 1 to 4.

IV.D. Protease Cleavage Sites

In related embodiments of the invention, the pIgR-targeted fusion proteins can be designed so as to contain a site (a “protease cleavage site” or simply “cleavage site”) that is amenable to being cleaved by agents or under conditions that cause or promote such cleavage. In some preferred embodiments of the invention, the cleavage site is contained within a spacer element, so that cleavage separates, e.g., the pIgR targeting element of a fusion protein from the biologically active polypeptide thereof, which is useful for in vivo therapeutic methods; or between an optional protein purification element and the remainder of the fusion protein, which is useful for removing extraneous and potentially interfering purification elements in the process of purifying the fusion protein in vitro.

The nature and arrangement of a cleavage site or of a spacer containing a cleavage site will depend on the nature of the in vivo or in vitro method(s) of interest. It is understood by those skilled in the art that the amino acids sequences of fusion proteins that one wishes to have cleaved by a protease must be designed so as to retain the protease cleavage site of choice. Non-limiting examples of in vitro and in vivo cleavage sites and systems are as follows.

IV.D.1. In Vivo Cleavage

Polypeptide fragments derived from the spacer and other optional fusion protein elements may be independently released from the cleaved fusion protein, or may remain associated with the pIgR targeting element or biologically active polypeptide. Most preferably, the cleavage reaction will predominantly occur after the fusion protein has been transported into or across an epithelial cell, or within a subcellular compartment, e.g., an organelle. For example, and for illustrative purposes only, the cleavage reaction might be carried out by a protease or esterase found in an epithelial cell, by the acidic conditions found near a tumor cell, by conditions in the blood that destabilize disulfide conjugation, or by a protease found in an organelle.

Preferred cleavage sites for in vivo applications include but are not limited to those that are recognized by caspases, which can be used, e.g., to cleave and activate a biologically active polypeptide from a fusion protein during early events in apoptosis; proteases specific for an organelle into which it is desired to deliver a fusion protein, with one intended result being that a biologically active portion of the cleaved fusion protein will be retained by the organelle (i.e, organellar leader sequences).

Caspases are intracellular cysteine proteases which have been shown to play an essential role in the initiation and execution phases of apoptotic cell death. For reviews, see Fadeel et al. (IUBMB Life 49:421-425, 2000), Anderson (Cell Death Differ. 7:589-602, 2000) and Earnshaw et al., Annu. Rev. Biochem. 68:383-424, 1999). Fusion proteins can be designed so as to require proteolytic activation before it becomes biologically active. Inclusion of a given caspase cleavage site in such a fusion protein can be used to design fusion proteins that are cleaved by a particular caspase and activated. In instances where the biologically active component of a fusion protein is not active until released from the fusion protein, the latter type of fusion proteins provide biologically polypeptides that act at specific times during the apoptotic process. Cathepsins may be used in the same way in other vesicular compartments of the cell. Organellar leader sequences include, by way of non-limiting example, mitochondrial leader peptides that are proteolytically removed from proteins after their transport into mitochondria.

Cathepsin B cleavage sites may be used. The following four peptides are known to be cleaved by cathepsin B: GFQGVQFAGV, GFGSVQFAGF, GLVGGAGAGF, GGFLGLGAGF, GFGSTFFAGF (Peterson and Meares, Bioconjugate Chem. 10:553-557, 1999). A peptide with any one the following sequences is modified to link a maleimido group at the amino terminal, where any one of the amino acids within the parenthesis may be used in combination with any one of the amino acids within any of the other amino acids in a parenthesis. There are 1,728 possible combinations. Non-limiting examples of such sequences are GFQGVQFAGV, GFGSVQFAGF, GLVGGAGAGF, GGFLGLGAGF, and GFGSTFFAGF.

The ability of the various linkers to be cleaved by cathepsin A, cathepsin B, cathepsin C, cathepsin D, cathepsin G, cathepsin S and other cathepsins are tested by the methods used by Peterson and Meares (Bioconjugate Chem. 9:618-626, 1998). G-[F, L or G]-[Q, G, V or F]-[G, S or L]-[V, G or T]-[Q, A, L or F]-[F or G]-A-G-[V or F]

Decapeptides having one of these amino acid combinations may be flanked with sequences at either the amino terminus or the carboxy terminus to provide additional flexiblity and spacing between the ligand, protein, peptide, or macromolecule. Such flanking sequences may be, for example, (GlyₓSery)_{z}, where x and y may be from 0 to 4 and z may be from 1 to 4. In some instances, x is 4, y is 1, and z is 1 to 2.

Such peptides may be synthesized by solid phase using chemistries well known to those skilled in the art. Such a peptide contains only one amino group, which is at the amino terminus of the peptide.

IV.D.2. In Vitro Cleavage

Cleavable spacers may also be used for other purposes, especially in protein purification schemes. Consider, as an example, the case of a fusion protein that has an amino terminal 6×His tag, and a protease cleavage site located immediately carboxy terminal from the His tag, i.e, between the His tag and the remainder of the fusion protein being produced. After the fusion protein has been purified using the His tag's affinity for nickel-coated surfaces, it is then cleaved with the appropriate protease in order to separate the His tag from the remainder of the protein. It is often desirable to remove elements such as His tags that are useful for protein purification purposes but might interfere with the biological activity of the fusion proteins. Cleavable spacers may be designed so as to regenerate the amino terminal amino acid sequence present in the original protein.

Preferred cleavage sites for in vitro applications include but are not limited to those that recognize a cleavage site, which may be introduced into a fusion protein by genetic manipulation, that is located between a portion of the fusion protein that is not required for, and may even be detrimental to, the in vivo uses for which the fusion protein is intended. Commercially available expression systems that may be used to introduce cleavage sites include by way of non-limiting example cleavage sites that are recognized by enterokinase, trypsin, Factor Xa, Factor IXa and thrombin.

Enterokinase may be used to cleave spacer elements (see U.S. Pat. No. 4,745,069). A preferred enterokinase is one that is produced via recombinant DNA techniques, as it is virtually free of other proteases and is able to efficiently cleave fusion proteins in partially purified preparations (Collins-Racie et al., Biotechnology 13:982-987, 1995). Moreover, enterokinase is relatively permissive regarding the amino acid residue downstream of the recognition sequence (Hosfield et al., Anal. Bochem. 269:10-16, 1999). Trypsin may also be used in this fashion (U.S. Pat. No. 6,037,143). In addition to providing cleavage sites for purification protein purposes, in vivo cleavage by gastrointestinal proteases such as enterokinase or trypsin may serve as a mechanism by which a fusion protein is released from a carrier in the gut.

Factor Xa (Peter et al., Circulation 101:1158-1164, 2000; U.S. Pat. No. 6,010,883) and thrombin are blood coagulation factors. Expression vectors may comprise a sequence encoding a cleavage site for thrombin or Factor Xa that can be used to remove a purification element (such as a His tag) from the fusion protein after it has served its purification purpose.

IV.E. Production of Fusion Proteins Via Recombinant DNA Expression Systems

In order to achieve recombinant expression of a fusion protein, an expression cassette or construct capable of expressing a chimeric reading frame is introduced into an appropriate host cell to generate an expression system. The expression cassettes and constructs of the invention may be introduced into a recipient prokaryotic or eukaryotic cell either as a nonreplicating DNA or RNA molecule, which may either be a linear molecule or, more preferably, a closed covalent circular molecule. Since such molecules are incapable of autonomous replication, the expression of the gene may occur through the transient expression of the introduced sequence. Alternatively, permanent expression may occur through the integration of the introduced DNA sequence into the host chromosome.

Host cells which may be used in the expression systems of the present invention are not strictly limited, provided that they are suitable for use in the expression of the chimeric pIgR-targeting peptide of interest. Suitable hosts may often include eukaryotic cells. Preferred eukaryotic hosts include, for example, yeast, fungi, insect cells, mammalian cells either in vivo, or in tissue culture.

Expression cassettes and constructs may be introduced into an appropriate host cell by any of a variety of suitable means, i.e, transformation, transfection, conjugation, protoplast fusion, electroporation, particle gun technology, calcium phosphate-precipitation, direct microinjection, and the like. After the introduction of the vector, recipient cells are grown in a selective medium, which selects for the growth of vector-containing cells. Expression of the cloned gene(s) results in the production of a chimeric pIgR-targeting peptide of the invention, or fragments thereof.

The introduced nucleic acid molecule can be incorporated into a plasmid or viral vector capable of autonomous replication in the recipient host. Any of a wide variety of vectors may be employed for this purpose. Factors of importance in selecting a particular plasmid or viral vector include: the ease with which recipient cells that contain the vector may be recognized and selected from those recipient cells which do not contain the vector; the number of copies of the vector which are desired in a particular host; and whether it is desirable to be able to “shuttle” the vector between host cells of different species.

A variety of recombinant DNA expression systems may be used to produce the fusion proteins of the invention. Expression systems of particular interest include prokaryotic systems, yeast expression systems, insect expression systems mammalian expression systems.

Prokaryotic Expression Systems utilize plasmid and viral (bacteriophage) expression vectors that contain replication sites and control sequences derived from a species compatible with the host may be used. Suitable phage or bacteriophage vectors may include λgt10, λgt11 and the like; and suitable virus vectors may include pMAM-neo, pKRC and the like. Appropriate prokaryotic plasmid vectors include those capable of replication in E. coli (such as, by way of non-limiting example, pBR322, pUC118, pUC119, ColE1, pSC101, pACYC 184, πVX; “Molecular Cloning: A Laboratory Manual”, 1989, supra). Bacillus plasmids include pC194, pC221, pT127, and the like (Gryczan, In: The Molecular Biology of the Bacilli, Academic Press, NY, pp. 307-329, 1982). Suitable Streptomyces plasmids include p1J101 (Kendall et al., J. Bacteriol. 169:4177-4183, 1987), and streptomyces bacteriophages such as ΦC31 (Chater et al., In: Sixth International Symposium on Actinomycetales Biology, Akademiai Kaido, Budapest, Hungary, pp. 45-54, 1986). Pseudomonas plasmids are reviewed by John et al. (Rev. Infect. Dis. 8:693-704, 1986), and Izaki (Jpn. J. Bacteriol. 33:729-742, 1978). See also Brent et al., “Vectors Derived From Plasmids,” Section II, and Lech et al. “Vectors derived from Lambda and Related Bacteriophages” Section III, in Chapter 1 of Short Protocols in Molecular Biology, 2nd Ed., Ausubel et al., eds., John Wiley and Sons, New York, 1992, pages 1-13 to 1-27; Lech et al. “Vectors derived from Lambda and Related Bacteriophages” Section III and Id. pages 1-28 to page 1-52.

Recognized prokaryotic hosts include bacteria such as E. coli, Bacillus, Streptomyces, Pseudomonas, Salmonella, Serratia, and the like. However, in such hosts, the fusion protein will not be glycosylated. In any event, the host cell must be compatible with the replicon and control sequences in the expression cassette.

To express a chimeric pIgR-targeting peptide of the invention (or a functional derivative thereof) in a prokaryotic cell, it is necessary to operably link the sequence encoding the chimeric pIgR-targeting peptide of the invention to a functional prokaryotic promoter. Such promoters may be either constitutive or, more preferably, regulatable (i.e, inducible or derepressible). Examples of constitutive promoters include the int promoter of bacteriophage λ, the b1a promoter of the β-lactamase gene sequence of pBR322, and the cat promoter of the chloramphenicol acetyl transferase gene sequence of pPR325, and the like. Examples of inducible prokaryotic promoters include the major right and left promoters of bacteriophage λ (PL and PR), the trp, recA, lacZ, lacI, and gal promoters of E. coli, the α-amylase (Ulmanen et al., J. Bacteriol. 162:176-182, 1985) and promoters of B. subtilis (Gilman et al., Gene Sequence 32:11-20, 1984), the promoters of the bacteriophages of Bacillus (Gryczan, in: The Molecular Biology of the Bacilli, Academic Press, Inc., NY, 1982), and Streptomyces promoters (Ward et al., Mol. Gen. Genet. 203:468-478, 1986). Prokaryotic promoters are reviewed by Glick (Ind. Microbiot. 1:277-282, 1987), Cenatiempo (Biochimie 68:505-516, 1986), and Gottesman (Ann. Rev. Genet. 18:415-442, 1984).

Proper expression in a prokaryotic cell also requires the presence of a ribosome-binding site upstream of the gene sequence-encoding sequence. Such ribosome-binding sites are disclosed, for example, by Gold et al. (Ann. Rev. Microbiol. 35:365-404, 1981). The selection of control sequences, expression vectors, transformation methods, and the like, are dependent on the type of host cell used to express the gene. As used herein, “cell”, “cell line”, and “cell culture” may be used interchangeably and all such designations include progeny. Thus, the words “transformants” or “transformed cells” include the primary subject cell and cultures derived therefrom, without regard to the number of transfers. It is also understood that all progeny may not be precisely identical in DNA content, due to deliberate or inadvertent mutations. However, as defined, mutant progeny have the same functionality as that of the originally transformed cell.

Bacterial systems may also be used to create and produce large amounts of shuttle vectors. Shuttle vectors are constructs designed to replicate in a prokaryotic host such as E. coli but which contain sequences that allow the shuttle vector and a chimeric reading frame incorporated therein to be transferred to a eukaryotic viral vector or other vector such as baculovirus or adenovirus.

Yeast Expression Systems can be utilized which incorporate promoter and termination elements from the actively expressed sequences coding for glycolytic enzymes that are produced in large quantities when yeast are grown in mediums rich in glucose. Known glycolytic gene sequences can also provide very efficient transcriptional control signals. Yeast cells provide a substantial advantage over prokarytoic expression systems in that they can carry out post-translational modifications of fusion proteins. A number of recombinant DNA strategies exist utilizing strong promoter sequences and high copy number plasmids which can be utilized for production of the desired proteins in yeast. Yeast recognizes leader sequences on cloned mammalian genes and secretes peptides bearing leader sequences (i.e, pre-peptides).

Preferred yeast expression vectors include those derived from the episomal element known as the 2-micron circle as well as derivatives of yeast integrating (YIp), yeast replicating (YRp), yeast centromeric (YCp), yeast episomal (YEp), and yeast linear (YLp) plasmids (Broach, in: The Molecular Biology of the Yeast Saccharomyces: Life Cycle and Inheritance, Cold Spring Harbor Laboratory, Cold Spring Harbor, N.Y., p. 445-470, 1981; Lundblad et al., Section II and, Becker et al., Section III, of Chapter 13 in: Short Protocols in Molecular Biology, 2nd Ed., Ausubel et al., eds., John Wiley and Sons, New York, 1992, pages 13-19 to 13-41).

Insect Expression Systems utilize insect host cells, e.g., Sf9 and Sf21 cells, both of which are derived from the iplbsf-21 cell line derive from the pupal ovarian tissue of the fall army worm spodoptera frugiperda (O'Reilly et al., Baculovirus expression vectors: A Laboratory Manual New York, N.Y., W.H. Freeman and Company. See also baculovirus expression protocols in Methods in Molecular Biology Vol. 39; Richardson ed. Humana Totowa N.J., 1992; and Vaughn et al., In vitro 13:213-217, 1977. The cell line bti-tn-5b1-4 (high 5 tm cell line), which originated from the ovarian cells of the cabbage luper, Trichoplusa ni (Davis et al., Biotechnology 10:1148-1150, 1992; Granados et al., J. Invertebr. Pathol. 64:260-266, 1994; Wickham et al., iotechnology Prog. 8:391-396, 1992; Wickham et al., Biotechnol. Prog. 9:25-30, 1993). Other insect cell lines that can be used to express baculovirus vectors have been described (Hink et al., Biotechnol. Prog. 7:9-14, 1991). See, also Piwnica-Worms “Expression of Proteins in Insect Cells Using Baculo Viral Vectors” section II in chapter 16 of Short Protocols in Molecular Biology, second edition, Ausubel et al, eds., John Wiley and Sons, New York, N.Y. 1992. Using insect cells as hosts, the Drosophila alcohol dehydrogenase promoter can be used (Rubin, Science 240:1453-1459, 1988). Alternatively, baculovirus vectors can be engineered to express large amounts of chimeric pIgR-targeting peptides of the invention in insect cells (Jasny, Science 238:1653, 1987; Miller et al., in: Genetic Engineering, Vol. 8, Plenum, Setlow et al., eds., pp. 277-297, 1986).

Mammalian Expression Systems utilize host cells such as HeLa cells, cells of fibroblast origin such as VERO, CV-1 monkey kidney cells and COS-1 (CV-1 cells transformed with large T antigen) or CHO-K1, or cells of lymphoid origin and their derivatives. Preferred mammalian host cells include SP2/0 and J558L, as well as neuroblastoma cell lines such as IMR 332, which may provide better capacities for correct post-translational processing.

Several expression vectors are available for the expression of chimeric pIgR-targeting peptides of the invention in a mammalian host. A wide variety of transcriptional and translational regulatory sequences may be employed, depending upon the nature of the host. The transcriptional and translational regulatory signals may be derived from viral sources, such as adenovirus, bovine papilloma virus, cytomegalovirus, simian virus, or the like, where the regulatory signals are associated with a particular gene sequence which has a high level of expression. Alternatively, promoters from mammalian expression products, such as actin, collagen, myosin, and the like, may be employed. Transcriptional initiation regulatory signals may be selected which allow for repression or activation, so that expression of the gene sequences can be modulated. Of interest are regulatory signals which are temperature-sensitive so that by varying the temperature, expression can be repressed or initiated, or are subject to chemical (such as metabolite) regulation.

Preferred eukaryotic plasmids include, for example, BPV, vaccinia, SV40, 2-micron circle, and the like, or their derivatives. Such plasmids are well known in the art (Botstein et al., Miami Wntr. Symp. 19:265-274, 1982; Broach, in: The Molecular Biology of the Yeast Saccharomyces: Life Cycle and Inheritance, Cold Spring Harbor Laboratory, Cold Spring Harbor, N.Y., p. 445-470, 1981; Broach, Cell 28:203-204, 1982; Bollon et al., J. Clin. Hematol. Oncol. 10:39-48, 1980; Maniatis, In: Cell Biology: A Comprehensive Treatise, Vol. 3, Gene Sequence Expression, Academic Press, NY, pp. 563-608, 1980).

Expression of chimeric pIgR-targeting peptides of the invention in eukaryotic hosts requires the use of eukaryotic regulatory regions. Such regions will, in general, include a promoter region sufficient to direct the initiation of RNA synthesis. Preferred eukaryotic promoters include, for example, the promoter of the mouse metallothionein I gene sequence (Hamer et al., J. Mol. Appl. Gen. 1:273-288, 1982); the TK promoter of Herpes virus (McKnight, Cell 31:355-365, 1982); the SV40 early promoter (Benoist et al., Nature (London) 290:304-31, 1981); and the yeast gal4 gene sequence promoter (Johnston et al., Proc. Natl. Acad. Sci. (USA) 79:6971-6975, 1982; Silver et al., Proc. Natl. Acad. Sci. (USA) 81:5951-5955, 1984).]

Translation of eukaryotic mRNA is initiated at the codon which encodes the first methionine. For this reason, it is preferable to ensure that the linkage between a eukaryotic promoter and a DNA sequence which encodes a chimeric pIgR-targeting peptide of the invention (or a functional derivative thereof) does not contain any intervening codons which are capable of encoding a methionine (i.e, AUG). The presence of such codons results either in the formation of a fusion protein (if the AUG codon is in the same reading frame as the chimeric pIgR-targeting peptide of the invention coding sequence) or a frame-shift mutation (if the AUG codon is not in the same reading frame as the chimeric pIgR-targeting peptide of the invention coding sequence).

V. Protein Conjugates

One type of compound of the invention is a protein conjugate, i.e., a biologically active polypeptide that is covalently linked to a targeting element that is also polypeptide.

V.A. Covalently Attaching Targeting Elements to Bioactive Compounds

Polypeptides that are pIgR-targeting elements, including but not limited to antibody derivatives and bacterial proteins that bind pIgR, can be linked to bioactive compounds in a varity of ways. In general, there are four ways that protein conjugate members are linked to other protein conjugate members. First, amino acid residues present in the natural sequence of a first protein member can be directly covalently linked to amino acid residues in the natural amino acid sequence of a second protein member as in, e.g., a disulfide bridge. Second, mutant amino acids useful for covalent linkages can be introduced into one or more protein members by using molecular genetics to alter the reading frame encoding such protein members or, in the case of synthetic oliogopeptides, directly during the in vitro synthesis thereof. Third, natural or mutant amino acid sequences present in isolated proteins can be “derivatized” (i.e, chemically modified in vitro) so as to include chemical groups not present in natural amino acids but useful for the chemical conjugation of oligopeptides, polypeptides, and proteins in a related methodology, unnatural amino acids having moities useful for chemical conjugation are introduced into oligopeptides or peptidomimetics during their synthesis in vitro. Fourth, a cross-linking reagent (a.k.a. “cross-linker”), typically a bifunctional (two-armed) chemical linker that forms covalent linkages between two or more conjugate members, can be used to covalently link conjugate members to each other. Such bifunctional linkers can be homobifunctional (wherein both “arms” of the linker are the same chemical moiety) or heterobifunctional (wherein each of the two “arms” is a different chemical moiety than the other).

Hermanson (Bioconjugate Techniques, Academic Press, 1996), herein incorporated by reference, summarizes many of the chemical methods used to link proteins and other molecules together using various reactive functional groups present on various cross-linking or derivatizing reagents. Polypeptide cross-linking agents are based on reactive functional groups that modify and couple to amino acid side chains of proteins and peptides, as well as to other side groups and other macromolecules. Bifunctional cross-linking reagents incorporate two or more functional reactive groups. The functional reactive groups in a bifunctional cross-linking reagent may be the same (homobifunctional) or different (heterobifunctional). Many different cross-linkers are available to cross-link various proteins, peptides, and macromolecules. Table 7 lists some of the cross-linkers that are available through commercial sources according to their class of chemical reactivity. Table 8 lists some of the properties of chemical cross-linkers and the types of functional groups with which they react.

TABLE 7
CLASSES OF CHEMICAL REACTIVITY OF CROSS-LINKERS

| Chemical reactivity | Abbreviation | Compound |
|---|---|---|
| Homobifunctional imidoesters | DMA | Dimethyl adipimidate.2 HCl |
| | DMP | Dimethyl pimelimidate.2 HCl |
| | DMS | Dimethyl suberimidate.2 HCl |
| | DTBP | Dimethyl 3,3′-dithiobispropionimidate.2HCl |
| Homobifunctional N- | DSG | Disuccinimidyl glutarate |
| hydroxysuccinimide esters (NHS- | | |
| esters) | | |
| | DMSC | Dimethyl succinimidate.2 HCl |
| | DSS | Disuccinimidyl suberate |
| | BS | |
| | DSP | Dithiobis(succinimidylpropionate) |
| | DTSSP | Dithiobis(sulfosuccinimidylpropionate) |
| | DTME | Dithio-bis-maleimidoethane |
| | EGS | Ethylene glycolbis(succinimidylsuccinate) |
| | Sulfo-EGS | Ethylene |
| | | glycolbis(sulfosuccinimidylsuccinate) |
| | DST | Disuccinimidyl tartrate |
| | Sulfo-DST | Disulfosuccinimidyl tartrate |
| | BSOCOES | Bis[2- |
| | | (succinimidooxycarbonyloxy)ethyl]sulfone |
| | Sulfo- | Bis[2- |
| | BSCOCOES | (sulfosuccinimidooxycarbonyloxy)ethyl]sulfone |
| heterobifunctional NHS-esters | BS3 | BIS-(sulfosuccinimidyl) suberate |
| | DMM | dimethyl malonimidate.2 HCl |
| | EMCS | N-[ε-maleimidocaproyloxy]succinimide |
| | | ester |
| | Sulfo-EMCS | N-[ε- |
| | | maleimidocaproyloxy]sulfosuccinimide |
| | | ester |
| | SMCC | Succinimidyl 4-(N- |
| | | maleimidomethyl)cyclohexane-1- |
| | | carboxylate |
| | LC-SMCC | Succiminidyl-4-(N- |
| | | maleimidomethyl)cyclohexane-1-carboxy- |
| | | (6-amido-caproate) |
| | Sulfo-MBS | m-maleimidobenzoyl-N- |
| | | hydoxysulfosuccinimide ester |
| | Sulfo-SMCC | Sulfosuccinimidyl 4-(N- |
| | | maleimidomethyl)cyclohexane-1- |
| | | carboxylate |
| | MBS | m-maleimidobenzoyl-N- |
| | | hydoxysuccinimide ester |
| | SMPB | Succinimidyl 4-[P-Maleimidophenyl] |
| | | butyrate |
| | Sulfo-SMPB | Sulfosuccinimidyl 4-[p- |
| | | maleimidophenyl]butyrate |
| | BMH | Bismaleimidohexane |
| | GMBS | N-[γ-Maleimidobutyryloxy] succinimide |
| | | ester |
| | Sulfo-GMBS | N-[γ-Maleimidobutyryloxy] |
| | | sulfosuccinimide ester |
| heterobifunctional haloacetyl NHS- | SIAB | N-succinimidyl(4- |
| esters | | iodoacetyl)aminobenzoate |
| | Sulfo-SIAB | Sulfo-SIAB sulfosuccinimidyl(4- |
| | | iodoacetyl)aminobenzoate |
| homobifunctional pyridyldithiols | DPDPB | 1,4-Di-[3′-(2′- |
| | | pyridyldithio)propionamido]butane |
| heterobifunctional pyridyldithiols | SMPT | 4-succinimidyloxycarbonyl-methyl-(2- |
| | | pyridyldithio)-toluene |
| | Sulfo-LC- | Sulfosuccinimidyl 6-[a-methyl-a-(2- |
| | SMPT | pyridyl-dithio)toluamido]hexanoate |
| | SPDP | N-succinimidyl 3-(2- |
| | | pyridyldithio)propionate |
| | LC-SPDP | N-succinimidyl 6-[3′-(2- |
| | | pyridyldithio)propionamido]hexanoate |
| | Sulfo-LC- | Sulfosuccinimidyl 6-[3′-(2-pyridyldithio)- |
| | SPDP | propionamido] hexanoate |
| carboxyl reactive | PDPH | 3-(2-Pyridyldithio) propionyl hydrazide |
| carbonyl reactive | | |
| | EDC | 1-ethyl-3-(3-dimethylaminopropyl)- |
| | | carbodiimide |
| | M2C2H | 4-(N-Maleimidomethyl)cyclohexane-1- |
| | | carboxyl hydrazide |
| | DCC | N,N-dicyclohexylcarbodimide |
| | MPBH | 4-(4-N-Maleimidophenyl)butyric acid |
| | | hydrazide hydrochloride |
| Photoreactive | ABH | Azidobenzoyl hydrazide |
| | ANB-NOS | N-5-azido-2-nitrobenzoyloxysuccinimide |
| | APDP | N-[4-(p-azidosalicylamido)butyl]-3′(2′- |
| | | pyridyldithio)propionamide |
| | APG | p-Azidophenylglyoxal monhydrate |
| | ASBA | 4-(p-Azidosalicylamido)butylamine |
| | ASIB | 1-(p-Azidosalicylamido)-4- |
| | | (iodoaceamido)butane |
| | BASED | Bis-[B-4- |
| | | azidosalicylamido)ethyl]disulfide |
| | HSAB | N-Hydroxysuccinimidyl-4-azidobenzoate |
| | Sulfo-HSAB | N-Hydroxysulfo-succinimdyl-4- |
| | | azidobenzoate |
| | NHS-ASA | N-Hydroxysuccinimidyl-4-azidosalicylic |
| | | acid |
| | Sulfo-NHS- | N-Hydroxysulfo-succinimidly-4- |
| | ASA | azidosalicylic acid |
| | Sulfo-NHS- | Sulfosuccinimidly-[4-azidosalicylamido)- |
| | LC-ASA | hexanoate |
| | PNP-DTP | p-Nitropheyno-2-diazo-3,3,3- |
| | | trifluoropropionate |
| | DTP | 2-Diazo-3,3,3-trifluoropropionylchloride |
| | SADP | N-succinimidyl-(4-azidopheynyl 1,3′ |
| | | dithiopropionate |
| | Sulfo-SADP | Sulfosuccinimidyl-(4- |
| | | azidophynyldithio)propionate |
| | SAED | Sulfosuccinimidyl 2(7-azido-4- |
| | | methylcoumarin-3-acetamide)ethyl-1,3- |
| | | dithiopropionate |
| | Sulfo-SAMCA | Sulfosuccinimidyl 7-azido-4- |
| | | methycoumarin-3-acetate |
| | SAND | Sulfosuccinimidyl 2-(m-azido-o- |
| | | nitrobenzamdio)-ethyl-1,3- |
| | | dithiopropionate |
| | SANPH | N-succinimidyl-6-(4′-azido-2′- |
| | | nitrophenylamino)hexanoate |
| | Sulfo-SANPH | Sulfosuccinimidyl 6-(4′-azido-2′- |
| | | nitrophenylamino)hexanoate |
| | SASD | Sulfosuccinimidyl 2-(p- |
| | | azdiosalicylamido)ethyl-1,3′- |
| | | dithiopropionate |
| | Sulfo-SAPB | Sulfosuccinimidyl 4-(p-azidophenyl)- |
| | | butyrate |
| Heterobifunctional amine reactive | SDBP | N-Hydroxysuccinimidyl 2,3- |
| | | dibromopropionate |
| Bifunctional aryl halide | DFDNB | 1,5-Difluoro-2.4-dinitrobenzene |
| heterobifunctional | mal-sac-HNSA | Maleimido-6-aminocaproyl-ester of |
| nitrophenylsulfonic acid ester | | 1-hydroxy-2-nitrobenzene-4-sulfonic acid |

TABLE 8
CHEMICAL CROSS-LINKERS AND SOME OF THEIR PROPERTIES

| | Pierce | Spacer Arm | | | | |
|---|---|---|---|---|---|---|
| | Product | Length | | Cleavable | Water | Membrane |
| Acronym | Number | (angstroms) | Links | By | Soluble | Permeable |
| Sulfo-LC-SMPT | 21568 | 20.0 | Amines To | Thiols | Yes | No |
| | | | Sulfhydryls | | | |
| SMPT | 21558 | 20.0 | Amines To | Thiols | Yes | No |
| | | | Sulfhydryls | | | |
| Sulfo-KMUS | 21111 | 19.0 | Amines To | non | Yes | No |
| | | | Sulfhydryls | | | |
| LC-SMCC | 22362 | 16.1 | Amines To | non | Yes | No |
| | | | Sulfhydryls | | | |
| KMUA | 22211 | 15.7 | Amines To | non | Yes | No |
| | | | Sulfhydryls | | | |
| LC-SPDP | 21651 | 15.6 | Amines To | non | No | N/d |
| | | | Sulfhydryls | | | |
| Sulfo-LC-SPDP | 21650 | 15.6 | Amines To | Thiols | Yes | No |
| | | | Sulfhydryls | | | |
| SMPB | 22416 | 14.5 | Amines To | non | No | Yes |
| | | | Sulfhydryls | | | |
| Sulfo-SMPB | 22317 | 14.5 | Amines To | non | Yes | No |
| | | | Sulfhydryls | | | |
| SMPH | 22363 | 14.3 | Amines To | non | No | N/d |
| | | | Sulfhydryls | | | |
| SMCC | 22360 | 11.6 | Amines to | non | No | Yes |
| | | | Sulfhydryls | | | |
| Sulfo-SMCC | 22322 | 11.6 | Amines to | non | Yes | No |
| | | | Sulfhydryls | | | |
| SIAB | 22329 | 10.6 | Amines to | non | No | Yes |
| | | | Sulfhydryls | | | |
| Sulfo-SIAB | 22327 | 10.6 | Amines To | non | Yes | No |
| | | | Sulfhydryls | | | |
| Sulfo-GMBS | 22324 | 10.2 | Amines To | non | Yes | No |
| | | | Sulfhydryls | | | |
| GMBS | 22309 | 10.2 | Amines To | non | No | Yes |
| | | | Sulfhydryls | | | |
| MBS | 22311 | 9.9 | Amines To | non | No | Yes |
| | | | Sulfhydryls | | | |
| Sulfo-MBS | 22312 | 9.9 | Amines To | non | Yes | No |
| | | | Sulfhydryls | | | |
| Sulfo-EMCS | 22307 | 9.4 | Amines To | non | Yes | No |
| | | | Sulfhydryls | | | |
| EMCA | 22306 | 9.4 | Amines To | non | Yes | No |
| | | | Sulfhydryls | | | |
| EMCS | 22308 | 9.4 | Amines To | non | No | Yes |
| | | | Sulfhydryls | | | |
| SVSB | 22358 | 8.3 | Amines To | non | No | Yes |
| | | | Sulfhydryls | | | |
| BMPS | 22298 | 6.9 | Amines To | non | No | N/d |
| | | | Sulfhydryls | | | |
| SPDP | 21857 | 6.8 | Amines To | Thiols | No | Yes |
| | | | Sulfhydryls | | | |
| SBAP | 22339 | 6.2 | Amines To | non | No | Yes |
| | | | Sulfhydryls | | | |
| BMPA | 22296 | 5.9 | Amines To | non | Yes | No |
| | | | Sulfhydryls | | | |
| AMAS | 22295 | 4.4 | Amines To | non | No | N/d |
| | | | Sulfhydryls | | | |
| SATP | 26100 | 4.1 | Amines To | non | No | Yes |
| | | | Sulfhydryls | | | |
| SIA | 22349 | 1.5 | Amines To | non | No | N/d |
| | | | Sulfhydryls | | | |
| Sulfo-LC-SMPT | 21568 | 20.0 | Sulfhydryls | Thiols | Yes | No |
| | | | to Amines | | | |
| SMPT | 21558 | 20.0 | Sulfhydryls | Thiols | No | Yes |
| | | | to Amines | | | |
| AEDP | 22101 | 9.5 | Carboxyls to | Thiols | Yes | No |
| | | | Amines | | | |
| EDC | 22980 | 0.0 | Carboxyls to | non | Yes | No |
| | | | Amines | | | |

Bifunctional cross-linking reagents may be classified according to their functional groups, chemical specificity, length of the cross bridge that they establish, the presence of similar functional groups or dissimilar functional groups, chemical or photochemical reactivity, ability to be cleaved internally by reduction or other means, and the ability of the reagent to be further modified by radiolabelling (i.e. radioiodination) or addition of detectable tags or labels. The selective groups on the cross-linking reagent can be present in a homobifunctional arrangement in which the selective groups are identical, or can be present in a heterobifunctional arrangement in which the selective groups are dissimilar.

The chemical modification may be done using cross-linking reagents containing selective groups that react with primary amines, sulfhydryl (thiol) groups, carbonyl, carboxyl groups, hydroxyl, or carbohydrates and other groups placed on a protein or peptide, especially by posttranslational modifications within the cell. The selective groups include, but are not limited to, imidoester, N-hydroxysuccinimide ester or sulfosuccinimidyl ester, ester of 1-hydroxy-2-nitrobenzene-4-sulfonic, maleimide, pyridyl disulfide, carbodiimide, hydrazide and α-haloacetyl groups.

Sulfhydryl reactive functional groups include maleimides, alkyl and aryl halides, α-haloacyls, and pyridyl disulfides. Maleimides, alkyl and aryl halides, and α-haloacyls react with thiols to form stable thioether bonds that are not reduced by reagents such as 2-mercaptoethanol and dithiothreitol. Pyridyl disulfides form mixed disulfides with thiol groups, mixed disulfides may be used as an intermediate for cross-linking two or more macromolecules. Cross-linkers that first react with a carboxyl group to form an activated intermediate and then reacts with an amino group, such as a ε-amino group of lysine or an α-amino group of an amino terminal amino acid, may be used.

A spacer arm or “cross-bridge” region, consisting of a spacer group or a functional group, such as a disulfide bond or hindered disulfide bond, may be used to connect the Biologically active polypeptide to the targeting element. The length of the spacer arm may be varied. The distance between the functional groups establishes the length of the spacer arm. Longer spacer arms may be required to diminish or eliminate steric hindrance between two molecules that are cross-linked together. Intermolecular cross-linking is more efficient with longer spacer arms. Short spacer arms favor intramolecular cross-linking, which is preferably avoided in the present invention.

Spacer arms may have reactive bonds within them that enable further modifications. For example, internal cleavable bonds may be placed within the spacer, such as disulfides or hindered disulfides, one or more ester bonds, or vicinal hydroxyl groups. Cleavage of internal disulfide bonds may be achieved using reduction with thiol containing reagents such as 2-mercaptoethanol and dithiothreitol. One or more metabolizable bonds may be inserted internally in the cross-linking reagent to provide the ability for the coupled entities to separate after the bond(s) is broken after the conjugate is transported into the cell and into the body.

Homobifunctional cross-linkers contain at least two identical functional groups. Heterobifunctional cross-linkers contain two or more functional reactive groups that react with different specificity. Because heterobifunctional cross-linkers contain different reactive groups, each end can be individually directed towards different functional groups on proteins, peptides, and macromolecules. This feature results in linking, for example, amino groups on one molecular entity to carboxyl groups on another entity, or amino groups on one entity to sulfhydryl groups on another entity.

Functional groups include reactive portions on proteins, peptides, and macromolecules that are capable of undergoing chemical reaction. Functional groups include amino and carboxyl groups, hydroxyl groups, phenolate hydroxyl groups, carbonyl groups, guanidinyl groups, and carbon-carbon double bonds. In addition, photoactive reagents that become reactive when exposed to light may be used. For example, arylazides may be activated to form activated intermediates, such as an aryl nitrene or a dehydroazepine intermediate, that non-selectively inserts into carbon-hydrogen bonds (i.e. by aryl nitrenes) or reacts with amines (dehydroazepines). Other examples include fluorinated aryl azides, benzophenones, certain diazo compounds, and diazrine derivatives.

V.A.1. N-Hydroxysuccinimide Esters

NHS-esters react efficiently with amino groups in aqueous buffers, preferably phosphate, bicarbonate/carbonate, HEPES, and borate buffers at concentrations between 10 and 200 mM. Buffers should not contain primary amines. Primary amines can be added to the reaction to stop or quench the NHS-ester reaction and thereby terminate further modification of amino groups on proteins, peptides, and macromolecules. The modification or coupling is typically carried out between pH 7 and pH 9, and preferably between pH 7.5 and 8.0. The time of reaction and temperature may depend on the particular molecule that is being modified. The time of modification may be between 10 and 180 minutes, preferably between 30 and 60 minutes at temperatures between 4° C. and 37° C., preferably between 4° C. and 25° C. The concentration of the NHS-ester may vary, but is between 1.1- to 100-fold molar excess, and preferably between 1.1- and 10-fold molar excess. The protein concentration may vary between 1 μM and 100 μM, preferably between 5 μM and 100 μM.

V.A.2. Ester of 1-Hydroxy-2-Nitrobenzene-4-Sulfonic Acid

A maleimido-aliphatic carboxylic acid may form an ester with the hydroxyl group of 1-hydroxy-2-nitrobenzene-4-sulfonic acid. A maleimide group may be placed at the end of a short, intermediate, or long aliphatic acid. An example of this is mal-sac-HNSA (U.S. Pat. No. 4,954,637). Mal-sac-HNSA may be used to acylate amino groups on proteins, peptides, and macromolecules. The maleimide may then be reacted with sulfhydryl groups on other proteins, peptides, and macromolecules to form a stable, noncleavable thioether bond. Aqueous buffers, such as sodium phosphate, and neutral to mildly alkaline conditions, pH 6.5 to 9, and preferably pH 7 to 8, may be used at temperatures from 0° C. to 37° C., and preferably from 4° C. to 25° C.

V.B. Moieties that may be Modified on Macromolecules for Chemical Cross-Linking

V.B.1. Naturally Occurring Modifiable Moieties

Proteins and peptides contain α-amino groups at the amino terminus, ε-amino groups on lysine, β-carboxyl groups on aspartic acid, γ-carboxyl groups on glutamic acid, imidazole rings on histidine, hydroxyl groups on serine and threonine, phenolate hydroxyl groups on tyrosine, sulfhydryl groups on cysteine, disulfide bonds between two cysteines, mercaptide bonds in methionine, and indole rings in tryptophan, all of which can be selectively modified by cross-linking reagents.

Carbohydrates or carbohydrate containing macromolecules contain ketone, aldehyde, hydroxyl, amine, carboxylate, sulfate, and phosphate groups as nonlimiting examples of functional groups with which cross-linkers may react. Carbohydrates containing vicinal hydroxyl groups (hydroxyl groups on adjacent carbon atoms) may be treated with sodium periodate so that the carbon-carbon bond is cleaved; this creates reactive formyl groups on the treated carbohydrate that may be used as a target for appropriately designed cross-linking reagents. Hermanson discloses other methods, which are herein incorporated by reference, for cross-linking carbohydrates.

Hermanson discloses the major sites on nucleic acids that are susceptible to chemical modification. Compositions and methods for synthesizing and conjugating oligonucleotides comprising a Cys residue have been described by Stetsenko and Gait (Nucleosides, Nucleotides and Nucleic Acids 19:1751-1764, 2000).

V.B.2. Substitution or Insertion of Cysteine into Polypeptides for Subsequent Chemical Modification

Ligands genetically fused to therapeutic and diagnostic biologically active proteins and peptides may not always produce a desired result. Genetic fusion is typically performed by attaching the ligand to either the amino or carboxy terminus of the biologically active protein or peptide using a spacer if necessary. Therefore, the geometrical arrangement of ligand and biologically active protein and peptide is necessarily limited. Linkage of the biologically active protein or peptide through surface cysteinyl groups presents more flexibility in designing a combination that allows both the ligand to recognize pIgR and the biologically active protein or peptide to carry out its functions after epithelial transport. If the desired sulfhydryl groups are not present on the protein, peptide or macromolecule, a sulfhydryl may be introduced by genetic modification. Therefore, the present invention provides a method for substituting or inserting a cysteine into the protein or peptide and using the cysteine for chemical conjugation by cross-linking.

An amino acid may be selected for substitution by cysteine, such selected amino acid should be on the surface of the molecule and positioned so as not to interfere or sterically hinder the function of any biologically active site or important site on the molecule that is required for a biological activity or function. The substitution of cysteine for an amino acid may be achieved by methods well-known to those skilled in the art, for example, by using methods described in Maniatis, Sambrook, and Fritsch (Molecular Cloning: A laboratory manual, Cold Spring Harbor Laboratory Press, 1989).

Regions within the crystallographic structure of a polypeptide are chosen so as to minimize potential steric hindrance imposed by coupling a relatively large molecule, such as a pIgR binding sFv, to the cysteinyl residue. Any of the amino acids in loops or unstructured regions may be substituted with a cysteine; however, preferred positions exist. Such preferred positions are at amino acids whose side chains are not hydrogen bonded to other amino acid side chains (or backbone atoms) or do not participate or contribute to the formation of salt or charge bridges with other amino acid side chains. Amino acids within helical regions may also be substituted if their side chains are oriented away from the main body of the protein and do not participate in interactions with other amino acid side chains that provide stability to the structure. A preferred position is an amino acid side chain that is fully exposed to bulk solvent and has no significant interaction with amino acids within the polypeptide's tertiary structure and does not participate in the biological activity or function of the molecule, including receptor binding, signal transduction, and the like.

Amino acids for possible substitution may be chosen by examining the crystallographic structure using software designed for the purpose of visualization of the three dimensional structure. Several programs are available for analysis, including Protein Explorer, Insight II, MDL, Tripos, Amber, Charm, Chem-X, Chime, DOCK, Homology, MAGE, SYBYL, Midas Plus, and others known to those skilled in the art. Both visual inspection and calculations and displays within these programs can be used by those skilled in the art to select substitution positions.

A protein or peptide surface is examined for sites at which substitution of an amino acid by cysteine or insertion of cysteine in the protein sequence does not change or modify the activity of the protein in a significant way. Examination of crystallographic data of the protein or peptide will reveal which amino acid residues and side chains are exposed, as judged by the ability of a water molecule to contact the amino acid or its side chain. Cysteine residues are inserted or substituted into loops, preferably loops that are not defined in crystallographic structures because they are so unstructured that they move during data collection. Cysteine residues are substituted for amino acids on the solvent side of helices. Those skilled in the art will know how to use software programs to analyze the surface features of a protein for the purpose of cysteine substitution or insertion (see, e.g., U.S. Pat. Nos. 4,853,871 and 4,908,773).

In some crystal structures the entirety of a loop is not observed because the flexibility of that region has prevented data from being recorded; therefore, the trace of backbone chain terminates as it enters the flexible region and then appears on the other side of the flexible loop. Such regions are suitable for cysteine replacement or insertion. Any amino acid that is in contact with water is a candidate for replacement by cysteine. Such amino acids may be replaced by cysteine, one by one, and the effect of the substitution examined on biological activity. Those substitutions that do not affect biological activity more than 0 to 20 percent, preferably 0 to 10 percent, and most preferably 0 to 5 percent may be used to cross-link to ligands that bind to pIgR and pIgR stalk.

Loops formed by a small stretch of 5 to 15 amino acids on the surface of a protein or peptide are used to insert a cysteine into the protein sequence. Examination of the surface is expected to reveal a site that has maximum exposure to the bulk solvent. Solvent accessible side chains are identified by examining the Connolly (Connolly/Richards) surfaces of the protein, which are essentially defined by the ability of the side chain to contact a water molecule ‘rolled’ around the surface of the molecule. Insertion of a cysteine at a site accessible to bulk solvent, or within 2 to 4 amino acid residues, is performed to produce a variant of the protein suitable for cross-linking to ligands that bind to pIgR or pIgR stalk. Loops are also identified by performing molecular dynamic analysis on the protein. Molecular dynamic analysis carried out over 50 to 250 picoseconds is expected to reveal flexible regions within the structure of the protein that are used for cysteine substitution or insertion. In such analyses, Cysteine residues are substituted, one at a time, between each pair of amino acids in the flexible loop.

Helical wheels are used to identify the side of the helix that faces bulk solvent. Looking down the barrel of a helix, one can identify residues on one side or the other of the helix. Where crystallographic solutions to the protein structure are available, residues on a helical wheel can be observed in the structure to estimate their access to bulk solvent. Residues on the bulk solvent side of the helical wheel often participate in receptor binding. Substitution of a cysteine for such a residue is undesirable. Substitution within a helix at a residue facing the bulk solvent is provided in this invention, provided that the residue does not participate in receptor binding or is otherwise involved in the biological activity of the molecule. Substitution or insertion of cysteine should not alter biological function and activity.

The effect of the cysteine insertion or substitution may be analyzed using biological assays that suitably and appropriately measure the function of the modified protein. Comparisons between the cysteine modified protein and the parent unmodified protein reveal the quantitative and qualitative effects of the modification on function. If data are available that identify, locate, or suggest where the important sites are located on the protein surface that contribute to biological activity, or which cannot be modified by mutagenesis, sites remote for those biologically and functionally sensitive regions may be avoided. For example, the cysteine substitution or insertion may be placed on the surface of the protein or peptide opposite from the functionally sensitive surfaces of the protein; i.e., spatially as far away as possible.

Cysteine substitutions or insertions for antibodies have been described (see U.S. Pat. No. 5,219,996). Methods for introducing Cys residues into the contant region of the IgG antibodies for use in site-specific conjugation of antibodies are described by Stimmel et al. (J. Biol. Chem 275:330445-30450, 2000).

V.B.3. Chemical Addition of Sulfhydryl Groups to Polypeptides and Other Macromolecules

If the desired sulfhydryl groups are not present on the protein, peptide or macromolecule, a sulfhydryl may be introduced by chemical modification. As a nonlimiting example, the sFv or a therapeutic macromolecule can be modified so as to introduce a thiol by chemical modification. A cysteine amino acid can be inserted or substituted on the surface of a protein or peptide by genetic manipulation. Sulfhydryl groups can be added by chemical modification using 2-iminothiolane (IT), also known as Traut's reagent. For example, a sulfhydryl can be introduced by incubating 0.1 to 10 mg/ml, preferably 1 to 5 mg/ml, of the target molecule, with a 1.1- to 100-fold, preferably 1.1- to 10-fold, molar excess of 2-iminothiolane in 50 mM triethanolamine, pH 8.0, containing 150 mM NaCl and 1 mM EDTA for three hours at 4° C. The excess 2-iminothiolane can then be removed by desalting on either a P 10 (Bio-Rad, Hercules, Calif.) or G25, G-50, or G-100 (Pharmacia, Piscataway, N.J.) size exclusion column equilibrated with 20 mM sodium phosphate containing 0.15 M NaCl and 1 mM EDTA, pH 7.3, (PBS-EDTA). The selection of either the P10, Sephadex G-25, or Sepharose G-100 columns for desalting is made according to the mass of the derivatized protein.

A protected sulfhydryl group can be added which allows storage of the derivatized protein without self-association through disulfide bond formation. IT and DTNB can be reacted together to form TNB-activated IT, which can then be directly added to the target molecule. Also, substituted IT's can be synthesized (Goff and Carroll, Bioconjugate Chem. 1:381-6, 1990), and using these to add sulfhydryl groups to target proteins can result in disulfide linked conjugates that exhibit increased stability in vivo (Carroll et al. Bioconjugate Chem. 5:248-56, 1994). Protected sulfhydryls can also be added by using a modification reagent that contains a protected thiol in addition to a group that selectively reacts with primary amines. For example, the N-hydroxy-succinimide ester of S-acetylthioacetic acid (SATA, Pierce Chemical Co., Rockford, Ill. can be used according to the manufacturer's instructions to introduce a protected thiol group on either the sFv or the therapeutic macromolecule. This can be accomplished by adding 5 μl of 15 mg/ml SATA in DMSO to 1.0 ml of 60 μM sFv or therapeutic macromolecule in 50 mM sodium phosphate, pH 7.5, containing 1 mM EDTA (P-EDTA). After incubation at room temperature for 30 minutes, the excess SATA can be removed by desalting on either a P10 or G25 size exclusion column equilibrated with P-EDTA. Deprotection of the thiol group can be done by incubating 1 ml of derivatized protein with 100 μl 50 mM sodium phosphate containing 25 mM EDTA and 0.5 M hydroxylamine, pH 7.5, for two hours at room temperature. The excess hydroxylamine can be removed by desalting on either a P10 or G25 size exclusion column equilibrated with PBS-EDTA. Alternatively, one could use N-succinimidyl S-acetylthiopropionate (SATP), which is similar to SATA, but has an additional carbon in the spacer arm. Its use is identical to SATA.

Sulfhydryl groups can also be added by using a modification reagent that contains a disulfide bond in addition to a group that selectively reacts with primary amines. For example, the heterobifunctional cross-linker sulfosuccinimidyl 6-[3′-(2-pyridyldithio)-propionamido] hexanoate (sulfo-LC-SPDP, Pierce Chemical Co.) will thiolate proteins when used according to the manufacturer's directions. Thiolation can also be performed by the addition of 300 μg sulfo-LC-SPDP per ml of 10 mg/ml sFv or therapeutic macromolecule in 20 mM sodium phosphate containing 0.15 M NaCl, pH 7.3 (PBS). Other, non-soluble, forms such as N-succinimidyl 3-(2-pyridyldithio)propionate (SPDP, Pierce Chemical Co.) or N-succinimidyl 6-[3′-(2-pyridyldithio)propionamido]hexanoate (LC-SPDP, Pierce Chemical Co.) can be used in these reactions by dissolving in DMSO to a concentration of 20 mM, and adding 25 μl to 1 ml of 10 mg/ml protein. Reducing the SPDP-derivatized protein under mild conditions will release pyridine-2-thione, leaving an aliphatic thiol. An example of a mild reducing condition is to add 1/100th volume of 1 M dithiothreitol (DTT) to the above SPDP-derivatized target protein and incubating for 30 minutes at room temperature, or incubate the SPDP-derivatized target protein with 50 mM 2-meraptoethylamine in PBS-EDTA for 90 minutes at 37° C. The excess SPDP, LC-SPDP or sulfo-LC-SPDP, and the pyridine-2-thione can then be removed by desalting on either a P10 (Bio-Rad, Hercules, Calif.) or G25 (PD10 column, Pharmacia, Piscataway, N.J.) column equilibrated with PBS-EDTA.

These modification reagents may also contain groups near the added thiol such that they form a hindered disulfide when oxidized. These reagents, such as 4-succinimidyloxycarbonyl-methyl-(2-pyridyldithio)-toluene (SMPT), may result in a conjugate that exhibits increased stability in vivo (Thorpe et al. Cancer Res. 47:5924-5931, 1987). Other cross-linking reagents can be used for protein thiolation and are known to those well versed in the art. Many of these reagents are described in the Pierce Chemical Co. catalog, or by Ji (Methods Enzymol. 91:580-609, 1983) and Hermanson (Bioconjugate Techniques, Academic Press, Inc., San Diego, 1-785, 1996).

V.B.4. Chemical Addition of Primary Amine Groups to the Surface of a Polypeptide or Macromolecule

If additional primary amines need to be added to either the sFv or the therapeutic macromolecule, they can be introduced through chemical modification or genetic manipulation. Chemical modification to add primary amines may either be reversible or non-reversible. For example, amination of cysteines can be performed using N-(iodoethyl) Trifluoroacetamide (Aminoethyl-8™, Pierce Chemical Co.) by a reaction in which the iodoalkyl group reacts specifically with sulfhydryl groups, forming a stable thioether bond and releasing free iodine. The trifluoroacetate protecting group can then be hydrolyzed to expose the introduced primary amine. A reversible amination of cysteines can be performed by using, for example, 2-aminoethyl-2′-aminoethanethiosulfonate (Pierce Chemical Co.). The primary amine generated by this compound can be removed by disulfide reducing agents.

V.B.5. Conjugation Between Sulfhydryl Residues

Most commonly, the sFv and therapeutic macromolecule will have either sulfhydryl or primary amines as the targets of the cross-linking reagents, and both sulfhydryl and primary amines can either exist naturally or be the result of chemical modification as described above. When both sFv and therapeutic macromolecule have a reduced sulfhydryl, a homobifunctional cross-linker that contains maleimide, pyridyl disulfide, or α-haloacetyl groups can be used for cross-linking. Examples of such cross-linking reagents include, but are not limited to, bismaleimidohexane (BMH) or 1,4-Di-[3′-(2′-pyridyldithio)propionamido]butane (DPDPB). Alternatively, a heterobifunctional cross-linker that contains a combination of maleimide, pyridyl disulfide, or α-haloacetyl groups can be used for cross-linking. Less preferably, the cross-linking reagent can contain thiophthalimide derivatives or disulfide dioxide derivatives. Also, extrinsic sulfhydryl groups can be introduced into the sFv and therapeutic macromolecule, and oxidized to cross-link by disulfide formation.

V.B.6. Conjugation Between Primary Amines

When primary amines are selected as the target both on sFv and therapeutic macromolecule, then a homobifunctional cross-linker that contains succinimide ester, imidoester, acylazide, or isocyanate groups can be used for cross-linking. Examples of such cross-linking reagents include, but are not limited to, Disuccinimidyl glutarate (DSG), Bis[2-(succinimidooxycarbonyloxy)ethyl]sulfone (BSOCOES), Bis[2-(sulfosuccinimidooxycarbonyloxy)ethyl]sulfone (sulfo-BSCOCOES), Disuccinimidyl suberate (DSS), Dithiobis(succinimidylpropionate) (DSP), BIS-(Sulfosuccinimidyl) Suberate (BS3), Dithiobis(sulfosuccinimidylpropionate) (DTSSP), Disuccinimidyl tartrate (DST), Disulfosuccinimidyl tartrate (sulfo-DST), Dithio-bis-maleimidoethane (DTME), Ethylene glycolbis(succinimidylsuccinate) (EGS), Ethylene glycolbis(sulfosuccinimidylsuccinate) (sulfo-EGS), Dimethyl malonimidate.2 HCl (DMM), Dimethyl succinimidate.2 HCl (DMSC), Dimethyl adipimidate.2 HCl (DMA), Dimethyl pimelimidate.2 HCl (DMP), Dimethyl suberimidate.2 HCl (DMS), and Dimethyl 3,3′-dithiobispropionimidate.2HCl (DTBP). Heterobifunctional cross-linkers that contains a combination of imidoester or succinimide ester groups can also be used for cross-linking.

V.B.7. Conjugation Between Sulfhydryls and Primary Amines

Heterobifunctional cross-linking reagents that combine selective groups against different targets are generally preferred because these allow reactions to be performed selectively and sequentially, minimizing self-association or polymerization. Also, heterobifunctional reagents allow selection of chemistry appropriate for the individual molecules to be joined, minimizing inhibition of enzymatic, binding, signaling or other activities required for the sFv-therapeutic macromolecule conjugate. For example, some enzymes have a primary amine present in the active site and modification of this amine will inhibit enzymatic function. These enzymes would be suitable prospects for alternative conjugation chemistry so that a thiol group is modified rather than the amine required for therapeutic activity. Examples of such cross-linking reagents include, but are not limited to, N-succinimidyl 3-(2-pyridyldithio)propionate (SPDP), N-succinimidyl 6-[3′-(2-pyridyldithio)propionamido]hexanoate (LC-SPDP), sulfosuccinimidyl 6-[3′-(2-pyridyldithio)-propionamido] hexanoate (sulfo-LC-SPDP), m-maleimidobenzoyl-N-hydoxysuccinimide ester (MBS), m-maleimidobenzoyl-N-hydoxysulfosuccinimide ester (sulfo-MBS), succinimidyl 4-[P-maleimidophenyl] butyrate (SMPB), sulfosuccinimidyl 4-[p-maleimidophenyl] butyrate (sulfo-SMPB), N-[γ-Maleimidobutyryloxy] succinimide ester (GMBS), N-[γ-maleimidobutyryloxy] sulfosuccinimide ester (sulfo-GMBS), N-[ε-maleimidocaproyloxy] succinimide ester (EMCS), N-[ε-maleimidocaproyloxy] sulfosuccinimide ester (sulfo-EMCS), N-succinimidyl(4-iodoacetyl)aminobenzoate (SIAB), sulfosuccinimidyl(4-iodoacetyl)aminobenzoate (sulfo-SIAB), succinimidyl 4-(N-maleimidomethyl)cyclohexane-1-carboxylate (SMCC), sulfosuccinimidyl 4-(N-maleimidomethyl)cyclohexane-1-carboxylate (sulfo-SMCC), succiminidyl-4-(N-maleimidomethyl)cyclohexane-1-carboxy-(6-amido-caproate) (LC-SMCC), 4-succinimidyloxycarbonyl-methyl-(2-pyridyldithio) toluene (SMPT), and sulfo-LC-SMPT.

V.C. Thiol Modifications and Disulfide Bridges

V.C.1. Formation of Disulfide Bridges (Reaction of Thiols with Ellman's Reagent, DTNB)

The disulfide within DTNB [5,5′-dithio-bis-(2-nitrobenzoic acid)] exchanges with the thiol group of proteins and peptides and other macromolecules. For each thiol group that undergoes disulfide exchange with DTNB to form a mixed disulfide, one molecule of 5-thio-2-nitrobenzoic acid (TNB) is released. At alkaline pH (pH above 7.5, and preferable above pH 8.0), the absorbance at 412 nm is measured to calculate the molar concentration of TNB using an extinction coefficient of 1.36×104 M−1 cm−1 (at pH 8.0). The protein is used at a concentration of 1 to 10 mg/ml in a solution of phosphate buffered saline, pH 8. Ellman's reagent may be dissolved in 0.1 M sodium phosphate, pH 8, at a concentration of 4 mg/ml. Four (4) μl of Ellman's reagent is mixed with each 100 μl protein solution and the absorbance at 412 nm measured. The concentration of TNB is determined using the extinction coefficient at pH 8.

The protein is separated as a mixed disulfide (protein —SS-TNB) from Ellman's reagent and TNB by gel filtration on a column of Sephadex G-25 or Biorad P10 in an aqueous buffer.

V.C.2. Blocking Thiols by Alkylation

Iodoacetate and iodoacetamide are used to react with free thiols on proteins and prevent them from forming unwanted disulfide bonds. Iodoacetamide is a highly reactive and selective reagent for thiols and may be used as a blocking reagent. Iodoacetamide will, however, react slowly with histidine on its imidazole side chain.

The protein, peptide, or macromolecule containing thiol groups that are to be blocked are present at a concentration of 0.1 to 10 mg/ml, preferably 1 to 5 mg/ml, in an aqueous buffer at neutral or slightly alkaline pH, pH 6.5 to 10, preferably pH 7.0 to 8.0. Freshly dissolved iodoacetamide is added to a final concentration of 0.1 to 10 mM, preferably 0.5 to 2 mM and reacted at room temperature for 1 hour. The reaction is preferably carried out in the dark. Iodoacetamide is preferably free from free iodine, whose presence can be detected by its color (yellowish) or other methods known to those skilled in the art.

V.C.3. Blocking Thiols by Maleimidation

N-ethylmaleimide reacts rapidly with thiols to form a stable thioether bond that is not cleaved by reduction with 2-mercaptoethanol or dithiothreitol. The protein, peptide, or macromolecule having a thiol group that is desired to be blocked is dissolved in an aqueous buffer at pH 6.5 to 8.0, preferably 6.5 to 7.5. Sodium phosphate buffer (0.01 to 0.1 M) at pH 7 to 7.5 is preferred. The buffer may also contain 0.01 to 0.5 M NaCl, and preferably 0.01 to 0.1 M. N-ethylmaleimide may be freshly dissolved in an aqueous buffer at a concentration so that after dilution the final concentration of N-ethylmaleimide is between 1.1- and 20-fold molar excess, and preferably 2- to 10-fold molar excess. After 5 to 120 minutes, and preferably 5 to 30 minutes, of reaction at room temperature, the modified protein may be separated from excess N-ethylmaleimide by gel flitration on a column of Sephadex G-25 or Biorad P10.

VI. Methods of Isolation and Purification

After synthesis, it is preferred that a composition or compound isolated or purified, preferably substantially purified. By “isolated” it is meant that the composition or compound has been separated from any molecule that interferes with the biological activity or pIgR-targeting capacity thereof. As used herein the term “substantially purified” means at least about 95%, preferably at least about 99%, free of other components used to produce and/or modify the protein conjugate. The term “purified” refers to a composition or compound that has been separated from at least about 50% of undesirable elements. Techniques and methods for the separation and isolation of functional conjugates comprising sFv5A are used herein as non-limiting examples, but the techniques any be applied to any stalk-binding protein conjugate of the invention.

The purification of the sFv's and the conjugated material is achieved using any of the methods that are known by those skilled in the art to purify proteins, peptides, and macromolecules. Such methods include gel filtration, HPLC using ion exchange chromatography, immobilized metal affinity chromatography, hydrophobic interaction chromatography, selective precipitation, and crystallization.

Chromatography methods are selected for their ability to remove unreacted reagents, including unreacted derivatized proteins, peptides, and macromolecules and unreacted pIgR binding ligands. Chromatography methods are also selected for their ability to separate conjugates having different molar rations or protein, peptide, or macromolecule to pIgR binding ligands. Such conjugates are often referred to as 1-'mers (1:1 conjugates), 2-'mers (2:1 conjugates), 3-'mers (3:1 conjugates), etc. The production of different 'mers is a function of the number of reactive groups present on each molecule incubated in the conjugation mixture.

VI.A. Purification Elements

Optional protein elements can be incorporated into a fusion protein, which may be a compound of the invention or a member of a protein conjugate of the invention, or which may be comprised in a composition of the invention, and used during its purification and/or preparation. For example, as is discussed in more detail above, a protein member may include a protein purification element such as, for example, a “His tag” (His6). A His-tagged protein member or conjugate thereof can be isolated, or at least partially purified, from a composition that further comprises undesirable compounds by contacting the composition with a column of nickel immobilized on a metal-binding matrix. The His-tagged protein member or conjugate will bind to the nickel column and will thus be retained in the column; undesirable compounds pass through the column. As is explained above in more detail, various methods may be used to remove the protein purification element from the protein member or conjugate after such steps.

Post-translational modifications to a polypeptide may be created in vitro or in vivo. Various chemical treatments can be used for in vitro modifications of pure or semi-pure proteins; whereas in vivo modifications result from the choice of expression system and host cells. Post-translational modifications include, by way of non-limiting example, glycosylation, cleavage, phophorylation, cross-linking, formation or reduction of disulfide bridges, and the like.

VI.B. Affinity Chromatography

Polypeptides that contain pIgR-derived amino acid sequences that are identical or similar to the epitopes to which sFv molecules that bind pIgR are prepared according to known methods. The epitope-containing polypeptides are covalently coupled to thiol Sepharose (activated thio Sepharose 4B contains a thiol group to which peptides may be attached covalently). A thiol containing peptide is reacted with Ellman's reagent (DTNB) to form a mixed disulfide. The TNB-peptide is separated from 2-nitro-5-thiobenzoic acid by gel sizing column chromatography. The TNB-peptide is reacted with thiol Sepharose to form a mixed disulfide of the peptide covalently bound to the resin.

As another example, a maleimido group is placed at the amino or carboxyl terminal of the peptide. The maleimido group on the peptide is reacted with thiol Sepharose to form a thioether bond. Alternatively, the epitope-containing polypeptides are covalently coupled to activated supports that react with primary amines present on the polypeptide. Such supports include cross-linked agarose or acrylic matrices that have functional groups such as N-hydroxysuccinimide. These activated supports include Affi-Gel 10 (Bio-Rad), Affi-Gel 15 (Bio-Rad), Affi-Prep 10 (Bio-Rad) and NHS-activated Sepharose 4 Fast Flow (Pharmacia). Immobilization of the polypeptide may also be performed with epoxy-activated matrices such as Epoxy-activated Sepharose 6B (Pharmacia) or cyanogen bromide-activated matrices such as CnBr-activated Sepharose 4 Fast Flow (Pharmacia).

The peptide-Sepharose resin is used to bind an sFv, or other antibody derivative that binds the epitope in pIgR that is recognized by the antibody, or a conjugate comprising such an antibody. Depending on the epitope to which the sFv binds in pIgR, the amino acid sequence may be modified to provide the epitope in an amino acid sequence that inlcudes a residue that may be covalently linked to thiol Sepharose.

In the case of sFv5 and its derivatives (sFv5AF and sFv5AF-Cys), the amino acid sequence of the epitope in pIgR is known, see U.S. patent application Ser. No. ______ (attorney docket No. 18062E-000900US), entitled “Ligands Directed To The Non-Secretory Component, Non-Stalk Region of pIgR and Methods of Use Thereof” filed Mar. 26, 2001 by Mostov et al. The amino acid sequence is, at a minimum, DPRLF. The maximum epitope amino acid sequence is QDPRLF in human and LDPRLF, which suggests that the most amino-terminal residue in the epitope sequence is not required for binding to sFv5.

After the sFv or conjugate has been applied to the column, unreactive material is washed through the column. The sFv's, or conjugates comprising sFv's, remain attached to the column through specific interaction with the peptide. The specifically bound sFv or conjugate is separated from the column by low pH (pH 3 to 4) treatment for a brief time (preferably less than 15 minutes and preferably less than 5 minutes), by passing free peptide over the column, or by reducing the covalently bound peptide with DTT or mercaptoethanol. When using a free peptide to obtain elution of the sFv or conjugate, the peptide need only contain the epitope to which the sFv binds or it may contain the same peptide sequence (without the cysteine) used to conjugate to the resin.

For maleimide conjugated peptide to the thiol Sepharose resin, reduction will not release the peptide:sFv or conjugate complex. Therefore, elution with free peptide or low pH may be used.

The sequence within the epitope may be varied such that the interaction is weakened compared to the native epitope. By substituting different amino acids within the sequence, a weaker binding peptide sequence may be identified. Weak binding to the immobilized peptide on thiol Sepharose is used to obtain some retention of sFv and conjugates on the column and to allow nonbinding components to pass straight through the column without binding. Therefore, no strenuous conditions may be required for elution and free peptide may not be required for elution. Tribbick et al. (J. Immunol. Methods 139: 155-166, 1991) have described a similar approach. A weak binding peptide epitope is identified by performing alanine scans on the epitope to identify the amino acid side chains that provide most of the binding specificity and strength.

A peptide epitope is identified using a set of peptides designed to explore all of the binding regions of a protein, a general net. All overlapping peptides of a defined length, homologous with the protein, are synthesised. The offset is set from 1 to 5 residues, and preferably 3 to 4 residues in the first trials. The peptides should be sufficiently long so as not to miss an epitope by ‘dividing it’ between two peptides in the nested set. The peptides should be preferably 8 to 12 amino acids in length and preferably 10 to 15 amino acids in length. The boundaries of the epitope may be more precisely identified using a process that examines the linear sequence of the protein through a series of moving windows of a different size—a window net. The contributions of each amino acid side chain in the epitope are estimated by substituting each amino acid position in the epitope with all of the other 19 amino acids and determining the effect on the binding characteristics of the sFv to the peptide—a replacement net. Such strategies are described by Geysen et al. (Mol. Immunol. 23: 7090715, 1986), Geysen et al. (J. Immunol. Methods 102: 259-274, 1987), Tribbick et al. (J. Immunol. Methods 139: 155-166, 1991), and Geysen et al. (J. Mol. Recog. 1: 32-41, 1988).

VI.C. Ion Exchange Chromatography

In ion exchange chromatography, charged substances are separated via column materials that carry a charge. In cation exchange, the solid phase carries a negative charge whereas, in anion exchange, the stationary phase carries a positive charge. The solid phase of the columns is composed of ionic groups that are covalently bound to a gel matrix. Before a sample is passed through the column, the ionic charges in the solid phase are compensated by small concentrations of counter-ions present in the column buffer. When a sample is added to the column, an exchange occurs between the weakly bound counter-ions in the column buffer and more strongly bound ions present in the sample. Bound molecules do not elute from the column until a solution of varying pH or ionic strength is passed through the column. If desired, the degree of separation may be improved by a change in the gradient slope. If a compound of interest does not bind to the column under the selected conditions, the concentration and/or the pH value of the starting buffer can be changed.

Ion chromatography of polypeptides occurs because polypeptides are multivalant anions or cations. Under strongly basic conditions, polypeptides are anions because the amino group is a free base and the carboxy group is dissociated. Under strongly acidic conditions polypeptides are cations as a result of suppression of the dissociation of the carboxy group and protonation of the amino group. Due to the net charge of the polypeptides it is possible to bind them to a corresponding charged stationary phase as long as the salt concentration is kept low.

Various ion-exchange resins, cellulose derivatives and large-pore gels are available for chromatographic use. Ion-exchange materials are generally water insoluble polymers containing cationic or anionic groups. Non-limiting examples of cation exchange matrices have anionic functional groups such as —SO₃⁻, —OPO₃⁻ and —COO⁻, and anion exchange matrices may contain the cationic tertiary and quaternary ammonium groups having the general formulae —NHR⁺⁺ and —NR⁺⁺⁺. Proteins become bound by exchange with the associated counter-ions.

For reviews of ion-exchange chromatography, see Bollag, Ion-exchange chromatography, Methods Mol Biol 36:11-22, 1994; Holthuis et al., Chromatographic techniques for the characterization of proteins, Pharm Biotechnol 7:243-99, 1995; and Kent, Purification of antibodies using ion-exchange chromatography, Methods Mol Biol 115:19-22, 1999.

VI.D. Hydrophobic Interaction Chromatography

Separation of polypeptides and other compounds by hydrophobic interaction chromatography (HIC) is based on the hydrophobicity of the compounds presented to the solvents. HIC separates compounds by mechanisms similar to reversed-phase chromatography (RPC) but under gentle reverse salt gradient elution conditions in aqueous buffers. Because no organic solvent is used in HIC, the biological activity of polypeptides and other compounds is more likely to be retained.

HIC involves sequential adsorption and desorption of protein from solid matrices mediated through non-covalent hydrophobic bonding. Generally, sample molecules in a high salt buffer are loaded on the HIC column. The salt in the buffer interacts with water molecules to reduce the solvation of the molecules in solution, thereby exposing hydrophobic regions in the sample molecules which are consequently adsorbed by the HIC column. The more hydrophobic the compound, the less salt needed to promote binding. A decreasing salt gradient may be used to elute samples from the column. As the ionic strength decreases, the exposure of the hydrophilic regions of the molecules increases, and compounds elute from the column in order of increasing hydrophobicity. Sample elution may also be achieved by the addition of mild organic modifiers or detergents to the elution buffer. Non-limiting examples of HIC-immobilized functional groups that can function to separate compounds include octyl groups, such as those on Octyl Sepharose CL4B media from Pharmacia, and propyl groups, such as those on High Propyl media from Baker. Alkoxy, butyl, and isoamyl functional group resins may also be used.

Hydrophilic interaction chromatography (HILIC) separates compounds by passing a hydrophobic or mostly organic mobile phase across a neutral hydrophilic stationary phase, causing solutes to elute in order of increasing hydrophilicity. For example, with neutral peptides one may use 15 mM ammonium formate and reverse organic conditions. Highly charged molecules require low amounts (e.g., 10 mM) of salt for ion suppression, and a slight perchlorate or sulfate gradient (in a high organic solvent concentration) to effect desorption. HILIC has been used successfully with phosphopeptides, crude extracts, peptide digests, membrane proteins, carbohydrates, histones, oligonucleotides and their antisense analogs, and polar lipids.

In hydrophobic-interaction chromatography, compounds of relatively greater hydrophobicity are retained longer on the column relative to those compounds that are more hydrophilic. Conversely, using hydrophilic-interaction chromatography, hydrophilic compounds are retained longer on the column relative to those compounds that are more hydrophobic.

For reviews and exemplary uses of hydrophobic interaction chromatography (HIC), see Ghosh, Separation of proteins using hydrophobic interaction membrane chromatography, J Chromatogr A 923(1-2):59-64, 2001; Queiroz et al., Hydrophobic interaction chromatography of proteins, J Biotechnol 87(2):143-59, 2001; Arakawa et al., Solvent modulation in hydrophobic interaction chromatography, Biotechnol Appl Biochem 13(2):151-72, 1991; el Rassi et al., Reversed-phase and hydrophobic interaction chromatography of peptides and proteins, Bioprocess Technol 9:447-94, 1990; Kato, High-performance hydrophobic interaction chromatography of proteins, Adv Chromatogr 26:97-115, 1987; Hjerten, Hydrophobic interaction chromatography of proteins, nucleic acids, viruses, and cells on noncharged amphiphilic gels, Methods Biochem Anal 27:89-108, 1981; Ochoa, Hydrophobic (interaction) chromatography, Biochimie 60(1):1-15, 1978; and in Protein Purification, 2d Ed., Springer-Verlag, New York, pgs 176-179 (1988).

For reviews and exemplary uses of hydrophilic interaction chromatography (HILIC), see Zhu et al., Hydrophilic-interaction chromatography of peptides on hydrophilic and strong cation-exchange columns, J Chromatogr 548(1-2):13-24, 1991; Olsen, Hydrophilic interaction chromatography using amino and silica columns for the determination of polar pharmaceuticals and impurities, J Chromatogr A. 913(1-2):113-22, 2001; Olsen, Hydrophilic interaction chromatography using amino and silica columns for the determination of polar pharmaceuticals and impurities, J Chromatogr A. 913(1-2): 113-22, 2001; and Alpert et al., Hydrophilic-interaction chromatography of complex carbohydrates, J Chromatogr A. 676(1):191-22, 1994; and Alpert, Hydrophilic-interaction chromatography for the separation of peptides, nucleic acids and other polar compounds, J Chromatogr. 499:177-96, 1990.

VI.E. Assaying Purity and Activity

During or after the purification process, it is often desirable to monitor both the amount and biological activity of the composition, complex or compound being purified. The amount of the composition or compound can be detected by using antibodies directed to an epitope thereof. Additionally or alternatively, a composition or compound of the invention may comprise a detectable polypeptide by which the protein conjugate may be monitored.

Some of the biological activities of a composition or compound of the invention will vary depending on the nature of the biologically active polypeptide(s) included therein, and assays specific for the biological activities of the parent proteins are used. The compositions or compounds are also assayed for their ability to bind pIgR and undergo various forms of cellular trafficking. Assays for these and pIgR-related attributes are described herein and are applicable to any of the compositions or compounds of the invention.

Purity can be assessed by any suitable method, including HPLC analysis and staining of gels through which an aliquot of the preparation containing the protein conjugate has been electrophoresed. Those practiced in the art will know what degree of isolation or purification is appropriate for a given application. For example, (in the U.S. at least) biologicals do not have to meet the same standard of purity for, e.g., a compound.

VII. Multivalent and Polyspecific Complexes and Compounds

In one aspect the invention encompasses multivalent and polyspecific complexes and compounds. By “multivalent” it is meant that the complex or compound has two or more targeting elements directed to the same target. The two or more targeting elements may, but need not, be identical. By “polyspecific” it is meant that the complex or compound has at least one targeting element that is directed to a first target, and a second targeting element directed to a second target. A variety of methods, including but not limited to the following, can be used to prepare multivalent and polyspecific complexes and compounds of the invention.

VII.A. Single-Chain Antibodies

Diabodies are dimeric antibody fragments (Hollinger et al., “Diabodies”: small bivalent and bispecific antibody fragments, Proc Natl Acad Sci USA 1993 Jul. 15; 90(14):6444-8). In each polypeptide, a heavy-chain variable domain V(H) is linked to a light-chain variable domain V(L) but unlike single-chain Fv fragments, each antigen-binding site is formed by pairing of one V(H) and one V(L) domain from the two different polypeptides. Diabodies thus have two antigen-binding sites, and can be bispecific or bivalent. (Perisic et al., Crystal structure of a diabody, a bivalent antibody fragment, Structure 1994 Dec. 15; 2(12):1217-26).

VII.A.1 Directing Multimerization of SFv's by Altering Linker Length in SFv Antibodies

The length of the linker(s) between V-domains influences the size, flexibility and valency of single chain Fv antibody fragments (sFv's). sFv molecules are predominantly monomeric when the V(H) and V(L) domains are joined by polypeptide linkers of at least 12 amino acid residues. An sFv molecule with a linker of 3 to 12 amino acid residues is less likely to fold into a monomer, i.e., a single chain Fv in which the V(H) and V(L) domains are paired intramolecularly. However, sFv's that do not easily form monomers may interact with a second sFv molecule to form a “diabody”. Diabodies may be bispecific (Müller et al., “A dimeric bispecific miniantibody combines two specificities with avidity”, Federation of European Biochemical Societies, 432 (1998), pp. 45-49) or bivalent. A bivalent diabody is formed from two sFv's that are identical to, or substantially the same as, each other; it has two binding [V(H)::V(L)] regions directed to the same target molecule. A bispecific diabody is formed from two sFv's that are different from each other, and has two binding [V(H)::V(L)] regions, each of which is directed to a different target molecule.

Reducing the linker length below three amino acid residues can force sFv molecules to associate to form multimers (e.g., trimers a.ka. triabodies, tetramers a.k.a., tetrabodies, etc.) depending on linker length, composition and V-domain orientation (see, e.g., U.S. Pat. No. 5,837,242). The increased valency in sFv multimers may result in higher avidity (low off-rates) (Hudson et al., High avidity scFv multimers; diabodies and triabodies, J Immunol Methods 1999 Dec. 10; 231(1-2):177-89; Todorovska et al., Design and application of diabodies, triabodies and tetrabodies for cancer targeting, J. Immunol Methods 2001 Feb. 1; 248(1-2):47-66; Hudson et al., High avidity scFv multimers; diabodies and triabodies, J Immunol Methods 1999 Dec. 10; 231(1-2):177-89).

Single-chain antibodies having V(H) and V(L) domains with 10-residue (Gly4Ser)2 or five-residue (Gly4Ser) linkers, or no linkers, have been examined. In one report (Kortt et al., Single-chain Fv fragments of anti-neuramimidase antibody NC10 containing five- and ten-residue linkers form dimers and with zero-residue linker a trimer, Protein Engineering, 10:423-433, 1997), the zero-residue linker sFv formed a trimer with three active antigen combining sites. BIAcore biosensor experiments showed that the affinity of each individual antigen combining site in both the 10- and five-residue linker sFv dimers and zero-residue liner sFv trimer was essentially the same when the sFvs were immobilized onto the sensor surface. However, when the sFv was used as the analyte, the dimeric and trimeric sFv's showed an apparent increase in binding affinity due to the avidity of binding the multivalent sFv's.

In general, sFv molecules in which the number of amino acid residues between the V(H) and V(L) domains is 0 to 15 are less likely to form monomers and are more likely to form some type of multimer. When the linker length is 1 or 2 amino acids, trimers and/or other multimers are more likely to form. Linker lengths of 3 to 12 amino acids favor the formation of dimers, where sFv's having linkers of 12 or more more amino acids are more likely to form monomers. These rules are not absolute, however, those skilled in the art can prepare and analyze sFv's with differing linker lengths and test them for the presence of monomers and various multimers.

Higher multimers of sFv molecules may be polyvalent, polyspecific, or both (see, e.g., Müller et al., “A dimeric bispecific miniantibody combines two specificities with avidity”, Federation of European Biochemical Societies, 432 (1998), pp. 45-49). Using triabodies as a non-limiting example of higher multimers of sFv's, it can be seen that there are three possible combinations of sFv molecules. First, a triabody may comprise three identical or substantially identical sFv molecules, each of which is directed to the same target molecule, and is thus a trivalent triabody. Second, a triabody may comprise three different sFv molecules, each of which is directed to a different target molecule, and is thus a trispecific triabody. Third, a triabody may comprise two types of sFv molecules, a pair of which (sFv1a and sFv1b) is directed to a target molecule #1, whereas the third sFv in the triabody is directed to target molecule #2. The latter triabody is both bispecific, as it specifically binds both target molecule #1 and target molecule #2, and bivalent, as it has two binding regions directed to target molecule #1.

VII.A.3. Disulfide-Stabilized Single-Chain Antibodies (dsFv's)

Disulfide-stabilized sFv's (dsFv's) are recombinant Fv fragments of antibodies in which the unstable variable heavy V(H) and variable light V(L) heterodimers are stabilized by disulfide bonds engineered at specific sites that do not appreciably alter the binding activity of the sFv. Such sites lie between structurally conserved framework positions of V(H) and V(L). It should be possible to use positions in conserved framework regions to disulfide-stabilize many different sFv's (Reiter et al., Stabilization of the Fv fragments in recombinant immunotoxins by disulfide bonds engineered into conserved framework regions, Biochemistry 1994 May 10; 33(18):5451-9). In addition to influencing the tendency of a sFv molecule to form monomers or multimers, sFv molecules into which Cys residues have been introduced into may in some instances have altered production and stability characteristics.

To improve the stability of Fv molecules, a cysteine residue is introduced into conserved framework regions of both the heavy and light variable domains at positions compatible with the formation of an interdomain disulfide linkage. A disulfide-stabilized Fv (dsFv) may be more resistant to denaturation by heat or urea treatment than the corresponding single-chain Fv (sFv). Moreover, the yield of dsFv may be higher than that of the sFv (Webber et al., Preparation and characterization of a disulfide-stabilized Fv fragment of the anti-Tac antibody: comparison with its single-chain analog, Mol Immunol 1995 March; 32(4):249-58; Reiter et al., Antitumor activity and pharmacokinetics in mice of a recombinant immunotoxin containing a disulfide-stabilized sFv fragment, Cancer Res 1994 May 15; 54(10):2714-8).

Molecular graphic modeling may be used to identify sites for the introduction of interchain disulfide bonds in the framework region of sFv molecules. Mutations that result in the Cys-modification of the sites are introduced in the reading frame that encodes the sFv molecule using any appropriate method, e.g., PCR-mediated mutagensis. The disulfide-stabilized Fv (dsFv) is expressed and tested for its binding activity (Luo et al., V1-linker-Vh orientation-dependent expression of single chain Fv-containing an engineered disulfide-stabilized bond in the framework regions, J Biochem (Tokyo) 1995 October; 118(4):825-31).

VII.B. Production of Polyspecific and Multivalent Antibody Fragments

VII.B.1. Production Via Recombinant DNA

Technologies that are suitable for the production of multivalent antibody derivatives include F(ab′)2 assembled from Fab′ fragments expressed in E. coli or isolated by limited proteolysis of a monoclonal antibody; F(ab′)2 assembled using leucine zippers; and diabodies (Carter et al., Toward the production of bispecific antibody fragments for clinical applications, J Hematother 1995 October; 4(5):463-70).

One method for the construction of diabodies uses a refolding system (Takemura et al., Construction of a diabody (small recombinant bispecific antibody) using a refolding system, Protein Eng 2000 August; 13(8):583-8). Multivalent disulfide-stabilized sFv's (dsFv's) can be prepared by a variety of methods, including but not limited to phage display (Brinkmann et al., Phage display of disulfide-stabilized Fv fragments, J Immunol Methods 1995 May 11; 182(1):41-50). Improved yields of multivalent sFv's may be achieved using the P. pastoris expression/secretion system (Goel et al., Divalent forms of CC49 single-chain antibody constructs in Pichia pastories: expression, purification, and characterization, J. Biochem (Tokyo) 2000 May; 127(5):829-36; Powers et al., Expression of single-chain Fv-Fc fusions in Pichia pastoris, J Immunol Methods 251(1-2):123-35, 2001).

Cloning strategies are known that can be used to create repertoires of diabody molecules having two different antigen binding sites (bispecific diabodies) or two of the same, or substantially the same, binding sites (bivalent diabodies) (McGuinness et al., Phage diabody repertoires for selection of large numbers of bispecific antibody fragments, Nat Biotechnol 1996 September; 14(9):1149-54; Pluckthun et al., New protein engineering approaches to multivalent and bispecific antibody fragments, Immunotechnology 1997 June; 3(2):83-105); Poljak, Production and structure of diabodies, Structure 1994 Dec. 15; 2(12):1121-3; and U.S. Pat. No. 6,071,515).

Phage displaying bivalent diabodies, or multiple copies of sFv monomers, are used to identify multivalent compounds and complexes that bind domain 6, the pIgR stalk, or any other portion or region of pIgR. Phage displaying bivalent diabodies, or multiple copies of sFv monomers, are used to identify multivalent compounds and complexes that are more efficiently endocytosed than phage displaying monomeric sFv. Measurement of phage recovery from within the cytosol as a function of applied phage titer is used to measure the relative endocytotic properties of phage displaying multivalent sFv's (Becerril et al., Toward selection of internalizing antibodies from phage libraries, Biochem Biophys Res Commun 1999 Feb. 16; 255(2):386-93). Methods of identifying phage displaying sFv molecules, and other polypeptide sequences, that confer transcytotic and/or paracellular transporting properties are described in U.S. patent application Ser. No. 60/266,182 (attorney docket No. 057220.0701) entitled “Compositions and Methods for Identifying, Characterizing, Optimizing and Using Ligands to Transcytotic Molecules” by Houston, L. L., and Sheridan, Philip L., filed Feb. 2, 2001, is drawn to the identification and use of ligands and targeting elements directed to transcytotic and transepithelial molecules.

Multivalent and/or polyspecific compounds and moieties derived from T-cell receptors may also be prepared. See, e.g., Golden et al., High-level production of a secreted, heterodimeric alpha beta murine T-cell receptor in Escherichia coli, J Immunol Methods 1997 Aug. 7; 206(1-2):163-9.

VII.B.2. Production by Chemical Treatment of Single Chain Antibodies

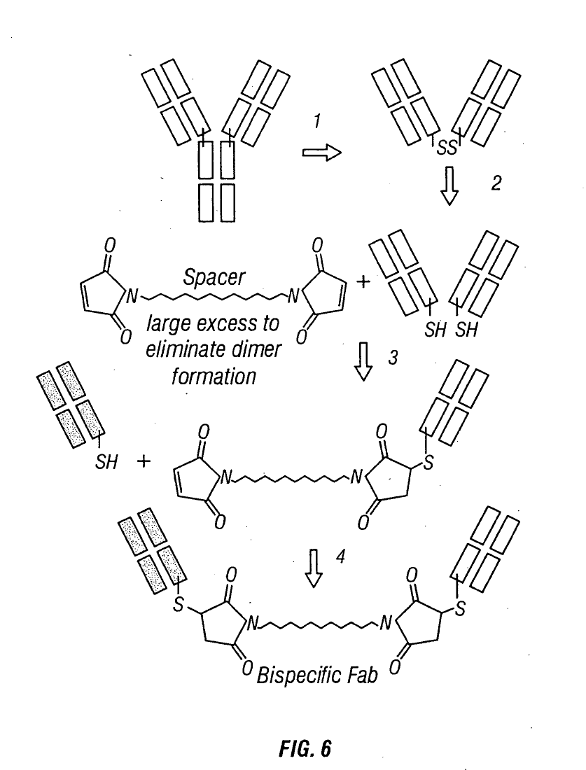

By substituting sFv molecules that contain an genetically inserted cysteine, either at the amino or carboxy terminus of the sequence or at some place on the surface of the sFv that does not interfere with its ability to recognize its antigen, a bispecific sFv can be formed using the reactions 3 and 4 in FIG. 6. A sFv that recognizes pIgR can be genetically modified to contain a cysteine residue. By reaction of that cysteine with a large molar excess of the bis-maleimido compound, a single bis-maleimido-sFv conjugate will be formed that has an additional reactive maleimide group. By purifying the conjugate away from the unreacted bis-maleimide, a pure sample of the conjugate can be obtained. This conjugate can be reacted in a second reaction with another sFv, having a different recognition specificity, that also contains a cysteine, which can be at the amino or carboxy terminus or on the surface of the sFv. In both types of sFvs, cysteines at the amino or carboxy terminus can be genetically attached using a spacer that provides sufficient distance between the surface of the sFv and the cysteine to facilitate chemical reactivity and to allow flexibility between the two conjugated sFv moieties. Either of the sFvs can be in any orientation, VL-linker-VH or VH-linker-VL. Either of the sFvs may contain the cysteine on a spacer (or within the spacer sequence) at the amino or carboxy terminus or on the surface of the sFv. The spacer can be comprised of (Gly4Ser)x, where x may be 1 to 5 and preferably 2 or 3. Other spacers may also be used, but the spacer should not be immunogenic.

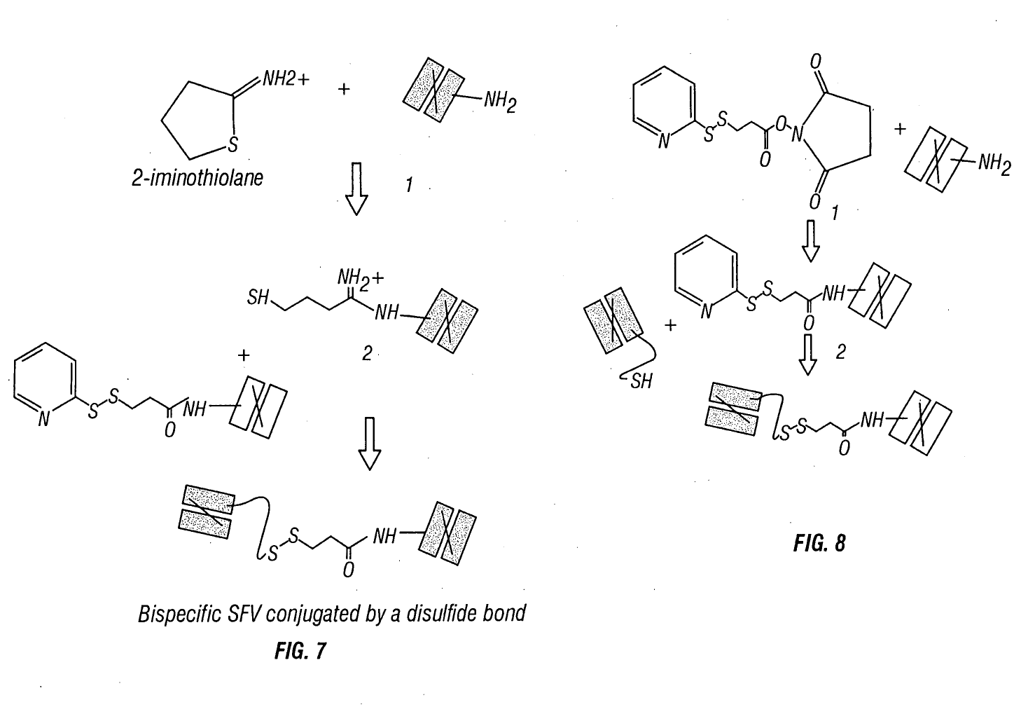

A reducible disulfide bond can be placed between two sFv molecules that have different recognition properties. FIG. 7 illustrates linkage using one sFv that has been derivatized with 2-iminothiolane, a reagent that reacts with amino groups (lysines predominantly and the alpha-amino group of proteins and peptides), and one sFv that has been derivatized with SPDP (N-succinimidyl-3-(2-pyridyldithio)-propionate, a reagent that also reacts with amino groups. A variety of other crosslinking reagents, such as those described in the Pierce Chemical Company 1994 catalogue (see

FIG. 8 illustrates another method of forming a disulfide bond between two different sFv molecules. One sFv is modified with SPDP. After separation of the excess reagents, a disulfide interchange is carried out by the addition of a sFv with a cysteine added to its surface by cysteine substitution or by addition of a spacer containing a cysteine.

VII.C. Production of Polyspecific and Multivalent Antibody Fragments by Chemical Treatment of Whole Antibodies

VII.C.1. F(ab′)2

F(ab′)2 fragments made from the bispecific antibody can be produced using conventional approaches. Enzymatic cleavage of Ig molecules depends on the characteristics of the individual molecule. For example, pepsin is not always successful in producing F(ab′)2 fragments, and the digestion conditions frequently needed optimization before acceptable yields are produced. In addition to pepsin, ficin can be used for F(ab′)2 production.

VII.C.2. Fab′-SH

F(ab′)2 fragments are held together by disulfide bond(s) between the H chains of the divalent molecule. By mild reduction, the Fab′ fragments, which are monovalent, can be released. 2-Mercaptoethylamine and other educing agents are used for this purpose.

VII.C.3. Fab

Fab fragments are monovalent antibody fragments that can be produced by papain digestion. A convenient method is to use papain immobilized on a resin so that the enzyme can be easily removed and the digestion terminated. Fab fragments do not have the disulfide bond(s) that are present in F(ab′)2 between the H chains.

VII.C.2.4. Fab Bispecific Entity

The reduction of a F(ab′)2 molecule, which can be formed by pepsin digestion or ficin digestion in the presence of 1 mM cysteine of an intact antibody, produces Fab′-SH. Fab′-SH can be converted to a mixed disulfide with Ellman's Reagent. An Fab′-SH that has a different specificity may then be used in a disulfide interchange to form a Fab bispecific entity that contains combining sites directed at two different antigens.

VII.C. Another method of linking Fab's uses a nonreducible covalent bond. One of the Fab′-SH partners can be modified with a bifunctional reagent, such as a reagent having two reactive maleimido groups (a bis-maleimido compound). If the maleimido reagent is in large molar excess over the Fab-SH, then only of the maleimido groups on the bis-maleimido will react to form a thioether bond. Therefore, a derivative such as Fab′-S-maleimide-spacer-maleimido group will be isolated. This derivative can be reacted with a nearly equal molar amount of another Fab′-SH (having a different antigen recognition specificity) to form another thioether bond. The product of this series of reactions is a divalent molecule that has two different recognition specificities that are held together by thioether bonds. The spacer may be relatively short, as in bis-maleimidohexane, or may be longer and more hydrophilic, as in (Gly4Ser)₃.

VII.D. Multivalent and Polyspecific Fusion Proteins

VII.D.1. Fusion Proteins Comprising Repeats of sFv Sequences

To increase the valency of fusion proteins of the invention, one or more tandem repeats of the DNA sequence that encode the [V(H)-V(L)] domains are introduced into the chimeric reading frame that encodes the fusion protein. For example, in the case of a dimer, two copies of each antibody variable domain, V(H) and V(L), are combined in a single chain construct (see, e.g., U.S. Pat. No. 6,121,424). After expression in E. coli, intramolecularly folded bivalent diabodies are prepared, preferably from soluble periplasmic extracts. The relative amounts of intramolecular diabodies, as opposed to intermolecular tetrabodies formed from the association of V(H) and V(L) domains from two separate diabodies, is dependent on the length of the linker in the middle of the chain and bacterial growth conditions (Kipriyanov et al., Bispecific tandem diabody for tumor therapy with improved antigen binding and pharmacokinetics, J Mol Biol 1999 Oct. 15; 293(1):41-56).

Fusion proteins comprising tetravalent single-chain antibodies, e.g., {[V(H)-V(L)]2}2 wherein each V(H) and V(L) can combine to form a sFv, may be prepared using similar strategies (Booth et al., Genetically Engineer Tetravalent Single-Chain Fv of the Pancarcinoma Monoclonal Antibody CC49: Improved Biodistribution and Potential for Therapeutic Application, Cancer Research 60, 6964-6971, Dec. 15, 2000). See also U.S. Pat. Nos. 5,869,620; 5,877,291; and 5,892,020.

In fusion proteins comprising single chain Fv (sFv) fragments, the orientations of the V(H) and V(L) domains relative to each other, and other fusion protein elements, influences the expression and activity of the sFv portion (Luo et al., V1-linker-Vh orientation-dependent expression of single chain Fv-containing an engineered disulfide-stabilized bond in the framework regions, J Biochem (Tokyo) 1995 October; 118(4):825-31).

VII.D.2. Fusion Proteins Comprising Other Targeting Elements

Multivalent fusion proteins can comprise other polypeptidic targeting elements. For example, fusion proteins may comprise polypeptides derived from bacterial proteins that bind to pIgR and/or pIgR stalk molecules. Derivatives of monoclonal antibodies directed to pIgR and/or pIgR stalk molecules, e.g., complementary determining sequences (CDR), (Fab)2 molecules and the like, may be prepared and incorporated into fusion proteins.

VII.E. Other Methods for Multimerization

VII.E.1. Chemical Bonds

Cysteine and other reactive amino acid residues that are naturally present or artificially introduced into a monomer molecule may be reacted in order to create chemically linked multimers. In the case of Cys residues, intermolecular disulfide (—S—S—) bonds may be formed to link monomers together. Such intermolecular disulfide bridges may be eliminated or reduced by addition of reducing agents, e.g., DTT. Chemical cross linkers, e.g., bifunctional linkers, can be used to form chemical bonds between monomers.

Thus, by way of non-limiting example, multivalent compounds may be prepared by the chemical linkage of two monovalent molecules, each of which comprises a targeting element. The multivalent conjugate may then be covalently or non-covalently associated with a bioactive molecule. As another example, multivalent bioactive compounds may be prepared by chemically conjugating two monovalent bioactive molecules (i.e., molecules comprising a bioactive moiety and a single targeting element) to each other. This is one way in which the ratio of bioactive molecules to targeting elements may be controlled; in the former case, the conjugate has 2 targeting elements and 1 bioactive moiety, whereas the latter conjugate comprises 2 targeting elements and 2 bioactive moieties.

VII.E.2. Leucine Zippers

A number of eukaryotic transcription factors comprise a dimerization motif called the “leucine zipper”. These leucine zipper proteins form homodimers and/or heterodimers with another protein containing a leucine zipper motif. Proteins that dimerize due to the presence or introduction of leucine zippers are said to be “leucine zipped.” See, Rieker et al., Molecular applications of fusions to leucine zippers, Methods Enzymol 2000; 328:282-96; Hoyne et al., High affinity insulin binding by soluble insulin receptor extracellular domain fused to a leucine zipper, FEBS Lett 2000 Aug. 11; 479(1-2):15-8; Behncken et al., Growth hormone (GH)-independent dimerization of GH receptor by a leucine zipper results in constitutive activation, J Biol Chem 2000 Jun. 2; 275(22):17000-7; Busch et al., Dimers, leucine zippers and DNA-binding domains, Trends Genet 1990 February; 6(2):36-40; Riley et al., Multimer formation as a consequence of separate homodimerization domains: the human c-Jun leucine zipper is a transplantable dimerization module, Erratum in: Protein Eng 1996 September; 9(9):831 Protein Eng 1996 February; 9(2):223-30; Schmidt-Dorr et al., Construction, purification, and characterization of a hybrid protein comprising the DNA binding domain of the LexA repressor and the Jun leucine zipper: a circular dichroism and mutagenesis study, Biochemistry 1991 Oct. 8; 30(40):9657-64; Dmitrova et al., A new LexA-based genetic system for monitoring and analyzing protein heterodimerization in Escherichia coli, Mol Gen Genet 1998 January; 257(2):205-12. Granger-Schnarr et al., Transformation and transactivation suppressor activity of the c-Jun leucine zipper fused to a bacterial repressor, Proc Natl Acad Sci USA 1992 May 15; 89(10):4236-9. Methods for preparing leucine-zipped multivalent sFv's have been described; de Kruif, Leucine zipper dimerized bivalent and bispecific sFv antibodies from a semi-synthetic antibody phage display library, J Biol Chem 1996 Mar. 29; 271(13):7630-4.

VII.E.3. Other Dimerization Domains

Coiled coil dimerization is described by Willcox et al. (Production of soluble alphabeta T-cell receptor heterodimers suitable for biophysical analysis of ligand binding, Protein Sci 1999 November; 8(11):2418-23).

The use of Protein A interactions with immunoglobulins to cause the dimerization of proteins has been described (De A et al., Use of protein A gene fusions for the analysis of structure-function relationship of the transactivator protein C of bacteriophage Mu, Protein Eng 1997 August; 10(8):935-41).

GST sequences can be used as dimerization sequences. Tudyka, Glutathione S-transferase can be used as a C-terminal, enzymatically active dimerization module for a recombinant protease inhibitor, and functionally secreted into the periplasm of Escherichia coli, Protein Sci 1997 October; 6(10):2180-7.

VII.E.2. Combinations

Any possible combination of covalent and non-covalent bonds may be used to generate the multivalent complexes of the invention. A fusion protein, in which multiple V(H) regions are covalently bonded, may be non-covalently associated with a second fusion protein having multiple V(L) regions that are covalently linked to each other (see, e.g., U.S. Pat. No. 6,239,259), a complex that has the structure found in the following diagram. 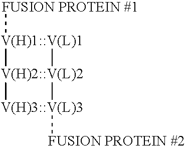
VIII. Calcitonin Polypeptides

Calcitonin is a biologically active protein that is discussed in the Examples. Calcitonin is a polypeptide hormone having 32 amino acids. Efforts to create a formulation, such as those that are oil-based or polymer-based delivery systems, appropriate for delivery of calcitonin via nasal, oral, vaginal and rectal routes are reviewed by Torres-Lugo et al. (Biomaterials 21:1191-1196, 2000). Despite such efforts, there is no widely used and approved non-invasive method for administering calcitonin. Nevertheless, calcitonin is of great interest as a potential therapeutic agent for diseases such as osteoporosis and osteoarthritis (Milot et al., Comp. Ther. 26:183-189, 2000; Kenny, Rheum. Dis. Clin. North Am. 26:569-591, 2000).

Calcitonin is a polypeptide hormone that is primarily produced and secreted by the parafollicular cells of the thyroid gland in mammals and by the ultimobranchial gland of birds and fish, but is also synthesized in a wide variety of other tissues, including the lung and intestinal tract.

Calcitonin is a hormone known to participate in calcium and phosphorus metabolism. Calcitonin controls the activity of osteoclasts (the cells that break down old and weakened bone), so it can be replaced by new bone. It has been shown that the calcitonins reduce calcium concentration in blood (Hirsch et al., Science Vol. 146, page 412, 1963), and inhibit feeding (Freed et al., Science Vol. 206, page 850, 1979) and gastric secretion (Hesch et al., Horm. Metab. Res. Vol. 3, page 140 (1971).

VIII.A. Calcitonin Structure

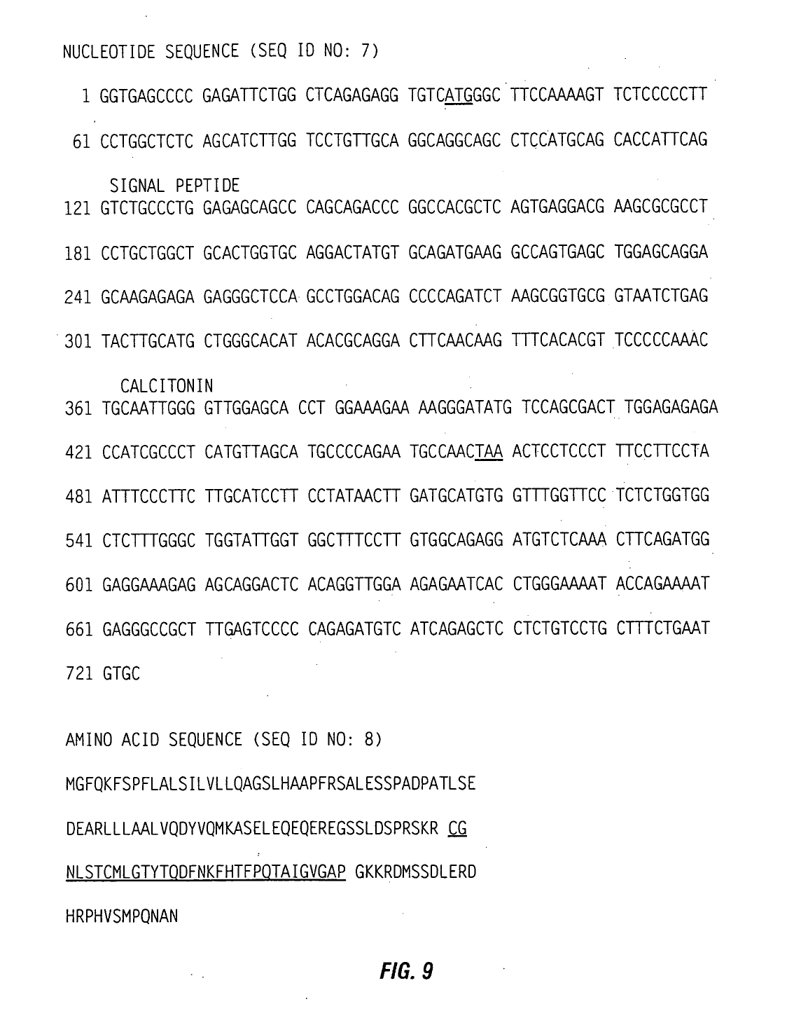

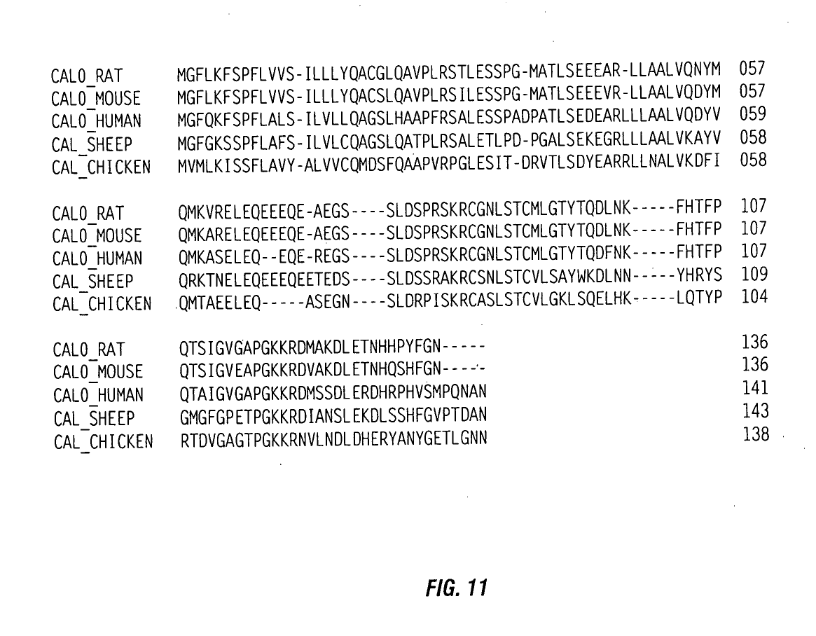

Structural features of calcitonins include a constant chain length of 32 amino acids, a disulfide bridge between the cysteine residues in positions 1 and 7, forming a ring of seven amino acid residues at the N-terminal, and a carboxy terminal proline amide. Amino acid residues common to all calcitonins are those in the 1st, 4th-7th, 28th and 32nd positions (see FIGS. 9-11). Thus, full length calcitonins may be characterized for example by a bridge generally between positions 1 and 7 of the polypeptide chain and, alternatively or additionally, by a leucine residue in position 9, and/or a glycine residue in position 28 and/or a proline residue in position 32.

Although not wishing to be bound by any particular theory, it is thought that the proline amide at C-terminal, common to all calcitonins, is indispensable for the biological activities thereof (Potts et al., Calcium, Parathyroid Hormone and the Calcitonins, page 121 printed by Excerpta Medica, Amsterdam (1971); Sieber, Calcitonin 1969, page 28, Proc. 2nd Symp., printed by Medical Books, London (1970); and Rittel et al., Experientia, Vol. 32, page 246 (1976).

VIII.B. Calcitonin Derivatives

Derivatives of calcitonins include but are not limited to calcitonin structures that have been altered relative to the natural protein, e.g., (a) one or more amino acid radicals are replaced by one or more other amino acid radicals (natural or synthetic) and/or (b) the disulfide bridge is replaced by an alkylene bridge and/or is open, and/or (c) one or several amino acid radicals are omitted (desaminoacyl derivatives).

Calcitonin homologs, i.e, polypeptides derived from non-human species that have amino acid sequences that are related to, but different from, the sequence of human calcitonin, are also calcitonin derivatives within the scope of the invention. Non-limiting examples of calcitonin homologs are listed in Table 9 and described in FIGS. 9-11. One skilled in the art will be able to select the appropriate source of DNA, sequence of primers, PCR conditions, etc. for each particular genetic sequence encoding an calcitonin homolog to generate other calcitonin fusion proteins.

TABLE 9
CALCITONIN HOMOLOGS AND ANALOGS

| | Accession | |
|---|---|---|
| Organism(s) | Number(s) | Citation(s) |
| Tobacco hornworm | | Reagan, J. Biol. Chem. 269: 9-12 (1994) |
| Cockroach | | Furuya et al., Proc. Natl. Acad. Sci. USA |
| | | 97: 6469-74 (2000) |
| Bony fishes | | Suzuki et al., Gen. Comp. Endocrinol. |
| (lungfish, sturgen, etc.) | | 113: 369-73 (1999) |
| Cartilaginous fish | | |
| (stingray) | | |
| Teleosts (eels) | | Suzuki et al., Gen. Comp. Endocrinol. |
| | | 113: 369-73 (1999); Hashimoto et al., |
| | | Biochemistry 38: 8366-84 (1999) |
| Reptiles (turtle, snake, | | Suzuki et al., Zoolog. Sci. 14: 833-6 (1997) |
| grass lizard and | | |
| oaimon) | | |
| Salmon | | Stevenson, J. Pept. Res. 55: 129-39 (2000); |
| | | Hong et al., Biochem. Biophys. Res. Commun. |
| | | 267: 362-7 (2000) |
| | | Recombinant production formulated for |
| | | implants |
| Human/Salmon hybrid | GI/2173732 | Takahashi et al., Peptides 18: 439-44(1997); |
| genes | | Miyake, et al., Patent: JP 1993255391-A 4 |
| | | Oct. 5, 1993 |
| Flounder | GI/2173730 | Suzuki et al., Gene 244: 81-8 (2000) |
| Chicken | GI/222801 | Minvielle et al., FEBS Lett. 203: 7-10 (1986) |
| Fish Calcitonin | GI/2169307 | Narishima et al., Patent: JP 1986291598-A 2; |
| Derivative | GI/2169306 | Dec. 22, 1986 |

Calcitonin analogs are also calcitonin derivatives are also within the scope of the invention. Such analogs may be, for example, polypeptides having amino acid sequences derived from a calcitonin protein. Calcitonin genes and proteins that are the combination of calcitonin sequences from one species to another are also calcitonin analogs as that term is used herein. An example of the latter type of calcitonin analog is one which has an amino-terminal human calcitonin precursor fused to a salmon calcitonin (Takahashi et al., Peptides 18:439-444, 1997). See also U.S. Pat. Nos. 6,265,534; 6,251,635; 6,124,299; 6,107,277; 5,977,298; 5,962,270; 5,710,244; and 5,541,159.

Hybrid or chimeric calcitonin polypeptides may also be prepared and calcitonin derivatives within the scope of the invention. See, e.g., Takahashi et al., Peptides 18:439-44 (1997); U.S. Pat. No. 5,831,000; and Japanese Patent JP 1993255391-A 4.

Various formulations may be preferred for calcitonin delivery depending on the mode of delivery and targeted tissue. See, e.g., U.S. Pat. Nos. 6,149,893; 6,087,338; 5,912,014; 5,726,154; 5,719,122; 5,571,788; 5,514,365; and Serres, et al., “Temperature and pH-sensitive Polymers for Human Calcitonin Delivery”, Pharmaceutical Research 13:196-201, 1996.

VIII.C. Testing of the Biological Activity of Calcitonin Polypeptides and Derivatives

The term calcitonin polypeptide embraces calcitonin derivatives having one or more biological activities of calcitonin. A variety of methods are known in the art that may be used to evaluate the biological activity of calcitonin derivatives. For example, the hypocalcemic rat model can be used to determine the effect of synthetic calcitonin mimetics on serum calcium, and the ovariectomized rat or mouse can be used as a model system for osteoporosis. Bone changes seen in these models and in humans during the early stages of estrogen deficiency are qualitatively similar. Calcitonin has been shown to be an effective agent for the prevention of bone loss in ovariectomized humans and also in rats (Mazzuoli, et al., Calcif. Tissue Int. 47:209-14, 1990; Wronski, et al., Endocrinology 129:2246-50, 1991).

Calcitonin acts directly on osteoclasts via a cell surface receptor, the calcitonin receptor (CRE). CRE is a member of the G-protein receptor family and transduces signal via activation of adenylate cyclase, leading to elevation of cellular cAMP levels (Lin, et al., Science 254:1022-4, 1991). Calcitonin-mediated receptor activation can be detected by: (1) measurement of adenylate cyclase activity (Salomon, et al., Anal. Biochem. 58:541-8, 1974; Alvarez and Daniels, Anal. Biochem. 187:98-103, 1990); (2) measurement of change in intracellular cAMP levels using conventional radioimmunoassay methods (Steiner, et al., J. Biol. Chem. 247:1106-13, 1972; Harper and Brooker, J. Cyc. Nucl. Res. 1:207-18, 1975); or (3) use of a cAMP scintillation proximity assay (SPA) method (Amersham Corp., Arlington Heights, Ill.).

VIII.D. Therapeutic Uses of Calcitonin

Calcitonin inhibits bone resorption through binding and activation of a specific calcitonin receptor on osteoclasts (The Calcitonins-Physiology and Pharmacology Azria (ed.), Karger, Basel, Su., 1989), with a resultant decrease in the amount of calcium released by bone into the serum. This inhibition of bone resorption has been exploited, for instance, by using calcitonin as a treatment for osteoporosis, a disease characterized by a decrease in the skeletal mass often resulting in debilitating and painful fractures, and prevention of fracture in osteogenesis imperfecta.

Calcitonin is also used in the treatment of Paget's disease where it provides rapid relief from bone pain, which is frequently the primary symptom associated with this disease. This analgesic effect has also been demonstrated in patients with osteoporosis or metastatic bone disease and has been reported to relieve pain associated with diabetic neuropathy, cancer, migraine and post-hysterectomy. Reduction in bone pain occurs before the reduction of bone resorption.

Other uses of calcitonin include but are not limited to treatment of hypercalcemia and Paget's disease, counteracting vasospasms, ischemia, renal failure, and treating male impotence.

IX. Interleukin Polypeptides

Interleukins are a class of biologically active proteins, certain members of which are discussed in the Examples. Interleukins are released from helper T-cells that promote lymphocyte proliferation. Interleukins of particular interest that can be adapted to the methods and compositions of the invention include interleukins-1, -2, -3, -4 and -5 (IL-1, IL-2, IL-3, IL-4 and IL-5, respectively).

IX.A. Interleukin-2 (IL-2)

IX.A.1. Biological Activity of IL-2

IL-2 is a central regulator of immune response that mediates proliferation of activated B cells and T cells, including anti-tumor T cells, and plays a role in anti-inflammatory reactions. Interleukin-2 (IL-2) is a lymphokine secreted by certain T lymphocytes after antigenic or mitogenic stimulation. The actions of IL-2 are mediated through the binding of the IL-2 protein to specific high affinity receptors which are present in the membranes of activated, but not resting, lymphocytes. The biology, biochemistry and molecular biology of IL-2 are reviewed by Trinchieri (Blood 84:4008-4027, 1994).

IX.A.2. Structure of IL-2

Human IL-2 is synthesized as a precursor protein of 153 amino acids, which includes a 20 amino acid hydrophobic leader sequence. The IL-2 molecule has a molecular weight of about 15.4 kD and a slightly basic pI. The protein comprises a single intramolecular disulfide bond (Cys58-Cys105) that is necessary for the biological activity of IL-2 (Yamada et al., Importance of disulfide linkage for constructing the biologically active human interleukin-2, Arch Biochem Biophys 257:194-199, 1987).

Some forms of IL-2 comprise chemical modifications. It has been reported that O-glycosylation occurs at Thr3 of bovine IL-2, and that variants with different masses due to glycosylation exist. However, non-glycosylated IL-2 remains biologically active (Kuhnle et al., Bovine interleukins 2 and 4 expressed in recombinant bovine herpesvirus 1 are biologically active secreted glycoproteins, J Gen Virol 77(Pt 9):2231-2240, 1996).

Recombinant human IL-2, expressed in either E. coli or COS cells, has been shown to be phosphorylated by protein kinase C in vitro (Kung et al., Phosphorylation of human interleukin-2 (IL-2), C Mol Cell Biochem 89:29-35, 1989). The phosphorylated tryptic peptide was identified as the N-terminal fragment containing a single phosphorylation site at the serine residue at position 7 (Ser7). There was no difference in biological activity between non-phosphorylated and phosphorylated IL-2, as determined by a T cell growth assay

IX.B. Interleukin-4 (IL-4)

IX.B.1. Structure of IL-4

Interleukin 4 (IL-4) is a potent and pleiotropic lymphokine that affects a variety of cells, especially those of hematopoietic origin. IL-4 performs important functions as a major regulator of the immune response and plays a role in allergy and asthma by directing the induction of TH2 phenotype in T-cells, activating B-cells, and stimulating the synthesis of IgE antibodies. The IL-4 receptor alpha chain (IL-4-BP, IL-4 binding protein) demonstrates a high affinity and specificity for IL-4. The crystal structure of interleukin-4 (IL-4) and its complex with the IL-4 receptor alpha chain has been published by Hage et al. (Cell 97:271-81, 1999). The crystal structure of the human IL-4 and IL-4-BP shows that specific surfaces and regions on each protein interact and contact each other. The crystal structure of IL-4 alone has been described (Powers et al. Science 256:1673-1677, 1992; Walter et al., J. Biol. Chem. 267:203-20376, 1992; and Wlodawer et al., FEBS Lett. 309:59-64, 1992) as has the solution structure (Smith et al., J. Mol. Biol. 224:899-904, 1992). The region of IL-4 that binds to the receptor alpha chain is the AC helix face (Kruse et al., EMBO J. 12:5121-5129, 1993). The functional amino acids that participate in contacts with IL-4 BP have been identified by Wang et al. (Proc. Natl. Acad. Sci. 94:1657-1662, 1997). The structure of IL-4 is very similar between the receptor bound and unbound form, but small, significant differences exist. Hage et al. disclosed that I11, N15, and Y124 are main contacts in receptor interaction. Morrison and Leder (J. Biol. Chem. 267:11957-11963, 1992) disclosed that E12, 114, L104, D106, F107, and L111 in murine IL-4 are important for function; the corresponding residues either homologous or identical in human IL-4 are E9, I11, L109, N111, F112, and L117, respectively.

The amino acid sequence of IL-4 (SEQ ID NO: ______) as reported in the Protein Data Bank as 1 H1K by Muller et al. (J. Mol. Biol. 247:360-372 1995) is as follows. HKCDITLQEIIKTLNSLTEQKTLCTELTVTDIFAASKNTTEKETFCRAATV LRQFYSHHEKDTRCLGATAQQFHRHKQLIRFLKRLDRNLWGLAGLNSCPVK EANQSTLENFLERLKTIMREKYSKCSS

IX.C. Interleukin Derivatives

Cysteine Substitution and Insertion Sites in IL-4

1HIK, a crystal structure of human IL-4, was obtained by accessing the Protein Data Base. By visual examination, four (4) loops were defined as follows: Loop 1 from Q20 to T25, Loop 2 from F33 to E41, Loop 3 from K61 to T29, and Loop 4 from A94 to A104. Some loops, Loop 4 in particular, had components similar to β structure. The crystallographic structure of IL-4 complexed with the extracellular domains of interleukin 2 δ-chain receptor and interleukin 4δ receptor (1ITE) as disclosed in the Protein Data Base was also examined for the surface accessibility of amino acid side chains of IL-4 that were not in contact with the receptors. Based on their visual exposure to the bulk solvent, certain amino acids were selected as sites at which cysteines may either be inserted on either side of the residue or cysteines may be substituted. In addition, other amino acids whose side chains were exposed to bulk solvent, and pointed away from the body of the protein, were chosen, many of which existed on helical regions of IL-4. The results of this visual inspection of human IL-4 in 1ITE and 1HIK are tabulated in Table 10.

TABLE 10
SITES IN IL-4 FOR CYSTEINE SUBSTITUTIONS OR INSERTIONS

| | Most Preferred Amino Acid | Preferred Amino Acid |
|---|---|---|
| | Suitable for Cysteine | Suitable for Substitution or |
| Loop | Substitution or Insertion | Cysteine Insertion |
| 1 | E19 | Q20 |
| | K21 | T22 |
| | L23 | T25 |
| | E26 | |
| 2 | T30 | F33 |
| | S36 | A34 |
| | K37 | A35 |
| | N38 | E41 |
| | T40 | |
| 3 | K61 | G67 |
| | D62 | |
| | T63 | |
| | R64 | |
| | L66 | |
| | A68 | |
| | T69 | |
| 4 | N97 | A94 |
| | S98 | G95 |
| | K102 | L96 |
| | A104 | V101 |
| | | E103 |
| Other | E26 | |
| | T28 | |
| | Q54 | |
| | S57 | |
| | H58 | |
| | E60 | |
| | D62 | |
| | R64 | |
| | A70 | |
| | Q72 | |
| | F73 | |
| | H74 | |
| | K77 | |
| | R81 | |
| | R85 | |
| | R88 | |
| | N105 | |
| | Q106 | |
| | E110 | |
| | N111 | |
| | E114 | |
| | R115 | |

Substitutions of cysteine within the human IL-4 sequence may be made for any residues contained in the segments 19-30, 31-40, 60-69, 95-103, and 104-109 (see Table 6). In addition, amino acids within helical regions may also be substituted if their side chains are oriented away from the main body of the protein and do not participate in interactions with other amino acid side chains that provide stability to the structure. Furthermore, these side chains should not participate in the biological activity or function of the molecule. Examples of amino acid positions for cysteine substitution include His 58, Arg 81, His 74, Gln 71, and Arg 53. The identification of specific residues within the IL-4 structure are not meant to be limiting, as other substitutions are possible.

IX.D. Other Interleukins

Homologous Cysteine Substitutions in Proteins that are Members of Families

Many proteins and peptides show homology with other proteins and peptides. Such homology is the basis for classification of proteins and peptides into families and subfamilies. Homology may be based on sequences or the proteins and peptides or on three dimensional structure of the proteins and peptides, or on a combination of both. It is likely that the same general areas and surface regions of proteins and peptides that are members of the same family or subfamily interact with receptors or are involved in other biological functions and activities. Therefore, identification of residues that may be substituted by cysteine in one family member may be translated to residues that may be substituted in other family members.

Information about cysteine substitution and insertion sites on one family member to perform cysteine substitution and insertion on additional family members or in all of the family members. This is especially relevant when the receptors with which members of the family are also homologous. For example, IL-13 is homologous with IL-4, and IL-13 elicits a subset of biological activities possessed by IL-4. The receptors for IL-4 and IL-13 share a common subunit. Antibodies directed against the alpha chain of IL-4 receptor, the primary binding subunit of the IL-4 receptor, blocks the function and binding of IL-13 to its receptor (Zurawski et al., J. Biol. Chem. 270:13869-13878, 1995). Although no crystallographic structure is currently available in the Protein Data Base, by comparing the sequences through homology programs, residues present in IL-4 that may be substituted by cysteine can be identified by homology in IL-13 and substituted by cysteine. In another example, gamma c receptor subunit is used in all of the receptors for IL-2, IL-7, IL-9, and IL-15. A theoretical three dimensional model has been made for IL-7 based on homology to human IL-2, IL-4, GM-CSF, and growth hormone. Kroemer et al., Protein Engineer 9:493-498, 1996. A part of the present invention is directed to using cysteine substitution and insertion sites in one of these cytokines to predict equivalent cysteine substitution and insertion sites in the remaining cytokines using their homologous structures to identify those sites. Cysteine substitution or insertion derivatives of IL-2 and IL-3 have been described (U.S. Pat. Nos. 5,206,344 and 5,166,322, respectively).

Functional amino acids and domains may be identified by various means, including chemical modification, site specific mutation, deletion analysis, alanine scans and so on.

X. Other Biologically Active Polypeptides

X.A. Hormones

Calcitonin is a polypeptide hormone having 32 amino acids (see above). Other hormones of interest include other calcintonins including but not limited to human salmon calcitonin, insulin; growth hormone (somatotropin); parathyroid hormone; leptin; melanocyte stimulating hormone; orexin; neuropeptide Y; adrenocorticostimulating hormone; corticotropin like intermediate lobe peptide; melanin; concentrating hormone; opioid peptides including but not limited to endorphins, enkaphalins, and dynomorphins; urotensins including but not limited to urotensin II; amylin related peptides including but not limtied to amylin; gonadotrophin-releasing hormone; follicle stimulating hormone, luteinizing hormone, any follicle stimulating hormone, and parathyroid hormone.

X.B. Growth Factors

Growth factors are proteins that induce, promote and otherwise mediate the growth, organization, differentation and/or maintenance of cells. Growth factors are often specific for a given tissue or cell type, and may be named accordingly. Examples of growth factors include but are not limited to various species of growth factor is selected from the group consisting of various species of NT3; various species of fibroblast growth factors, such as, but not limited to, FGF-1, FGF-2, FGF-3, FGF-4, FGF-5, FGF-6 and FGF-7; platelet derived growth factor (PDGF); epidermal growth factor (EDGF); endothelial growth factor; various species of vascular endothelial growth factor (VEGF) and vascular permeability factors, including but not limited to VEGF-1, VEGF-2, VEGF-121, VEGF-165, VEGF-189; nerve growth factor (NGF), placenta growth factors (PGF) including but not limited to PGF-1 and PGF2; hepatocyte growth factor; hepatocyte growth factor/scatter factor; brain derived neurotropic factor (BDNF); various insulin-like growth factors (ILGF), including but not limited to ILGF-1 and ILGF-2; macrophage stimulating protein; oncostatin M; milk derived peptide growth factors; eye derived growth factors; various forms of transforming growth factor (TGF), including but not limited to TGF-alpha and TGF-beta; latent transforming growth factor beta binding protein; various forms of transforming growth factor (TGF) including but not limited to TGF-beta 1, TGF-beta 2, TGF-beta 3; various forms of bone morphogenetic protein (BMP) including but not limited to BMP-1 and BMP-7; osteogenic protein 1; endostatin; angiostatin; and ciliary neurotropic factor.

X.C. Enzymes

Enzymes are proteins that catalyze biochemical reactions and may serve as biologically active proteins. Enzymes of interest include but are not limited to glucocerbrosidase, for the management of Type 1 Gaucher disease; Alglucerase, which is a modified form of glucocerbrosidase; ββ glucocerebrosidase (ββ-D-glucosyl-N-acylsphingosine glucohydrolase); intracellular molecules (synthases, phosphatases, kinases such as MAP kinases, glycogen synthase kinase); membrane-bound growth factor receptor (such as the CD45 receptor) kinases and phosphatases; nucleotide exchange factors (mSOS). Examples of inhibitors of enzymes include inhibitors of caspases, kinases, phosphatases and the like.

X.D. Factors Mediating Apoptotis

Mediators of and participants in apoptosis are another type of biologically active protein. Non-limiting examples of such proteins include death domain proteins such as TNF Receptor associated death domain (TRAAD); FAS associated death domain (FADD); TNF associated factor 1 to 3 (TRAF 1 to 3); leukemia inhibitory factor; receptor interacting proteins including receptor interacting protein associated Ich-1/CED-3; caspases; cathepsins; members of the bcl-2 family of proteins; and nucleases.

X.E. Anticancer Agents

A biologically active protein of the invention may also be an anti-proliferation agent or an anticancer agent. Non-limiting examples of such proteins include internal kringle fragments of plasminogen including kringle 1 to 3 (angiostatin) and kringle 1 to 4; amino terminal fragment of urokinase; fragments of basement membrane collagen XVIII including endostatin; soluble FLT-1 receptor; and interferon alpha inducible protein 10.

X.F. Receptor Fragments

A biologically active polypeptide may also be a receptor or fragment thereof. Non-limiting examples of such receptors include a TNF-alpha receptor; a cytokine receptor; and a hormone receptor.

X.G. Factors Mediating Angiogeneisis

Some biologically active polypeptides of particular interest are those that are a mediator, inhibitor or participant in angiogenesis. Non-limiting examples of such proteins include vascular endothelial growth factor (VEGF), including but not limited to VEGF-1, VEGF-2, VEGF-121, VEGF-165, VEGF-189; and vascular permeability factors; metaloproteases, antibodies to integrins; plasminogen; plasminogen activator; and urokinase.

X.H. Cytokines

Cytokines are proteins involved in signaling between cells during an immune response or involved in an inflammatory response. Lymphokines are a class of cytokines produced by lymphocytes. Representative cytokines and growth factors include, for example, interferons (IFNs; e.g., IFNα, IFNβ, and IFNγ); interleukins (including IL-1 through IL-15); and colony stimulating factors (e.g., those involved in the division and differentiation of bone marrow stem cells and their progeny, for example, stem cell factor (SCF), granulocyte colony stimulating factor (G-CSF), erythropoietin (EPO), granulocyte macrophage colony stimulating factor (GM-SCF), fibroblast growth factors (e.g. FGF1 and FGF2), PDGF, EDGF, various species of VEGF, NT3, and NGF, BDNF, factor VIII, actor IX and insulin-like growth factor.

X.I. Antigens

A biologically active protein may be an antigenic polypeptide, i.e, one designed to elicit an immune response. For example, a biologically active antigenic protein may be an antigen, super-antigen, epitope or other polypeptide that is derived from a protein that is a part of, is derived from or is associated with a pathogen. The term biologically active antigenic protein also encompasses proteins produced by cells of the body in response to a pathogenic infection. The term further encompasses proteins produced by cells of the body that have an inappropriate pattern of growth or undesirable activity such as, e.g., cancer cells and cells that mediate autoimmune diseases. Fusion proteins of the invention that include an antigenic portion are intended to elicit an immune response once introduced into the body of an animal; if the response provides a prophylactic effect, the fusion protein may be formulated into a vaccine. Non-limiting examples of antigenic polypeptides include a protein from a pathogen including but not limited to a bacterium, a virus, a rickettsial species or a chlamydia species; a protein that is a tumor antigen; and a protein that is required for reproduction. Of particular interest are viral proteins from human immunodeficiciency virus, respiratory synctial virus, parainfluenza virus, influenza virus, hepatitis A virus, hepatitis B virus, hepatitis C.

X.J. Antiviral Proteins

A biologically active protein may also be an antiviral protein. Non-limiting examples of antiviral proteins include peptides that inhibit HIV replication and infection by human immunodeficiciency virus, respiratory synctial virus, parainfluenza virus, influenza virus, hepatitis A virus, hepatitis B virus, hepatitis C and fusion of human immunodeficiency virus infected cells including peptides related to amino acid sequences in HIV-1 glycoprotein 41 and glycoprotein gp120.

X.K. Therapeutic and Diagnostic Monoclonal Antibodies

Currently, a variety of monoclonal antibodies have been approved for use by the U.S. Federal Drug Administration, or the corresponding agencies of other countries. More Mab's are in clinical trials, and even more are being developed for testing. Table 11, although by no means comprehensive or limiting, lists therapeutic and diagnostic monoclonal antibodies that may be used in the compositions, compounds and methods of the invention.

TABLE 11
MONOCLONAL ANTIBODIES AND APPLICATIONS THEREOF

| Name(s) | Description | Uses | Manufacturer |
|---|---|---|---|
| BEC2 | Anti/idiotypic Mab | Treatment of small | ImClone |
| | | lung cancer and | Systems/Merck |
| | | melanoma | |
| Cetuximab | Chimeric anti-EGFR | Cancer treatment | ImClone |
| (Mab C225; IMC- | Mab | | systems/Merck |
| C225) | | | |
| LDP-01 | Humanized Mab | Inflammation | LeukoSite |
| | directed to b2 integrin | following stroke, | |
| | receptor | heart attack and | |
| | | transplantation | |
| LDP-02 | Humanized Mab | Crohn's disease, | LeukoSite |
| | directed to a4b7 | inflammatory bowel | |
| | integrin receptor on | disease, ulcerative | |
| | leukocytes | colitis | |
| Alemtuzumab | Humanized Mab | Chronic lymphotic | LeukoSite |
| (LDP-03; Campath ®) | | leukemia | (approved 2001) |
| ABX-IL-8 | Human Mab directed | Psoriasis | Abgenix |
| | to IL-8 | | |
| ABX-CBL | Murine Mab | Treatment of graft vs. | Abgenix |
| | | host disease (GVD) | |
| ABX-EGF | Xenomouse - | Anticancer (renal, | Abgenix |
| | produced monoclonal | prostage, lung and | |
| | antibody to EGF | breast tumors) | |
| | receptor | | |
| Oncolym | ¹³¹I-labeled murine | Non-Hodgkin's | Techniclone |
| | Lym-1 Mab directed | lymphoma | |
| | to HLA-Dr10, a cell | | |
| | surface marker on | | |
| | 80% of lymphoma | | |
| | cells | | |
| Tositumomab | ¹³¹I-labeled Mab | Non-Hodgkin's | Coulter |
| (Bexxar ™) | directed to mature B- | lymphoma | Pharmaceuticals |
| | cells | | |
| Daclizumab | Mab directed to | Transplantation. | Protein Design Labs |
| | CD25 (TAC submit | Autoimmune diseases | (PDL) (approved |
| | of IL-2 receptor) | | 1997) |
| SMART ™ M195 | IgG1 Ab directed to | Acute myeloid | PDL |
| | CD33 antigen of T | leukemia (AML) | |
| | cells | | |
| Nuvion ™ | IgG2 Ab directed to | Prevention of | PDL |
| (SMART ™ Anti- | CD3 antigen of T | cytokine release | |
| CD3) | cells | syndrome of | |
| | | Muromonoab-CD3 | |
| Ostavir | Human anti-hepatitis | Treatment of chronic | PDL |
| | B Mab | hepatitis B infections | |
| Vitaxin | Antibody to av b-3 | Inhibitis angiogenesis | Ixys Inc. |
| | integrin | in cancer | |
| XTL 001 | 2 Mab's directed to | Treatment of hepatitis | XTL |
| | multiple sites on HBV | B viral infections | Biopharmaceuticals |
| | surface antigen | | |
| AFP-Cide ™ Y-90 | Humanized Mab | Treatment of liver | Immunomedics |
| | directed to α- | cancer | |
| | fetoprotein | | |
| LymphoCide ™ Y-90 | Y-90 labeled directed | Treatment of B-cell | Immunomedics |
| | humanized Mab to | lymphoma receptors | |
| | CD22 | | |
| CEA-Cide ™ Y-90 | Humanized Mab | Treatment of CEA- | Immunomedics |
| | directed to | expressing sold | |
| | carcinoembryonic | tumours | |
| | antigen (CEA) | | |
| MDX-CD4 | Humanized anti-CD4 | Treatment of | Medarex |
| | Mab | rheumatoid arthritis, | |
| | | autoimmune diseases, | |
| | | and inflammatory | |
| | | disorders | |
| MDX-33 | Humanized Mab | Treatment of | Medarex/Centeon |
| | directed to FC | idiopathic | |
| | receptor | thrombocithopenia | |
| | | purpura and blood | |
| | | disorders | |
| MDX-44 | MDX-33 Ricin A | Treatment of | Medarex |
| | conjugate that is an | inflammatory | |
| | immunotoxin directed | diseases, rheumatoid | |
| | to CD64 (class IgG | arthritis | |
| | receptor) ( ) | | |
| D2E7 | Human Mab directed | Rheumatoid arthritis | CAT-BASF |
| | to TNF-α | | |
| ING-1 | Humanized anti-EP- | Adenocarcinomas | Xoma |
| | Cam Mab | | |
| rhuMab-E25 | Humanized anti-IgE | Allergies, asthma, | Genentech/Novartis |
| | Mab | rheumatoid arthritis | |
| CDP 870 | Humanized antibody | Rheumatoid arthritis | Celltech |
| | fragment directed to | | |
| | TNF-α | | |
| Reslizumab | Humanized pan Mab | Asthma | |
| (SCH55700, | directed to IL-5 | | |
| CDP835) | | | |
| Mepolizumab | Humanized Mab | Asthma | |
| (SB240563) | directed to human and | | |
| | primate IL-5 | | |
| Ibritumomab | Mab conjugated to | Lymphomas | IDEC |
| (Tinxetan; Y2D8) | 90Y | | |
| Trastuzumab | Humanized anti-Her2 | Treatment of HER2 | Genentech |
| (Herceptin ®) | Mab | overexpressing | (approved 1998) |
| | | metastatic breast | |
| | | cancer | |
| Nerelimomab | Anti-TNF Mab | Treatment of septic | Bayer/Caltech |
| (BAYX-1351) | | shock | |
| Edrecolomab | Anti-EGP-2 rodent | Treatment of colon | Centocor/Glaxo- |
| (Panorex ®) | Mab | carcinoma | Wellcome (approved |
| | | | 1994) |
| Infliximab | Chimeric monoclonal | Treatment of Crohn's | Centocor |
| (Remicade ®) | antibody directed | disease and colon | (approved 1998) |
| | against TNF-α | carcinoma | |
| Abciximab | Chimeric Mab that | An adjunct to | Centocor/Lilly |
| (ReoPro ®) | binds selectively to | percutaneous | (approved 1994) |
| | platelet GPIIb/IIIa | transluminal coronary | |
| | receptors, blocking | angioplasty (balloon | |
| | the binding of | angioplasty, or | |
| | fibrinogen, Von | PTCA) for the | |
| | Willebrand factor, | prevention of acute | |
| | and other adhesive | cardiac ischemic | |
| | factors, thereby | complications in | |
| | inhibiting platelet | patients at high risk | |
| | aggregation | for the sudden closure | |
| | | of the treated | |
| | | coronary vessel. | |
| Rituximab | Chimeric antibody | Treatment of patients | Genentech/IDEC |
| (Rituxan ®, Mabthera) | with human gamma-1 | with relapsed or | (approved 1994) |
| | and kappa constant | refactory low-grade | |
| | regions and murine | or follicular, CD20 | |
| | variable regions | B-cell non-Hodgkins | |
| | | lymphoma. | |
| Palivizumab | Humanized | Prevention of serious | MedImmune |
| (Synagis ®) | monoclonal antibody | lower respiratory | (approved 1998) |
| | that binds to the F- | tract disease caused | |
| | protein of RSV | by respiratory | |
| | | syncytial virus (RSV) | |
| | | in pediatric patients at | |
| | | high risk of RSV | |
| | | disease. | |
| Daclizumab | Humanized anti- | Prophylaxis of acute | PDL/Roche |
| (Zenapax ®) | CD25 Mab | rejection episodes in | (approved 1997) |
| | | patients receiving | |
| | | renal transplants. | |
| Basiliximab | Chimeric anti-CD25 | Transplantion | Novartis (approved |
| (Simulect ®) | Mab | rejection | 1997) |
| Muromonoab-CD3 | Anti-CD3 rodent Mab | Transplantation | Johnson & Johnson |
| (OKT3 ® Orthoclone) | | rejection | |
| Efalizumab | Humanized anti- | Inhibiting T cell | XOMA/Genentech |
| (Xanelin ™) | CD11a monoclonal | proliferation; | |
| | antibody engineered | mediating graft/host | |
| | to bind to and block | rejection; psoriasis. | |
| | the activity of CD11a | | |
| IDEC-131 | Primatized ® | Lupus (SLE); | IDEC |
| | monoclonal antibody | rheumatoid arthritis | |
| | to CD4 receptor | | |
| Clenoliximab (IDEC- | Primatized ® | Rheumatoid arthritis | IDEC-SKB |
| 151/BB-217969) | monoclonal antibody | | |
| | to CD4 receptor | | |
| ID11 | Rodent pan Mab | Retinopathy; diffuse | CAT |
| | directed to TGF- | scleroderma; | |
| | βI, TG-F-β2 and | pulmonary, renal and | |
| | TGF-β3 | liver fibrosis | |
| CAT-152 | Human anti-TGF-β2 | Retinopahty; diffuse | CAT |
| | Mab | scleroderma; | |
| | | pulmonary, renal and | |
| | | liver fibrosis | |
| CAT-192 | Human anti-TGF-β2 | Retinopathy; diffuse | CAT |
| | Mab | scleroderma; | |
| | | pulmonary, renal and | |
| | | liver fibrosis | |

In some aspects of the invention, the monoclonal antibody is directed to a cytokine. A “cytokine” is a protein, generally having a molecular weight in the range of 5 to 20 kD, that is released by cells and that affect the behavior of other cells. Technically, cytokines are hormones, but the term tends to be used as a convenient generic shorthand for interleukins, lymphokines and several related signalling molecules such as TNF and interferons. Generally growth factors would not be classified as cytokines, though TGF is an exception. Chemokines are a subset of cytokines.

XI. Pharmaceutical Compositions and Therapeutic Methods

Another aspect of the invention is drawn to compositions, including but not limited to pharmaceutical compositions. According to the invention, a “composition” refers to a mixture comprising at least one carrier, preferably a physiologically acceptable carrier, and one or more compositions or compounds of the invention. The term “carrier” defines a chemical compound that does not inhibit or prevent the incorporation of the compositions or compounds into cells or tissues. A carrier typically is an inert substance that allows an active ingredient to be formulated or compounded into a suitable dosage form (e.g., a pill, a capsule, a gel, a film, a tablet, a microparticle (e.g., a microsphere), a solution; an ointment; a paste, an aerosol, a droplet, a colloid or an emulsion etc.). A “physiologically acceptable carrier” is a carrier suitable for use under physiological conditions that does not abrogate (reduce, inhibit, or prevent) the biological activity and properties of the composition or compound of the invention. For example, dimethyl sulfoxide (DMSO) is a carrier as it facilitates the uptake of many organic compounds into the cells or tissues of an organism. Preferably, the carrier is a physiologically acceptable carrier, preferably a pharmaceutically or veterinarily acceptable carrier, in which the composition or compound of the invention is disposed.

XI.A. Types of Drugs

Drugs are agents (compounds and complexes) that are administered to (brought into contact with) an animal, including a human, in any of a variety of therapeutic modalities. The term “therapeutic” encompasses modalities including, but not limited to, prophylactic uses that prevent disease; curative uses that eliminate a disease; palliative and ameliorative uses that alleviate, make better, or more tolerable, but do not cure a disease; regressive uses that slow, prevent, or reverse the progress of a disease; and remissive uses that cause a temporary or permanent decrease of a manifestion of the disease. Therapeutic uses also include those that do not involve a disease per se but nonetheless effect the health of an animal. Examples of such agents are antitoxins, analgesics, anesthesia-inducing agents, agents that are used to treat physical and emotional trauma, and psychoactive drugs, e.g., antidepressants, mood stabilizers, and anxiolytic agents.

Some drugs are administered taken on an as needed basis while other agents must be taken at regular intervals. Regardless of the type, timing or course of administration, a therapeutically effective amount must be administered. The term “therapeutically effective amount” indicates the amount of drug which is effective to achieve an intended purpose without undue undesirable side effects (such as toxicity, irritation or allergic response). What constitutes a therapeutically effective amount of a drug will depend on a variety of factors which the knowledgeable practitioner will take into account in arriving at the desired dosage regimen.

Prophylactic uses of drugs include, but are not limited to, the prevention of infections due to bacteria, viruses, and other infective agents, the prevention or inhibition of further hyperproliferation of cells (i.e., the regression of tumors), and prevention of recurrence of diseases that have been treated but may recur. Prophylactic effects may, but need not, result from the induction of an immune response to the drug, i.e., the drug is an immunogen. Prophylactic drugs can be administered to a population or targeted to a subpopulation of high risk individuals. As used herein, the term “high risk individual” is meant to refer to an individual for whom it has been determined, via, e.g., individual or family history or genetic testing, has a significantly higher than normal probability of being susceptible to the onset or recurrence of a disease or disorder. As art of treatment regimen for a high risk individual, the individual can be prophylactically treated to prevent the onset or recurrence of the disease or disorder. The term “prophylactically effective amount” is meant to refer to an amount of drug that produces an effect observed as the prevention of the onset or recurrence of a disease or disorder. Prophylactically effective amounts of a pharmaceutical composition are typically determined by the effect they have compared to the effect observed when a second pharmaceutical composition lacking the active agent is administered to a similarly situated individual.

The term “drug” is meant to encompass prodrugs as well. A “prodrug” is a drug that is administered in a form or as a compound that has little or none of the desired biological activity. A prodrug is altered by processes in vivo to produce a more active agent. In the case of a compound, a prodrug is typically metabolized in vivo in order to product an active agent. The active agent is thus a metabolite of the compound that has been administered to a patient. A well-known example of a prodrug is AZT. In the case of a complex, a compound may be inactive when held in a complex. However, a portion of the complex (a compound) separates from the complex in vivo, thereby generating the active agent. A common example is a salt of a drug wherein the drug becomes active upon dissolution of the salt in vivo. The term “drug” thus encompasses agents that are active in vitro as well as those that become active in vivo.

X.II.B. Routes of Administration

Drugs are typically administered parenterally or enterally. Enteral refers to the administration of the drug into the gastrointestinal tract, preferable via oral administration. Parenteral administration is the administration of the drug via any other route, e.g., intravenous injection directly into the bloodstream. In either-case, the goal of the drug administration is to move the drug from the site of administration to the site in the body where the drug acts to produce its effect, or to administer a systemic therapeutically effective amount of the drug.

Oral administration of drugs is by far the most common method. When administered orally, drug absorption usually occurs due to the transport across the membranes of the epithelial cells within the gastrointestinal tract. Absorption after oral administration is confounded by numerous factors that vary along the length of the gastrointestinal (GI) tract, including but not limited to the luminal pH, surface area per luminal volume, perfusion of tissue, bile and mucus flow, and the epithelial barrier. Pulmonary administration of drugs, i.e., delivery via the respiratory system, is also known.

Although parenteral administration does provide a method for eliminating a number of the variables that are present with oral administration, parenteral administration is not a preferable route. This is because parenteral administration usually requires the use of medical personnel and is not practical for the administration of many drugs. Even when required, parenteral administration is not preferred due to concerns such as patient discomfort, risk of infection, etc., as well as the equipment and costs involved. However, in some cases, despite various attempts, certain therapies require parenterally delivered drugs. Such drugs include polypeptides and other macromolecules that are degraded in the body, which occurs to a large degree in the GI tract. Despite such obstacles, it is desired to, for example, deliver insulin, growth hormones, interleukins, and monoclonal and other antibodies, by non-parenteral forms of administration. Epithelial barriers must be overcome to achieve non-parenteral routes of administration, such as oral and pulmonary administration.

In these and other routes of administration, a drug must traverse several semipermeable cell membranes before reaching general circulation or their targeted site of action. These membranes act as a biological barrier that inhibits the passage of drug molecules. In many instances, the barrier comprises epithelial cells and is thus an epithelial barrier. Epithelial barriers include, by way of non-limiting example, those that line the lumen of an organ. Epithelial barriers thus include, but are not limited to, surfaces that line the gastrointestinal lumen, the pulmonary lumen, the nasal lumen, the nasopharyngeal lumen, the pharyngeal lumen, the buccal lumen, the sublingual lumen, the vaginal lumen, a urogenital lumen, an ocular lumen, a tympanic lumen, and an ocular surface.

XI.B. Pharmaceutical Compositions

A “pharmaceutical composition” refers to a composition comprising a drug wherein the carrier is a pharmaceutically acceptable carrier, while a “veterinary composition” is one wherein the carrier is a veterinarily acceptable carrier. The term “pharmaceutically acceptable carrier” or “veterinarily acceptable carrier” includes any medium or material that is not biologically or otherwise undesirable, i.e, the carrier may be administered to an organism along with a composition or compound of the invention without causing any undesirable biological effects or interacting in a deleterious manner with the complex or any of its components or the organism. Examples of pharmaceutically acceptable reagents are provided in The United States Pharmacopeia, The National Formulary, United States Pharmacopeial Convention, Inc., Rockville, Md. 1990, hereby incorporated in its entirety by reference herein into the present application, as is Pharmaceutical Dosage Forms & Drug Delivery Systems, 7th Edition, Ansel et al., editors, Lippincott Williams & Wilkins, 1999.

The drug is included in the pharmaceutical composition in an amount sufficient to produce the desired effect upon the patient. The pharmaceutical compositions of the invention can further comprise other chemical components, such as diluents and excipients. A “diluent” is a chemical compound diluted in a solvent, preferably an aqueous solvent, that facilitates dissolution of the drug in the solvent, and it may also serve to stabilize the biologically active form of the drug or one or more of its components. Salts dissolved in buffered solutions are utilized as diluents in the art. For example, preferred diluents are buffered solutions containing one or more different salts. A preferred buffered solution is phosphate buffered saline (particularly in conjunction with compositions intended for pharmaceutical administration), as it mimics the salt conditions of human blood. Since buffer salts can control the pH of a solution at low concentrations, a buffered diluent rarely modifies the biological activity of a biologically active peptide.

An “excipient” is any more or less inert substance that can be added to a composition in order to confer a suitable property, for example, a suitable consistency or to form a drug. Suitable excipients and carriers include, in particular, fillers such as sugars, including lactose, sucrose, mannitol, or sorbitol cellulose preparations such as, for example, maize starch, wheat starch, rice starch, agar, pectin, xanthan gum, guar gum, locust bean gum, hyaluronic acid, casein potato starch, gelatin, gum tragacanth, polyacrylate, methyl cellulose, hydroxypropylmethyl-cellulose, sodium carboxymethylcellulose, and/or polyvinylpyrrolidone (PVP). If desired, disintegrating agents can also be included, such as cross-linked polyvinylpyrrolidone, agar, or alginic acid or a salt thereof such as sodium alginate. Other suitable excipients and carriers include hydrogels, gellable hydrocolloids, and chitosan. Chitosan microspheres and microcapsules can be used as carriers. See WO 98/52547 (which describes microsphere formulations for targeting compounds to the stomach, the formulations comprising an inner core (optionally including a gelled hydrocolloid) containing one or more active ingredients, a membrane comprised of a water insoluble polymer (e.g., ethylcellulose) to control the release rate of the active ingredient(s), and an outer layer comprised of a bioadhesive cationic polymer, for example, a cationic polysaccharide, a cationic protein, and/or a synthetic cationic polymer; U.S. Pat. No. 4,895,724. Typically, chitosan is cross-linked using a suitable agent, for example, glutaraldehyde, glyoxal, epichlorohydrin, and succinaldehyde. Compositions employing chitosan as a carrier can be formulated into a variety of dosage forms, including pills, tablets, microparticles, and microspheres, including those providing for controlled release of the active ingredient(s). Other suitable bioadhesive cationic polymers include acidic gelatin, polygalactosamine, polyamino acids such as polylysine, polyhistidine, polyornithine, polyquaternary compounds, prolamine, polyimine, diethylaminoethyldextran (DEAE), DEAE-imine, DEAE-methacrylate, DEAE-acrylamide, DEAE-dextran, DEAE-cellulose, poly-p-aminostyrene, polyoxethane, copolymethacrylates, polyamidoamines, cationic starches, polyvinylpyridine, and polythiodiethylaminomethylethylene.

XI.D. Formulation of Pharmaceutical Compositions

The compositions and compounds of the invention can be formulated in any suitable manner. The compositions or compounds may be uniformly (homogeneously) or non-uniformly (heterogenously) dispersed in the carrier. Suitable formulations include dry and liquid formulations. Dry formulations include freeze dried and lyophilized powders, which are particularly well suited for aerosol delivery to the sinuses or lung, or for long term storage followed by reconstitution in a suitable diluent prior to administration. Other preferred dry formulations include those wherein a pharmaceutical composition according to the invention is compressed into tablet or pill form suitable for oral administration or compounded into a sustained release formulation. When the pharmaceutical composition is intended for oral administration but the composition or compound of the invention is to be delivered to epithelium in the intestines, it is preferred that the formulation be encapsulated with an enteric coating to protect the formulation and prevent premature release of the drugs included therein. As those in the art will appreciate, the pharmaceutical compositions of the invention can be placed into any suitable dosage form. Pills and tablets represent some of such dosage forms. The pharmaceutical compositions can also be encapsulated into any suitable capsule or other coating material, for example, by compression, dipping, pan coating, spray drying, etc. Suitable capsules include those made from gelatin and starch. In turn, such capsules can be coated with one or more additional materials, for example, and enteric coating, if desired. Liquid formulations include aqueous formulations, gels, and emulsions.

Some preferred embodiments concern compositions that comprise a bioadhesive, preferably a mucoadhesive, coating. A “bioadhesive coating” is a coating that allows a drug to adhere to a biological surface or substance better than occurs absent the coating. A “mucoadhesive coating” is a preferred bioadhesive coating that allows a substance, for example, a composition according to the invention, to adhere better to mucosa occurs absent the coating. For example, micronized particles (e.g., particles having a mean diameter of about 5, 10, 25, 50, or 100 μm) can be coated with a mucoadhesive. The coated particles can then be assembled into a dosage form suitable for delivery to an organism. Preferably, and depending upon the location where the cell surface transport moiety to be targeted is expressed, the dosage form is then coated with another coating to protect the formulation until it reaches the desired location, where the mucoadhesive enables the formulation to be retained while the compositions or compounds of the invention interact with the target cell surface transport moiety.

XI.E. Administration of Pharmaceutical Compositions

The pharmaceutical compositions of the invention facilitate administration of monoclonal antibodies to an organism, preferably an animal, preferably a mammal, bird, fish, insect, or arachnid. Preferred mammals include bovine, canine, equine, feline, ovine, and porcine animals, and non-human primates. Humans are particularly preferred. Multiple techniques of administering or delivering a compound exist in the art including, but not limited to, oral, rectal (e.g., an enema or suppository) aerosol (e.g., for nasal or pulmonary delivery), parenteral, and topical administration. Preferably, sufficient quantities of the composition or compound of the invention are delivered to achieve the intended effect. The particular amount of composition or compound to be delivered will depend on many factors, including the effect to be achieved, the type of organism to which the composition is delivered, delivery route, dosage regimen, and the age, health, and sex of the organism. As such, the particular dosage of a composition or compound of the invention included in a given formulation is left to the ordinarily skilled artisan's discretion.

Those skilled in the art will appreciate that when the pharmaceutical compositions of the present invention are administered as agents to achieve a particular desired biological result, which may include a therapeutic or protective effect(s) (including vaccination), it may be necessary to combine the composition or compound of the invention with a suitable pharmaceutical carrier. The choice of pharmaceutical carrier and the preparation of the composition or compound as a therapeutic or protective agent will depend on the intended use and mode of administration. Suitable formulations and methods of administration of therapeutic agents include, but are not limited to, those for oral, pulmonary, nasal, buccal, ocular, dermal, rectal, or vaginal delivery.

Depending on the mode of delivery employed, the context-dependent functional entity can be delivered in a variety of pharmaceutically acceptable forms. For example, the context-dependent functional entity can be delivered in the form of a solid, solution, emulsion, dispersion, micelle, liposome, and the like, incorporated into a pill, capsule, tablet, suppository, areosol, droplet, or spray. Pills, tablets, suppositories, areosols, powders, droplets, and sprays may have complex, multilayer structures and have a large range of sizes. Aerosols, powders, droplets, and sprays may range from small (1 micron) to large (200 micron) in size.

Pharmaceutical compositions of the present invention can be used in the form of a solid, a lyophilized powder, a solution, an emulsion, a dispersion, a micelle, a liposome, and the like, wherein the resulting composition contains one or more of the compounds of the present invention, as an active ingredient, in admixture with an organic or inorganic carrier or excipient suitable for enteral or parenteral applications. The active ingredient may be compounded, for example, with the usual non-toxic, pharmaceutically acceptable carriers for tablets, pellets, capsules, suppositories, solutions, emulsions, suspensions, and any other form suitable for use. The carriers which can be used include glucose, lactose, mannose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition auxiliary, stabilizing, thickening and coloring agents and perfumes may be used. Examples of a stabilizing dry agent includes triulose, preferably at concentrations of 0.1% or greater (See, e.g., U.S. Pat. No. 5,314,695).

XI.F. Dosages

Although individual needs may vary, determination of optimal ranges for effective amounts of pharmaceutical compositions is within the skill of the art. Human doses can be extrapolated from animal studies (Katocs et al., Chapter 27 In: Remington's Pharmaceutical Sciences, 18th Ed., Gennaro, ed., Mack Publishing Co., Easton, Pa., 1990). Generally, the dosage required to provide an effective amount of a pharmaceutical composition, which can be adjusted by one skilled in the art, will vary depending on the age, health, physical condition, weight, type and extent of the disease or disorder of the recipient, frequency of treatment, the nature of concurrent therapy (if any) and the nature and scope of the desired effect(s). See, for example, Nies et al., Chapter 3 In: Goodman & Gilman's The Pharmacological Basis of Therapeutics, 9th Ed., Hardman et al., eds., McGraw-Hill, New York, N.Y., 1996)

Dosing of therapeutic compositions is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The term “patient” is intended to encompass animals (e.g., cats, dogs and horses) as well as humans. Persons of ordinary skill can easily determine optimum dosages, dosing methodologies and repetition rates. Optimum dosages may vary depending on the relative potency of individual therapeutic agents, and can generally be estimated based on EC50 found to be effective in in vitro and in vivo animal models.

The range of doses (the amount of drug administered) is broad, since in general the efficacy of a therapeutic effect for different mammals varies widely with doses typically being 20, 30 or even 40 times smaller (per unit body weight) in man than in the rat. In general, dosage is from 0.01 ug to 100 g per kg of body weight, preferably 0.01 ug to 10 g/kg of body weight, 0.01 ug to 1000 mg/kg of body weight, 0.01 ug to 100 mg/kg of body weight, 0.01 ug to 10 mg/kg of body weight, 0.01 ug to 1 mg/kg of body weight, 0.01 ug to to 100 ug/kg of body weight, 0.01 ug to to 10 ug/kg of body weight, 0.01 ug to 1 ug/kg of body weight, 0.01 ug to 10 ug/kg of body weight, 0.01 ug to 1 ug/kg of body weight, 0.01 ug to 0.1 ug/kg of body weight, and ranges based on the boundaries of the preceding ranges of concentrations. Thus, for example, the preceding description of dosages encompasses dosages within the range of 100 to 10 g per kg of body weight, 10 g to 1000 mg/kg of body weight, 1000 mg to 100 mg, etc.

Doses may be given once or more daily, weekly, monthly or yearly, or even once every 2 to 20 years. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent the recurrence of the disease state, wherein the therapeutic agent is administered in maintenance doses, ranging from 0.01 ug to 100 g per kg of body weight, once or more daily, to once every 20 years. Some drugs, such as vaccines, may be administered once in a lifetime, or with booster shots only as circumstances warrant.

The specific dose is calculated according to the approximate body weight or surface area of the patient. Other factors in determining the appropriate dosage can include the disease or condition to be treated or prevented, the severity of the disease, the route of administration, and the age, sex and medical condition of the patient. Further refinement of the calculations necessary to determine the appropriate dosage for treatment is routinely made by those skilled in the art, especially in light of the dosage information and assays disclosed herein. The dosage can also be determined through the use of known assays for determining dosages used in conjunction with appropriate dose-response data.

An individual patient's dosage can be adjusted as the progress of the disease is monitored. Blood levels of the drug in a patient can be measured to see if the dosage needs to be adjusted to reach or maintain an effective concentration. Pharmacogenomics may be used to determine which drugs and dosages thereof are most likely to be effective for a given individual (Schmitz et al., Clinica Chimica Acta 308:43-53, 2001; Steimer et al., Clinica Chimica Acta 308:33-41, 2001).

XI.G. Uses of Pharmaceutical Compositions

The pharmaceutical compositions of the invention facilitate administration of biologically active complexes and compounds to an organism, preferably an animal, preferably a mammal, bird, fish, insect, or arachnid. Preferred mammals include bovine, canine, equine, feline, ovine, and porcine animals, and non-human primates. Humans are particularly preferred. Multiple techniques of administering or delivering a pharmaceutical composition exist in the art including, but not limited to, oral, aerosol (e.g., for nasal or pulmonary delivery), parenteral, and topical administration. Preferably, a sufficient quantity of the biologically active complex or compound, or a bioactive portion or metabolite thereof, of the pharmaceutical composition is delivered to achieve the intended effect. The particular amount of the biologically active complex or compound to be delivered will depend on many factors, including the effect to be achieved, the type of organism to which the pharmaceutical composition is delivered, delivery route, dosage regimen, and the age, health, and sex of the organism. As such, the particular dosage of composition or compound of the invention included in a given formulation is left to the ordinarily skilled artisan's discretion.

In another therapeutic context, the pharmaceutical compositions of the invention allow a biologically active complex or compound, or a bioactive portion or metabolite thereof, to be efficaciously delivered as part of a pIgR-targeting composition or compound. Because pIgR-ligands are delivered into cells by active transport, the instant pharmaceutical compositions afford better control over bioavailability of monoclonal antibodies as compared to passive transport mechanisms. As such, the pIgR-targeting protein conjugates and compositions of the invention enable improved uptake and utilization of the monoclonal antibody.

The compositions and compounds of the invention are also useful in diagnostic and related applications. One aspect of the invention involves the diagnosis and monitoring of certain diseases, preferably in kit form. This aspect is useful for assaying and monitoring the course of the diagnosis and prognosis of disease, for monitoring the effectiveness and/or distribution of a therapeutic agent or an endogenous compound, in a patient as well as other related functions.

In this aspect of the invention, it may be desirable to monitor or determine if, or determine the degree to which, a patient's pIgR-displaying cells are capable of, or presently are, endocytosing a detectably labeled composition or compound of the invention. Such methods are used in a variety of systems depending on the nature of the pIgR-targeting element(s) of a given protein conjugate.

For example, the degree to which a patient, or a biological sample therefrom, endocytoses a composition or compound that has a pIgR-targeting element derived from a bacterial protein that binds pIgR is a measure of a patient's susceptibility to infection by bacteria having that element. A higher degree or rate of uptake of the detectable label indicates that the patient is more susceptible to such infection.

As another example, the activity, distribution and/or concentration of endogenous pIgR proteins may be altered in various ways during the course of a disease or disorder. The pIgR proteins in a patient are measured over the course of a disease for diagnostic and prognostic purposes, as well as over the course of treatment of a disease or disorder, in order to monitor the effects on pIgR proteins. Diseases to which this aspect of the invention can be applied include but are not limited to diseases that involve the respiratory system, such as lung cancer and tumors, asthma, pathogenic infections, allergy-related disorders, and the like; the gastrointestinal tract, including cancers, tumors, pathogenic infections, disorders relating to gastroinstestinal hormones, Chron's disease, eating disorders, and the like; and any disease or disorder that is known or suspected to involve pIgR-displaying cells.

Compositions and compounds of the invention may be detectably labeled by virtue of comprising a detectable polypeptide such as, e.g., a green fluorescent protein (GFP) or a derivative thereof. If the protein conjugate comprises an epitope for which antibodies are available (including but not limited to commercially available ones such as c-myc epitope and the FLAG-tag), it may be detected using any of a variety of immunoassays such as enzyme-linked immunosorbent assay (ELISA) or a radioimmunoassay (RIA).

XII. Pharmacological Properties

Those skilled in the art are aware of pharmacological properties that influence the efficacy of drugs, and how to determine parameters that reflect these properties. See, generally, The Merck Manual of Diagnosis and Therapy, 15th Ed., Berkow et al., eds., 1987, Rahway, N.J., which is hereby incorporated by reference in its entirety. Some of pharmacological properties are as follows.

XII.A. Targeting

A drug that is designed to be specifically or preferentially delivered to its intended site of action is said to be targeted. That is, such drugs comprise targeting elements directed to the desired site of action. Antibodies, particularly single-chain antibodies, directed to surface antigens specific for a particular cell type have been associated with and used to target drugs. See, for example, Kuroki et al., “Specific Targeting Strategies of Cancer Gene Therapy Using a Single-Chain Variable Fragment (scFv) with a High Affinity for CEA,” Anticancer Res., pp. 4067-71, 2000; U.S. Pat. No. 6,146,885, to Dornburg, entitled “Cell-Type Specific Gene Transfer Using Retroviral Vectors Containing Antibody-Envelope Fusion Proteins”; Jiang et al., “In Vivo Cell Type-Specific Gene Delivery With Retroviral Vectors That Display Single Chain Antibodies,” Gene Ther. 1999, 6:1982-7; Engelstadter et al., “Targeting Human T Cells By Retroviral Vectors Displaying Antibody Domains Selected From A Phage Display Library,” Hum. Gene Ther. 2000, 11:293-303; Jiang et al., “Cell-Type-Specific Gene Transfer Into Human Cells With Retroviral Vectors That Display Single-Chain Antibodies,” J. Virol 1998, 72:10148-56; Chu et al., “Toward Highly Efficient Cell-Type-Specific Gene Transfer With Retroviral Vectors Displaying Single-Chain Antibodies,” J. Virol 1997, 71:720-5; Chu et al., “Retroviral Vector Particles Displaying The Antigen-Binding Site Of An Antibody Enable Cell-Type-Specific Gene Transfer,” J. Virol 1995, 69:2659-63; Chu et al., “Cell Targeting With Retroviral Vector Particles Containing Antibody-Envelope Fusion Proteins,” Gene Ther. 1994, 1:292-9; Einfeld et al., “Construction of a Pseudoreceptor That Mediates Transduction by Adenoviruses Expressing a Ligand in Fiber or Penton Base,” J. Virol. 1999, 73:9130-9136; Marin et al., “Targeted Infection of Human Cells via Major Histocompatibility Complex Class I Molecules by Moloney Murine Leukemia Virus-Derived Viruses Displaying Single-Chain Antibody Fragment-Envelope Fusion Proteins,” J. Virol., 1996, 70:2957-2962; Somia et al., “Generation of targeted retroviral vectors by using single-chain variable fragment: An approach to in vivo gene delivery,” Proc. Natl. Acad. Sci. USA, 1995, 92:7570-7574; Liu et al., “Treatment of B-Cell Lymphoma With Chimeric IgG and Single-Chain Fv Antibody-Interleukin-2 Fusion Proteins,” Blood, 1998, 92:2103-2112; Martin et al., “Retrovirus Targeting by Tropism Restriction to Melanoma Cells,” J. Virol., 1999, 73:6923-6929; Ramjiawan et al., “Noninvasive Localization of Tumors by Immunofluorescence Imaging Using a Single Chain Fv Fragment of a Human Monoclonal Antibody with Broad Cancer Specificity,” Amer. Cancer Society, 2000, 89:1134-1144; Snitkovsky et al., “A TVA-Single-Chain Antibody Fusion Protein Mediates Specific Targeting of a Subgroup A Avian Leukosis Virus Vector to Cells Expressing a Tumor-Specific Form of Epidermal Growth Factor Receptor,” J. Virol., 2000, 74:9540-9545; Chu et al., “Toward Highly Efficient Cell-Type-Specific Gene Transfer with Retroviral Vectors Displaying Single-Chain Antibodies,” J. Virol., 1997, 71:720-725; Kulkarni et al., Programmed cell death signaling via cell-surface expression of a single-chain antibody transgene, Transplantation 2000 Mar. 27; 69(6):1209-17.

Targeting elements directed to the pIgR stalk or other pIgR domains and regions described herein may serve an additional purpose beyond penetrating epithelial barriers. Some cancer cells aberrantly express pIgR (Phillips-Quagliata et al., J. Immunol. 165:2544-2555, 2000). Targeting elements directed to the pIgR stalk or other pIgR domains and regions serve as targeting elements to such cancer cells, or other cells that aberrantly express pIgR. In these instances, targeting elements directed to the pIgR stalk or other pIgR domains and regions can be associated with cytotoxins and delivered to the cancer cells for therapeutic benefit.

XII.B. Rate of Absorption

The absorption rate constant expresses the speed of drug absorption. Drug absorption refers to the process of drug movement from the site(s) of administration of the drug into the body of an animal. Various factors, including the formulation of the drug, influence the efficacy of rate of absorption of a drug. For example, most orally administered drugs are in the form of tablets or capsules, for reasons such as convenience, economy, stability, and patient acceptance and compliance. These capsules or tablets must disintegrate or dissolve before absorption of the drug can occur. There are a variety of factors capable of varying or retarding disintegration of solid dosage forms, and effecting the dissolution rate, thereby determining the availability of the drug for absorption.

The absorption of some drugs is further influenced by factors that result from the consumption of food. For example, the presence of fiber or other substances in the GI tract may limit the absorption of drugs, and the secretion of fluids that occur in response to ingestion or during digestion may also impact their absorption. Once such fluid is bile, which enhances absorption of many substances, including some drugs. The release of digestive enzymes may be induced by ingestion, and these enzymes may effect the rate of dissolution of pills, tablets, and the like, and/or degrade the drug.

XII.C. Bioavailability

The bioavailability of a drug is another pharmacological property: Bioavailability is defined as the rate at which and the extent to which a drug, or a biologically active metabolite or portion thereof, enters the general circulation and/or its targeted site of action. Bioavailability is influenced by a number of factors, including how the drug product is designed and manufactured, its physicochemical properties, the rate at which the drug is eliminated from the body, and factors that relate to the physiology and pathology of the patient. Reactions that compete with absorption can reduce bioavailability. They include complex formation (eg, between tetracycline and polyvalent metal ions), hydrolysis by gastric acid or digestive enzymes (e.g., penicillin and chloramphenicol palmitate hydrolysis), conjugation in the gut wall (e.g., sulfoconjugation of isoproterenol), adsorption to other drugs (e.g., digoxin and cholestyramine), and metabolism by luminal microflora. Any of these factors can be changed to influence bioavailability, which is a pharmacological property that can be adjusted to achieve or enhance desirable attributes.

Assessment of bioavailability from plasma concentration-time data usually involves determining the maximum (peak) plasma drug concentration, the time at which maximum plasma drug concentration occurs (peak time), and the area under the plasma concentration-time curve (AUC). The plasma drug concentration increases with the extent of absorption; the peak is reached when the drug elimination rate equals absorption rate. Bioavailability determinations based on the peak plasma concentration can be misleading, because drug elimination begins as soon as the drug enters the bloodstream. The most widely used general index of absorption rate is peak time; the slower the absorption, the later the peak time. However, peak time is often not a good statistical measure because it is a discrete value that depends on frequency of blood sampling and, in the case of relatively flat concentrations near the peak, on assay reproducibility. AUC is a more reliable measure of bioavailability, as it is directly proportional to the total amount of unchanged drug that reaches the systemic circulation.

XII.D. Elimination and Clearance

The rate of elimination of a drug from the body varies and effects its efficacy. A higher rate of elimination corresponds to decreased bioavailability. Thus, lower rates of elimination are generally preferred, although higher rates may be preferable for drugs having undesirable effects, such as toxicity. One parameter relating elimination rate to plasma concentration is total clearance, which equals renal clearance plus extrarenal (metabolic) clearance. The elimination rate constant is a function of how a drug is cleared from the blood by the eliminating organs and how the drug distributes throughout the body. Another factor relating to elimination is the fraction excreted unchanged, which reflects the amount of drug that is excreted relative to the amount that is metabolized. A low fraction indicates that hepatic metabolism is the likely mechanism of elimination, whereas higher fractions indicate that renal excretion is the predominant form of drug elimination.

The rate of elimination is desirably increased or decreased depending on the nature and use of the drug in question. Often, a decreased rate of elimination is desirable, as this increases bioavailability. However, in the case of some agents, an increased rate of elimination may be preferable. For example, not every molecule of a targeted drug that is introduced will find its intended site of action, and it may be desirable to remove these molecules from the body before they cause an undesirable effect at some other site in the body.

XII.E. Therapeutic Index

Another pharmacological property involves the therapeutic index, which is a measure of the relative desirability of a drug for the attaining of a particular therapeutic result. The therapeutic index is usually expressed as the ratio of the largest dose producing no toxic symptoms to the smallest dose that results in a desired therapeutic result. Higher therapeutic indicia are preferred and an index of <1 is unacceptable, except in the case of some terminal diseases.

XII.G. First-Pass Effects

Following oral administration, many drugs are absorbed intact from the GI tract and transported first via the portal system to the liver, where they undergo extensive metabolism. Such metabolism may deactivate or degrade the drug, thus lowering or eliminating its biological activity, which in turn reduces bioavailability. Such processes, which typically but need not occur in the liver, are called first-pass effects. First-pass effects may so greatly limit the bioavailability of an orally administered drug that alternative routes of administration must be employed in order to achieve a therapeutically effective dose of the drug. Drugs transported through epithelial tissues may bypass first-pass effects, which is a pharmacological property that is a desirable attribute.

XII.F. Half-Life

The half-life of a drug is the time required for drug concentration or the amount of drug in the body to decrease by 50%. For most drugs, half-life remains the same regardless of how much drug is in the body, but there are exceptions (e.g., phenytoin, theophylline, and heparin). Generally, a higher half-life is preferred, as this reduces the amount and lowers the frequency of administration of the drug necessary to achieve its intended therapeutic effect. However, there are times when a decreased half life is preferred, particularly when the drug has undesirable side-effects, e.g., toxicity.

### EXAMPLES

### Example 1

### Molecular Reagents

1.1. Preparation of a Polyclonal Anti-sFv5AF-Cys Antibody

In the Examples, polyclonal antibodies directed to sFv5AF are used to simultaneously detect the single-chain antibodies sFv5AF and sFv5AF-Cys, and conjugates comprising these sFv's. The anti-sFv5AF polyclonal antibodies were prepared as follows.

FLAG-tagged sFv5AF was used as an immunogen for the production of antisera (polyclonal antibodies). The antisera was commercially prepared by HTI Bio-Products (Ramona, Calif.). In brief, 200 μg of FLAG-tagged sFv5AF was used for the initial injection (Day 1) with Complete Freund's Adjuvant, followed by boosts of 200 μg fusion protein with Incomplete Freund's Adjuvant every 2 weeks. The injections were subcutaneous. Bleeds were taken at approximately 7 weeks and 9 weeks.

The sera was screened for reactivity with sFv5AF using an ELISA. Sera that tested positive in the ELISA were examined by Western blot to confirm the presence of polyclonal antibodies reactive with sFv5AF.

### Example 2

### Cloning of a Simian pIgR

2.1. Isolation of pIgR cDNA from Monkey Intestinal Tissue

Rhesus and Cynomolgus monkey intestinal tissue was obtained from Yerkes Regional Primate Center (Atlanta, Ga.). At least 30 grams of tissue specimens were each prepared from ileum and colon sections where the tissue was excised within one-half hour postmortem, rinsed free of feces with PBS, and then rapidly frozen using liquid nitrogen, shipped overnight on dry ice and stored frozen at −80° C.

A section of cynomolgus colon weighing 5.3 grams (wet weight) was placed in a 50 ml conical tube and rapidly washed 3-5 times with approximately a 30 ml volume of PBS to remove residual fecal material. The colon segment was removed to a very small plastic weigh boat and a longitudinal incision was made exposing the luminal surface, which was quickly and gently rinsed with ˜50 mls of PBS. One (1) ml of TRizol reagent (Life Technologies) was layered and massaged on the luminal surface, collected in a 15 ml conical tube, and total cellular RNA isolated as per manufacturer's instructions. Briefly, the RNA solution was centrifuged at 12,000×g to remove insoluble cellular debri, and 700 uls of total solution transferred to an microfuge tube. 140 uls of chloroform was added the solution centrifuged at 14,000 rpms for 15 minutes at 4° C. 430 uls of aqueous phase was collected, 215 uls of isopropanol added, incubated at room temperature for 10 minutes, and the RNA precipitated by centrifugation at 14,000 rpms for 10 minutes at 4° C. The white pellet was washed with 1 ml of 75% ethanol, air dried for 5-10 minutes, and the RNA pellet resuspended in 50 uls of DEPC-treated water. Quantitation of total RNA was determined by spectrometry using the value of 1 OD₂₆₀ value=40 μg RNA/ml.

The sequences of the synthetic degenerate DNA primers (prepared by Genset, Inc., Paris, France) that were used in the first strand cDNA synthesis (RT-PCR) and PCR amplification of the cynomolgus monkey partial cDNA are as follows.

(SEQ ID NO: _{——})

RT-PCR primer: EPKKAKRS-Low Reverse primer
5′-GTATCGATCTTTTTGCCTTCTTGGGYTC-3′
PCR Forward primer: EKYWCKW Forward primer
5′-GGAATTCGARAARTAYTGGTGYAARTGG-3′

Note: “R” designates either an A or G purine base; and Y designates either an C or T pyrimidine base.

PCR Reverse primer: EPKKAK-Low Reverse primer (SEQ ID NO:______)

5′-GTATCGATCXRTTXGCRTTRTTNGGRTC-3′

Note: “N” designates either of the A, C, G or T bases; “R” designates either an A or G purine base; “Y” designates a either an C or T pyrimidine base; “X” designates a nucleotide analog.

An oligonucleotide primer (SEQ ID: ______ RT-PCR primer) was used together with the SuperScript First Strand Synthesis Kit (Life Technologies) to synthesize the first strand cDNA from 5 ug of total cynomolgus monkey RNA as per manufacturer's instructions. Briefly, 100 pmols of primer (SEQ ID: ______ RT-PCR primer) and 5 ug of total RNA was included in a 10 ul RT-PCR reaction, heated to 70° C. for 10 minutes, then cooled to 4° C. A 9 ul 10×RT-buffer mixture was then added to the RT-PCR reaction and incubated at 42° C. for 2 minutes, followed by the addition of 1 ul of SuperScript II enzyme to each reaction. The reverse transcription reaction was allowed to proceed at 42° C. for 50 minutes. Proper control reactions were also assembled and run simultaneously. The reactions were terminated by heating to 70° C. for 15 minutes. To prevent interference of the RNA in the subsequent PCR amplification step, 1 ul of RNase. H was added and the reaction incubated at 37° C. for 20 minutes before storing the single stranded cDNA material at −20° C.

2.2. Isolation, Identification and Sequencing of Simian pIgR Sequences

A 2 ul aliquot of the cynomolgus monkey cDNA reaction was used in a 50 ul PCR reaction and a partial cynomolgous double stranded cDNA amplified using 0.2 uM concentration of the Forward (SEQ ID NO:______ PCR Forward primer) and Reverse (SEQ ID NO:______ PCR Reverse primer) primers together with 2.5 units of High Fidelity Platinum Taq (Life Technologies). Amplification was carried out as per manufacturer's instructions and thermocycling conditions as follows: 1) denaturation at 94° C. for 10 minutes; 2) 30 cycles of denaturation for 1 minute at 94° C., primer annealing for 1 minute at 60° C., primer extension for 30 seconds at 72° C., and 3) a final 4° C. storage step. The correct size of the 730 bp PCR product was confirmed by agarose gel electrophoresis. The entire PCR reaction was run on a preparative agarose gel and the 730 bp partial cDNA fragment separated from contaminating primers and purified using the Qiagen QIAquick purification kit. The purified partial cDNA fragment was re-amplified and purified as described above. Due to the utilization of Taq DNA polymerase, all PCR products contained a 3′-A overhang and were be easily ligated into an intermediate vector using the TOPO TA Cloning Kit (Invitrogen). The resulting PCR product was ligated into the pCR-II vector (Invitrogen) as per manufacturer's instructions and the ligation reactions transformed into TOPO One-shot competent cells (Invitrogen). Colonies were selected and 3 ml mini-cultures grown, miniprep DNA prepared using the Qiagen Miniprep Kit (Qiagen), and positive clones identified by an Eco RI restriction enzyme analysis.

Eco RI digestion identified 4 positive clones containing the PCR DNA product. Maxiprep DNA was prepared (Qiagen DNA Maxikit) for two (2) clones and the DNA nucleotide sequence determined following sequencing of the DNA with both Sp6 (SEQ NO:______ Sp6) and T7 (SEQ. ID NO:______ T7) sequencing primers (SDSU Microchemical Core Facility). A plasmid having the correct sequence was selected and designated “pTA-CynMonk-pIgR”.

2.3. Results

Degenerate oligonucleotides were used to clone a 730 nucleotide region of cynomolgus monkey pIgR cDNA from monkey intestinal tissue. This partial cDNA sequence encodes for most of domain 5 through the cytoplasmic domain (homologous to a region of the human pIgR molecule corresponding to amino acids Glu474 through Ser717). Detailed sequence and alignment analysis comparing the human and cynomolgus monkey pIgR cDNAs demonstrate that the sequences differ in 18 amino acids within this 242 amino acid region (Glu474 through Ser717). The amino acid sequences for a simian pIgR are shown in FIG. 2B.

2.4. Sequences of pIgR Homologs from Different Species

Nucleic acids and polypeptides, having nucleotide and amino acid sequences, respectively, of pIgR from cynomolgus, or portions of these sequences, are incorporated into a variety of methods and compositions. The cynomolgus cDNA is used to exemplify the production and uses of these methods and compositions.

Nucleic acids are used to produce pIgR polypeptides via recombinant DNA technology. The nucleic acids are used to generate chimeric reading frames that incorporate pIgR or a portion, domain or region thereof. Chimeric reading frames encode fusion proteins (e.g., a GST-domain 6 fusion protein), chimeric proteins (e.g., a rat/rabbit hybrid pIgR), amino acid substituted polypeptides and other derivatives of pIgR, and polypeptides that have a therapeutic benefit when expressed within the cells of an animal, including a human. The nucleic acids, or synthetic oligonucleotides having sequences derived therefrom, are used as probes for the identification and/or amplification of pIgR-encoding nucleic acids from other species, and other members of the pIgR family of proteins that are present in the genome of the same species from which the nuclic acids originated. Nucleic acids having a sequence, or a portion of a sequence, that is the reverse complement of the sense strand of the nucleic acids can be used as antisense molecules. Derivatives of a polypeptide that has the amino acid sequence set forth including without limitation oligopeptides, proteolytic fragments, fusion proteins, and peptiomimetics. These polypeptides are therapeutic agents and/or are used as target molecules in the methods of the invention.

### Example 3

### Cloning of pIgR Genes from Other Species

3.1. Cloning of Rat pIgR cDNA

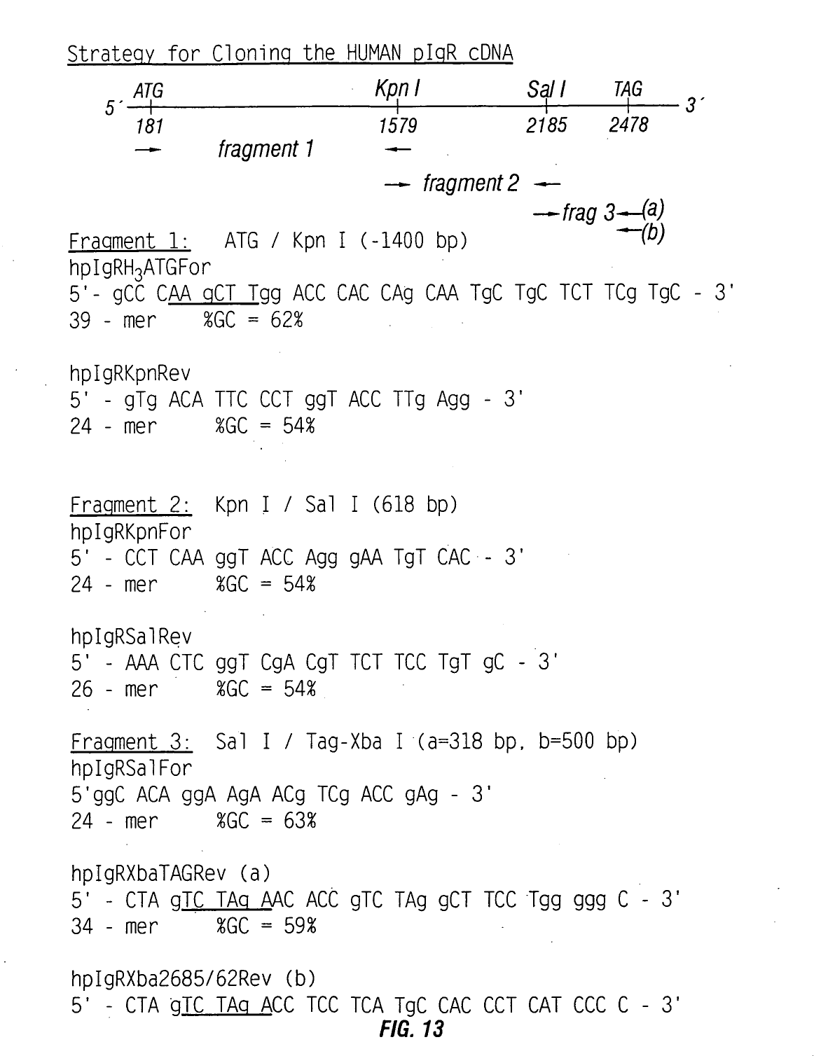

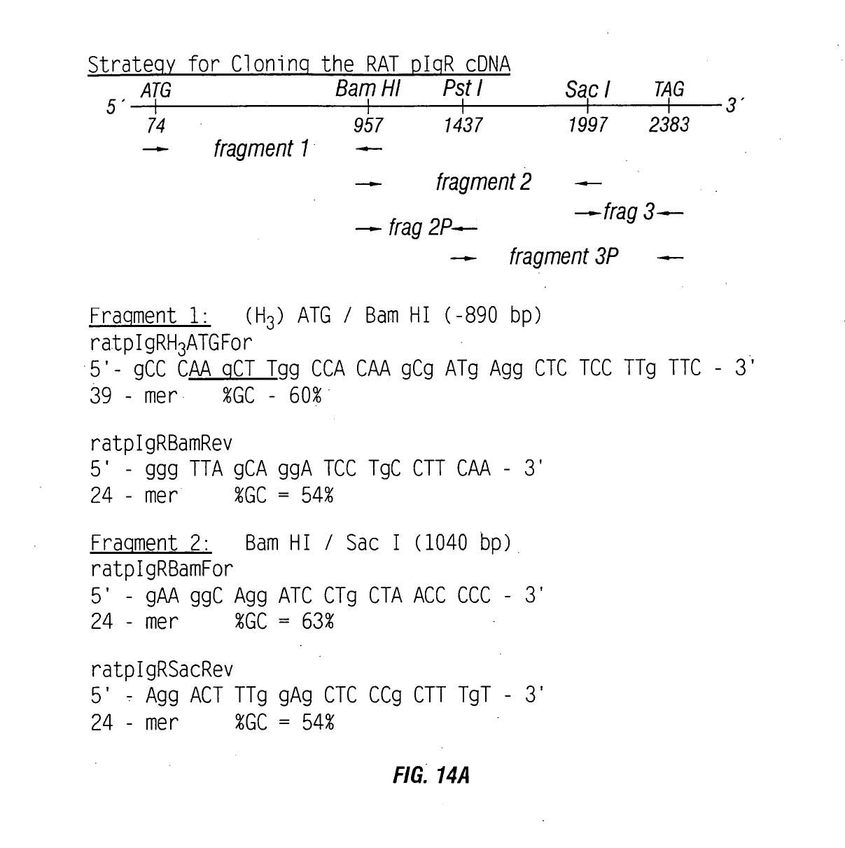

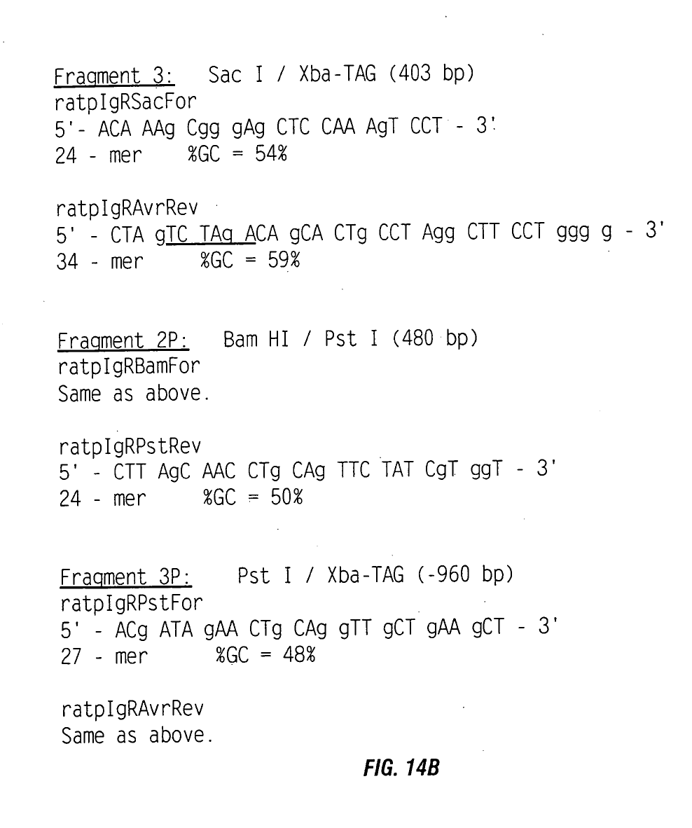

A rat liver cDNA library (Clontech) was used as a source for template for the amplification of rat pIgR sequences. The pIgR cDNA was amplified as 5 separate fragments which can be combined to regenerate the entire rat pIgR sequence (see FIG. 14). Alternatively, the sequences contained within separately cloned cDNA's may be used as a source for sequences that encode a rat stalk molecule or sequences derived therefrom.

As can be seen in FIG. 14, the primers used to amplify the rat cDNA regenerated or introduced restriction enzyme sites into the cDNA for ease of subcloning and other subsequent manipulations. Each fragment was treated with the appropriate restriction enzymes and ligated into a cloning vector (e.g., pBluescript from Stratagene or pUC19 from NEB) in order to generate an “intermediate vector”. The sequence of the inserted cDNA was determined in order to confirm the sequence of the amplified DNA.

3.2. Cloning of Mouse pIgR cDNA

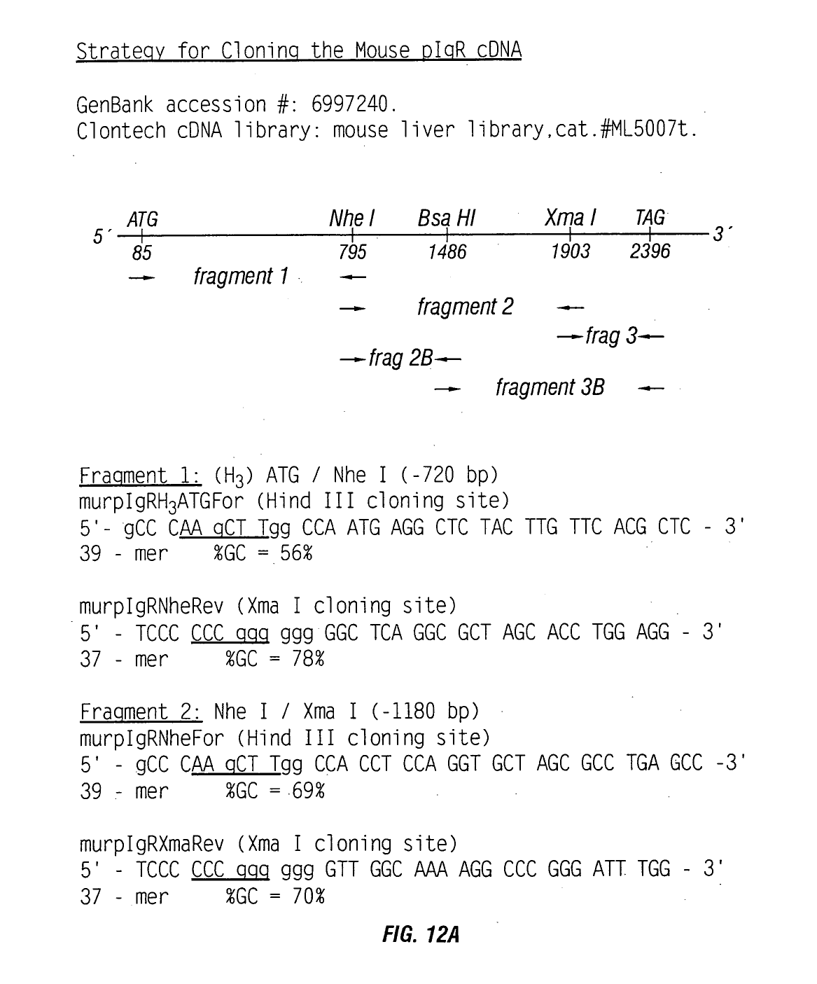

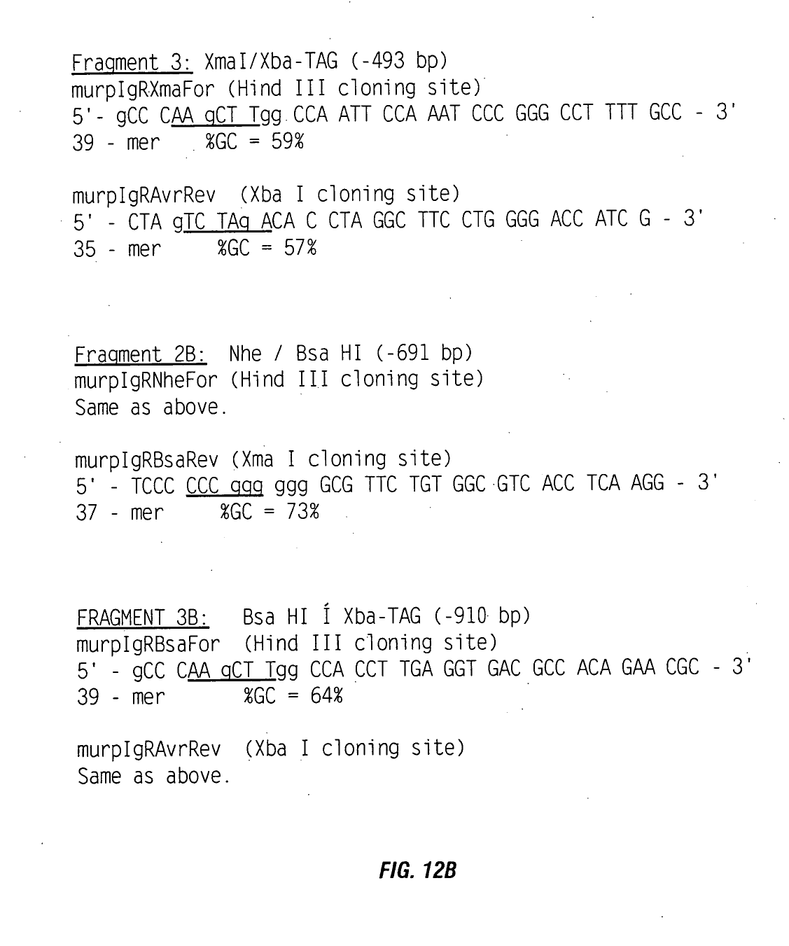

A mouse liver cDNA library (Clontech) is used as a source for template for the amplification of mouse pIgR sequences. As was the case for the rat pIgR cDNA's, the mouse cDNA is amplified as 5 separate fragments which can be combined to regenerate the entire mouse pIgR sequence (see FIG. 12). Alternatively, the sequences contained within separately cloned cDNA's may be used as a source for sequences that encode a mouse stalk molecule or sequences derived therefrom. As can be seen in FIG. 12, the primers used to amplify the mouse cDNA are designed to introduce restriction enzyme sites into the cDNA for ease of subcloning and other subsequent manipulations. Each fragment is treated with the appropriate restriction enzymes and ligated into a cloning vector in order to generate an “intermediate vector”. The sequence of the cDNA in the intermediate vector was determined in order to confirm the sequence of the amplified DNA.

3.3. Cloning of Human pIgR cDNA

A human colon cDNA library (Clontech) was used as a source for template for the amplification of human pIgR sequences. The human cDNA sequences were amplified as 3 separate fragments which were inserted into intermediate vectors and assembled as described above (see FIG. 13).

3.4. Construction of Rabbit/Rat Chimeric pIgR

Expression of pIgR in Madin-Darby canine kidney (MDCK) cells using retroviral vectors has been described by Breitfeld et al. (Methods Cell Biol 32:329-37, 1989). The expression of rabbit and human pIgR in MDCK cells has been described, respectively, by Barroso et al. (J Cell Biol 1994 124:83-100) and Tamer et al. (J. Immunol 1995 155:707-14, 1995). Because rats are useful for in vivo assays, initial in vitro transcytosis assays used MDCK cells transfected with rat pIgR. However, the expression of rat pIgR in transfected MDCK cells was reduced relative to results obtained with rabbit pIgR transfected MDCK cells. Without wishing to be bound by any particular theory, the relatively reduced expression of rat pIgR may be a consequence of an unusual structure in the 5′ untranslated region of the rat pIgR cDNA (Fabregat et al., Physiol Genomics 5:53-65, 2001; Fodor et al., DNA Cell Biol 16:215-25, 1997; Koch et al., Nucleic Acids Res 23:1098-112, 1995).

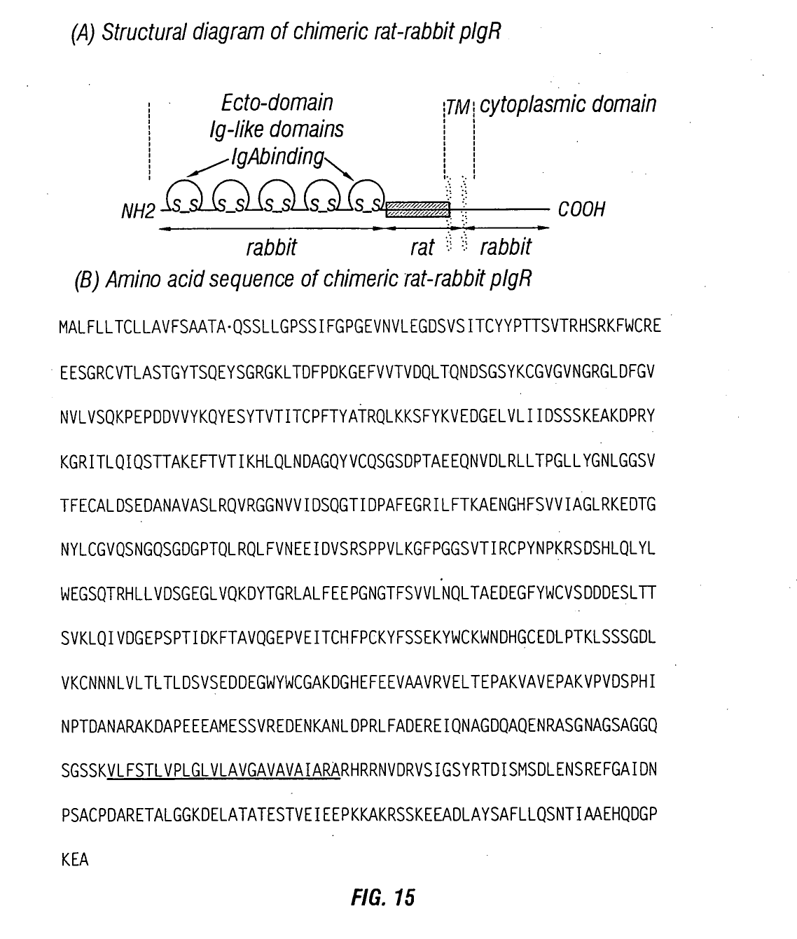

To enhance the production of a rat-like pIgR in transfected MDCK cells, a chimeric protein was produced via PCR using primers to rat and rabbit pIgR cDNA sequences and methods known in the art. The chimeric protein consists of amino acids 1-554 of rabbit pIgR, followed by amino acids 553-645 of rat pIgR, then amino acids 651-756 of rabbit pIgR. This chimeric protein contains the transmembrane and membrane proximal regions of rat pIgR, whereas the remainder of the molecule is derived from rabbit pIgR. The chimeric pIgR has the same activity as wild type rabbit pIgR in pIgR assays such as transcytosis of IgA from the basolateral to the apical surface (forward transcytosis). The structure and amino acid sequence of the chimeric pIgR protein is shown in FIGS. 15A and 15B, respectively. The chimeric protein was expressed from an expression construct comprising the expression vector pCB7.

### Example 4

### GST-Stalk Fusion Proteins

4.1. GST-Stalk Fusion Proteins

GST-stalk fusion proteins are one type of pIgR target molecule. The GST (glutathionine-5-transferase, from Schistosoma japonica, unless otherwise indicated) polypeptide has several illustrative desirable attributes. It specifically binds glutathione, and with a sufficiently high affinity that it can be used to attach fusion proteins to solid surfaces coated with glutathione, and many such surfaces are commercially available; detectably labeled antibodies directed to GST epitopes are commercially available; and the GST amino acid sequences allow some fusion proteins to have enhanced attributes such as, e.g., enhanced solubility, biologically active conformations, and the like.

GST fusion proteins may optionally comprise elements useful for the detection, isolation, purification and manipulation of the GST fusion protein. Non-limiting examples of such elements include elements such as a 6×His tag, a FLAG tag, a c-myc epitope, a fluorescent polypeptide (e.g., GFP), a detectable enzymatic polypeptide (e.g. horse radish peroxidase, beta-galactosidase), or a biotin-binding polypeptide (e.g., avidin or streptavidin) polypeptide. GST fusion proteins are expressed in E. coli, purified on a glutathione column and attached to solid surfaces by known techniques (see, e.g., Smith et al., Unit 16.7, “Expression and Purification of Glutathione-S-Transferase Fusion Proteins” in Short Protocols in Molecular Biology, 2nd Ed., Ausubel et al., Editors, John Wiley & Sons, pp. 16-28 to 16-31, 1992).

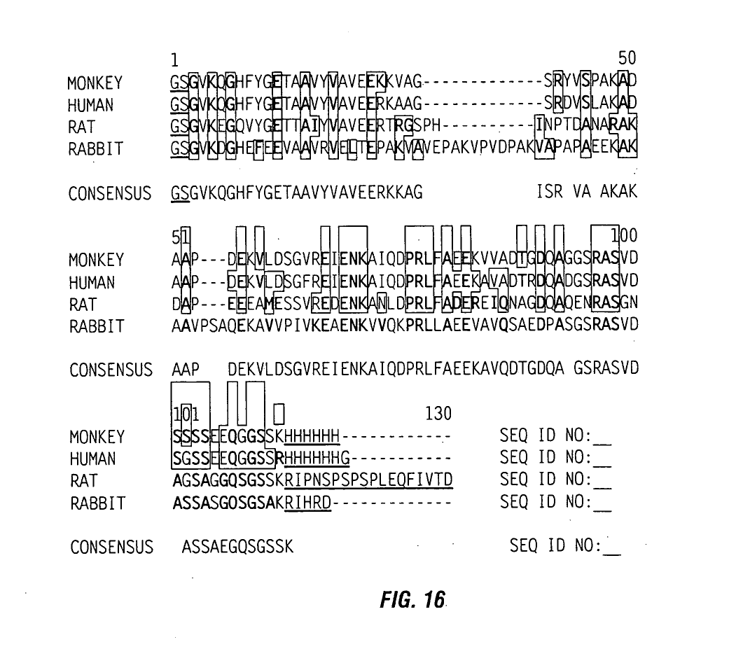

Non-limiting examples of GST fusion proteins include those that comprise a portion of the stalk that contains the desired sites of reaction, e.g., domain 5 and domain 6, domain 6, or smaller portions of domain 6; or of any other regions of pIgR and stalk molecules such as those described herein in Tables 1 and 4. The fragment of pIgR or stalk molecule used in a GST fusion protein may change depending on the nature of a particular use of the GST fusion protein, but those skilled in the art will know what amino acid sequences are appropriate to include in a given GST fusion protein. Table 12 summarizes the general characteristics of GST-stalk fusion proteins that are described in more detail in the subsequent subsections and in FIG. 16.

TABLE 12
GST-STALK FUSION PROTEINS

| GST Fusion | | Molecular | | Binds to single |
|---|---|---|---|---|
| Protein | Origin of Stalk | Weight of | 6xHis Tag | chain antibody |
| Description | Sequences | Fusion Protein | present? | sFv5? |
| GST-Cyn | Cynomolgus Monkey | ˜37.6 kD | Yes | Yes |
| monkey-stalk | partial cDNA | | | |
| GST-Human-stalk | Human cDNA | ˜37.8 kD | Yes | Yes |
| GST-Rat-stalk | Rat cDNA | ˜39.3 kD | No | Yes |
| GST-Rabbit-stalk | Rabbit cDNA | ˜38.8 kD | No | No |

4.2. GST—(Cynmonkey Stalk) Fusion Protein

A plasmid comprising cynomolgus monkey pIgR sequences (“pTA-CynMonk-pIgR,” which is a derivative of the pCR-II plasmid (Invitrogen) having simian pIgR sequences) was used as a template for PCR amplification of the cynomolgus monkey pIgR stalk region using the CynMpIgRstalk-5′FOR and CynMpIgRstalk-3′REV sequencing primers. These primers allow for the use of a directional cloning strategy (BglII to EcoRI ligation) and result in the incorporation of a C-terminal 6×His tag that can be used to isolate or attach the fusion protein to a solid surface.

CynMpIgRstalkGST 5′FOR, a 5′-Forward PCR primer containing a BglII site (underlined) and having the sequence (SEQ ID NO:______):

5′-CGGGAAGATCTGGAGTGAAGCAGGGCCACTTCTATGG-3′

CynMpIgRstalkGST 3′REV, a 3′-Reverse PCR primer containing an in-frame 6×His tag and an Eco RI site (underlined) (SEQ ID NO:______):

5′-CGGAATTCCTAGTGATGGTGATGGTGATGTTTGGAGCTCCCAC-
CTTGTTCCTCAGAGC-3′

The 309 bp PCR fragment was gel-purified and subjected to restriction digestion using BglII and EcoRI enzymes, and the resulting 305 bp fragment was gel-purified. The purified BglII-EcoRI fragment was cloned into BamHI- and EcoRI-digested pGEX-2TK (Amersham Pharmacia), a plasmid that has a GST-encoding nucleic acid sequence that can be fused in-frame with a cloned DNA. The resulting plasmid was subjected to DNA sequence analysis to confirm the absence of any PCR-induced mutations and to verify that the GST and pIgR sequences were linked in-frame with each other.

4.3. Other GST-Stalk Fusion Proteins

GST fusion proteins derived from human, rat and rabbit stalk sequences were prepared essentially according to the methods and methods used in the preceding subsections the preparation of a GST—(Cynmonkey stalk) fusion protein. One exception is that the stalk sequences were, in some cases, amplified from the above-described cDNA intermediate vectors comprising fragments of the pIgR human, rat and rabbit sequences, respectively.

For example, in the case of the GST-(rabbit stalk) fusion protein, a plasmid comprising rabbit pIgR sequences (“pGST-RabpIgRStalk”) was digested with BamHI and EcoRI, which liberates a 312 bp fragment. The 312 bp fragment was cloned into BamHI-EcoRI-treated pGEX-2TK vector, a plasmid that has a GST-encoding nucleic acid sequence that can be fused in-frame with a cloned DNA. The resulting plasmid was subjected to DNA sequence analysis to confirm the absence of any PCR-induced mutations and to verify that the GST and pIgR sequences were linked in-frame with each other.

### Example 5

### Preparation of Ligands Directed to Domain 6 and pIgR Stalk Molecules

5.1. Assays for Ligands

An assay is prepared by applying purified pIgR stalk molecules or GST-pIgR stalk molecules, or any other pIgR target, to multiwell (48-well, 96-well and other size lates and allowing the protein to adhere to the wells of the plates during overnight incubation. The plates are washed to remove unbound proteins. Samples of the serum from the immunized mice are incubated with the pIgR or GST-pIgR coated plates. After 1 to 2 hours of incubation (gentle shaking at room temperature), the plate is washed free of unreacted immune serum proteins. Mouse antibodies that react with an immobilized GST-pIgR protein are detected by adding to each well a sample of a goat antibody that has been raised against and is directed to mouse immunoglobulin, i.e., all subclasses of murine immunoglobulins. The goat antibody is conjugated to an enzyme that is used for detection; non-limiting examples include horse radish peroxidase and alkaline phosphatase. After unreacted horse radish peroxidase or alkaline phosphatase conjugated goat anti-mouse immunoglobulin has been washed from the wells, the substrate of horse radish peroxidase or alkaline phosphatase is added. When the color is sufficiently developed, the reaction is stopped and quantitated using a spectrophotometer. In the positive wells, antibodies against the GST-pIgR protein will be present. Some of these antibodies are directed to the GST portion of the protein if GST-pIgR is used. By assaying against other GST fusion proteins, it is determined if the antibodies are against GST or pIgR. This assay is also used to identify antibody producing cells and clones in 96-well plates that are part of the process of isolating clones of hybridomas that produce the desired monoclonal antibody.

Beads that bind GST moieties on GST-fusion proteins are also used for assays. GST-pIgR bound to beads is reacted with sera that contain antibodies directed against pIgR. The antibodies that react with and bind to pIgR can then be detected by an anti-antibody conjugated to horse radish peroxidase or alkaline phosphatase. If the antibodies that react with pIgR are derived from mice, then the antibodies that detected the presence of the mouse antibody is obtained from another animal species, such as goat or sheep. Those skilled in the art will know how to adjust the source and specificity of the detecting antibody conjugates (i.e. horse radish peroxidase or alkaline phosphatase conjugated to anti-FLAG tag antibody) to obtain the desired results.

5.2. Preparation of Monoclonal Antibodies (Mabs)

Monoclonal antibodies are created by immunizing mice with portions of pIgR, generally prepared as oligopeptides having defined amino acid sequences. For example, a nucleic acid encoding an amino acid sequence found in a conserved region of pIgR, such as those described in Table 1, or an amino acid sequence that varies between homologs, such as, e.g., R1, R2a, R2b, R3a, R3b, R3c (etc.) (Table 4) is used to create a pIgR-target-GST fusion protein that is expressed in a host cell such as E. coli. The GST portion of the fusion protein is used to isolate the fusion protein, and the purified GST-pIgR protein is mixed with adjuvant and injected into mice to produce an immune response. The extent of the immune response is measured over time by removing blood from the immunized mice at regular intervals and measuring the level of antibodies directed to the GST-pIgR fusion protein using an immunoassay, e.g., an ELISA.

Once the immunized mouse has been shown to be producing antibodies directed to the GST-pIgR fusion protein, the spleen of the mouse is harvested, and cells therefrom are prepared for fusion with immortalized fusion partners, such as the NS/1 cell line, according to Kohler and Milstein, in order to create Mab-producing hybridoma cell lines. Independently isolated clones and subclones are grown to an appropriate density, the cell supernatant is assayed using an ELISA to determine if antibodies that react with the GST-pIgR fusion proteins are produced by each clone or subclone. Positive wells are assayed using limiting dilution, and clonal and subclonal cell lines are eventually obtained that produce Mabs against either the GST-pIgR fusion protein.

By assaying and comparing results from assays using commercially available monoclonal antibodies directed to GST, and GST fusion proteins that do not contain pIgR, as well as polyclonal antibodies directed to pIgR, it is possible to identify isolated Mabs that either are pIgR specific or are specific to an epitope not present in either pIgR or GST but which occurs at the junction thereof. The Mabs can additionally be tested for specificity using MDCK cells and MDCK cells that have been transfected with different species of pIgR (human, rat, mouse, pig, rabbit, monkey, etc.).

A collection of monoclonal antibodies and sFvs that cumulatively bind to many, preferably every, epitope of pIgR domain 6, which includes the pIgR stalk, is prepared. Each of the sFvs and the Mabs are epitope mapped using the nested set of overlapping oligopeptides (each comprising 5 to 20 amino acids). Linear epitopes and conformational epitopes are identified on the strength of their binding and the location of the peptides in the nested set.

5.3. Single Chain Antibodies

One type of pIgR-targeting element is an antibody, or an antibody derivative, directed to a transcytotic molecule such as the pIgR stalk. As a non-limiting example, single chain Fv antibody fragments (sFv) directed to epitopes in defined regions in the pIgR amino acid sequence may be used. Non-limiting examples of such sFv antibodies are shown in FIGS. 3 to 5.

A derivative of sFv5A that incorporates an epitope known as a “FLAG tag” is designated “sFv5AF” (FIG. 3). Due to the way in which it was constructed, the amino acid sequence of sFv5AF has a mutation relative to sFv5A that is denoted “Q5V” (Gln at position 5 changed to Val).

A derivative of sFv5AF that contains a cysteinyl residue near its carboxyl terminus is designated “sFv5AF-Cys” (FIG. 5). This derivative of sFv5AF has a cysteine residue at the carboxy terminal region was introduced into the reading frame encoding sFv5AF by PCR mutagenesis (see Example 5).

5.4. Targeting Elements Directed to Intracellular Molecular Targets

Another source of amino acid sequences that provide ligands for pIgR are targeting elements that bind intracellular portions, regions or domains of the pIgR stalk. One source of such targeting elements is a protein known as calmodulin. There is evidence that calnodulin binds pIgR and it is thus expected that amino acid sequences within calmodulin interact with pIgR and may be isolated and used to prepare polypeptide ligands to pIgR (Enrich et al., Hepatology 24:226-232; 1996; Chapin et al., J. Biol. Chem. 271:1335-1342; 1996). The AP-1 clathrin adaptor complex of the trans-Golgi network is another protein that has been reported to bind an intracellular part of the pIgR stalk and can thus serve as a source of targeting elements (Orzech et al., Interactions of the AP-1 Golgi adaptor with the polymeric immunoglobulin receptor and their possible role in mediating brefeldin A-sensitive basolateral targeting from the trans-Golgi network, J Biol Chem 274(4):2201-15, 1999).

Because these targeting elements are directed to an intracellular portion of the pIgR stalk (the intracellular or cytoplasmic domain), another element that facilitates cellular uptake may be needed in order to direct complexes or compounds comprising them to these portions of the pIgR stalk. Once inside the cell, the targeting elements are able to bind the intracerllular portion of pIgR and thus be transported with it. Exemplary examples of such cellular uptake elements include, but are not limited to, PTD and MTS sequences.

The most actively studied approach uses a special class of peptides that are 10-35 amino acids long and are called “protein transduction domains” (PTD) or “membrane transport signals” (MTS). The PTD are derived from HIV-TAT, HSV-VP22 and Antenapedia (the source of Penetratin), and are characterized by having a high content of positively charged arginine (Arg) and lysine (Lys) residues, which might be important for contact with negatively charged cellular membrane lipids (Schwarze et al., Protein transduction: unrestricted delivery into all cells?, Trends Cell Biol 10(7):290-5, 2000; Schwarze et al., In vivo protein transduction: delivery of a biologically active protein into the mouse, Science 285(5433): 1569-72, 1999). The MTS are very hydrophobic peptides derived from secretory signal sequences, which may be able to spontaneously partition into the hydrophobic region of membrane lipid bilayers (Rojas et al., Genetic engineering of proteins with cell membrane permeability, Nat Biotechnol 16(4):370-5, 1998; Rojas et al., Controlling epidermal growth factor (EGF)-stimulated Ras activation in intact cells by a cell-permeable peptide mimicking phosphorylated EGF receptor, J Biol Chem, 1996. 271(44):27456-61, 1996). In some cases PTD and MTS peptides are able to confer membrane permeability to proteins that would otherwise not enter cells by cloning them together as a fusion construct.

Cloning vectors that simplify the incorporation of PTD and MTS sequences into recombinantly produced proteins are commercially available (invitrogen). Synthetic versions of these sequences may also be used and covalently or non-covalently associated with a complex or compound of the invention; in the case of TAT, these include, but are not limited to, poly-Arg, poly-Lys, poly-ornithine, and polymers of Arg, Lys and ornithine.

5.5. pIgR-Targeting Elements Derived from Bacterial Proteins

Zhang et al. (Cell 102:827-837, 2000) have published studies that indicate that pIgR is exploited by bacteria to provide a mechanism by which bacterial cells have enhanced abilities to adhere, invade, and undergo apical to basolateral transmigration. These results provide pIgR-targeting elements that are derived from surface proteins of bacteria.

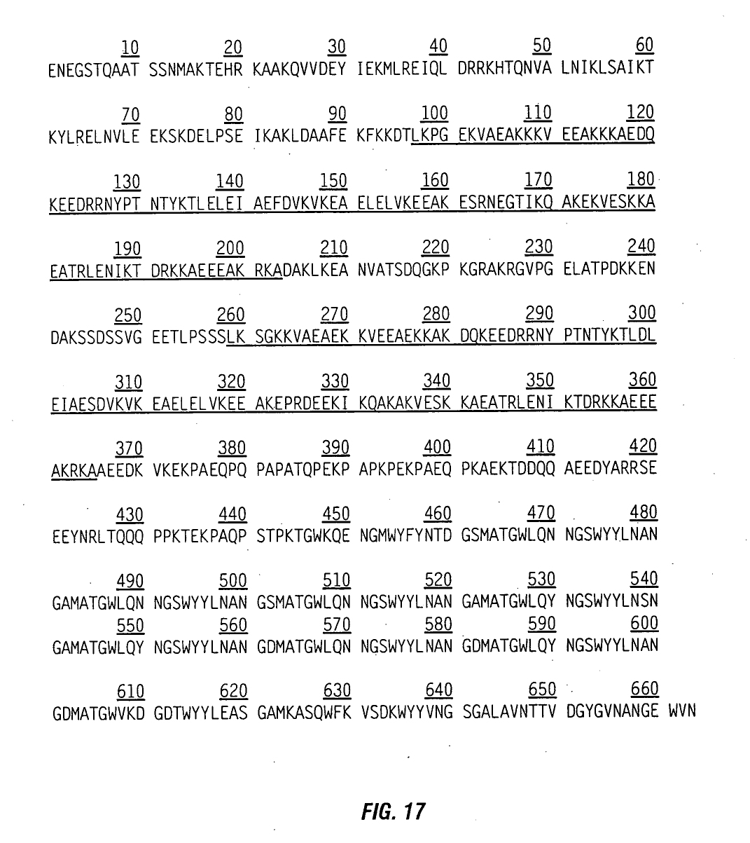

Zhang et al. present evidence that the pneumococcal adhesin protein CpbA interacts with human pIgR (hpIgR) as either a part of the outer surface of a bacterial cell or as a free molecule. The regions of CpbA:hpIgR interaction were mapped using a series of large peptide fragments derived from CpbA. CpbA (Swiss-Prot Accession No. O30874) contains a choline binding domain containing residues 454-663 and two N-terminal repetitive regions called R1 and R2 (SEQ ID NOS:______ and ______, respectively) that are contained in residues 97-203 and 259-365, respectively. Zhang et al. demonstrated that polypeptides containing R1 (107 amino acid residues) and R2 (see FIG. 17) interact with the SC portion of hpIgR, whereas a polypeptide containing residues 1-101 of CpbA does not bind to hpIgR.

Small polypeptides that retain the ability to interact with human and animal species of pIgR are utilized as pIgR targeting elements in the present invention. Such polypeptides may include those identified by phage display of disulfide constrained peptides as described above or polypeptides including but are not limited to the CbpA1, CbpA2, and CbpA3 polypeptides described by Zhang et al. In addition, other polypeptides from bacterial proteins homologous with CpbA, the pneumococcal adhesin protein in Streptococcus pneumoniae studied by Zhang et al., are part of the present invention. Such homologous proteins are present in virtually all pneumococcal serotypes. Those skilled in the art will be able to identify additional homologous proteins from genomic and protein databases such as Swiss-Prot, Entrez, and GenBank.

A search of Swiss-Prot revealed the following list of proteins (listed by Accession Number) that have sequences homologous with R1 and R2: O30874, O69188, O33741, O33742, Q9RQT5, AAF73779, AAF73781, AAF73788, AAF73814, AAF73790, Q9RQT3, Q9RQT2, AAF73776, AAF73786, AAF73792, AAF73798, AAF73807, AAF73810, AAF73812, AAF73822, AAF73795, Q9RQT6, AAF73785, Q9ZAY5, Q9RQT4, Q9RQT1, AAF73777, AAF73799, AAF73801, AAF73809, AAF73784, AAF73817, AAF73778, AAF73811, AAF73813; O33753, AAF73787, AAF73808, AAF73773, AAF73780, AAF73797, AAF73775, AAF73791, AAF73804, AAF73816, BAB01952, O58288, Q9Y102, and Q54972.

Smaller polypeptides comprising portions of the entire sequence of CbpA and proteins homologous to CbpA, and preferably portions of R1 and R2 and polypeptides homologous to R1 and R2, are identified based on their ability to bind to animal species of pIgR, preferably human pIgR. An overlapping, nested set of peptides can be synthesized and their ability to interact with pIgR can be tested to identify peptides that may be used to transport biologically active polypeptides, including vaccines, into (apical and basolateral endocytosis) and across (forward or reverse transcytosis) epithelial cell barriers. The peptides may be tested for their ability (i) to prevent SC binding to pIgR coated beads or (ii) to prevent adherence, invasion, or transmigration by S. pneumoniae R6x to Detroit cells, both methods being described by Zhang et al. The peptides may be from 5 to 100 amino acids long, preferably from 6 to 50, and most preferably from 6 to 20. An offset of 1 to 5 amino acids and preferably 3 to 4 amino acids may be used. A nested, overlapping set of peptides 15 amino acids long with an offset of 3 amino acids that would contain residues 1-15, 4-18, 7-21, 10-24, 13-27, etc., until the last residue in the polypeptide sequence is reached. By comparing the amino acids in peptides that are contiguous in CpbA and that show positive binding to pIgR, the core linear sequence that is required for binding to pIgR may be identified. A large peptide may be systematically reduced in size until the smallest peptide that produces a positive binding to pIgR is identified. Methods for identifying the core linear sequence have been described by Geysen et al. (J. Immunol. Methods 102:259-274, 1987), Tribbick et al. (J. Immunol. Methods 139:155-166, 1991), Geysen et al. (J. Molecular Recognition 1:32-41, 1988), Tainer et al. (Mol. Immunol. 23:709-715, 1986).

### Example 6

### Genetic Manipulation of a Transcytosing Single Chain Antibody

In vitro genetic manipulation has been used to alter the reading frame of sFv5A so as to create derivatives that have substitutions or insertions of amino acids with reactive sites. For example, sFv5AF-Cys is a derivative of sFv5AF into which a reactive Cys residue has been inserted, which also has one GGGGS linker between the newly introduced Cys residue and the sFv portion of the polypeptide (see FIGS. 3 to 5). The Cys residue contains a side group, —SH, that can react with the —SH side group of another Cys residue to form a disfulfide bond (—S—S—) that links the two Cys residues and the amino acids to which each Cys is attached. The positioning of a Cys residue in a sFv derivative influences whether it will react with a Cys residue in the same molecule (thus producing a monomer having an intramolecular disulfide bond) or a Cys residue in another sFv molecule (thus producing a multimeric sFv molecule having an intermolecular disulfide bond).

6.1. Introduction of Cysteine Residue into SFv5AF

The sFv single-chain molecule sFvSAF was altered via PCR mutagenesis in order to incorporate a cysteine residue at the carboxy terminal region. The template, a pSyn expression vector encoding sFv5AF, was amplified using a first oligonucleotide primer, “LMB3,” that has a sequence (5′-CAGGAAACAGCTAGAC-3′, SEQ ID NO:______) that is complementary to regions 5′ of the sFv5AF coding region in pSyn), and “cys-long,” a second oligonucleotide primer having the sequence:

(SEQ ID NO: _{——})

5′-AGTTGCGGCCGCGGCAGGAGCCACCGCCACCACCTAGGACGG-
TGACCTT-3′.

The latter primer is complementary to the last 4 codons of sFv5AF, with the 5′ end of the primer encoding the amino acid sequence GGGGSC in frame with sFv5AF, followed by a NotI restriction site.

Amplification was performed using the Taq-plus precision polymerase (Stratagene) according to the manufacturer's instructions. The PCR product was cleaved with NcoI and NotI, and then ligated into pSyn expression vector DNA that had been cleaved with NcoI and NotI. The resultant expression construct encodes sFv5AF-Cys, which has, from an amino- to carboxy-terminal direction, a pelb leader sequence (for secretion in E. coli) and a FLAG epitope tag, both encoded by vector sequences; sFv5AF-Cys, i.e., a heavy chain variable region, a spacer sequence [GGGGS repeated three times, i.e., (G₄S)₃], a light chain variable region, another (G₄S)₃ linker, a cysteine residue (emboldened “C”) that has been introduced into the sFv relative to sFv5AF; and c-myc epitope and 6×His tags encoded by vector sequences (see FIG. 4).

The amino acid sequence of any protein, including the single chain antibody sFv5A and its derivatives (sFv5AF, sFv5AF-Cys, etc.), is encoded by a nucleotide sequence, the reading frame. In vitro genetic manipulation is used to alter the amino acid sequence of sFv5A so as to favor the formation of dimers, trimers and other multimers; to add or enhance desirable attributes of sFv5A, and/or to reduce or remove undesirable attributes.

6.2. Introduction of Cysteine Residue into SFv5A

The sFv single-chain molecule sFv5A was altered via PCR amplification in order to substitute the myc-6×His-tags at the carboxy terminal region (FIG. 4) with a GGGG-Cys C-terminal tail. For this construct, PCR amplification reactions were assembled using High Fidelity Platinum Taq (Life Technologies) according to manufacturer's instructions (1× High Fidelity PCR buffer, 0.2 mM each dNTP, 2 mM MgSO4, 0.2 μM of each primer, 2.5 units Platinum Taq High Fidelity, and template DNA as required), which allows for “hot start” PCR to minimize the generation of early stage nonspecific priming events. Amplification was carried out using a modified procedure adapted from Roux and Hecker (PCR Cloning Protocols, B. A. White, eds., Humana Press, 1997, pp. 39-45), where thermocycling reactions were run using linked files in a PCR program as follows: 1) denaturation at 94° C. for 10 minutes; 2) 30 cycles of denaturation for 1 minute at 94° C., primer annealing for 1 minute at 60° C., primer extension for 60 seconds at 72° C., and 3) a final 4° C. chill step (until analyzed). The size of the PCR products were confirmed by agarose gel electrophoresis and then purified away from contaminating primers by spin column chromatography (Qiagen QIAquick purification kit).

The template, a pSyn expression vector encoding sFv5A, was amplified using a “Forward” oligonucleotide primer, “Pelb-5 Forward,” (SEQ ID NO:______) that is complementary to the 5′-portion of the pelb-coding sequence in the pSyn5A vector, and “pSynG4Cys Antisense,” a “Reverse”-oligonucleotide primer with the sequence listed below.

Pelb-5′ Forward Primer (SEQ ID NO:______).

5′-AAATACCTATTGCCTACGGCAGCC-3′

pSynG4Cys Antisense Reverse Primer (SEQ ID NO:______).

5′-CGGAATTCCTACTAGCAGCCACCGCCACCTGCGGCCGCTAGGA-
CGGTGACCTTGGTCCC-3′

The latter primer is complementary to 7 codons near the C-terminus of the sFv5A coding region, with the 5′ end of the primer encoding the NotI restriction site followed by the amino acid sequence GGGGC in frame with sFv5A, followed by a two (2) tandem TAG stop codons and an EcoRI restriction site.

The PCR product was cleaved with BamHI and EcoRI, and then ligated into pSyn expression vector DNA that had been cleaved with BamHI and EcoRI. The resultant expression construct encodes sFv5A-G₄Cys, which has, from an amino- to carboxy-terminal direction, a pelb leader sequence (for secretion in E. coli) encoded by vector sequences; sFv5A-Cys, i.e., a heavy chain variable region, a spacer sequence [GGGGS repeated three times, i.e., (G₄S)₃], a light chain variable region, another G₄S linker, and a C-terminal cysteine residue that has been introduced into the sFv relative to sFv5A, replacing the c-myc epitope and 6×His tags encoded by vector sequences shown in FIG. 5.

6.3. Introduction of a C-Terminal Cysteine Residue into SFv5A and SFv5AF

The sFv single-chain molecules sFv5A and sFv5AF were altered via PCR amplification in order to remove the NotI restriction site and substitute the myc-6×His-tags at the carboxy terminal region with a GGGG-Cys C-terminal tail. For this construct, PCR amplification reactions were assembled using High Fidelity Platinum Taq (Life Technologies) according to manufacturer's instructions as described above.

The template, a pSyn expression vector encoding sFv5A, was amplified using a “Forward” oligonucleotide primer, “Pelb-5 Forward,” (SEQ ID NO:______) that is complementary to the 5′-portion of the pelb-coding sequence in the pSyn5A vector), and “5A-(deltaN)Gly4-Cys,” a “Reverse” oligonucleotide primer (SEQ ID NO:______) with the sequence listed below.

Pelb-5′ Forward Primer (SEQ ID NO:______)

5′-AAATACCTATTGCCTACGGCAGCC-3′

5A-(deltaN)Gly4-Cys Reverse Primer (SEQ ID NO:______).

5′-CCGGAATTCGTCGACTCATCAGCAGCCTCCACCGCCACCTAGG-
ACGGTGACCTTGGTCCC-3′

The latter primer is complementary to the 7 C-terminal codons of the sFv5A coding region, followed by the amino acid sequence GGGGC in frame with sFv5A, followed by a two (2) tandem TAG stop codons and sequential SalI and EcoRI restriction sites.

The PCR product was cleaved with BamHI and EcoRI, and then ligated into either the pSyn-5A or pSyn-5AF expression vector DNA that had been cleaved with BamHI and EcoRI. The resultant expression constructs encode sFv5A-(deltaN)Gly₄-Cys or sFv5AF-(deltaN)Gly₄-Cys, respectively. The amino acid sequence contains, from the amino- to carboxy-terminal direction, a pelb leader sequence (for secretion in E. coli) plus/minus a FLAG epitope tag encoded by vector sequences; sFv5A-Cys, i.e., a heavy chain variable region, a spacer sequence [GGGGS repeated three times, i.e., (G₄S)₃], a light chain variable region, another G₄S linker, and a C-terminal cysteine residue that has been introduced into the sFv relative to sFv5A, replacing the NotI restriction site and the c-myc epitope and 6×His tags encoded by vector sequences shown in FIG. 4.

6.4. Length, Composition and Number of Linkers

The two variable regions of a sFv that combine to form a ligand binding site are known as V(H) and V(L). In a monomeric sFv, the V(H) and V(L) of each molecule are associated with each other. In one type of dimeric sFv, the V(H) of one monomer [V(H)1] is associated with the V(L) of another monomer [V(L)₂], and vice versa [i.e., V(H)₂ is associated with V(L)1].

The length and composition of the linker between the V(H) and V(L) regions in an sFv is one factor that influences the tendency of an sFv to form monomers or multimers (Todorovska et al., Design and application of diabodies, triabodies and tetrabodies for cancer targeting, J Immunol Methods 2001 Feb. 1; 248(1-2):47-66; Arndt et al., “Factors Influencing the Dimer to Monomer Transition of an Antibody Single-Chain Fv Fragment”, American Chemical Society, Biochemistry 1993, 37, pp. 12918-12926). For example, a sFv molecule in which there is a relatively short linker between the V(H) and V(L) regions may be less likely to fold back upon itself and form a monomer. Thus, “short linker” sFv derivatives are often more likely to form dimers, as their V(H) and V(L) regions must pair with, respectively, the V(L) and V(H) regions of a second sFv molecule. Often, sFv derivatives with relatively long linkers between the V(H) and V(L) regions may fold back upon themselves, and therefore may have a greater tendency to form monomers. However, some sFv derivatives with long linkers between V(H) and V(L) may have some tendency to form multimers.

The number of linkers between the V(H) and V(L) regions of sFv5A has been altered to produce a set of sFv derivatives that are screened and assayed for desirable attributes. That is, the sFv5A derivatives are assayed for their ability to form either monomers or multimers, and multimeric forms are analyzed to determine whether dimers, trimers, and the like, or mixtures thereof, are present. Assays, including by way of non-limiting example those described herein, are performed on the derivatives in order to determine their paracellular transporting and transcytotic properties, pharmacokinetics, stability and the like, in absolute terms as well as compared to the unaltered sFv5A molecule.

Various amino acid sequences are known that may serve as suitable spacers in the compounds of the invention (for a review, see Simons, Spacers, probability, and yields, Bioconjug Chem 1999 January-February; 10(1):3-8). Some non-limiting examples of sequences that have been used in sFv's include include EGKSSGSGSESKEF, one or more copies of GGGGS [a.k.a. (G₄S)ₓ] (Newton et al., Angiogenin single-chain immunofusions: influence of peptide linkers and spacers between fusion protein domains, Biochemistry 1996 Jan. 16; 35(2):545-53), GSGS [a.k.a. (GSGS)ₓ] and GSSG [a.k.a. (GSSG)ₓ].

Derivatives of sFv5A with varying V(H) to V(L) distances, the distance varying with the number of times the linker sequence GGGGS is present, have been prepared using an overlapping PCR technique in which the heavy and light chains [V(H) and V(L), respectively] are generated separately by PCR amplification. The V(H) and V(L) PCR products are engineered to contain overlapping complimentary sequences at their 3′ and 5′ ends, respectively. The PCR products are mixed, heated to 95° C. to melt the DNA, then cooled to 58° C., resulting in the annealing of the two (2) products through their complimentary overlapping linker (alternate length) sequences. The annealed and connected PCR products now serve as a full-length heavy-light chain DNA template for a second round of PCR amplification using a primer set complementary to the 5′- and 3′-sequences of V(H) and V(L), respectively. PCR amplification results in a full-length sFv which has an altered linker length incorporated between the heavy and light chains.

The parent sFv was either pSyn5A, pSyn-5AF or pSyn-5AF-Cys (FIGS. 3 and 4). sFv5AF and sFv5AF-Cys contain three repeat linkers, i.e., (GGGGS)₃ between their V(H) and V(L). Derivatives with one linker (GGGGS), four linkers, i.e., (GGGGS)₄ and five linkers, i.e. (GGGGS)₅ have been prepared, and derivatives with 2 linkers can be prepared in like fashion.

In order to generate heavy chain regions with varied C-terminal linker sequences, templates (the pSyn expression vectors encoding sFv5A or sFv5AF) were amplified using a “Forward” oligonucleotide primer, “Pelb-5 Forward,” (SEQ ID NO:______) that is complementary to the 5′-portion of the pelb-coding sequence in both the pSyn5A or pSyn5AF vectors, and a “Reverse” oligonucleotide primer corresponding to the 8 C-terminal codons of the 5A heavy chain variable sequence in-frame with either of the GGGGS (SEQ ID NO:______), (GGGGS)₄ (SEQ ID NO:______) or (GGGGS)₅ (SEQ ID NO:______) linker sequence as listed below.

The following oligonucleotides were used for generating heavy chain regions with varied C-terminal linker lengths.

Pelb-5′ Forward Primer (SEQ ID NO:______).

5′-AAATACCTATTGCCTACGGCAGCC-3′

Single GGGGS linker: 5A-GS-1 Reverse Primer (SEQ ID NO:______)

5′-TGACCCTCCGCCACCTGAGGAGACGGTGACCAGGGTGCC-3′

(SEQ ID NO: _{——})

(GGGGS)4 linker: 5A AGS4-S2/G Linker Reverse
Primer
5′-GGACCCTCCGCCTCCTGAGGAGACGGTGACCAGGGTGCCACGGCC-
3′

(SEQ ID NO: _{——})

(GGGGS)5 linker: 5A AGS5-S2/G Linker Reverse
Primer
5′-GCTCCCTCCGCCTCCGGACCCTCCGCCTCCTGAGGAGACGGTGAC-
CAGGGTGCCACGGCC-3′

In order to generate light chain regions with varied N-terminal linker sequences, template, the pSyn expression vector encoding sFv5A, was amplified using a “Reverse” oligonucleotide primer, “5A-Sal-H6-Sal,Xho,Eco Reverse Primer” (SEQ ID NO: ______) that is complementary to the 8 C-terminal codons of the 5A light chain variable sequence in the pSyn5A vector, and is in-frame with sequences coding for NotI and SalI restriction sites, a 6×His epitope tag, a SalI site, tandem TAG stop codons, and XhoI and EcoRI restriction sites. This reverse primer was used with one of the three (3) “Forward” oligonucleotide primers corresponding to either the GGGGS (SEQ ID NO:______), (GGGGS)₄ (SEQ ID NO:______) or (GGGGS)₅ (SEQ ID NO:______) linker sequence in-frame with the 8 N-terminal codons of the 5A light chain variable sequence as listed below.

The following oligonucleotides were used for generating light chain regions with varied N-terminal linker lengths:

(SEQ ID NO: _{——})

5A-Sal-H6-Sal,Xho,Eco Reverse Primer
5′-CGGAATTCCTCGAGCTACTAGTCGACCTAGTGATGGTGGTGAT-
GGTGGTCGACTGCGGCCGCACCTAGGACGGTGACCTTGGTCCC-3′

(SEQ ID NO: _{——})

Single GGGGS linker: 5A-GS-1 Forward Primer
5′-GGTGGCGGAGGGTCATCTGAGCTGACTCAGGACCCTGCT-3′

(SEQ ID NO: _{——})

(GGGGS)4 linker: AGS-4 5A-Linker Forward Primer
5′-GGAGGCGGAGGGTCCGGTGGAGGCGGTTCAGGCGGAGGTGGCTCT-
GGCGGTGGCGGATCGTCTGAGCTGACTCAGGACCC-3′

(SEQ ID NO: _{——})

(GGGGS)5 linker: AGS-5 5A-Linker Forward Primer
5′-
GGAGGCGGAGGGTCCGGAGGCGGAGGGAGCGGTGGAGGCGGTTCAGG-
CGGAGGTGGCTCTGGCGGTGGCGGATCGTCTGAGCTGACTCAGGACCC-
3′

To generate the heavy and light chains described above, PCR amplification reactions were assembled using the proofreading ProofStart DNA polymerase (Qiagen) according to manufacturer's instructions (1× ProofStart PCR buffer, 0.3 mM each dNTP, 0.1 μM of each primer, 2.5 units ProofStart DNA polymerase, and template DNA as required), which allows for “hot start” PCR to minimize the generation of early stage nonspecific priming events. Amplification was carried out using a modified procedure adapted from Roux and Hecker (PCR Cloning Protocols, B. A. White, eds., Humana Press, 1997, pp. 39-45) as described above. The sizes of the PCR products were confirmed by agarose gel electrophoresis and then purified away from contaminating primers by spin column chromatography (Qiagen QIAquick purification kit).

Following purification of the individual heavy and light chain PCR products, 50 ng of each corresponding fragment was mixed and subjected to a second round of PCR amplification using the Pelb-5′ Forward (SEQ ID NO:______) and the 5A-Sal-H6-Sal,Xho,Eco Reverse (SEQ ID NO:______) primers, and the ProofStart DNA polymerase in a 50 μl reaction as described above, except that the annealing step was carried out at 58° C. The full-length PCR products comprising the heavy and light chain variable regions separated by either a single GGGGS linker, or a (GGGGS)₄ or (GGGGS)₅ linker, were gel purified and digested with either NcoI and BamHI, or NcoI and EcoRI.

The 2^{nd}-step full-length PCR products containing either the single GGGGS linker, or the (GGGGS)₄ and (GGGGS)₅ linker versions cleaved with NcoI and BamHI, were then ligated into any derivatized sFv5A expression vector DNA (such as pSyn-5A, pSyn-5AF, sFv5AF-Cys, sFv5AF-G₄Cys, sFv5A-(deltaN)Gly₄-Cys, sFv5AF-(deltaN)Gly₄-Cys) that had been cleaved with NcoI and BamHI. The resultant expression construct encodes an sFv5A derivative which has incorporated either the single GGGGS, (GGGGS)₄ or (GGGGS)₅ alternative linker between the heavy and light chain variable regions and maintains the integrity of the C-terminal amino acids of the parent vector. For various linker versions cut with Nco1 and EcoRI, the resulting expression constructs will have alternative linkers between the heavy and light chain variable regions and a C-terminal 6×His epitope tag. The amino acid sequence contains, from the amino- to carboxy-terminal direction, a pelb leader sequence (for secretion in E. coli) plus/minus a FLAG epitope tag encoded by vector sequences; sFv5A-Cys, i.e., a heavy chain variable region, a linker region of GGGGS, (GGGGS)₄ or (GGGGS)₅, a light chain variable region; and either a C-terminal Cys or a single 6×His epitope tag at C-terminus.

The sFv5A and sFv5AF derivatives are expressed in E. coli bacterial cells and prepared from the periplasmic space of the bacterial cells using the same techniques and materials as those used for sFv5AF. Monomers and, if present, dimers and other multimers, are prepared and separated as described in the Examples and throughout the disclosure.

Similarly, derivatives with from 5 to 30 linkers are prepared. Other sFv5A derivatives may have varying numbers of other linkers. Any number or type of linker may be incorporated into an sFv derivative that is produced and tested for desirable properties. The spacing between the sFv sequences (the combined sequences of V(H) and V(L)) and other elements is altered for various properties. For example, it may be desirable to alter the positioning of polypeptide purification or detectable elements, or reactive groups, further from or closer to the sFv portion of the fusion protein depending on a particular purification strategy or intended use.

### Example 7

### Purification and Evaluation of Monomeric and Dimeric sFv5AF-Cys Molecules

7.1. Reduction of SFv5AF-Cys

A preparation of monomeric sFv5AF-Cys was reduced by adding dithiothreitol (DTT) to a final concentration of 10 mM, and incubating at 25° C. for 30 minutes.

The concentration of DTT (10 mM) used in the reactions was chosen because it does not cause quantitative reduction of the disulfides in the sFv5AF-Cys molecule. Rather, it is enough to reduce disulfide bonds at the C-terminal cysteine without reducing disulfides between internal cysteine residues. This disfavors formation of sFv dimers but allows the native structure, and biological activy dependent thereon, of the sFv molecule to be retained.

7.2. Size Exclusion Chromatography

Monomeric sFv5AF-Cys molecules were separated from dimers (and higher multimers if any are present) by size exclusion chromatography (SEC) on a 1×44 cm Superdex 75 column with 0.1 M PO₄ containing 1 mM EDTA, pH 6.25. Fractions 29-34 were collected as dimer, and fractions 38-43 were collected as monomer.

7.3. Transcytosis Assay Design

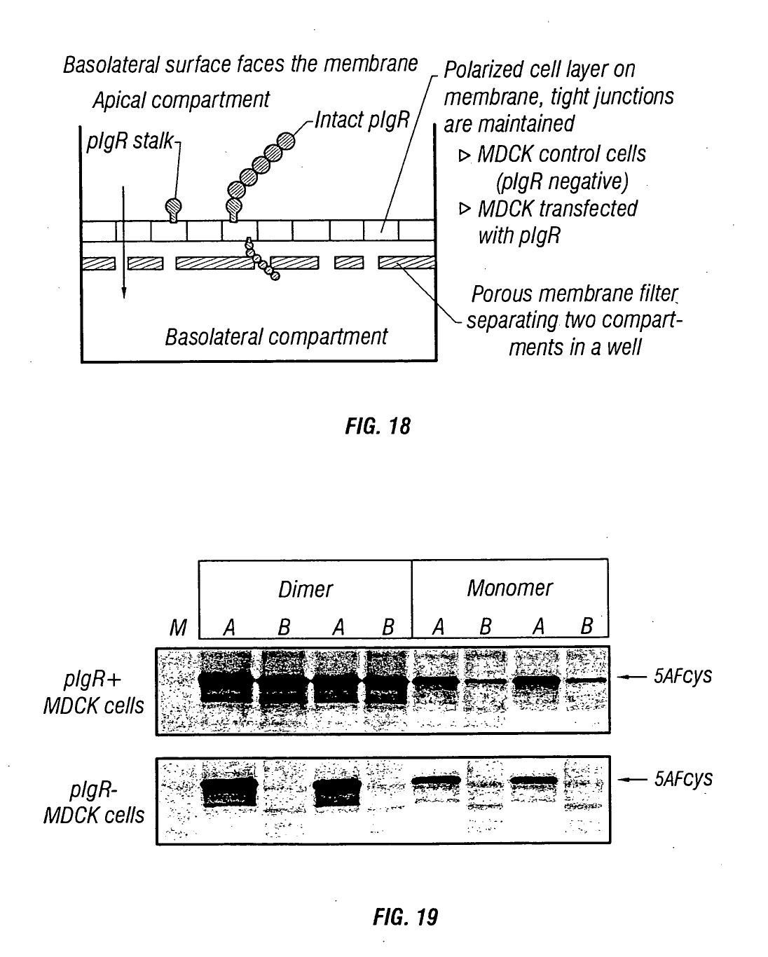

The assay was performed in the transwell system as shown in FIG. 18. The cells are grown on a porous membrane that separates the apical and the basolateral compartment. Complexes and compounds to be tested are placed in the apical compartment and then assayed by removing samples from the basolateral compartment. The direction of normal IgA transport is from basolateral to apical; however, preferred complexes and compounds of the invention undergo “reverse” (apical to the basolateral) transcytosis.

The complex or compound is placed in the apical compartment of the transwell and, after a period of time, a sample from the apical compartment and a sample from the basolateral compartment are removed and separated by SDS-PAGE. After gel electrophoresis, the proteins are transferred to PVDF membranes which are probed as Western blots with a polyclonal anti-sFv antibody, or an antibody to an epitope in the complex or compound being tested, followed by anti-rabbit IgG-alkaline phosphatase detection with nitro blue tetrazolium and 5-bromo-4-chloro-3-idoly phosphate, toludinium salt (NBT/BCIP). Western blotting with anti-sFv5A detects any molecular species that contains the sFv; regardless of whether the sFv is present either as part of a composition or compound of the invention or as a “free” sFv molecule.

Control (untransfected) MDCK cells do not contain significant levels or any pIgR. Therefore, one expects to observe no transcytosis of pIgR- or stalk-targeted complexes or compounds in these cells. Thus, a sample of the apical compartment will contain the complex or compound that has been added thereto, whereas a sample of the basolateral compartment should not show any sFv or conjugate thereof.

In contrast, MDCK cells that have been transfected with an expression construct that encodes and expresses a pIgR or stalk molecule, or derivatives thereof, have the capacity for pIgR-specific transcytosis. In these cells, one expects to observe transcytosis from the apical to basolateral compartments. Accordingly, bands corresponding to complexes or compounds will be present in the basolateral compartment if the molecules are capable of trancytosis.

Typically, four-day old cultures of MDCK cells expressing pIgR or stalk molecules (or derivatives thereof), or control (untransfected) MDCK cells, are incubated in the presence of 10 ng/ml to 10 mg/ml of the complex or compound to be tested in the apical chamber of a Transwell transcytosis chamber. The cells are incubated for at various times, typically about 20 hours unless otherwise indicated.

Both the apical and basal chambers are harvested at the end of the incubation period. Typically, one-third of the volume of media from the apical chamber, and all of the media from the basal chamber, are incubated with Protein A-Sepharose beads. Protein A binds to the Fc regions of IgG molecules, and binds to some sFv's through their VHIII domain (Akerstrom et al., On the interaction between single chain Fv antibodies and bacterial immunoglobulin-binding proteins, J Immunol Methods 177(1-2):151-163, 1994). The sFv's that bind to protein A can be purified from culture media or other sources by affinity chromatography on a protein A matrix (such as are available from Pierce Chemical Co., Rockford, Ill.). Although it binds sFv5 and its derivatives, Protein A does not bind to some sFv's, including other sFv's that may be used in compounds of the invention. However, Protein A derivatives having an increased range of binding spectra are known (Svensson et al., Protein LA, a novel hybrid protein with unique single-chain Fv antibody- and Fab-binding properties, Eur J Biochem 258(2):890-896, 1998). Moreover, immunoprecipitation can be used as an alternative to Protein A. For example, the polyclonal antibody directed to sFv5 and its derivatives could be used to immunoprecipitated with sFv5 molecules.

The beads are washed, resuspended in SDS-PAGE sample buffer and the proteins subjected to SDS-PAGE. The proteins are transferred to PVDF and the membranes are probed as Western blots with a polyclonal anti-sFv antibody (or other antibody as appropriate, as described herein) followed by detected with, e.g., anti-rabbit IgG-alkaline phosphatase and the colorimetric substrate NBT/BCIP.

In some experiments, the dimeric form of sFv5AF-Cys runs as doublet. This is likely due to the loss of a carboxy terminal polypeptide that comprises c-myc epitope and His tag amino acid sequences. When the sFv5AF-Cys dimer is probed with a monoclonal antibody directed to an epitope in the c-myc tag (9E10, Cambridge Bioscience), which is located on the carboxy terminus of the protein, the lower band of the doublet is not detected.

7.4. Transcytosis Assay

The transcytotic properties of sFv5AF (monomer) and sFv5AF-Cys (monomer and dimer) were evaluated and compared in MDCK cells. As shown in FIG. 19, sFv5AF efficiently transcytosed from the apical to basolateral media in a pIgR-dependent fashion. That is, reverse transcytosis of sFv5AF occurred in MDCK cells transfected with and expressing pIgR, but not in untransfected cells.

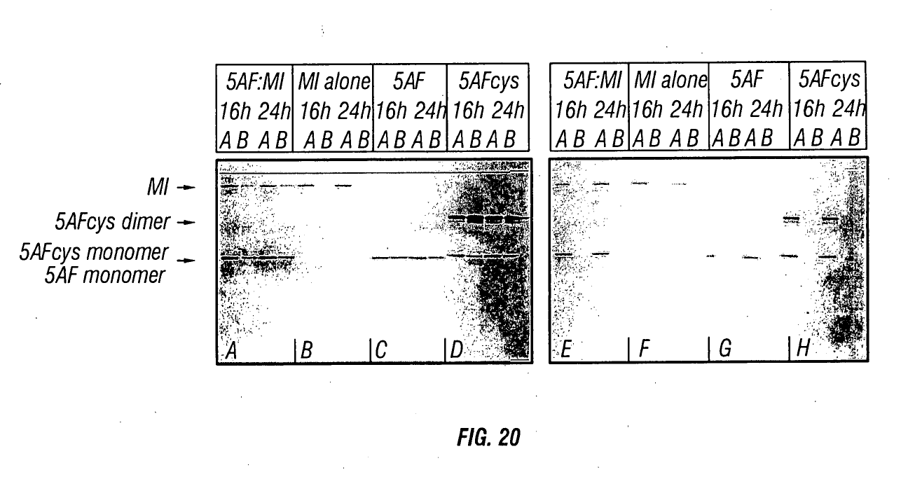

Transcytosis of a preparation of sFv5AF-Cys that contained both monomers and dimers was also evaluated. The results are shown in FIG. 20 (panels “D” and “H”; note that the sFv5AF-Cys monomer has a slightly higher apparent molecular weight than the monomer of sFv5AF due to the relative addition of a Cys residue and a GGGGS linker). Transcytosis of the monomeric and dimeric forms of the sFv5AF-Cys molecule was pIgR-specific. Comparison of intensity of the dimer and monomer bands in the basolateral media in pIgR-expressing cells at 16 and 24 hours indicates that, compared to the monomer, more of the dimer has transcytosed at these time points.

7.5. Time Course of Transcytosis

A mixture of monomers and dimers of sFv5AF was assayed as described above in pIgR-expressing MDCK cells over defined periods of time. The periods chosen were 0-2, 2-4, 4-6, 6-8, 8-12 and 12-24 hours after the introduction of material to the apical chamber.

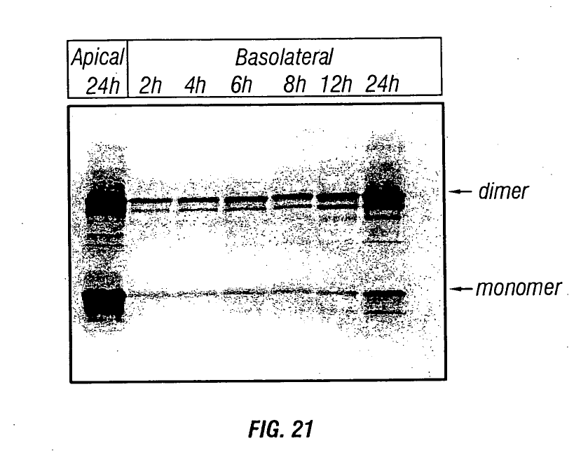

The results are shown in FIG. 21. The doublet band, which represents dimers, underwent apical to basolateral transcytosis at a faster rate than monomers. The transcytosis of both species is relatively constant over the time course.

7.6. Forward and Reverse Transcytosis Compared

The single-chain antibody sFv5AF-Cys was applied to either the apical compartment or the basolateral compartment of pIgR expressing or control MDCK cells at a concentration of 6 ug/ml. After 16 hours, apical and basolateral media were collected and 10% of the volume of the side to which ligand was added and 100% of the volume of the side representing transcytosed ligand were affinity-purified using Protein A sepharose and subjected to SDS-PAGE and Western blotting with anti-sFv5AF polyclonal antiserum. Thus, equal intensity bands in the apical and basolateral lanes represents 10% transcytosis.

Purified s5AF-Cys monomer and dimer were added to the apical chamber of transwells containing pIgR expressing MDCK cells or control (pIgR negative) MDCK cells. After 16 hours at 37° C., 100% of the basolateral media, and 10% of the apical media were analyzed by protein A pull-down, reducing SDS-PAGE, and western blotting with anti-sFv5AF antisera, essentially as described above. The inclusion of the reducing agent (beta-mercaptoethanol) breaks covalent disulfide linkages between sFv5AF-Cys molecules. Thus, all sFv5AF-Cys species tend to migrate at the monomer position, regardless of whether they were monomer of dimer prior to reduction.

The results are shown in FIG. 20. As can be seen by comparing the apical and basolateral lanes in the pIgR expressing cells, approximately 10% of the sFv5AF-Cys dimer underwent transcytosis, while less than 5% of the monomer transcytosed. The control MDCK cells show no significant transcytosis of dimer or monomer.

In other experiments, basolateral to apical (forward) transcytosis of the sFv5AF-Cys dimers and monomers was examined. Forward transcytosis of the sFv5AF-Cys dimer was less than 10%, while transcytosis of monomer sFv5AF-Cys was undetectable.

### Example 8

### Preparation of Fusion Proteins Comprising Growth Hormone (GH) Polypeptides

8.1. Preparation of Nucleic Acids Encoding Human Growth Hormone

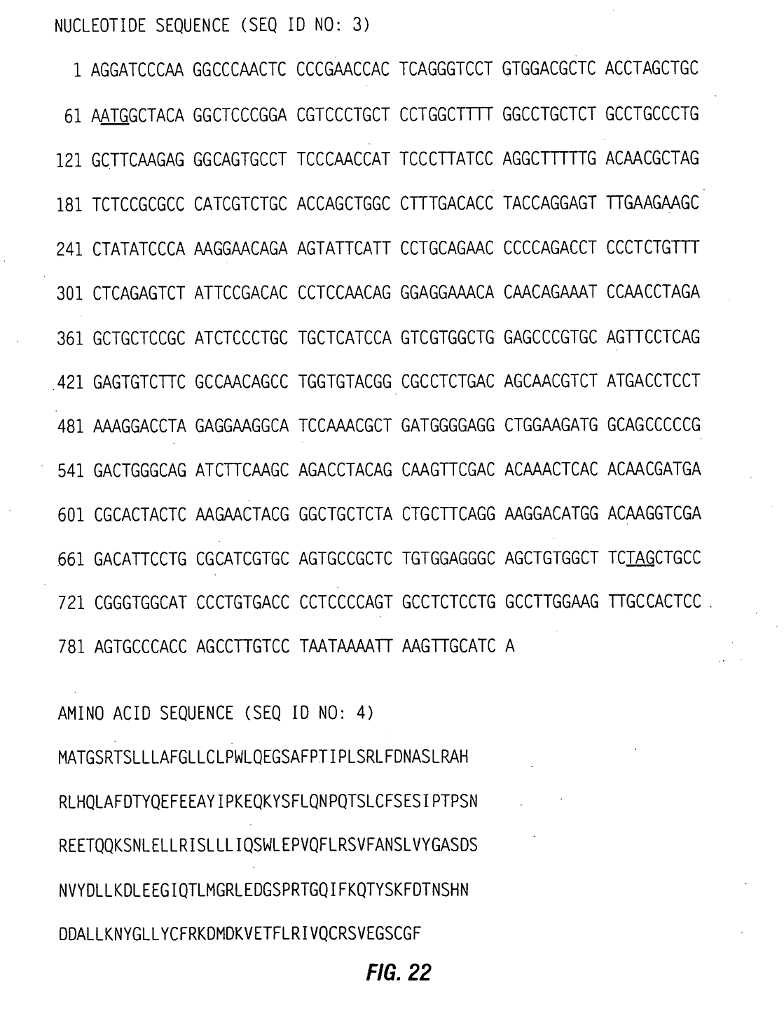

In order to prepare DNA molecules that encode human growth hormone (hGH), a two-step cloning procedure is used. In the first step, sequences encoding human growth hormone (hGH) (FIG. 22; SEQ ID NO:______) are amplified via PCR, treated with restriction endonucleases and, through the use of T4 ligase, are inserted into an intermediate cloning vector. PCR amplification reactions are assembled using High Fidelity Platinum Taq (Life Technologies) according to manufacturer's instructions (1× High Fidelity PCR buffer, 0.2 mM each dNTP, 2 mM MgSO4, 0.2 μm of each primer, 2.5 units Platinum Taq High Fidelity, and template DNA as required), which allows for “hot start” PCR to minimize the generation of early stage nonspecific priming events. Amplification is carried out using a modified procedure adapted from that of Roux and Hecker (PCR Cloning Protocols, B. A. White, eds., Humana Press, 1997, pp. 39-45). Thermocycling reactions are run using linked files in a PCR program as follows: (1) denaturation at 94° C. for 10 minutes; (2) 30 cycles of denaturation for 1 minute at 94° C., primer annealing for 1 minute at 60° C., and primer extension for 60 seconds at 72° C.; and (3) chilling to 4° C. until analyzed. The PCR amplification step uses primers designed to amplify the hGH cDNA sequence from human cDNA libraries purchased from a commercial source (Clontech, human pituitary gland HL1139a or HL1139b) and inserted either 5′ (amino-terminal) or 3′ (carboxy-terminal) into an expression construct encoding an sFv fusion protein (such as pSyn5A or pSyn5AF).

The size of the PCR products are confirmed by agarose slab gel electrophoresis, and the PCR products are then purified away from contaminating primers by spin column chromatography (Qiagen QIAquick purification kit). Due to the utilization of Taq DNA polymerase, all PCR products contain a 3′-A overhang and are easily ligated into the pCR-TOPO intermediate vector (Invitrogen). Alternatively, the PCR product is digested with appropriate restriction enzymes, and ligated into the pBluescript II KS(+) vector (Stratagene) to create an intermediate cloning vector.

The intermediate vector is used to confirm that the hGH-encoding cDNA sequences inserted therein are correct by DNA sequencing. Once the cloned hGH nucleotide sequence is confirmed, the hGH-encoding DNA is excised from the intermediate cloning vector using restriction enzymes and is inserted into a vector encoding an sFv in such a manner as to be in frame with the sFv reading frame. The PCR primers are designed so that, after the PCR product is inserted into the expression vector, the hGH amino acid coding sequence is in-frame with the amino acid reading frame of the sFv protein expression construct, and the resulting chimeric reading frame encodes a hGH-sFv or sFv-hGH fusion protein. The primers used in this example are designed for cloning sequences encoding a biologically active protein of interest into pSyn5A or pSyn5AF, both of which are E. coli expression plasmids. However, similar primers can be used to engineer sFv fusion constructs for production in other expression systems (such as, e.g., yeast, insect, viral or mammalian expression systems). Expression of a structurally intact and functional sFv fusion protein is confirmed using protein analytical techniques (SDS-PAGE, Western blotting and ELISA analysis) and commercially available in vitro diagnostic kits. In instances where an epitope is present as an optional fusion protein element, commercially available antibodies to this epitope are used. Similar biochemical analytical techniques are used to screen for functional fusion protein in blood and serum samples obtained from in vivo animal studies.

8.2. Human Growth Hormone (hGH) Amino-Terminal Fusion Constructs

In order to generate fusion proteins having an amino-terminal hGH polypeptide, PCR amplification reactions are carried out as described above using 2 μL of the human pituitary gland cDNA library (Clontech, HL1139a or HL1139b) as template DNA and the hGH-NH2-For (SEQ ID NO:26) and hGH-NH2-Rev (SEQ ID NO:27) primers. The forward primer (SEQ ID NO:26) includes a 5′ NcoI restriction site. The reverse primer (SEQ ID NO:27) has a sequence encoding an internal in-frame Gly4-Ser linker sequence and a 3′ Sal I site.

The PCR product is ligated into the pCR-TOPO vector (Invitrogen) and the cDNA sequence is confirmed. The hGH encoding sequences are then excised from the intermediate vector by digestion with Nco I and Sal I, gel purified and ligated into Nco I/Xho I-digested pSyn5A-5′/3′-MCS-6×His expression vector. This vector has extended multitple cloning sites (MCS) incorporated in-frame within the 5′- and 3′-regions flanking the 5A coding sequence. Joining the Sal I overhang with the Xho I overhang abolishes both sites within the new chimeric expression vector. The resulting chimeric reading frame encodes a fusion protein with a hGH protein-G4S linker peptide fused in-frame to the amino-terminus of a sFv. This fusion protein is called hGH-sFv.

hGH-NH2-For (SEQ ID NO: _{——})
5′-CATGCCATGGCCTTCCCAACCATTCCCTTATCCAGGCTTTTTGAC-
3′
hGH-NH2-Rev (SEQ ID NO: _{——})
5′-CCGCGGCCGCTATGGCCGACGTCGACTGACCCTCCGCCACCG-
AAGCCACAGCTGCCCTCCACAGAGCGGCACTG-3′

8.3. Human Growth Hormone (HGH) Carboxy-Terminal Fusion Constructs

In order to generate fusion proteins having a carboxy-terminal hGH polypeptide, PCR amplification reactions are carried out as described above using 2 μL of the human pituitary gland cDNA library (Clontech: HL1139a or HL1139b) as template DNA and either the first (1st) or second (2nd) generation hGH-COOH-For (SEQ ID NO:______) and hGH-COOH-Rev (SEQ ID NO:______) primer sets. The sequence of the forward primers (SEQ ID NO:28 and NO: 2nd generation FOR) includes a 5′ Not I restriction site followed by a sequence that encodes an internal Gly4-Ser linker for the in-frame insertion with the carboxy terminus of the sFv in the pSyn5A or pSyn5AF constructs. The sequence of the reverse primer (SEQ ID NO:______) contains an in-frame 6×His tag followed by tandem TAA stop codons and a 3′ Eco RI site. The sequence of the reverse primer (SEQ ID NO: ______, 2nd generation REV) contains in-frame sequences encoding the following: Sal I-(histidine)₆-tag-Sal I-(TAG stop codon)₂-Xho I-Eco RI site.

The PCR product is ligated into the pBluescript II vector (Stratagene), and the cDNA sequence of this intermediate vector is confirmed. The hGH-encoding DNA is then excised from the intermediate vector by digestion with Not I and EcoRI, gel purified and ligated into Not I/Eco RI-digested pSyn5A or pSyn5AF expression vectors. The resulting chimeric reading frame encodes a fusion protein of apparent molecular weight of about 56 kD on SDS-PAGE (calculated MW, 50.5 kD) with a G₄S linker peptide-hGH protein fused in-frame with the carboxy terminus of a sFv (5A or 5AF). These fusion proteins are called sFv-hGH (1st-generation) and sFv-hGH-H6 (2nd-generation), respectively.

hGH-COOH-For (SEQ ID NO: _{——})
5′-ATAAGAATGCGGCCGCCGGTGGAGGCGGTTCAATGGCTACAGG-
CTCCCGGACGTCCCTG-3′
hGH-COOH-Rev (SEQ ID NO: _{——})
5′-CGGAATTCCTACTAATGATGGTGATGATGGTGTGCGGCCGC-
GAAGCCACAGCTGCCCTCCACAGAGCG-3′
hGH-COOH-H6 FORWARD (SEQ ID NO: _{——}, 2nd generation
FOR)
5′-ATAAGAATGCGGCCGCAGGTGGCGGAGGGTCATTCCCAA-
CCATTCCCTTATCCAGGCTTTTTGAC-3′
hGH-COOH-H6 REVERSE (SEQ ID NO: 2nd generation
REV)
5′-CGGAATTCCTCGAGCTACTAGTCGACCTAGTGATGGTGGTGATGGT-
GGTCGACGAAGCCACAGCTGCCCTCCACAGAGCGGCACTG-3′

8.4. Purification of SFv5-HGH-6×His Fusion Proteins

Large scale cultures (6-12 liters) of bacterial cells transformed with the sFv-hGH-6×His plasmid can be induced to express the hGH fusion protein. The fusion protein is isolated from the soluble periplasmic fraction, and/or from the insoluble pellet (IP) using a modified osmotic lysis protocol and denaturation/renaturation extraction methods. Solubilized sFv-HGH fusion protein can then be further purified using sequential column affinity chromatography employing Protein A, metal ion-affinity (e.g., 6×His tag binding to Ni⁺⁺), and/or size-exclusion (e.g., sephadex/sephacryl) resins.

A solubilized periplasmic fraction (170 mls) from a 6 liter sFv-hGH preparation was subjected to immobilized metal ion-affinity chromatography (IMAC) and eluted from the Ni⁺⁺-affinity resin using 250 mM imidazole. Twenty (20) μl aliquots of the input, flow through, wash, and sequential column fractions were subjected to SDS-PAGE and analyzed by Western blotting using sFv-specific and hGH-specific antisera.

The sFv-polyclonal antisera was used to track the 56 kDa sFv-hGH fusion protein, which is concentrated from the input fraction through its specific binding to the Ni⁺⁺-resin. The partially purified and concentrated fusion protein material eluted in fractions 16-20, while cleaved sFv material is found in the column flow through (˜33 kDa). The hGH-polyclonal antisera shows an identical elution pattern for the 56 kDa sFv-hGH fusion protein. Additionally, triplet hGH-6×His bands, representing the proteolyzed C-terminal half of the fusion molecule, co-fractionated with the full-length fusion protein molecule. The latter result is expected in the sense that any polypeptide, be it the fusion protein or breakdown product thereof, that includes the 6×His tag will bind Ni⁺⁺.

A single hGH species representing the sFv-hGH fusion protein was affinity precipitated by Protein A beads or the Ni⁺⁺-resin. Both the Protein A bead IP-material and Ni⁺⁺-resin eluate were positive in the immunofunctional IFhGH ELISA (see below).

### Example 9

### Preparation of Protein Conjugates Comprising Growth Hormone Polypeptides

The sFv5AF-Cys molecule, a sFv5 derivative that comprises a Cys residue that has been introduced by genetic engineering, is used. Alternatively, the single chain antibody sFv5AF, which does not contain a cysteinyl group on its surface is thiolated using N-succinimidyl S-acetylthiopropionate (SATP) (Molecular Biosciences, Inc., Boulder, Colo.). SATP reacts with primary amines to add protected sulfhydryls. The thioacetyl groups can be deprotected with 0.02 M hydroxylamine hydrochloride in order to generate free sulfhydryl groups (Duncan et. al., Anal. Biochem. 132:68-73, 1983).

A protected sulfhydryl group is introduced into the sFv5AF protein by adding 20 μl of 4.4 mg/ml SATP in DMSO for each ml of 4 mg/ml sFv5AF in 0.1 M sodium phosphate containing 1 mM EDTA, pH 7.25 (PE). The reaction is incubated for 1 hour at 25° C., after which excess SATP is removed by desalting on a Sephadex G-25 column equilibrated with PE.

The sulfhydryl group on sFv5AF is activated by the addition of 100 μl of 1.1 mg/ml of 5,5′-dithiobis(2-nitrobenzoic acid) (DTNB, a.k.a. Ellman's reagent) in 0.1 M NaPO₄, pH 7.5, and incubating for 1 hour at 25° C. Excess DTNB is removed by desalting on a Sephadex G-25 column equilibrated in PE. The thiol is deprotected by the addition of 1/10th volume of 0.5 M hydroxylamine in PE, pH 7.5, to the SATP-derivatized sFv5AF, followed by incubation at 30 minutes at 25° C. The thiolated sFv5AF is then desalted on Sephadex G-25 in PE.

Human growth hormone is thiolated using, for example, any of the thiolation procedures described herein for sFv5AF and other molecules. The thiolated growth hormone is then added to the thiolated sFv5AF, and the reaction mix is incubated for 2 hours at 25° C. The reaction is stopped and conjugate molecules are purified accoring to procedures described herein.

### Example 10

### Assays of Molecules Comprising Growth Hormone Polypeptides

10.1. Transcytosis Assays

Transcytosis assays were performed in the transwell system essentially as described above. When sFv-hGH fusion protein is placed in the apical compartment using control cells (MDCK cells that do not express pIgR), in which transcytosis should not occur, the basolateral compartment should not contain any significant amounts of the 56 kDa sFv-hGH fusion protein (or a cleaved sFv fragment which co-purifies with the sFv-hGH fractions); all of the sFv-hGH should remain in the apical compartment. There is, however, always some low level of fluid phase transport of proteins When MDCK cells expressing pIgR (Breitfeld et al., Methods Cell Biol 32:329-37, 1989) are used, sFv-hGH molecules appear in the basolateral compartment. The samples are subjected to SDS-PAGE and detection using polyclonal anti-sFV5 or anti-hGH during Western blotting.

Partially purified sFv-hGH fractions were compared to purified sFv5 (positive control) and hGH (negative control) proteins in the transwell assay. Equal molar amounts of purified sFv (positive control), partially purified sFv-HGH fractions (PBB1 and PPB2), and purified HGH (negative control) were placed in the apical compartment of transwells wells plated with either control MDCK cells or cells expressing pIgR. The transwells were incubated at 37° C. for 16-20 hours, and the media collected from the basal compartments.

In the control (nontransfected) MDCK cells, no transcytosis was observed for the sFv-hGH fractions (PPB1 and PPB2) and HGH negative control sample. A minor amount of transport was seen with the sFv internal control. In the MDCK cells expressing pIgR, ˜10-15% transcytosis of the control sFv protein was seen, representing transcytosis. Transcytosis was also observed for the two partially purified sFv-hGH fusion proteins. Transcytosis was also observed was observed for the cleaved sFv fragment which co-purifies with and is present in some sFv-hGH preparations. The sFv fragment likely competes with the sFv-hGH fusion protein, thus dimishing the apparent amount of transcytosis of the fusion protein.

10.2. “Pull-Down” Assays

In pull-down assays, a [GST]-[stalk] or [GST]-[domain 6] fusion protein (see above) is coupled to glutathione-sepharose beads through the GST-moiety. The sFv5 polypeptide, whether present as a free molecule or as a portion of a compound, should specifically bind to the [GST]-[stalk] or [GST]-[domain 6] fusion protein coupled to the beads. The beads are harvested and then washed 3× with PBS buffer to remove unbound material. Material that is retained on the beads is boiled in SDS-loading dye and subjected to SDS-PAGE and Western blotting.

A crude soluble periplasmic fraction of sFv-HGH was incubated with glutathione beads that had been coupled to a [GST]-[rat domain 6] fusion protein or, as a control, to GST protein. Protein A-coupled beads were also used in parallel experiments. The binding reactions were allowed to proceed overnight at 4° C. The beads were then washed 3 times with 1×PBS and boiled in SDS-loading dye and subjected to SDS-PAGE and Western blotting. Equal volumes of the periplasmic (I), post-precipitation supernatant (FT), wash (W) and bead elution (E) fractions were analyzed. The GST-coupled and Protein-A-coupled beads serve as negative and total protein controls, respectively.

The sFv5-hGH fusion protein from the soluble periplasmic fraction bound [GST]-[rat domain 6] beads, but not GST-beads (negative control). In addition, the [GST]-[rat domain 6]-coupled beads bound similar levels of the sFv-hGH fusion protein as was bound by the sFv Protein-A-coupled beads positive control, which suggests that the majority of the sFv-HGH fusion proteins are active, i.e., bind to the [GST]-[rat domain 6] fusion protein.

### Example 11

### Biological Activity of SFv5AF-HGH Fusion Proteins

11.1. Immunofunctional Assay

A functional immunoassay was used to evaluate the biological activity of the sFv5-GH fusion proteins. Immunofunctional hGH assay kits were purchased from Diagnostic Systems Laboraties, Inc. (Webster, Tex.). The assays were carried essentially according to the manufacturer's instructions.

In brief, the immunofunctional assay works as follows. For details, see Strasburger et al., Immunofunctional assay of human growth hormone (hGH) in serum: A possible consensus for quantitative hGH measurement. J Clin Endocrinol Metab 81: 2613, 1996; Mitchell et al., The immunofunctional assay for growth hormone (GH) compared to immuno- and bio-assays. J Endocrinology 160 (Supplement): Abstract P99, 1999; and Strasburger, Laboratory assessment of GH, Growth Horm IGF Res 8:41-46, 1998.

The membrane-bound hGH receptor is a member of the cytokine receptor family which are coupled to non-receptor protein tyrosine kinases. The hGH molecule sequentially binds one GH receptor molecule via the larger site 1, and this 1:1 complex associates with a second GH receptor via binding to the smaller site 2 on the N-terminus of hGH. In vivo, this results in receptor dimerization and activation of a non-receptor tyrosine kinase (JAK/STAT) signal transduction pathway.

The human growth hormone immunofunctional ELISA assay is a “capture” ELISA in which hGH molecules in a sample are bound (“captured”) by an immobilized monoclonal antibody. The immobilized Mab is specific for an N-terminally located epitope overlapping binding site 2 of hGH. In a second incubation step, biotinylated recombinant human GH-binding protein (b*rhGHBP), which is structurally identical to the hGH receptor domain that binds hGH, binds to binding site 1 of hGH to create a “sandwich”. In a third incubation step, detectable streptavidin molecules are added, and the streptavidin molecules tightly bind to the captured biotin moieties. The streptavidin molecules are attached to horse radish peroxidase (HRP). An HRP substrate, 3,3′,5,5′-tetramethylbenzidine (TMB) (KPL, Kirkegaard & Perry Laboratories, Inc., Gaithersburg, Md.) is then added, and the 4 component complex is detected and quantitated by determining the amount of TMB that has been converted into the reaction product, which has a deep blue color that is read using a spectometer.

Only those hGH molecules having a tertiary structure in which both GHBP binding sites are properly folded can bind a b*rhGHBP molecule and establish a detectable biotin-streptavidin linkage. The assay is thus a conformational ELISA as it uses an antibody to capture a molecule out of solution but uses the growth hormone receptor recombinant protein to recognize a 31 amino acid determinant. In order for a GH molecule or moiety to give a signal, it has to have both binding sites present in the correct conformation.

11.2 Large Scale Preparation of SFv5AF-HGH Fusion Protein

A bacterial glycerol stock containing the pSyn5AF-hGH-H6 plasmid was used to inoculate a 20 ml starter culture which was grown overnight at 37° C. Large scale cultures (20 L total in 2 or 4 L flasks were grown the next day at 37° C. and protein expression induced with 1 mM IPTG. Induction was carried out overnight at 25° C. after which the bacterial cultures were harvested. The cells were pelleted by centrifugation, and the pellets were resuspended in 500 mls of PBB buffer (30 mM Tris, pH 8, 1 mM EDTA, 20% sucrose, 0.02% NaN₃). The suspension of cells was mixed on a rotator at 4° C. for 30 minutes, after which MgSO₄ was added to a final concentration of 1.25 mM, and rotation then continued at 4° C. for an additional 20 minutes.

The mixture was centrifugated at 14,300×g for 30 minutes and the soluble protein fraction collected. The extraction step was repeated and the soluble protein fraction (˜1 L) was adjusted to pH 8.0 with 10N NaOH prior to fractionation on a Ultraflow Protein-A sepharose column (Stratagene). Protein fractions were analyzed by SDS-PAGE and Coomassie staining or combined gel electrophoresis and western blotting with a 5AF-specific polyclonal antisera. Selected Protein-A sepharose fractions containing the sFv5AF-hGH-6×His fusion protein were identified, pooled, and subjected to immobilized metal-affinity chromatography (IMAC) using a Ni⁺⁺-NTA Superflow matrix (Qiagen). Active column fractions containing purified 5AF-hGH-H6 fusion protein were identified by SDS-PAGE and Coomassie staining, or combined gel electrophoresis and western blotting using either sFv5AF-specific or hGH-specific (ThermoShandon, Pittsburgh, Pa.) polyclonal antisera. The pooled IMAC fractions were dialyzed in PBS (EM Science, pH 7.2) and sterile filtered through a 0.2 μm Millex-GV filter (Millipore). The protein concentration was determined to be 0.66 mg/ml using a Coomassie Plus Protein Assay (Pierce Chemical Co.). The purified protein was concentrated from 41 mls to 14 mls using a Centriprep YM-10 column (Millipore). Quantitation using the Coomassie Plus Assay was repeated and the final concentration determined to be 1.6 mg/ml. This lot, AZ-hGH-091201 (Lot #2), was used in in vivo studies and is representative of methods for preparing this and other recombinantly-produced polypeptides of the invention.

11.3. In Vivo Studies in Rats

Male Sprague Dawley rats (280-300 gram body weight) pre-implanted with cannulae in the jejunum and/or jugular vein were used. For intravenous (IV) administration, each rat was given 0.1 ml of a solution containing 1.0 mg/ml of the 5AF-hGH fusion protein in phosphate buffered saline, pH 7.2. For intrajejunual delivery, a dosing solution containing 0.333 mg/ml of 5AF-hGH was prepared in Hank's balanced salt solution adjusted to pH 6.5 with Hepes buffer and supplemented with protease inhibitors (leupeptin, aprotinin and chymostatin). Each rat was dosed with 1.8 ml of this dosing solution into the jejunum. Blood samples were collected through the jugular vein at various times. Plasma samples were prepared by centrifugation and analyzed using an hGH specific ELISA kit obtained from Diagnostic System Laboratories (Webster, Tex.).

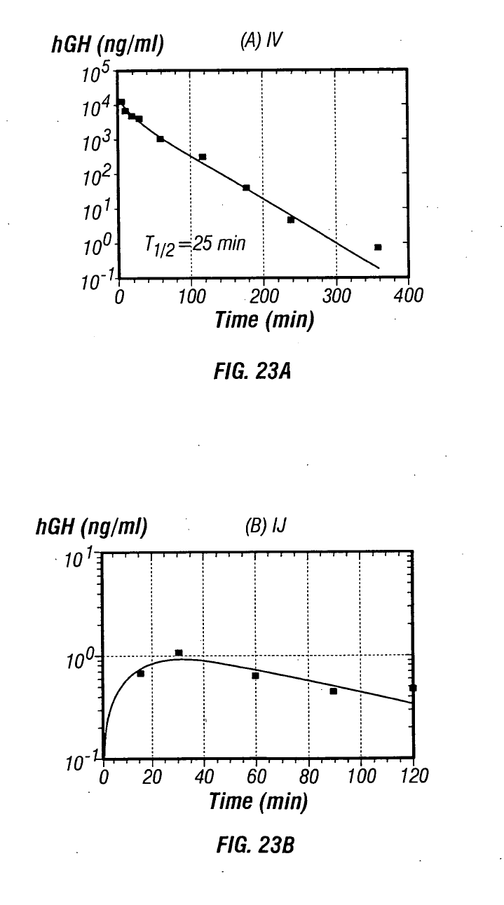

Following IV administration, 5AF-hGH fusion protein was eliminated from the central compartment in a biphasic manner. After a initial sharp distribution phase, 5AF-hGH was eliminated with a terminal half-life of about 25 min (FIG. 23A). After direct administration of the protein into the small intestine, the 5AF-hGh fusion protein was absorbed rapidly with maximum concentration observed at about 30 min post dosing (FIG. 23B).

### Example 12

### Preparation of Fusion Proteins Comprising Interleukin-2

The preparation and assessment of fusion proteins comprising sFv5A and IL-2 are described in this Example. These fusion proteins are loosely referred to as “IL-2-5A” fusion proteins. This connotation reflects the fact that the IL-2 polypeptide is positioned on the amino terminal portion of the fusion proteins and the sFv5A polypeptide is at the carboxy terminus. One skilled in the art will be able to prepare other fusion proteins, e.g., ones in which the IL-2 polypeptide is positioned on the carboxy terminal portion of the fusion protein, or ones having more robust or different biological activities, using the methods described herein.

12.1. Preparation of IL-2 cDNA

In order to generate and isolate mRNAs encoding IL-2, peripheral blood mononuclear cells (PBMC) were prepared and transferred into plates the wells of which had been precoated with mouse anti-human CD3 monoclonal antibody (BD PharMingen, San Diego, Calif.). The plates had been treated with 10 ug/ml of anti-CD3 and washed 3 times before cells were added to the wells; commercially available plates that have been coated with anti-CD3 before sale may also be used (BD BioCoat T-cell Activation Plates, BD PharMingen). Mouse anti-human CD28 monoclonal antibody (BD PharMingen) was then added to 1 ug/ml, and the plates were incubated at 37° C. for 6 hours.

Total cellular RNA was extracted from the stimulated cells using Trizol (LifeTechnologies, Gaithersburg, Md.) essentially according to the manufacturer's instructions. Single strand cDNA copies of the IL-2 message were generated using oligo(dT) primers and the ThermoScript RT-PCR system (Life Technologies) essentially according to the manufacturer's recommendations.

Sequences encoding IL-2 and part of the synthetic linker were amplified via the PCR with the primers “IL-2FormMut3” and “IL-2_Rev2”:

IL-2ForMut3 (SEQ ID NO:______):

5′-CACCATGTACAGGATGCAACTGCTGTCTTG-3′

IL-2_Rev2 (SEQ ID NO:______):

5′-GATTTGCCGCTACCGGAAGTCGACCCAGTTAGTGTTGAGATGATGCTTTGA-3′

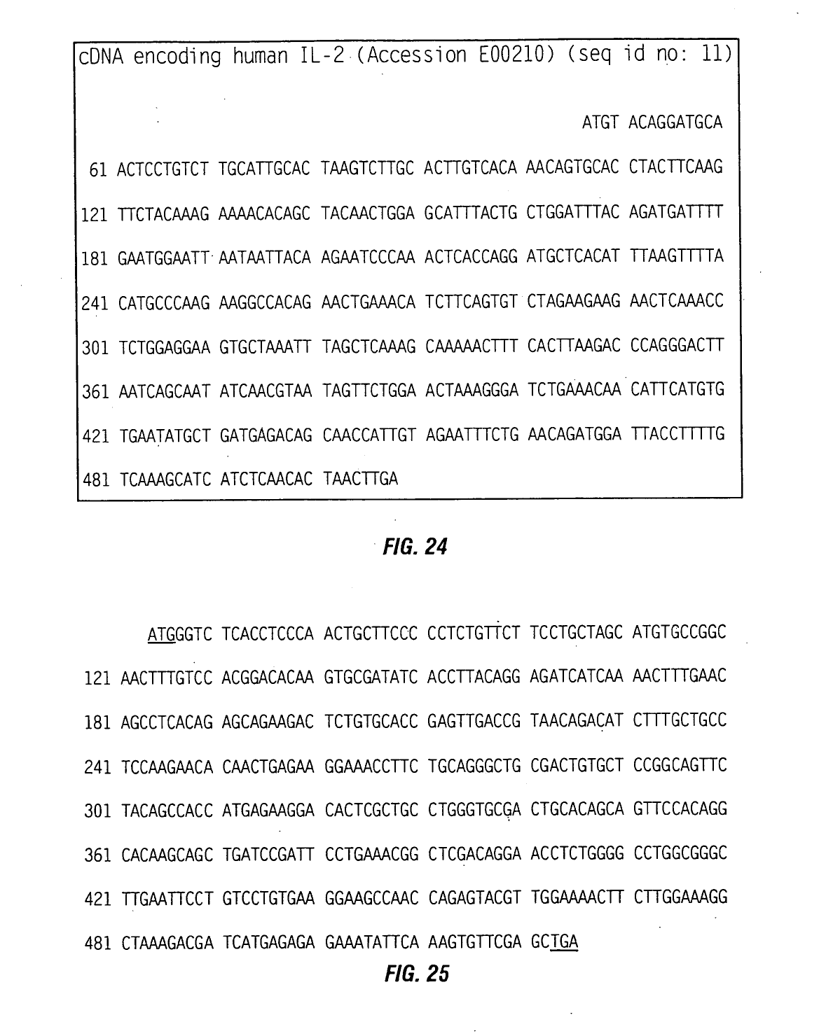

The sequence of IL-2 cDNA is shown in FIG. 24.

12.2. Preparation of Expression Constructs Encoding IL-2-5A Fusion Proteins

The IL-2 PCR product was combined with an sFv5A-encoding PCR product using overlap PCR, a form of PCR that joins two PCR products together. In this method, the intended junction sequence is designed into the PCR primers (at their 5′ ends). Following the initial amplification of each individual polypeptide-encoding sequence, the various products are diluted and combined, denatured, annealed, and extended. An otherwise standard PCR is then performed using “final” forward and reverse primers.

The primers used for the overlap PCR were designed to include sequences encoding a synthetic linker that is connected to the sFv5A polypeptide. The linker includes a 13 amino acid spacer (Gly-Ser-Thr-Ser-Gly-Ser-Gly-Lys-Ser-Ser-Glu-Gly-Lys; SEQ ID NO:______) that has previously been shown to facilitate the correct folding of the fusion protein between IL-2 and a sFv directed against the alpha-folate receptor (Melani et al., Targeting of interleukin 2 to human ovarian carcinoma by fusion with a single-chain Fv of antifolate receptor antibody, Cancer Res 58(18):4146-4154, 1998). The primers used were as follows.

scFvFor (SEQ ID NO:______):

5′-GTAGCGGCAAATCCTCTGAAGGCAAACAGGTGCAGCTGGTGC-
AATCAGGGGGA-3′

scFvRev4 (SEQ ID NO:______):

5′-ACCTAGGACGGTGACCTTGGTCCC-3′

This PCR was performed at about 60° C. for about 25 cycles.

The IL-2, linker and sFv sequence is amplified from a mixture of the IL-2 and sFv5 PCR products using the “IL-2For” primer (SEQ ID NO:______; see above) and the “scFvRev primer” (SEQ ID NO:______; see above). Three cycles of PCR were performed at about 45° C. followed by about 25 cycles performed at about 68° C.

12.3. Preparation of Expression Constructs Encoding IL-2-5A Fusion Proteins

The PCR product from the overlap PCR was gel purified and cloned directly into the mammalian expression vector pcDNA3.1D/V5-His-TOPO® expression vector (Invitrogen, Carlsbad, Calif.). This expression vector includes a CMV-derived promoter for high-level, constitutive expression; a C-terminal V5 epitope tag that can be detected with anti-V5 antibody; and a further C-terminal 6×His tag that can be detected with an anti-6×His tag antibody or used to purify the IL-2-5A fusion protein. Anti-V5 and anti-6×His antibodies are available from Invitrogen.

The DNA was used to transform E. coli, and transformants were selected for using ampicillin as the vector comprises an amipicillin resistance gene. Individual colonies were selected and grown in LB media containing ampicillin. Small scale preparations (mini-preps) of plasmid DNA from 8 colonies were prepared. The predicted structures of four independently selected plasmids was confirmed by digestion with XbaI and gel electrophoresis of the digested DNA. All four of the candidates showed a electrophoresis pattern consistent with the expected product. The nucleotide sequence of the chimeric reading frame that is found in the expression constructs and which encodes the IL-2-sFv fusion protein was determined in order to confirm the accuracy and fidelity of the PCR reactions.

12.4. Transfection and Expression in Eukaryotic Cells

A large scale preparation of plasmid DNA from one of the sequence-confirmed transformants was prepared and used to transiently transfect COS-1 cells using LipofectAMINE 2000 (Life Technologies, Gaithersburg, Mass.) essentially according to the manufacturer's instructions (see Whitt et al., Unit 9.4, pages 9-11 to 9-12, and Unit 16.13, Aruffo, pages 16-53 to 16-55 in: Short Protocols in Molecular Biology, 2nd Ed., Ausubel et al., editors, John Wiley and Sons, New York, 1992). Anti-sFv5A polyclonal antibody was used to detect fusion proteins containing the sFv5A polypeptide. Transfectants are also screened for production and the secretion of the IL-2-5A fusion protein by ELISA or Western analysis using antibodies to human IL-2 (Genzyme) and antibodies to the V5 epitope. Antibodies to human IL-2 are commercially available from, e.g., Research Diagnostics, Inc. (Flanders, N.J.) and Sigma Chemical Corp. (St. Louis, Mo.). The desired fusion protein will be detected by all three of the antibodies. Supernatant from transfected cells, in some instances at least semi-purified by IMAC chromatography, was used in further experiments.

12.4. Purification

IMAC chromatography was used to purify IL-2-5A fusion protein from transiently transfected cells. In brief, about 400 ml of media from transfected COS-1 cells incubated for 48 to 144 hours was harvested. The media was pooled and Imidazole was added to a final concentration of 10 mM. A Pellicon cassette System (Millipore Bioscience, Bedford, Mass.) was used to concentrate the pool to a final volume of ˜75 ml. The concentrated sample was then purified using a nickel column, to which the 6×His tag binds.

12.5.1. Assays of IL-2-5A Fusion Proteins

12.1.5.1. Transcytosis Assay

Transcytosis assays were performed essentially as described above. Fusion protein that had been prepared from the supernatant of transfected COS-1 cells was used in transcytosis assays. Polyclonal antibody to sFv, or monoclonal antibody to the V5 epitope, was used to detect the IL-2-5A fusion protein in both apical and basolateral media. When anti-sFv5A was used, the presence of non-sFv material that was detected by the polyclonal antibodies obscured the results of the transcytosis assay. However, when monoclonal anti-V5 was used to detect the fusion protein in the assay, the results were more straightforward, and a slight but reproducible amount of apical to basolateral transcytosis of the fusion protein was seen. The transcytosis was dependent on the presence of the pIgR stalk as demonstrated by the fact that transcytosis was not observed in control (non-transfected) MDCK cells.

12.1.5.2. Pull-Down Assay

The pull-down assay, described in more detail in preceding Examples, was used to examine the binding of the IL-2-5A fusion protein to a [GST]-[rat domain 6] fusion protein. Although slight, binding of the IL-2-5A fusion protein to the [GST]-[rat domain 6] fusion protein was observed. This result is consistent with the transcytosis assays. That is, the IL-2-5A fusion protein binds weakly to the [GST]-[rat domain 6] fusion protein and undergoes only a relatively small degree of apical to basolateral transcytosis. Fusion proteins comprising IL-2 polypeptide sequences and sFv5 sequences are prepared and tested according to the methods described herein in order to identify IL-2 fusion proteins that have improved desirable characteristics, i.e., bind more tightly to stalk molecules and transcytose to a higher degree.

12.1.5.3. Detection and Quantification

A variety of methods and compositions may be used to detect and quantify the IL-2-5A fusion protein. These include, by way of non-limiting example, a commercially available IL-2 ELISA (DuoSet ELISA Development Kit, R & D Systems, Inc., Minneapolis, Minn.) may be used. A variety of monoclonal antibodies to IL-2 are known and can be used (see for example, Redmond et al., Monoclonal antibodies for purification and assay of IL-2, 17: Lymphokine 5:S29-S34, 1986). The IL-2-5A fusion protein will also carry the V5 epitope and 6×His tag, and there are numerous polyclonal and monoclonal antibodies directed to each of these epitopes that are commercially available. The anti-sFv5A polyclonal antibody described herein can be used to detect the fusion protein.). The IL-2-5A fusion protein is detected by all three types of antibodies.

12.1.5.4 Biological Activity

The IL-2 biological activity of the IL-2-5A fusion protein was tested by evaluating the ability to sustain proliferation of the IL-2-dependent murine cytotoxic T cell line, CTLL-2 (Melani et al., Targeting of interleukin 2 to human ovarian carcinoma by fusion with a single-chain Fv of antifolate receptor antibody, Cancer Res 58(18):4146-4154, 1998). The IL-2 fusion protein supported proliferation of the T cells in this assay in a concentration-dependent manner. The IL-4-5A fusion protein was evaluated in the same assay. Unlike IL-2, which supports cellular proliferation in this assay, IL-4 does not. As expected, the IL-4-5A fusion protein behaves like IL-4, i.e., it fails to support cellular proliferation in this assay.

The ability of fusion proteins to bind ligands, such as soluble IL-2-receptor polypeptides (Dracheva et al., Expression of soluble human interleukin-2 receptor alpha-chain in Escherichia coli, Protein Expr Purif 6:737-47, 1995; Junghans et al., Biophysical characterization of a recombinant soluble interleukin 2 receptor (Tac). Evidence for a monomeric structure, J Biol Chem 271:10453-60, 1996) or lipoteichoic acid (Plitnick et al., Lipoteichoic acid inhibits interleukin-2 (IL-2) function by direct binding to IL-2, Clin Diagn Lab Immunol 8(5):972-9, 2001) can be measured either directly when immobilized on a surface or indirectly by their ability to competitively inhibit IL-2 binding to antibody in ELISA assays. Other methods for measuring the amount and biological activity of IL-2 are described by Gately et al. (Unit 6.16 in: Current Protocols in Immunology, John Wiley and Sons, New York, 2000; Indrova et al., Folla Biol. (Praha) 43:45-47, 1997).

### Example 13

### Preparation of Fusion Proteins Comprising Interleukin-4

The preparation and assessment of fusion proteins comprising sFv5A and IL-4 are described in this Example. These fusion proteins are loosely referred to as “IL-4-5A” fusion proteins. This connotation reflects the fact that the IL-2 polypeptide is positioned on the amino terminal portion of the fusion proteins and the sFv5A polypeptide is at the carboxy terminus. One skilled in the art will be able to prepare other fusion proteins, e.g., ones in which the IL-4 polypeptide is positioned on the carboxy terminal portion of the fusion protein, or ones having more robust or different biological activities, using the methods described herein.

13.1. Preparation of IL-4 cDNA

A nucleotide sequence encoding an IL-4 polypeptide (see FIG. 25; SEQ ID NO:______) was amplified from plasmid pcD-hIL-4 (ATCC #57593) using the following primers:

IL-4For2 (SEQ ID NO:______):

5′-CACGATGGGTCTCACCTCCCAACTGCTT-3′

IL-4RevMut2 (SEQ ID NO:______):

5′-GATTTGCCGCTACCGGAAGTCGACCCGCTCGAACACTTTGAGTATTTCTCT-3′

The IL-4 PCR product was combined with the above-described sFv5A PCR product in overlap PCR. The resulting PCR product, which includes a chimeric reading frame that encodes a [IL-4]-[sFv5A] fusion protein, was gel purified and cloned directly into the mammalian expression vector pcDNA3.1D/V5-His-TOPO® expression vector (Invitrogen, Carlsbad, Calif.) using a strategy similar to that used for the [IL-2]-[sFv5A] fusion protein.

The transcytotic properties of the IL-4-5A fusion protein were evaluated in the transwell assay, and a slight but reproducible amount of apical to basolateral transcytosis of the fusion protein was seen. The transcytosis was dependent on the presence of the pIgR stalk as demonstrated by the fact that transcytosis was not observed in control (non-transfected) MDCK cells.

13.2. Fusion Proteins of Interleukin Derivatives

The above-described methods can be used to prepare fusion proteins of the invention in which the interleukin-derived portion thereof is or is derived from an interleukin homolog or derivative.

As non-limiting examples, homologs of human IL-2 that may be used include murine IL-2 (Robbens et al., Protein Expr Purif 6(4):481-486, 1995; Steidler et al., Appl Environ Microbiol 61(4):1627-1629, 1995; Guisez et al., Protein Expr Purif 4(3):240-246, 1993) ovine IL-2 (Seow et al., Vet. Immunol. Immunopathol. 56:107-117, 1997, Wood et al., Vet. Immunol. Immunopathol. 54:33-44, 1996), cervine IL-2 (Indrova et al., Folia. Biol. (Praha) 43:45-47, 1997), bovine IL-2 (Kashima et al., J. Vet. Med. Sci. 61:171-173, 1999, Kashima et al., J. Vet. Med. Sci. 61:705-707, 1999; Brown et al., J Immunol 135(5):3184-3190, 1985), and cynomolyns monkey IL-2 (Yabe et al., Int. Arch. Allergy Immunol. 113:417-423 1997) all of which have been produced in various expression systems. One skilled in the art will be able to select the appropriate source of DNA, sequence of primers, PCR conditions, etc. for each particular genetic sequence encoding a given IL-2 homolog or derivative. As non-limiting examples, derivatives of IL-2 that may be used include functional oligopeptides derived therefrom (Bonne et al., Construction, purification and biological activities of recombinant human interleukin-2 analogs, Dev Biol Stand 69:157-168, 1988).

As non-limiting examples, homologs of human IL-4 that may be used include canine, caprine, ovine, and bottle-nosed dolphin IL-4 sequences (see, respectively, van der Kaaij et al., Molecular cloning and sequencing of the cDNA for dog interleukin-4, Immunogenetics 49(2):142-3, 1999; Snekvik et al., Characterization of caprine interleukin-4, Vet Immunol Immunopathol 78:219-29, 2001; Chaplin et al., The expression and biologic effects of ovine interleukin-4 on T and B cell proliferation, J Interferon Cytokine Res 20:419-25, 2000; and Inoue et al., Cloning and sequencing of a bottle-nosed dolphin (Tursiops truncatus) interleukin-4-encoding cDNA, and J Vet Med Sci 61:693-6, 1999). As non-limiting examples, derivatives of IL-4 that may be used include IL-4 variants that arise due to alternate splicing (Sakkas et al., Increased levels of alternatively spliced interleukin 4 (IL-4delta2) transcripts in peripheral blood mononuclear cells from patients with systemic sclerosis, Clin Diagn Lab Immunol 6:660-4, 1999).

### Example 14

### Fusion Proteins Comprising Insulin Polypeptides

14.1. Preparation of Fusion Proteins Comprising Insulin Polypeptides

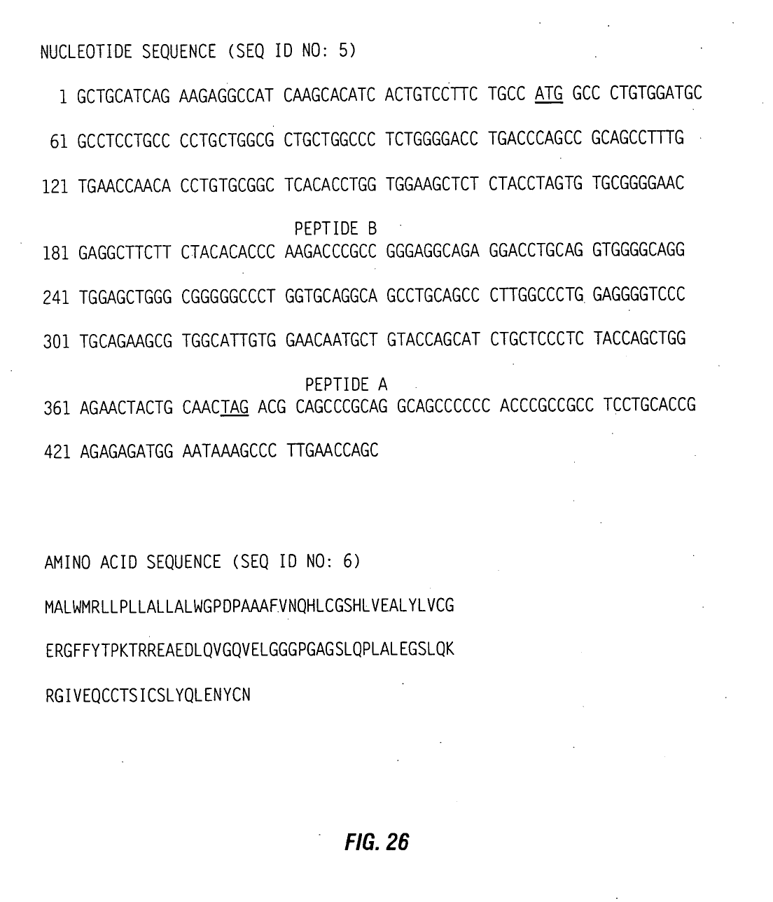

In order to prepare DNA molecules that encode human insulin (hInsulin), a two-step cloning procedure is used. In the first step, PCR is used to amplify insulin cDNA sequences (FIG. 26; SEQ ID NO:______) from a human cDNA library purchased from a commercial source (Clontech: human pancrease HL5032t). The primers are designed so that, after the PCR product is inserted into an sFv expression vector, the insulin amino acid coding sequence is in-frame with the amino acid reading frame of the sFv protein expression construct, and thus encodes either an sFv-insulin fusion protein.

DNA amplification, PCR product analysis, purification, cloning into the intermediate vector, and sequence confirmation are carried out as described above. The sequence of the “Forward” primer (SEQ ID NO: 2nd generation FOR) includes a 5′ Not I restriction site followed by a sequence that encodes an internal Gly₄-Ser linker for the in-frame insertion with the carboxy terminus of the sFv in the pSyn5A or pSyn5AF constructs. The sequence of the “reverse” primer (SEQ ID NO: 2nd generation REV) contains in-frame sequences encoding the following: Sal I-6×His tag-Sal I-(TAG stop codon)₂-Xho I-Eco RI site. Once the insulin-encoding nucleotide sequence in the intermediate vector is confirmed, the insulin reading frame is excised using the Not I and Eco RI restriction enzymes, gel purified and ligated into Not I/Eco RI-digested pSyn5A or pSyn5AF expression vectors. The resulting chimeric reading frame encodes a fusion protein with a G₄S linker peptide-hInsulin protein fused in-frame with the carboxy terminus of a sFv (5A or 5AF). The resulting fusion proteins are called sFv-hINS-H6 (in pSyn5A or pSyn5AF, respectively).

Human INSULIN-COOH-H6Forward (SEQ ID NO: ______)

5′-ATAAGAATGCGGCCGCAGGTGGCGGAGGGTCATTTGTGAACCAACA-
CCTGTGCGGCTCACAC-3′

Human INSULIN-COOH-H6 Reverse (SEQ ID NO: ______)

5′-CGGAATTCCTCGAGCTACTAGTCGACCTAGTGATGGTGGTGATGGT-
GGTCGACGTTGCAGTAGTTCTCCAGCTGGTAGAGGGAGC-3′

14.2. Assays for SFv-Insulin Fusion Proteins

ELISA assays for insulin are commercially available from, e.g., Diagnostic Systems Laboratories, Inc., Webster, Tex. These assays can be used to detect whole insulin in a variety of formats.

An ELISA to the C-peptide of Insulin is commerically available (DSL, Inc.) and is used in some instances. Although it is biologically inactive, the C-peptide has a longer half-life in blood and undergoes relatively little hepatic metabolism. Thus, assays of the C-peptide may offer may sensitive measurements of the amount of insulin delivered to an animal.

### Example 15

### Preparation of Fusion Proteins Comprising Calcitonin Polypeptides

15.1. Fusion Proteins Comprising Human Calcitonin Peptides

15.1.1. Preparation of Nucleic Acids Encoding Human Calcitonin

In order to prepare DNA molecules that encode human calcitonin (hCalcitonin), a two-step cloning procedure is used. In the first step, PCR is used to amplify sequences encoding human calcitonin (hCalcitonin, FIG. 9; SEQ ID NO:______) from a human cDNA library purchased from a commercial source (Clontech: human pituitary gland HL1139a or HL1139b; human placenta HL5020t). The primers are designed so that, after the PCR product is inserted into an sFv expression vector, the calcitonin amino acid coding sequence is in-frame with the amino acid reading frame of the sFv protein expression construct, and thus encodes either a calcitonin-sFv or sFv-calcitonin fusion protein.

The PCR products are inserted into an intermediate vector as described above. DNA amplification, PCR product analysis, purification and sequence confirmation are carried out as described above.

Once the calcitonin-encoding nucleotide sequence in the intermediate vector is confirmed, the calcitonin reading frame is excised using restriction enzymes and is inserted in frame with a sFv reading frame in, e.g., pSyn5A or pSyn5AF. The resulting chimeric reading frame encodes either a calcitonin-sFv or sFv-calcitonin fusion protein.

15.1.2. Human Calcitonin Amino-Terminal Fusion Proteins

PCR amplification reactions are carried out as described above using 2 μL of the human pituitary gland or placental cDNA library (Clontech: HL1139a, HL1139b or HL5020t) as template DNA and the huCalc-NH2-For (SEQ ID NO:______) and huCalc-NH2-Rev (SEQ ID NO:______) primers for engineering amino-terminal human calcitonin fusion constructs. The forward primer (SEQ ID NO:______) includes a NcoI restriction site for the in-frame insertion of the calcitonin-encoding sequences amino-terminal to the sFv in the pSyn5A or 5AF constructs. The reverse primer (SEQ ID NO:______) has a sequence encoding an internal in-frame Gly₄Ser linker sequence and an NcoI site for insertion of the calcitonin gene amino-terminal to sFv5A or sFv5AF.

The PCR product is ligated into the pCR-TOPO vector (Invitrogen) and the cDNA sequence is confirmed. The hCalcitonin encoding sequences are then excised from the intermediate vector by digestion with Nco I, gel purified and ligated into Nco I-digested pSyn5A or pSyn5Af expression vectors. The resulting chimeric reading frame encodes a fusion protein with a hCalcitonin protein-G₄S linker peptide fused in-frame to the amino-terminus of a sFv (e.g., sc5A or sc5AF). This fusion protein is called hCalcitonin-sFv.

huCalc-NH2-For (SEQ ID NO:______)

5′-CATGCCATGGCCATGGGCTTCCAAAAGTTCTCCCCC-3′

huCalc-NH2-Rev (SEQ ID NO:______)

5′-CATGCCATGGCTGAACCGCCTCCACCGTTGGCATTCTGGGGCATGCTAAC-3′

15.1.3. Human Calcitonin Carboxy-Terminal Fusion Proteins

In order to generate fusion proteins having a carboxy-terminal human calcitonin polypeptide, PCR amplification reactions are carried out as described above using 2 μL of a human pituitary gland or placental cDNA library (Clontech: HL1139a, HL1139b or HL5020t) and the huCalc-COOH-For (SEQ ID NO:______) and huCalc-COOH-Rev (SEQ ID NO:______) for engineering carboxy-terminal (COOH) human calcitonin fusion constructs. The forward primer (SEQ ID NO:______) has incorporated a Not I restriction site followed by an internal Gly₄Ser linker for insertion in-frame with the C-terminus of the sFv5A. The reverse primer (SEQ ID NO:______) sequence contains tandem in-frame TAA stop codons followed by an Eco RI site for insertion of the human calcitonin gene carboxyl-terminal to sFv (5A or 5AF).

The PCR product is ligated into the pBluescript II vector (Stratagene), and the cDNA sequence of this intermediate vector is confirmed. The hCalcitonin-encoding DNA is then excised from the intermediate vector by digestion with Not I and Eco RI, gel purified and ligated into Not I/Eco RI-digested pSyn5A or pSyn5AF expression vectors. The resulting chimeric reading frame encodes a fusion protein with G₄S linker peptide-hCalcitonin protein fused in-frame with the carboxy terminus of a sFv (sFv5A or sFv5AF). These fusion proteins are called, respectively, sFv5A-hCalcitonin and sFv5AF-hCalcitonin.

huCalc-COOH-For (SEQ ID NO:______)

5′-ATAAGAATGCGGCCGCCGGTGGAGGCGGTTCAATGGGCTTCCAAAAGTTCTCCCCC

huCalc-COOH-Rev (SEQ ID NO:______)

5′-CGAATTCTAATAAGTTGGCATTCTGGGGCATGCTAAC-3′

15.2. Fusion Proteins Comprising Salmon Calcitonin Peptides

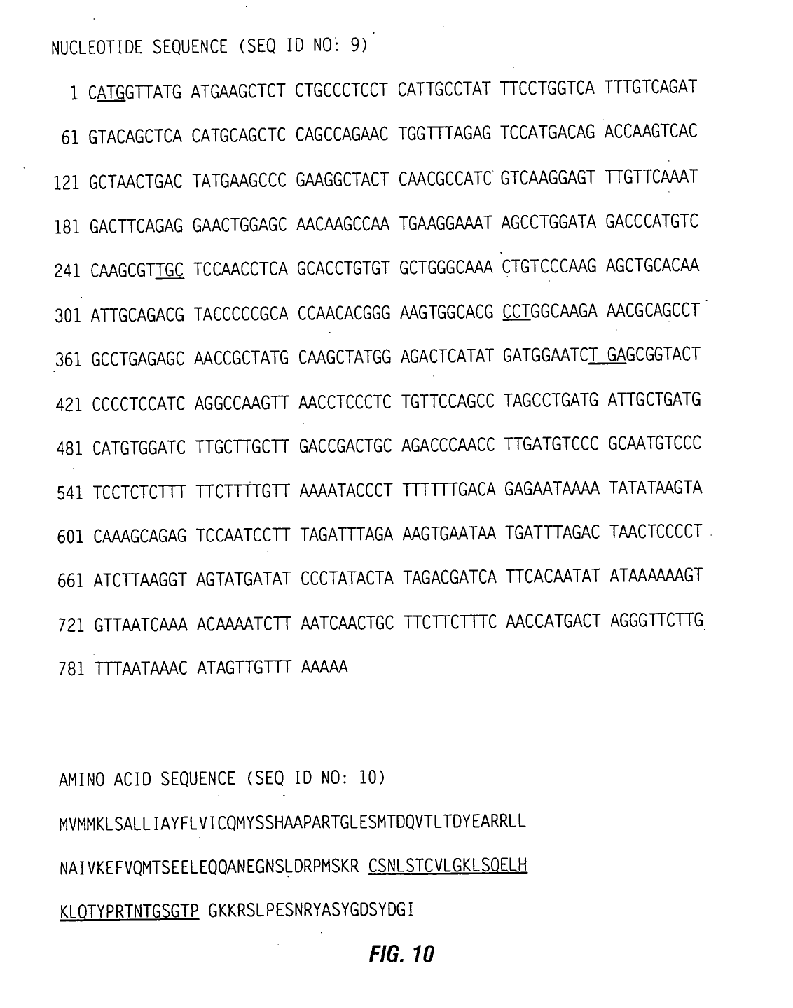

Salmon calcitonin (FIG. 10; SEQ ID NO:) sFv fusion proteins are prepared using synthetic oligonucleotides that are annealed to each other and then ligated into either NcoI- or NotI-EcoRI-digested pSyn5A or pSyn5AF plasmid DNA, resulting in a chimeric reading frame in which sequences encoding the mature active salmon calcitonin peptide are fused to either the amino-terminal or carboxy-terminal side of the sFv DNA sequence, respectively.

The phosphorylated synthetic oligonucleotides are made and purchased commercially (MWG Biotech), annealed by heating to 95° C. and slowly cooling to room temperature, and then directly ligated into the digested vector using T4 DNA ligase essentially according to the manufacturer's (NEB Labs) instructions. The SEQ ID NO:______ oligonucleotide contains cDNA sequence corresponding to nucleotides 248-343 of the sense strand of salmon calcitonin gene (which encodes for the mature active peptide underlined in the amino acid sequence shown in FIG. 10) and contains a 5′-Nco I overhang sequence. The SEQ ID NO:______ oligonucleotide is annealed to SEQ ID NO:______, which contains complementary (antisense) cDNA sequence corresponding to nucleotides 343-248, as well as a G₄S linker sequence and a terminal 3′-NcoI overhang sequence. The above manpulations result is an in-frame insertion of the salmon calcitonin gene sequence into the amino terminal side of sFv5A or sFv 5AF.

SEQ ID NO:______ contains a 5′-NotI overhang sequence followed by a G₄S linker-encoding sequence; the cDNA sequence corresponds to nucleotides 248-343 of the sense strand of salmon calcitonin gene (encoding for the mature active peptide underlined in the amino acid sequence shown in FIG. 10; SEQ ID NO:______). The SEQ ID NO:______ oligonucleotide contains sequence corresponding to the complementary (antisense) cDNA sequence corresponding to nucleotides 343-248, tandem stop codon sequences, and a terminal 3′-Eco RI overhang sequence. The SEQ ID NO:______ oligonucleotide is annealed to SEQ ID NO:______, and inserted into a NotI/EcoRI digested pSyn5A or pSyn5AF vector to generate in-frame insertions of salmon calcitonin encoding sequences on the carboxy terminal side of sFv5A or sFv5AF.

NH2-salCalcitonin Sense (SEQ ID NO:______)

5′-CATGGCCTGCTCCAACCTCAGCACCTGTGTGCTGGGCAAACTGTCCCAAGAGCTG-
CACAAATTGCAGACGTACCCCCGCACCAACACGGGAAGTGGCACGCCTGGTGGAGGCG-
GTTCAGC-3′

NH2-salCalcitonin Antisense (SEQ ID NO:______)

5′-CATGGCTGAACCGCCTCCACCAGGCGTGCCACTTCCCGTGTTGGTGCGGGGG-
TACGTCTGCAATTTGTGCAGCTCTTGGGACAGTTTGCCCAGCACACAGGTGCTGA-
GGTTGGAGCAGGC-3′

COOH-salCalcitonin Sense (SEQ ID NO:______)

COOH-salCalcitonin Sense (SEQ ID NO:______)

5′-GGCCGGTGGAGGCGGTTCATGCTCCAACCTCAGCACCTGTGTGCTGGGCAAAC-TGTCCCAAGAGCTGCACAAATTGCAGACGTACCCCCGCACCAACACGGGAAGTGGC-ACGCCTTAATAAG-3′

COOH-salCalcitonin Antisense (SEQ ID NO:______)

5′-AATTCTAATAAAGGCGTGCCACTTCCCGTGTTGGTGCGGGGGTACGTCTGCAAT-TTGTGCAGCTCTTGGGACAGTTTGCCCAGCACACAGGTGCTGAGGTTGGAGCATGAA-CCGCCTCCACC-3′

### Example 16

### Preparation of Protein Conjugates Comprising Calcitonin Polypeptides

16.1. Screening of Cross-Linking Agents Suitable for Salmon Calcitonin

Different cross-linkers were screened for the ability to derivatize salmon calcitonin (sCalcitonin) without forming a precipitate of the protein. It was found that addition of acetonitrile to the derivatization reaction helped prevent protein precipitation. A list of the cross-linkers screened and whether a precipitate formed is shown in Table 13, followed by diagrams showing the chemical structure of each cross-linker. On the basis of the cross-linker screening, mal-sac-HNSA was chosen as the cross-linker to be used in the sFv-calcitonin conjugation.

TABLE 13
CROSSLINKERS USED TO DERIVATIZE SALMON CALCITONIN

| | |
|---|---|
| Chemical Nature of | Preciptation With: |

| Crosslinker | Crosslinker | Aqueous | w/Acetonitrile |
|---|---|---|---|
| SPDP | NHS ester & pyridyldithio | Ppt | Ppt |
| Sulfo-LC-SPDP | NHS ester & pyridyldithio | Ppt | Ppt |
| SMPT | NHS ester & pyridyldithio | Ppt | Ppt |
| Sulfo-LC-SMPT | NHS ester & pyridyldithio | Ppt | Ppt |
| LC-SMCC | NHS ester & maleimide | Ppt | Ppt |
| Mal-Sac-HNSA | HNSA & maleimide | No ppt | No ppt |
| | (noncleavable, H₂O sol.) | | |
| SATP | Thiolation reagent (protected) | Ppt | Ppt |

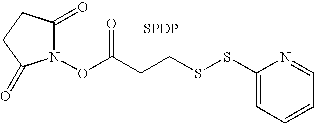

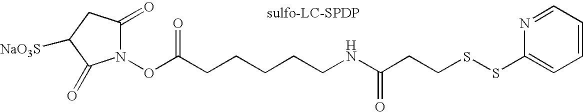

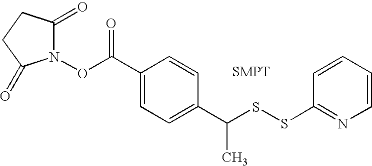

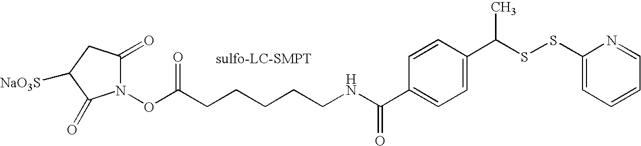

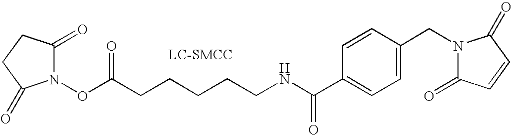

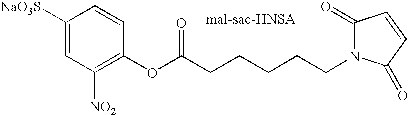

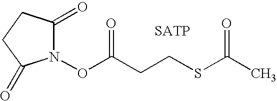

16.2. Conjugation of Salmon Calcitonin to SFv5AG₄-Cys

16.2.1. Preparation of SFv5AG₄-Cys

A preparation of sFv5AG₄-Cys was reduced by the addition of DTT to a final concentration of 10 mM, and the monomer and dimer fractions were separated by SEC on a 1×44 cm Superdex 75 column in 10 mM PO₄ containing 100 mM NaCl and 1 mM EDTA, pH 6.25.

16.2.2. SFv5AG₄-Cys-Malsac-sCalcitonin Conjugation

Salmon calcitonin (sCT) was derivatized with a 5-fold molar excess of mal-sac-HNSA until a calculated substitution ratio of 0.94 was reached, as observed by monitoring OD₄₀₆. The excess mal-sac-HNSA was removed by desalting on a 5 ml G25 column (Pharmacia HiTrap). The derivatized sCT was added to the sFv5AG₄Cys monomer and the reaction was incubated at 25° C. for 3 hours. The chemical conjugation reactions are shown below.

16.2.3. Purification of the SFv5AG₄-Cys-Malsac-sCalcitonin Conjugates

The sFv5AG₄-Cys-malsac-sCalcitonin conjugate was purified by SEC using a 1×44 cm Superdex 75 column in 0.1 M PO₄, pH 7.5, containing 1 mM EDTA. Peak fractions were collected and analyzed by Coomassie-stained SDS-PAGE. A significant amount of the monomer sFv5AG₄-Cys-sCT conjugate had dimerized, and the dimer conjugate was collected included in the transcytosis assay as well as the monomer sFv5AG₄-Cys-sCT conjugate.

16.3. Conjugation of Salmon Calcitonin to SFv5AF-Cys

16.3.1. Preparation of SFv5AF-Cys

A solution of sFv5AF-Cys (9.5 ml, 10 mg/ml, 0.36 mM) was incubated with 10 mM DTT for 30 minutes and then was purified using size exclusion chromatography (SEC) on a 1.6×60 cm Superdex 75™ column in 100 mM PO₄, 100 mM NaCl and 1 mM EDTA, pH 6.25. Three sequential runs were performed to purify monomer and dimer sFv5AF-Cys. The sFv5AF-Cys was resolved into monomer and dimer sFv5AF-Cys peaks. Dimer and monomer sFv5AF-Cys were quantitated after pooling by measuring A₂₈₀ and the protein yield of dimer sFv5AF-Cys was calculated to be 26.4 mg with a concentration of 1.2 mg/ml or 0.043 mM. Monomer sFv5AF-Cys was calculated to have a protein yield of 30 mg with a concentration of 1.18 mg/ml or 0.042 mM.

16.2.2. Derivatization of Salmon Calcitonin (sCT)

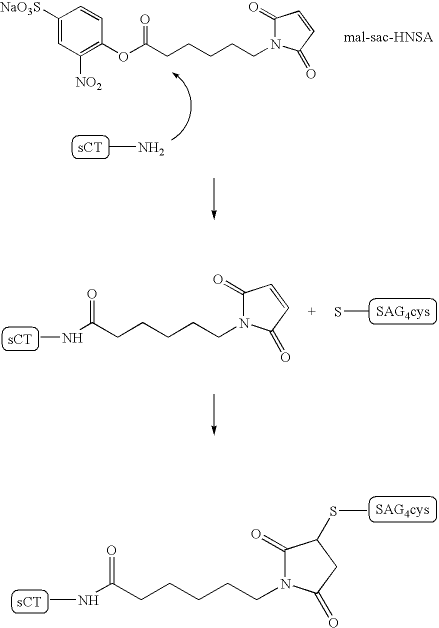

Salmon calcitonin was desalted on a 24 ml P2™ column in 30% acetonitrile, 100 mM PO₄, 1 mM EDTA, pH 7.25. The concentration of sCT was calculated to be 22.3 mg/ml by measuring A₂₈₀. Since sCT is known to be soluble to at least 9.0 mg/ml, the 22.3 mg/ml sCT was diluted down to 9.0 mg/ml to prevent precipitation from occurring. 5.5 ml (9.0 mg/ml) sCT was used for the monomer conjugation and another 5.5 ml (9.0 mg/ml) sCT was used for the dimer conjugation. The 5.5 ml (9.0 mg/ml) sCT for the monomer conjugation was derivatized with a 5-fold molar excess of mal-sac-HNSA. The reaction was incubated at 25° C. for an hour until a 1:1 substitution ratio was reached by monitoring A₄₀₆. The mal-sac-sCT reaction was desalted immediately on the 24 ml P2™ column in 100 mM PO₄, 1 mM EDTA, pH 7.25. The derivatization was repeated for the 5.5 ml (9.0 mg/ml) dimer conjugation.

The protein concentration of both the monomer and dimer mal-sac-sCT derivatization was determined by measuring A₂₈₀. The mal-sac-sCT from the monomer conjugation was determined to have a protein yield of 14.9 mg (2.7 mg/ml, 0.79 mM). The yield of mal-sac-sCT for the dimer conjugation was determined to be 22 mg (3.7 mg/ml, 1.08 mM).

16.2.3. Conjugation of Monomer and Dimer SFv5AF-Cys to Mal-Sac-sCT

An 8-fold molar excess (14.9 mg) of mal-sac-sCT, for the monomer conjugation, was added to 14.9 mg monomer sFv5AF-Cys with a final volume of 18 ml. An 8-fold molar excess (22.0 mg) of mal-sac-sCT, for the dimer conjugation, was added to 22.0 mg dimer sFv5AF-Cys with a final volume of 24 ml. The reactions for both monomer and dimer conjugation were incubated at 4° C. overnight. After the incubation, both of the reactions were concentrated down to 3 ml using a Pall Gelman 10K Centricon™. The monomer and dimer conjugates were purified on a 1.6×60 cm SEC Superdex 75™ column in PBS.

### Example 17

### Assays of Molecules Comprising Calcitonin Polypeptides

17.1. Transcytosis Assays

Chemical conjugates of salmon calcitonin and sFv5AF-Cys or sFv5AG₄-Cys monomers or dimers were prepared. These conjugates are examples of a biologically active molecule (calcitonin) covalently associated with monovalent (sFv5AF monomers) and multivalent (sFv5AF dimers) ligands directed to a molecule that confers transcytotic properties to complexes bound thereto.

17.2. Procedure

Transcytosis assays were performed essentially as described above. Duplicate assays were carried out in which 1 μg of sFv or conjugate molecules was added to the apical chamber of MDCK cells expressing chimeric pIgR or control MDCK cells. Transcytosis was carried out for 11 hours at 37 degrees. Apical and basolateral media were collected, volumes adjusted to 1 ml, and 500 μl of the basolateral media and 50 μl of the apical media were subjected to protein A sepharose precipitation. Samples were analyzed by non-reducing SDS-PAGE and Western blotting with an anti-5AF antibody.

17.3. SDS-PAGE Analysis

Both sFv5AG₄-Cys and conjugates thereof show bands corresponding to monomeric and dimeric forms on non-reducing SDS-PAGE (“gel-monomer” and “gel-dimer”, respectively). Most of the gel-dimer molecules are probably disulfide linked. In the case of sFv5AG₄-Cys, a disulfide bridge could be formed between the engineered C-terminal cysteines and/or between internal cysteines. The latter event might occur during boiling of the samples in SDS prior to sample loading.

In the case of the conjugates, since one of the c-terminal cysteines is linked via a thioether bond to calcitonin, forms migrating as dimer on SDS-PAGE are probably due to disulfide bridges forming between internal cysteines in the sFv during boiling in SDS, or between a C-terminal cysteine on one sFv molecule and an internal cysteine on another sFv molecule. About half of the dimer conjugate preparation migrates as dimer, probably reflecting the efficiency of cross-linking of the dimeric molecules during boiling. In the monomer conjugate preparation, a small fraction of the conjugate migrates as dimer. The dimeric material is enriched in the basolateral media.

17.3.1. SFv5AG₄-Cys

The sFv5AG₄-Cys preparation is a mixture of monomers and dimers. A substantial portion of the sFv preparation migrates as a dimer on non-reducing SDS-PAGE. The dimer species was probably produced by covalent or non-covalent interactions that occurred prior to boiling in SDS. Thus, by comparing the monomer and dimer bands on the gel, one can monitor monomer and dimer transcytosis in the same sample. Transcytosis of sFv5AG₄-Cys dimers is typically greater than 10%, whereas transcytosis sFv5AG₄-Cys monomers is usually less than 10%, often less than 5%.

17.3.2. SFv5AG₄-Cys-Calcitonin Conjugates

The preparation of monomer sFv5AG₄-Cys-calcitonin that was tested shows 2 conjugate species on SDS-PAGE. These species of conjugates behave differently. Transcytosis of the gel-monomer conjugate is relatively inefficient, resembling that of the sFv5AG₄-Cys monomer. In contrast, transcytosis of the gel-dimer conjugate is relatively efficient.

Taken together, these results suggest that for sFv5AG₄-Cys, and for sFv5AG₄-Cys-calcitonin conjugates, dimeric forms of sFv5AG₄-Cys show more efficient transcytosis than the corresponding monomeric forms.

### Example 18

### Conjugation of sFv5AF-Cys to Infliximab

18.1 Chemical Conjugation

The chemical reactions that take place during the conjugation of Infliximab (Remicade®, a product of Centocor, Malvern Pa.) to sFv5AF-Cys using N-succinimidyl 3-(2-pyridyldithio)propionate (SPDP) are shown below.

In the first reaction, one or more primary amines of Infliximab reacts with SPDP. The primary amine(s) may be found on the side chain of a lysine residue in the Infliximab molecule, or at the proteins amino terminus. A primary amine is modified by the first reaction to produce an intermediate in which the primary amine is attached to a chemical structure having a disulfide bond to an aromatic leaving group.

In the second reaction, In the second reaction, the sFv5AF-Cys molecule is reduced with 10 mM DTT. Any Cys residue may be reduced, but the C-terminal Cys residue is generally more accessible for reduction. Unreacted DTT is removed by size exclusion chromatography and/or dialysis.

The reduced sFv5AF-Cys reacts with the Infliximab intermediate. The aromatic leaving group in the intermediate is displaced by the activated Cys residue, forming a disulfide bond in the process.

The approximate molecular weights of the conjugation recatants and products are as follows: sFv5AF-Cys monomer, 28 kDa; Infliximab, 150 kDa; [sFv5AF-Cys monomer:Infliximab] conjugate, 178 kDa. The structure of the conjugate of monomeric sFv-5AF-Cys is represented as follows:

18.2 Titration of SPDP to Determine the Molar Ratio Needed For a 1:1 SPDP: Infliximab Derivatization.

Infliximab (Remicade®) was dissolved to 10 mg/ml, and aliquots of 10 μl (250 μg) were prepared for derivatization with SPDP. SPDP was added to the Infliximab aliquots in 1.0 to 3.5-fold molar excess. The reactions were incubated for about 20 to 30 minutes at 25° C., and each reaction was desalted on a 5 ml HiTrap G25 desalting column (Pharmacia) using desalting buffer (100 mM sodium phosphate, pH 7.5, containing 1 mM EDTA). The peak fractions were pooled and 100 μl was taken for spectrophotometry at OD₂₈₀ to calculate the Infliximab concentration. The amount of SPDP added to the Infliximab was determined by taking 100 μl of the derivatized Infliximab and adding DTT to 10 mM, then using spectrophotometry at OD₃₄₃ to calculate the amount of the pyridine-2-thione released (ε=8,080 M−1 cm−1). The results, which are shown below, demonstrate that a molar ratio of 1:1 SPDP: Infliximab yields a derivatization ratio of 1.0. This derivitization ratio is desirable because it limits or prevents the formation of large quantities of multimers that have varying ratios of Infliximab to sFv-AF-Cys.

RESULTS OF SPDP TITRATION

| Molar Excess of SPDP Added | Ratio of SPDP:Infliximab |
|---|---|
| 1.0 | 1.13 |
| 1.5 | 1.53 |
| 2.0 | 2.12 |
| 3.0 | 3.11 |
| 3.5 | 3.30 |

SPDP was added to 10 mg of Infliximab (10 mg/ml) at a molar ratio of 1:1. The SPDP was dissolved in DMSO and was added in a volume that was 2.5% of the volume of Infliximab. The reaction was incubated at 25° C. for 30 minutes, desalted, and the amount of derivatization determined as described above. An equal mass of derivatized Infliximab and purified 5AF-Cys monomer (see Example 7) were added together to give an approximate 5-fold molar excess of 5AF-Cys. The conjugation reaction was incubated overnight at 4° C.

18.4. Purification of Monomeric SFv5AFcys-SPDP-Infliximab Conjugates

The conjugation reactions were concentrated to 750 μl and loaded on a 1×44 cm Superdex 75 SEC column running at 0.3 ml/min.

Monomeric (sFv5AF-Cys)-SPDP-Infliximab was purified by SEC on a 1×44 cm Superdex 75 column with 0.1 M PO₄ containing 1 mM EDTA, pH 7.5. Fractions 20-29 and 38-46 were collected.

Quantitation of pooled SEC fractions from both conjugates. Pooled fractions were concentrated using Centricon YM-10 concentrators. The protein concentrations of the pooled fractions were determined by BCA protein assay. Pooled fractions 20-29 had a protein concentration of 782 μg/ml, and pooled fractions 38-46 had a protein concentration of 1042 μg/ml.

18.5. SDS-PAGE Analysis of Purified Fractions

Three (3) μg or 0.3 μg of each pooled fraction was subjected to non-reduced SDS-PAGE. The gels were stained with colloidal Coomassie Blue (Bio-RAD). The gels were also transferred to polyvinylidene fluoride (PVDF) membranes and probed with an anti-5AF-Cys antibody for analysis in Western blots. Bound anti-5AF-Cys was detected by adding anti-rabbit IgG-alkaline phophatase and then nitro blue tetrazolium (NBT) and 5-bromo-4-chloro-3-idoly phosphate, toludinium salt (BCIP). The epitope and purification tags present in the recombinant sFv protein are somewhat unstable, and are lost to a varying extent when the sFv is stored in an unconjugated state for extended periods of time.

### Example 19

### Assay and Characterization of Mab Conjugates

19.1 Transcytosis Assay Design

The assay was performed in the transwell system as shown in FIG. 18. The cells are grown on a porous membrane that separates the apical and the basolateral compartment. The polyspecific binding molecules are placed in the apical compartment and then assayed by removing samples from the basolateral compartment.

Transcytosis assays were performed essentially as described above. The Mab-comprising composition or compound is placed in the apical compartment of the transwell and after a period of time, a sample from the apical compartment and a sample from the basolateral compartment are removed and separated by SDS-PAGE. After gel electrophoresis, the proteins are transferred to PVDF membranes which are probed as Western blots with a polyclonal anti-sFv antibody followed by anti-rabbit IgG-alkaline phosphatase detection with nitro blue tetrazolium and 5-bromo-4-chloro-3-idoly phosphate, toludinium salt. (NBT/BCIP). Western blotting detects any molecular species that contains the sFv; whether the sFv is present either as part of a composition or compound of the invention or as a “free” sFv molecule.

Control (untransfected) MDCK cells do not contain significant levels or any pIgR. Therefore, one expects to observe no transcytosis of the sFv or conjugate in these cells. A sample of the apical compartment should show the presence of sFv5AF-Cys and conjugates thereof, whereas a sample of the basolateral compartment should not show any sFv or conjugate thereof.

In contrast, MDCK cells that have been transfected with an expression construct that encodes and expresses a portion of pIgR have the capacity for pIgR-specific transcytosis. Thus, one expects to observe transcytosis from the apical to basolateral compartments. Accordingly, bands corresponding to sFv or conjugates thereof will be present in the basolateral compartment if the molecules are capable of trancytosis.

19.2. Transcytosis of a SFv5AF-Cys-IgG Conjugate in pIgR-Expressing MDCK Cells

Four-day old cultures of MDCK cells expressing pIgR stalk, or control MDCK cells lacking pIgR, were incubated in the presence of 20 μg sFv5AF-Cys-Infliximab in the apical chamber of a Transwell transcytosis chamber. The cells were incubated in the presence of conjugate for 20 hours, and both apical and basal chambers were harvested at the end of the incubation period. The conjugate was isolated from one-third of the media from the apical chamber, and from all of the media from the basal chamber, by incubation with Protein A-Sepharose beads. The beads were washed, resuspended in SDS-PAGE sample buffer and the proteins subjected to SDS-PAGE. The proteins were transferred to PVDF and the membranes were probed as Western blots with a polyclonal anti-sFv antibody followed by anti-rabbit IgG-alkaline phophatase and detection with NBT/BCIP.

In the case of control MDCK cells, in samples from apical compartments, bands were observed in the Western lanes, but virtually no detectable bands were observed in the lanes for samples removed from the basolateral compartments. In the pIgR stalk-expressing cells, however, bands were observed in both compartments, demonstrating that some of the molecules has undergone reverse (apical to basolateral) transcytosis.

### Example 20

### Transcytosis of Agents Non-Covalently Bound SFv5AF

M1 is a murine anti-FLAG-Tag Monoclonal Antibody that is commercially available (Sigma-Aldrich, St. Louis, Mo.). M1 may be used for detecting amino-terminal FLAG fusion proteins by immunoprecipitation, immunoblotting, or EIA, as it binds to the FLAG epitope when it is located at the free amino-terminus of a fusion protein. The sFv5AF-Cys molecule includes a FLAG epitope on its amino terminal end (FIG. 4). Thus, M1 was non-covalently bound to sFv5AF-Cys to create an [M1]::[sFv5AF-Cys] molecular complex, and the ability of this molecular complex to trancytose was examined.

M1 (0.4 ug per filter) was combined with sFv5AF (2 μg per filter) or buffer alone. After a 1 hour incubation at room temperature, transcytosis assays were carried out, essentially as described above, for 16 to 24 hours. Equilibrium appears to have been reached by 16 hours of transcytosis. Transcytosis of sFv5AF alone and sFv5AF-Cys was also assayed. Proteins were concentrated by affinity concentration 100% of the basolateral media, or 10% of the apical media. Samples containing sFv5AF or sFv5AF-Cys were concentrated using protein A. Samples containing M1 were concentrated with protein A and protein G. Samples were analyzed by non-reducing SDS-PAGE and western blotting. M1 was used to probe sFv5AF and sFv5AF-Cys. Alkaline phosphatase-conjugated anti mouse IgG was then used as a probe to detect sFv5AF and sFv5AF-Cys, as well as the M1 from the transcytosis assay.

As is shown in FIG. 20 (panels “A”, “B”, “E” and “F”), the sFv5AF (a mixture of ˜20% dimer and ˜80% monomer) causes or enhances the reverse transcytosis of M1 in a pIgR-dependent manner. No M1 is detected in the basolateral compartment when sFv5A is not present, whether pIgR is present or not.

The transcytosis of sFv5AF-Cys was also examined in this experiment. A mixture of monomers and dimers of sFv5AF-Cys was used. Although transcytosis of both monomers and dimers was pIgR-dependent, the % of transcytosed dimers was greater than that of monomers present in the same sample.

### Example 21

### Fusion Proteins Comprising Tandem Single-Chain Antibodies

21.1. Multivalent Single-Chain Antibodies

Reading frames are prepared that encode two or more copies of a single-chain antibody amino acid sequence and are used to prepare a molecule that is a single polypeptide chain that comprises two or more copies of the sFv. The multivalent single-chain antibodies are prepared using recombinant DNA expression systems as described herein. The multivalent single-chain antibodies are noncovalently associated or chemically bonded to a biologically active molecule to produce compounds of the invention.

One skilled in the art will be able to assay a variety of multivalent single-chain antibodies for desirable and undesirable properties in order to identify and produce those that are optimized for particular applications. Optimized multivalent single-chain antibodies are associated with or bonded to a biologically active molecule to produce compounds of the invention.

21.2. Fusion Proteins Comprising Multivalent Single-Chain Antibodies

In instances where the biologically active molecule is a polypeptide, a reading frame that encodes the two or more tandem copies of the sFv and the biologically active polypeptide is prepared. Expression of this reading frame in recombinant DNA expression systems leads to the production of a fusion protein that comprises a biologically active polypeptide and a multivalent sFv in a single polypeptide chain.

It may be appropriate to alter the distance and orientation of the biologically active molecule polypeptide and the two or more V(H) and V(L) regions of the sFv to prepare fusion proteins that are optimized for one or more desirable attributes. Any order or arrangement of elements may be used. By way of non-limiting example, a fusion protein having two tandem repeats of a sFv and a biologically active polypeptide (BAP) could have any of the following structures:
VH1-VL1-BAP-VH2-LV2
VH1-VL1-VH2-VL2-BAP
BAP-VH1-VL1-VH2-LV2
BAP-VH1-VH2-VL2-LV1
VH1-VH2-VL2-VL1-BAP, etc.

Molecules having multiple, for example two, BAP's are also within the scope of the invention:
BAP1-VH1-VL1-BAP2-VH2-VL2
BAP1-VH1-VL1-VH2-VL2-BAP2
VH1-VL1-VH2-VL2-BAP 1-BAP2
BAP1-BAP2-VH1-VL1-VH2-VL2, etc.

### Example 22

### Stability of sFv5AF-Cys and Complexes Thereof

22.1. Protease Stability Assays

In order to assess the relative stability of multivalent complexes and compounds, the following experiments were carried out.

Substrate, typically 20 μM, is incubated at a 1:40 substrate:enzyme ratio with trypsin, chymotrypsin, or elastase, in buffer containing 180 mM Tris, pH 7.4, and 10 mM CaCl₂ in a total volume of 200 uL. Effectiveness of protease inhibitors can be assessed by including them in the assay at the desired concentration. After incubation on ice, or various times at room temp. or 37 degrees (usually 15, 90 and 240 minutes), 10 ul of the sample is added to 30 ul of hot 2× Laemmli SDS sample buffer (160 mM Tris, pH 6.8, 2% SDS, 20% glycerol, 10.6% β-mercaptoethanol) and heated to >90 degrees in a heating block for 5 minutes. Beta-mercaptoethanol can be eliminated if desired (e.g. for conjugates with cleavable cross-linkers). After all samples are collected, they are anlayzed by SDS-PAGE and Coomassie staining or western blot with the appropriate antibodies.

The substrates tested were sFv5AF, monoclonal antibody M1, and sFv5AF:M1 complexes. The proteases and extracts that were used were trypsin, chymotrypsin, monkey, rat and human intestinal juices. Trypsin and chymotrypsin were purchased from a commercial vendor (Worthington Biochemical). Samples were taken prior to addition of proteases or extracts (S, starting material), at t=0 min, 15 min, 90 min and 4 hr. The t=0 sample was taken from samples set in ice; subsequent time points were taken during incubation at the specified temperatures. Samples were subject to SDS-PAGE, and the gels were transferred to nitrocellulose and probed with polyclonal anti-sFv5AF antibody, M1 (which is directed to FLAG tag present at the N-terminus of sFv5AF), and HRP conjugated anti-mouse IgG, which detects M1. Incubations were carried out at the specified temperatures and with or without various protease inhibitors.

22.1.1. Room Temperature Incubation

Immunoblotting with anti 5AF showed little degradation over 4 hr. A size shift, which likely represents the loss of the myc 6×His tag from the sFv5AF molecule, is seen even in the t=0 samples, indicating the tag is lost during pre-incubation at 4 degrees, during sample heating in SDS sample buffer, and/or during SDS-PAGE.

Immunoblotting with M1 detects the FLAG tag at the N-terminus of sFv5AF. The FLAG tag is lost in the t=0 trypsin samples, but the tag is lost slowly in chymotrypsin and human intestinal juices. This result is consistent with the fact that the FLAG tag contains 2 lysines residues, which makes it a better substrate for trypsin than other enzymes.

22.1.2. Incubations with or without Protease Inhibitors

Incubations at 37° C. were carried out as described for the room temperature incubations described above. 5AF stability was assayed in the presence of trypsin, chymotrypsin, human intestinal juice, and monkey jejunal juice at room temperature. One set of samples comprised the protease inhibitors chymostatin, leupeptin, and aprotinin, whereas a different set of samples contained no protease inhibitors.

Immunoblotting with anti 5AF detects sFv5AF. Even at 37 degrees, substantial degradation of sFv5AF by chymotrypsin and trypsin is not observed. A size shift (reduction in apparent Mw), suggestive of a loss of the myc 6×HIS tag is seen in all cases except for the t=0 chymotrypsin sample, and in all of the chymotrypsin samples with the protease inhibitors.

Immunoblotting with M1 detects the FLAG tag at the N-terminus of 5AF. The FLAG tag is lost in the t=0 trypsin samples, but is more stable in chymotrypsin and intestinal juices.

22.2. Detergent Stability Assays

One hundred and twenty-five (125) μg of sFv5AF-Cys, alone or in the presence of either 0.1% Tween 20 or 0.1% Triton X-100, was subjected to size exclusion chromatography on a 1×44 cm Superdex 75 column in PBS. The relative amounts of monomer and dimer remained unchanged with the detergent treatment.

The results, as represented by the percent area under the peaks, are as follows.
sFv5AF-Cys (no detergent) 47.2% dimer 38.8% monomer
sFv5AF-Cys+0.1% Tween 20 49.8% dimer 38.2% monomer
sFv5AF-Cys+0.1% Triton X-100 49.4% dimer 37.9% monomer

### Example 23

### Functional Screening and Purification

Compositions comprising an immobilized ligand that binds stalk molecules and/or pIgR domains are used to characterize and assay targeting elements directed to such molecules or domains. As is described in this Example, such compositions are also used to select and screen for targeting elements from preparations comprising a plurality of targeting elements.

23.1. General Strategies

Collections of compounds containing candidate targeting elements are prepared for selection and/or screening by the folowing exemplary methods.

23.1.1. Ligand Retention

Immobilized polypeptides that are known to bind stalk molecules and/or pIgR domains and/or regions are used to characterize, assay and identify novel targeting elements. A compound containing a polypeptide that is derived from a stalk molecule or pIgR domain or region is immobilized on a solid or semisolid surface. In an exemplary mode, the solid or semisolid is prepared as a column through which fluids may be passed.

Compounds are passed through the column, and those that are retained on the column after washing are candidate ligands that may be used as targeting elements directed to a stalk molecule or pIgR domain or region. Specific binding of candidate ligands is examined by adding molecules comprising a polypeptide that is derived from a stalk molecule or pIgR domain or region to a column to which the candidate compound is bound. When an excess of the candidate targeting element's non-immobilized target molecule is present, the target molecules in solution bind the candidate ligand, freeing it from the immobilized target molecules and allowing it to flow through the column. By determining the structure of compounds that bind to such columns, novel targeting elements are prepared.

In the case of polypeptide ligands/targeting elements, their amino acid sequences are determined and aligned in order to generate one or more consensus sequences that may be used as targeting elements. The polypeptide sequences are also be used to design a peptidomimetic that binds to pIgR. Those skilled in the art may model the structure of the peptide and convert bonds within the peptide to nonpeptide bonds.

23.1.2. Competitve Inhibition of Binding

The exemplary column that is described in the preceding section is used in other ways to prepare novel ligands directed to a stalk molecule or pIgR domain and/or region. In this mode, previously characterized compounds known to bind to stalk molecules or pIgR domains and/or regions is added to the column and are allowed to bind to the immobilized target molecule. Compounds are passed through the column, and those that displace the precharacterized binding compound are candidate novel targeting elements.

23.2. Ligands Directed to an Epitope Recognized by SFv5A

The peptide epitope for sFv5A has been determined to be QDPRLF (SEQ ID NO:______; see U.S. patent application Ser. No. ______, attorney docket no. 18062E-009000US, filed Mar. 26, 2001). This amino acid sequence represents an epitope to which sFv5A binds.

A polypeptide having an amino acid sequence that includes QDPRLF is prepared and attached to a solid or semisolid medium. Oligopeptides that can be synthesized in vitro are generally preferred. An oligopeptide having an amino acid sequence such as IENKAIQDPRLFAEEKAV (SEQ ID NO:______) is attached to a thiol Sepharose surface using a disulfide linkage or a maleimide linkage.

Alternatively, an amino terminal or carboxyl terminal cysteine residue may be incorporated into a peptide in order to provide a reactive thiol group that can be used to attach the polypetide to the column. Such peptides have amino acid sequences such as CGGGGIENKAIQDPRLFAEEKAV (SEQ ID NO:______) or IENKAIQDPRLFAEEKAVGGGGGC (SEQ ID NO:______). A cysteine-containing peptide is reacted with a 10-fold molar excess of DTNB in a 10 to 200 mM sodium phosphate buffer, pH from about 7 to about 8, containing from 0 to about 200 mM NaCl. Unreacted DTNB and TNB are removed by gel sizing chromatography on a column of Sephadex G-25 or Bio-Gel P-10 in a 10 to 200 mM sodium phosphate buffer, pH from about 6 to about 8. The TNB-peptide is then reacted with thiol Sepharose in sodium phosphate, pH 7.5, buffer containing 150 mM NaCl.

The modified Sepharose is washed free of unreacted peptide and packed into a column in preparation for passing sFv and conjugates that are specific for the QDPRLF epitope. Molecules of sFv5A, or sFv5AF, sFv5AF-Cys and other derivatives of sFv5A, or compounds or compositions comprising sFv5 or a derivative thereof, are added to the column. Preferred sFv and sFv compounds or compositions conjugates recognize and bind to the immobilized peptide. After washing the column free of nonbinding components, the column is treated with (a) 10 mM DTT to reduce the peptide disulfide bond between the peptide and the resin, (b) 25 mM free peptide that contains the epitope, such as QDPRLF, AIQDPRLFAE (SEQ ID NO:______), or a peptide that has been modified according to the results of a replacement net, or (c) a solution of 10 mM glycine adjusted to pH of about 3 to about 4 and containing 150 mM NaCl. The eluted protein is collected and immediately brought to neutral pH with Tris base if low pH elution is used. The preparation is then dialyzed and, additionally or alternatively, passed through a column of Sephadex G-25 or Bio-Gel P-10 to remove free peptide if a peptide was used to elute the sFv or sFv conjugate. Potential contaminating microbial agents are removed from a composition comprising the conjugate by passing it through a 0.2 micron filter that has been sterilized and provided in a sterile package.

23.3. Ligands Derived from Bacteria that Bind the Stalk or a pIgR Domain

Zhang et al. present evidence that the pneumococcal adhesin protein CpbA interacts with human pIgR (hpIgR) as either a part of the outer surface of a bacterial cell or as a free molecule. The regions of CpbA:hpIgR interaction were mapped using a series of large peptide fragments derived from CpbA. CpbA (Swiss-Prot Accession No. 030874) contains a choline binding domain containing residues 454-663 and two N-terminal repetitive regions called R1 and R2 (SEQ ID NOS:______ and ______, respectively) that are contained in residues 97-203 and 259-365, respectively. Zhang et al. demonstrated that polypeptides containing R1 and R2 (see FIG. 17) interact with a portion of hpIgR, whereas a polypeptide containing residues 1-101 of CpbA does not bind to hpIgR. Sequences contained within pneumococcal CpbA may be used, as well as similar sequences in CpbA homologs in other species, as sources of polypeptides that comprise novel ligands/targeting elements. Polypeptides having amino acids derived from regions R1 and R2 of CpbA are prepared and screened for ligands as described above.

23.4. Functional Purification of Compounds and Compositions

Compounds that bind to a stalk molecule or pIgR domain or region are purified using columns comprising the target molecule or a derivative thereof. For example, a sFv5A or sFv5A-comrpising compound is bound to the column and eluted. The eluate is stored under suitable conditions. These conditions include storage at −80° C., 2° to 8° C., and/or lyophilization to a dry state, e.g., a powder. For example, conjugates purified by this method are enriched for those species of compounds capable of binding to the target molecule and are relatively depleted of unconjugated sFv5A molecules, conjugates comprising non-functional sFv portions, and conjugates that lack the ability to bind to the immobilized target molecule. Compounds that are depleted in this fashion include sFv5A conjugate molecules that (a) have been chemically modified in an undesirable way, i.e., so that binding to pIgR is impaired; (b) are alternate and undesired conjugates products (such as, e.g., multimeric vs. monomeric conjugates, or conjugates formed from alternate chemical linkages); or (c) are otherwise unable to bind to the target molecule.

In this fashion, compositions that are enriched for functional (target-binding) protein conjugates are prepared. Such compositions may have many advantages over compositions prepared in other ways. These advantages include but are not limited to a reduction or elimination of undesired side-effects caused by non-functional conjugates, improved shelf life, an enhancement of the therapeutic potency of compositions comprising the compounds of the invention, and an improved level of consistency of preparations of the compounds.

A number of compositions and compounds comprising binding ligands and biologically active molecules or moieties, may be prepared. However, some compositions and compounds will be preferred based on properties and characteristics that may be selcted or screened for. For example, a preferred property of a functional molecules is the ability to bind a target molecule. For the compositions and compounds of the invention, the domain 6-GST fusion proteins described herein may be used. The GST fusion proteins are puririfed and prepared essentially as described herein and/or according to known techniques (see, e.g., Smith et al., Unit 16.7 of Chapter 16 in: Short Protocols in Molecular Biology, 2nd Ed., Ausubel et al., eds., John Wiley and Sons, New York, 1992, pages 16-28 to 16-31) and attached to a glutathione column.

### Example 24

### Evaluation of Binding Characteristics Via Surface Plasmon Resonance

24.1. Experimental Procedure

Each sFv or sFv-conjugate was tested for its ability to bind recombinant pIgR Domain 6 GST (D6-GST) fusion proteins. The pIgR Domain 6 fusion proteins were constructed from human, cynomologous monkey and rat cDNA sequences (see Example 3) and expressed in E. coli. A BIAcore® biosensor (Pharmacia Biosensor AB, Uppsala, Sweden and Piscataway, N.J.) was used to measure 5AF antibody fusion or antibody conjugate specific binding in real time to domain 6 in a capture format. The Biacore analysis was performed using a capture protocol in which an anti-GST antibody was immobilized to the Biacore chip surface, and a particular D6-GST fusion protein was bound to the antibody-coated surface. The sFv5AF or sFv5AF-containing conjugate was assayed typically over a concentration range of 15.6 to 500 nM.

BIAcore® kinetic evaluation software (BIAevaluation version 3.1) was used to determine the association on rate (ka), dissociation off rate (kd) as well as the affinity constant (K_{A}=ka/kd or K_{D}=kd/ka). Binding was quantified by global fitting the data for the antibody concentration range using a 1:1 Langmuir binding model.

24.2. Comparison of the Binding Activities of SFv5AF-Cys to Purified Monomer or Dimer SFv5AF-Cys

There is little variation in the K_{D} among species as reflected in Table 14. When comparing the binding of sFv forms to one particular species of D6-GST fusion, the purified dimer exhibits a higher affinity than the purified monomer. The difference is ˜3-fold for each species (human, rat, simian). The sFv5AF-Cys starting material, which is a mixture of monomer and dimer, has an affinity that is similar to the purified dimer. This may be due to the single binding site modeling of the data when the sFv5AF-Cys starting material is a mixture of monomer and dimer forms of sFv5AF-Cys. When comparing the purified monomer and purified dimer forms of sFv5AF-Cys, the differences in K_{D} appear to be due to changes in the association rate constant (ka). The dissociation rate constant (kd) does not vary between the monomer and dimer forms of sFv5AF-Cys.

TABLE 14
COMPARISON OF 5AF-CYS BINDING TO PURIFIED MONOMER OR DIMER 5AF-CYS BINDING VIA
SURFACE PLASMON RESONANCE
1:1 binding model

| | Mono/ | | | | | | KA | | | MW |
|---|---|---|---|---|---|---|---|---|---|---|
| Analyte | Dimer | Ligand | ka (1/Ms) | kd (1/s) | Rmax | Conc | (1/M) | KD (M) | chi\hat{ }2 | Analyte |
| 5Afcys | | Human | 9.76E+05 | 1.45E−03 | 96.5 | global fit* | 6.71E+08 | 1.49E−09 | 2.20E+01 | 28884 |
| 5Afcys | Monomer | Human | 4.28E+05 | 2.41E−03 | 85.4 | global fit* | 1.77E+08 | 5.64E−09 | 7.39E+00 | 28884 |
| 5Afcys | Dimer | Human | 1.09E+06 | 2.17E−03 | 90.5 | global fit* | 5.02E+08 | 1.99E−09 | 4.69E+00 | 57768 |
| 5Afcys | | Cyno | 1.06E+06 | 1.37E−03 | 82.6 | global fit* | 7.70E+08 | 1.30E−09 | 1.88E+01 | 28884 |
| 5Afcys | Monomer | Cyno | 4.43E+05 | 2.56E−03 | 71 | global fit* | 1.73E+08 | 5.77E−09 | 7.29E+00 | 28884 |
| 5Afcys | Dimer | Cyno | 1.12E+06 | 2.44E−03 | 77.7 | global fit* | 4.61E+08 | 2.17E−09 | 6.05E+00 | 57768 |
| 5Afcys | | Rat | 1.23E+06 | 2.82E−03 | 43.4 | global fit* | 4.36E+08 | 2.30E−09 | 1.08E+01 | 28884 |
| 5Afcys | Monomer | Rat | 5.38E+05 | 3.03E−03 | 42.3 | global fit* | 1.77E+08 | 5.63E−09 | 4.74E+00 | 28884 |
| 5Afcys | Dimer | Rat | 1.71E+06 | 3.41E−03 | 48.9 | global fit* | 5.01E+08 | 2.00E−09 | 1.31E+01 | 57768 |

In sum, the affinity of the sFv for the receptor is 3-fold higher for the purified dimer sFv compared to the purified monomer sFv; the differences are mostly due to differences in ka; and there is no significant variation in binding to D6-GST fusions from the different species tested.

24.3 Comparison of Monomer and Dimer SFv5AF-Cys-sCalcitonin Conjugate Binding to pIgR D6-GST

These experiments used the same capture protocol described above. An anti-GST antibody was immobilized to the Biacore chip surface, and the D6-GST fusion protein was bound to the antibody. The analytes were sFvAG₄-Cys salmon calcitonin conjugates tested over a concentration range of 15.6 to 500 nM. The conjugates were prepared using the non-cleavable mal-sac linker. The conjugate prepared from sFv5AG₄-Cys monomer is designated Az014; the comparable dimer conjugate is designated Az015.

TABLE 15
RESULTS OF BIACORE ASSAY OF sCALCITONIN-sFv CONJUGATES
1:1 binding model nM.

| Analyte | Ligand | ka (1/Ms) | kd (1/s) | Rmax | conc | KA (1/M) | KD (M) | Chi\hat{ }2 | Expt. |
|---|---|---|---|---|---|---|---|---|---|
| AZ014 | D6 human | 3.87E+04 | 7.26E−03 | 89.1 | global fit* | 5.33E+06 | 1.88E−07 | 1.73 | 1st |
| AZ015 | D6 human | 1.29E+05 | 1.27E−03 | 97.6 | global fit* | 1.01E+08 | 9.89E−09 | 2.4 | 1st |
| AZ014 | D6 cyno | 3.97E+04 | 7.47E−03 | 75.4 | global fit* | 5.31E+06 | 1.88E−07 | 1.23 | 1st |
| AZ015 | D6 cyno | 1.27E+05 | 1.75E−03 | 82.3 | global fit* | 7.25E+07 | 1.38E−08 | 2.9 | 1st |
| AZ014 | D6 rat | 3.42E+04 | 3.91E−03 | 33.5 | global fit* | 8.76E+06 | 1.14E−07 | 0.614 | 1st |
| AZ015 | D6 rat | 1.70E+05 | 2.68E−03 | 47.8 | global fit* | 6.35E+07 | 1.57E−08 | 1.53 | 1st |
| AZ014 | D6 human | 3.65E+04 | 6.97E−03 | 90.5 | global fit* | 5.23E+06 | 1.91E−07 | 1.8 | 2nd |
| AZ015 | D6 human | 1.22E+05 | 1.54E−03 | 102 | global fit* | 7.93E+07 | 1.26E−08 | 2.22 | 2nd |
| AZ014 | D6 cyno | 4.16E+04 | 6.54E−03 | 69.7 | global fit* | 6.36E+06 | 1.57E−07 | 1.35 | 2nd |
| AZ015 | D6 cyno | 1.28E+05 | 1.78E−03 | 85.3 | global fit* | 7.19E+07 | 1.39E−08 | 1.24 | 2nd |
| AZ014 | D6 rat | 3.79E+04 | 2.77E−03 | 29.7 | global fit* | 1.37E+07 | 7.32E−08 | 0.471 | 2nd |
| AZ015 | D6 rat | 1.77E+05 | 2.23E−03 | 46.3 | global fit* | 7.94E+07 | 1.26E−08 | 0.949 | 2nd |

*Data were fitted globally over an analyte concentration range of 15.6 to 500

There is little variation in the K_{D} among different species (human, simian, rat) as reflected in Table 15, and the variation from experiment to experiment is small. When comparing the binding of sFv forms to one species of D6-GST fusion, the dimer conjugate exhibits a higher affinity than the monomer conjugate. The difference is 7 to 19-fold for each species. The difference in K_{D} between the monomer and dimer conjugates for the D6-GST fusion is 7-19-fold, with the dimer conjugate having higher affinity. When comparing the monomer and dimer conjugates, the differences in K_{D} appear to be due to changes in both the association rate constant (ka) and the dissociation rate constant (kd).

In sum, the dimers Calcitonin conjugate has an affinity for the pIgR Domain 6 GST (D6-GST) that is approximately 10-fold higher than the affinity exhibited by the monomer conjugate; the affinity of the monomer and dimer conjugates for the D6-GST constructs do not vary significantly between different species; and the variation between experiments is small, demonstrating the reproducibility of this method.

### Example 25

### Conjugation of SFv5AF-Cys to a 15 kD Protein Using Mal-Sac-HNSA

25.1 Description of Cross-Linking Agent

The non-cleavable heterobifunctional crosslinking reagent, N-Maleimido-6-aminocaproyl-(2′-nitro,4′-sulfonic acid)-phenyl ester Na+ (mal-sac-HNSA) (BACHEM Bioscience Inc., King of Prussia, Pa.) has been synthesized and used to conjugate sulfhydryl (cysteine)-containing peptides to carrier proteins via a thioether bond (Aldwin et al., A water-soluble, monitorable peptide and protein crosslinking agent, Anal Biochem 1987 Aug. 1; 164(2):494-501). Because mal-sac-HNSA is water soluble, its concentration can be easily adjusted to maximize parameters of the conjugation reaction. The reaction of mal-sac-HNSA with amino groups releases the dianion phenolate, 1-hydroxy-2-nitro-4-benzene sulfonic acid (HNSA), which is a yellow chromophore. The concentration of HNSA may be monitored to provide spectrophotometric quantification of, e.g., the extent or rate of the conjugation reaction in progress or of parallel reactions with varying conditions. This method of detecting HNSA is also be used as an aid in monitoring the separation of activated peptide from free crosslinking reagents during purification.

25.2 Preparation of Monomeric and Dimeric SFv5AF-Cys

Monomeric and dimeric sFv5AF-Cys were isolated from 17.6 mg purified sFv5AF-Cys. Two monomer/dimer isolation runs were performed by reducing 8.8 mg aliquots with 10 mM DTT and performing size exclusion chromatography (SEC) on a 1.6×60 cm Superdex 75 column in 10 mM sodium phosphate, 0.1 M NaCl, 1 mM EDTA, pH 6.25. Monomer and dimer sFv5AF-Cys fractions from each run were pooled. The protein yield after isolation was 11.04 mg, 6.08 mg (55%) of which was the monomer species and 4.96 mg (45%) of which was Dimer sFv5AF-Cys. The recovery of sFv5AF-Cys from the column was 63%.

25.3 Preparation of Derivatized 15 kD Protein

The 15 kD protein was prepared for Mal-Sac-HNSA derivatization by desalting in 0.1 M sodium phosphate with 1 mM EDTA, pH 7.25. Seven desalting runs on a Pharmacia G-25 HiTrap desalting column were performed with 0.9 ml of 11.6 mg/ml of the 15 kD protein per run. The fractions from all runs were pooled and the OD₂₈₀ was measured. The calculated concentration of the 15 kD protein was 3.46 mg/ml and the calculated yield of the 15 kD protein was 40.8 mg in 11.8 ml.

The desalted 15 kD protein was concentrated to 2.7 ml in a Centriprep YM-10 and the OD₂₈₀ was measured. The calculated concentration of the 15 kD protein was 13.0 mg/ml and the calculated protein yield was 35.1 mg. After desalting and concentration the final recovery of the 15 kD protein was 45.6%. In some experiments, overnight dialysis was used as an addition or alternative to the desalting/concentration step.

25.4 Conjugation Reaction

The 15 kD protein was incubated with a 2.5 molar excess of mal-sac-HNSA for thirty minutes at room temperature. The reaction was stopped by the addition of an amount of glycine equimolar to mal-sac-HNSA. The 15 kD protein that had been derivatized with mal-sac-HNSA was desalted on a 5 ml G25 column (Pharmacia HiTrap) to remove excess mal-sac-HNSA. Nine tenths (0.9) ml of [15 kD protein]-[mal-sac] (13 mg/ml) was desalted into 0.1 M sodium phosphate with 1 mM EDTA, pH 7.25. Four desalting runs were performed, and one-half the [15 kD protein]-[mal-sac] preparation from each run was added to an equivalent volume of either monomer or dimer sFv5AF-Cys until all of the [15 kD protein]-[mal-sac] was desalted.

The pH of the reaction was raised from pH 6.25 to pH 7.25 by the addition of 0.5 M sodium phosphate, pH 8.0, and the remaining sFv was added to each appropriate reaction vessel. The final volume for each reaction was ˜18 ml. Both reactions were concentrated at 25° C. until the [monomer sFv5AF-Cys]-[15 kD protein] reaction volume reached 1.65 ml and [dimer sFv5AF-Cys]-[15 kD protein] reached 1.55 ml. The reactions were at room temperature for 50 minutes and at 25° C. for 2 hours, then stored at 4° C. until purified.

25.5. Purification of [SFv5AF-Cys]-[Mal-Sac-HNSA]-[15 kD Protein] Conjugates

25.5.1 Monomer Conjugate (Conjugate Preparation Az008)

[Monomer sFv5AF-Cys]-[mal-sac]-[15 kD protein] conjugates were purified by SEC on a 1.6×60 cm Superdex 75 column with a solution that was 0.1 M PO₄, pH 7.25, and 1 mM EDTA. The chromatograph of the eluent indicated that various fractions contained conjugate material. Fractions 32-43, 45, 51, 56 and 66 were selected for further characterization. The protein content, and approximate molecular weight thereof, of the selected fractions was examined by running 15 μl of selected fractions on a SDS-PAGE gel and staining the gel with colloidal Coomassie blue. Molecular weight markers were electrophoresed in a separate lane in order to estimate the size of the proteins visualized on the stained gel. Based on the results generated from the stained gels, fractions 39-45 were pooled, and the pool is referred to as “purified monomer conjugate” (i.e., [monomer sFv5AF-Cys]-[mal-sac]-[15 kD protein]) in the following sections. The protein concentrations of the pooled fractions were determined using the BCA protein assay. These determinations were used in further characterization, e.g., to calculate SDS-PAGE loads for Western analysis.

25.5.2 Dimer Conjugate (Conjugate Preparation Az009)

[Dimer sFv5AF-Cys]-[mal-sac]-[15 kD protein] was purified by SEC on a 1.6×60 cm Superdex 75 column with 0.1 M sodium phosphate with 1 mM EDTA, pH 7.25. Fractions 26-39, 51 and 59 were selected for Coomassie-stained SDS-PAGE analysis, which was carried out as described above. Fractions 28-33 were pooled to generate a “purified dimer conjugate” preparation. Fractions 36-39, 47-55, and 59-61 were also pooled for further analysis. Protein concentrations of the pooled fractions were determined using the BCA protein assay.

25.6. Conjugate Purification by Hydrophobic Interaction Chromatography

Hydrophobic interaction chromatography (HIC) on Phenyl Sepharose is used to purify the conjugation reaction proteins from the desired conjugate. Two hundred (200) μl of an unpurified sFv5AF-Cys to 15 kD protein conjugation reaction is loaded onto a 1 ml Phenyl Sepharose column in 0.1 M Na Phosphate, pH 5.5, containing 3.0 M (NH₄)₂SO₄, and a gradient was run to 0.1 M Na Phosphate, pH 5.5, containing 15% ethylene glycol. A chromatogram of the elution profile is used to determine which fractions should be pooled for further analysis.

### Example 26

### Characterization of [SFv5AF-Cys]-[Mal-Sac]-[15 kD Protein] Conjugates

26.1 Coomassie-Stained Gels

The pooled fractions were subjected to SDS-PAGE and Coomassie staining of the gels. The Coomassie-stained gels indicate that there is very little unreacted sFv5AF-Cys in the monomer conjugate preparation. There is a slight amount of unreacted sFv5AF-Cys in the dimer conjugate preparation. This result suggests that most of the dimer conjugate preparation is present in the form of two associated [monomer sFv5AF-Cys]-[mal-sac]-[15 kD protein] molecules, i.e., {[monomer sFv5AF-Cys]-[mal-sac]-[15 kD protein]}₂. However, there may be some dimer conjugate consisting of one [monomer sFv5AF-Cys]-[mal-sac]-[15 kD protein] molecule associated with one sFv5AF-Cys molecule, i.e., ([monomer sFv5AF-Cys]2-[mal-sac]-[15 kD protein]).

26.2. Mass Spectrometry

Conjugates and conjugation reactions were dialyzed into H₂O and subjected to MALDI-TOF mass spectrometry.

26.2.1 Mass Spectrometry of the Monomer Conjugate

[Monomer SFv5AF-Cys]-[Mal-Sac]-[15 kD protein] was subjected to maldi-TOF. A peak of 46748 molecular mass, corresponding to the monomer conjugate, and a peak of 29107 molecular mass, representing sFv5AF-Cys, were present. Also present were peaks below 18000 molecular mass, which correspond to free 15 kD protein.

26.3.2 Mass Spectrometry of the Dimer Conjugate

[Dimer sFv5AF-Cys]-[mal-sac]-[15 kD protein] was subjected to maldi-TOF. A peak of 45022 molecular mass, corresponding to the monomer conjugate, and a peak of 26517 molecular mass, representing sFv5AF-Cys, were present. Also present were peaks below 18000 molecular mass, which correspond to free 15 kD protein.

26.4 Transcytosis of [SFv5AF-Cys]-[Mal-Sac]-[15 kD Protein] Conjugates

The conjugates were tested in transcytosis assays, which were carried out essentially as described above. SFv5AF-Cys, monomer conjugate, dimer conjugate or 15 kD protein were incubated in the apical chamber of a transwell containing pIgR-expressing- or control MDCK cells for 17 hours at 37° C. Five (5) μl of the apical media (1.7% of the total volume) or 500 μl of the basal media (62.5% of the total volume) were affinity precipitated by incubating with 50 μl of a 10% Protein A-Sepharose slurry overnight at 4° C. The beads were washed three times and eluted with 50 μl SDS-PAGE sample buffer. Gel electrophoresis was performed with 8-16% gradient SDS-PAGE, and the gels were transferred to PVDF and probed as Western blots either with anti-sFv5AF polyclonal antibody, followed by an anti-IgG secondary antibody coupled to horse radish peroxidase (HRP) and NBT/BCIP visualization.

In pIgR Stalk-Expressing MDCK cells, samples from the apical compartments show the presence of bands having the molecular weight expected for [sFv5AF-Cys]-[15 kD protein] conjugates, as do lanes containing samples removed from the basolateral compartment. These results demonstrate that the [sFv5AF-Cys]-[15 kD protein] conjugates are transcytosed into the basolateral compartment. In contrast, when control (non pIgR expressing) MDCK cells are used, samples from the apical compartments show the presence of the conjugate, but samples of the basolateral compartments do not. Thus, the observed transcytosis is pIgR-dependent.

26.5. Binding Parameters

Surface plasmon resonance was used to characterize binding properties of the conjugates. The experiments were carried out using essentially the same procedures as described above. The results are shown in Table 17.

TABLE 17
CONJUGATE PREPARATIONS Az008 AND Az009 VIA SURFACE
PLASMON RESONANCE

| | | | | Analyte | | | |
|---|---|---|---|---|---|---|---|
| Analyte | ka (1/Ms) | kd (1/s) | Rmax | Conc | KA (1/M) | KD (M) | Chi2 |
| SFv5A | 2.93 × 10⁴ | 4.64 × 10⁻³ | 24.4 | Global fit* | 6.31 × 10⁶ | 1.58 × 10⁻⁷ | 0.590 |
| SFv5AF | 2.71 × 10⁴ | 4.64 × 10⁻³ | 42.4 | Global fit* | 4.47 × 10⁶ | 2.24 × 10⁻⁷ | 0.613 |
| SFv5AFcys | 3.76 × 10⁴ | 4.64 × 10⁻³ | 68.6 | Global fit* | 9.84 × 10⁶ | 1.02 × 10⁻⁷ | 5.32 |
| Az009 | 5.87 × 10⁴ | 4.64 × 10⁻³ | 34.9 | Global fit* | 1.46 × 10⁷ | 6.85 × 10⁻⁸ | 3.87 |
| Az008 | 3.07 × 10⁴ | 4.64 × 10⁻³ | 26.7 | 341n | 8.56 × 10⁶ | 1.17 × 10⁻⁷ | 0.165 |

*Data were fitted globally over a range (62.5 nM to 1000 nM) of analyte concentrations. Data were fitted using the 1:1 Langmuir binding model.

### Example 27

### Conjugation of SFv5AF-Cys to the 15 kD Protein Using SPDP

27.1 Description of Crosslinker

A 2-pyridyl disulfide residue in a crosslinker can react with an aliphatic thiol to form a disulfide bridge, which can be disrupted by the introduction of a reducing agent such as DTT (Carlsson et al., Protein thiolation and reversible protein-protein conjugation, Biochem J 173: 723-737, 1978). The heterobifunctional crosslinking agent N-succinimidyl 3-(2-pyridyldithio) propionate (SPDP) (available from Pierce Chemical Co., Rockford, Ill.) has been used to activate (introduce sulfhydryl groups into) proteins destined to be conjugated to thiolated proteins. It is a relatively efficient reaction that yields a pyridyl leaving group that may be easily monitored. Compounds that are chemically related to SPDP (e.g., LC-SPDP, Sulfo-LC-SPDP) may also be used.

SPDP has been used to link proteins to Fab′ fragments single-chain antibodies and monoclonal antibodies. See, e.g., Bode et al., Conjugation to antifibrin Fab′ enhances fibrinolytic potency of single-chain urokinase plasminogen activator, Circulation 1990 June; 81(6):1974-1980; Pietersz et al., In vitro and in vivo evaluation of human tumor necrosis factor-alpha (hTNFalpha) chemically conjugated to monoclonal antibody, J Drug Target 1998; 5(2):109-120; Gupta et al., Single chain Fv: a ligand in receptor-mediated gene delivery, Gene Ther 2001 April; 8(8):586-592; Woo et al., Ricin A immunotoxins of IgG and Fab of anti-CALLA monoclonal antibody: effect of water soluble long-chain SPDP on conjugate yield, immunoselectivity and cytotoxicity, Arch Pharm Res 1994 December; 17(6):452-457; and Woo et al., Stability and cytotoxicity of Fab-ricin A immunotoxins prepared with water soluble long chain heterobifunctional crosslinking agents, Arch Pharm Res 1999 October; 22(5):459-463.

27.2 Preparation of Monomeric and Dimeric SFv5AF-Cys

Preparations of sFv5AF-Cys were pooled from several sources and concentrated to 2 ml. The sFv5AF-Cys pool was incubated with 10 mM DTT for 30 minutes and then a 1.95 ml aliquot was subjected to size exclusion chromatography (SEC) on a 1.6×60 cm Superdex 75 column. The sFv5AF-Cys was resolved into two main peaks, and the later peak had a saddle. Sixteen fractions were selected across the peaks and valleys and aliquots were subjected to SDS-PAGE for Coomassie staining to determine which fractions should be pooled. The fractions pooled were fractions 27-34 (dimer), 36-40 (monomer) and 42-50 (monomer). The OD₂₈₀ was measured and sFv5AF-Cys concentration determined. Fractions 27-34 (dimer) contained 5.0 mg protein at 0.66 mg/ml, fractions 36-40 (monomer) contained 4.6 mg protein at 0.97 mg/ml and fractions 42-50 (monomer) contained 6.9 mg protein at 0.56 mg/ml.

27.3 Preparation of 15 kD Protein

A 15 kD protein was dialyzed into 100 mM sodium phosphate with 1 mM EDTA, pH 7.5. This was supplemented with 15 kD protein tha had been desalted on a 5 ml HiTrap desalting column to boost the final amount of the 15 kD protein to 59 mg, at a concentration 5.3 mg/ml. SPDP was added to 15 kD protein in a 4-fold molar excess, and the reaction was incubated at 25° C. for 30 minutes. After incubation, 1.5 ml 100 mM glycine, pH 7.5, was added to stop the substitution reaction. The [15 kD protein]-SPDP was dialyzed into 100 mM sodium phosphate with 1 mM EDTA, pH 7.5. The OD₂₈₀ was measured and the concentration of the [15 kD protein]-SPDP was determined (7.3 mg/ml). Also, an aliquot of the 15 kD Protein-SPDP was taken and 10 mM DTT added and OD₃₄₃ determined. The calculated molarity of free pyridine 2-thione leaving group was compared to the molarity of 15 kD protein-SPDP to determine the ratio of SPDP to the 15 kD protein (ratio=0.83).

27.4 Conjugation Reaction

Three separate conjugation reactions were performed due to the presence of three different species of sFv5AF-Cys. For each sFv5AF-Cys sample, 0.5 M PO₄ pH 8.0 was added to bring the pH up to 7.25 and then 15 kD protein-SPDP was added (Table 16 shows volumes and masses of reagents added in order: sFv5AF-Cys, 0.5 M PO₄, and 15 kD protein). Each reaction was placed into a centriprep YM-10 (Millipore) and the reactions were incubated while concentrating them at 25° C. for 2 hours. The reactions were stored overnight at 4° C.

TABLE 16
REACTANTS AND ORDER OF ADDITION FOR SPDP REACTIONS

| | ml 5AF- | ml 0.5M | ml 15 kD- | mg 5AF- | | mg 15 kD- | Molar |
|---|---|---|---|---|---|---|---|
| Reaction | Cys | PO₄ | SPDP | Cys | mg 15 kD | SPDP | Ratio |
| 36-40 | 4.7 | 0.197 | 2.8 | 4.6 | 13.2 | 11.0 | 4.5 |
| 42-50 | 8.6 | 0.361 | 5.2 | 6.9 | 24.4 | 20.3 | 5.5 |
| 15 kD: s5AF- | 7.6 | 0.319 | 4.5 | 5.0 | 21.1 | 17.6 | 6.6 |
| Cys Dimer | | | | | | | |

27.5 Screening of Conjugation Reactions

The final volumes for the reactions were: reaction 36-40, 2 ml; reaction 42-50, 3 ml; and the dimer reaction, 3 ml. Each concentrated reaction was subjected to SEC on a 1.6×60 cm Superdex 75 column. The fractions were screened by SDS-PAGE, loading equal volume samples from selected fractions, and the fractions containing protein peaks were pooled. The protein concentration in each fraction pool were quantitated using the BCA assay. Equal protein loads from each pool were subjected to SDS-PAGE on 5 separate 8-16% SDS-PAGE gels (I gel for Coomassie staining, 3 μg/lane; and 4 gels for Western blot analysis, 0.3 μg/lane). The fractions containing purified conjugates were identified, and final yields were calculated as above.

27.6 Purification of Monomer and Dimer Conjugates

27.6.1 Monomer Conjugate in Pooled Fractions 36-40 (Conjugate Preparation Az018A)

Two (2) ml of the 36-40 conjugation reaction was purified by SEC using a 1.6×60 cm Superdex 75 column. A large peak, corresponding to [sFv5AF-Cys]-SPDP-[15 kD protein] was present, as was a second peak corresponding to unreacted sFv5AF-Cys and the 15 kD protein. Fractions 22, 23, 25, 26, 27, 28, 29, 30, 31, 32, 33, 36, 40, 43, and 51 were selected for SDS-PAGE analysis. Fifteen (15) μl was removed from each fraction and added to 15 μl of non-reducing sample buffer. This sample was subjected to 8-16% SDS-PAGE and stained with Coomassie blue. Fractions 22-27, 29-36 (conjugate), 39-40, and 42-49 were pooled independently.

27.6.2 Monomer Conjugate in Pooled Fractions 42-50 (Conjugate Preparation Az018B)

Two (2) ml of the 42-50 (monomer) conjugation reaction was purified by SEC using a 1.6×60 cm Superdex 75 column. A large peak, representing the conjugate, was present, as was a second peak representing unreacted 15 kD protein. Fractions 10, 12, 14, 15, 16, 17, 18, 19, 20, 21, 22, 24, 26, 30, 34, and 43 were selected for SDS-PAGE analysis.

27.6.3 Dimer Conjugate (Conjugate Preparation Az019)

Three (3) ml of dimer reaction was purified by SEC using a 1.6×60 cm Superdex 75 column. A large peak (dimer sFv5AF-Cys-SPDP-15 kD protein) was present, as was a second peak corresponding to unreacted dimer sFv5AF-Cys, and a third peak corresponding to unreacted 15 kD protein. Fractions 72, 74, 76, 78, 79, 80, 81, 82, 83, 84, 86, 88, 91, 96, and 98 were selected for SDS-PAGE analysis.

### Example 28

### Characterization of [SFv5AF-Cys]-[SPDP]-[15 kD Protein] Conjugates

28.1 Coomassie-Stained Gels and Western Analysis

Western analyses were carried out to confirm the identity of material contained within the fractions. Three (3) μg of the pooled peak fractions were subjected to SDS-PAGE, and then transferred to PVDF filters. The filters were probed with polyclonal anti-sFv5AF, followed by incubation with an anti-IgG secondary antibody coupled to horse radish peroxidase (HRP), and then visualized by addition of NBT/BCIP.

The colloidal Coomassie and Western data confirm that fractions 29-36 (Az018a) contain the monomer conjugate with the c-myc tag from reactions using the 36-40 pool. Fractions 18-25 (Az018b) contain the monomer conjugate without c-myc tag from the 42-50 pool, and fractions 72-79 (Az018ab) are dimer conjugates with and without the c-myc tag.

28.2 Transcytosis Assay of [SFv5AF-Cys]-SPDP-[15 kD Protein] Conjugates

Transcytosis of conjugate preparations Az008, Az009, Az0018b, and Az0019 was analyzed on pIgR expressing MDCK cells overnight. Both sFv5AF-Cys and a non-covalent sFv5AF:M1 complex were analyzed for comparison. Az0019 transcytosis was more efficient than Az008 (non-cleavable monomer conjugate), but less efficient than Az009 (non-cleavable dimer conjugate). Az018b transcytosis was slightly less efficient than Az008. Transcytosis of Az008, Az019, and Az018b were all less efficient than sFv5AF-Cys dimer (>10%), but in a comparable range with sFv5AF:M1 complex, sFv5AF, and sFv5AF-Cys monomer (˜2%). Transcytosis was specific, as demonstrated by lack of sFv5AF:M1 transcytosis by control MDCK cells.

28.3 Binding Parameters

Surface plasmon resonance was used to characterize binding properties of the conjugates. The experiments were carried out using essentially the same procedures as described above. The results are shown in Table 18.

TABLE 18
BIACORE RESULTS WITH CONJUGATE PREPARATIONS Az018a, Az018b and Az019

| Analyte | Ligand | ka (1/Ms) | kd (1/s) | Rmax | conc | KA (1/M) | KD (M) | chi\hat{ }2 |
|---|---|---|---|---|---|---|---|---|
| 5A | D6 human | 7.29E+04 | 3.37E−03 | 96 | global fit* | 2.16E+07 | 4.63E−08 | 3.44 |
| Az018a | D6 human | 4.32E+04 | 4.49E−03 | 118 | global fit* | 9.62E+06 | 1.04E−07 | 3.67 |
| Az018b | D6 human | 4.32E+04 | 4.65E−03 | 127 | global fit* | 9.30E+06 | 1.07E−07 | 5.07 |
| Az019 | D6 human | 1.52E+05 | 1.09E−03 | 144 | global fit* | 1.39E+08 | 7.18E−09 | 4.73 |
| 5A | D6 cyno | 7.67E+04 | 3.97E−03 | 101 | global fit* | 1.93E+07 | 5.18E−08 | 3.51 |
| Az018a | D6 cyno | 5.18E+04 | 5.35E−03 | 122 | global fit* | 9.69E+06 | 1.03E−07 | 2.94 |
| Az018b | D6 cyno | 5.29E+04 | 5.57E−03 | 129 | global fit* | 9.50E+06 | 1.05E−07 | 3.7 |
| Az019 | D6 cyno | 1.64E+05 | 1.08E−03 | 142 | global fit* | 1.52E+08 | 6.57E−09 | 4.62 |
| 5A | D6 rat | 7.73E+04 | 2.62E−03 | 59.9 | global fit* | 2.95E+07 | 3.39E−08 | 2.02 |
| Az018a | D6 rat | 4.71E+04 | 3.80E−03 | 62.4 | global fit* | 1.24E+07 | 8.06E−08 | 1.89 |
| Az018b | D6 rat | 4.94E+04 | 3.85E−03 | 70.8 | global fit* | 1.28E+07 | 7.79E−08 | 3.03 |
| Az019 | D6 rat | 2.09E+05 | 1.96E−03 | 91.6 | global fit* | 1.06E+08 | 9.39E−09 | 3 |

*Data were fitted globally over a analyte concentration range of 15.6 to 500 nM

### Example 29

### Conjugation of SFv5AF to SATP-Thiolated 15 kD Protein

29.1 Description of Thiolation Agent

N-succinimidyl S-acetylthiopropionate (SATP) (Molecular Biosciences, Inc., Boulder Colo.) reacts with primary amines to add protected sulfhydryl groups (Duncan et al., Anal. Biochem. 132:68-73, 1983). The thioioacetyl group can be deprotected with 0.02 M hydroxylamine hydrochloride to render free sulfhydryl.

29.2. Thiolation of the 15 kD Protein

Reactions were carried out in 0.1 M PO₄, pH 7.25, 1 mM EDTA. The 15 kD protein and SATP were then added, typically, at concentrations of 50 mM SATP and 35 mM 15 kD protein. The reaction was allowed to proceed for 30 to 60 minutes at 20° C. A strong base, such as hydroxylamine, was then added to deprotect the thiol group, leaving it free for conjugation to sFv5AF.

29.3. Conjugation of sFv5AF to SATP-Thiolated 15 kD Protein Using Mal-Sac HNSA

29.3.1. Conjugation Reaction

The single-chain antibody sFv5AF was conjugated to the 15 kD protein using mal-sac-HNSA as the cross-linker. A preparation of sFv5AF was derivatized with mal-sac-HNSA, a heterobifunctional cross-linker with an amino-reactive HNSA group and a thiol-reactive maleimide group bridged by a non-cleavable linker region. The mal-sac-HNSA HNSA was reacted with primary amines on sFv5AF, and 15 kD protein that had been thiolated with SATP was added to the derivatized sFv5AF to conjugate via the maleimide group on sFv5AF-mal-sac.

29.3.2. Purification of [SFv5AF]-[Mal-Sac]-[SATP]-[15 kD Protein] Protein Conjugates

The sFv5AF-15 kD protein conjugate was purified by size exclusion chromatography (SEC). SEC was performed on a 1×30 cm Superdex 75 column with 0.1 M PO₄ containing 1 mM EDTA and 0.4 M Arginine, pH 7.5, at 0.3 m/min.

29.3.3. Coomassie-Stained Gels and Western Analysis

The fractions were analyzed using Coomassie-stained gels and Western analysis in order to determine which fractions, and pools of fractions, contained the conjugate, as well as the approximate amount (concentration) of the conjugate therein. The yield from the conjugation reaction was 948 μg of conjugate in 237 μl.

29.4. Conjugation of SFv5AF to SATP-Thiolated 15 kD Protein Using LC SMPT

29.4.1. Description of Crosslinker

The crosslinking agent 4-succinimidyloxycarbonyl-a-methyl-a-(2-pyridyldithio)-toluene (SMPT) is a thiol reactive and cleavable NHS ester. SMPT has a benzene ring and a methyl group adjacent to a carbon next to the disulfide bond. These functional groups hinder the disulfide linkage and thus protect the disulfide bond from being readily reduced by thiolate anions. A water soluble version of SMPT is Sulfo-LC-SMPT [sulfocuccinimidyl 6-[a-methyl-a-(2-pyridyl-dithio)toluamido]hexanoate, which does not require dissolution in organic solvents such as DMF or DMSO before addition to the conjugation buffer. An extended spacer arm (20.0 Å) has been incorporated into Sulfo-LC-SMPT to reduce steric hindrance effects which may occur during the conjugation of antibody to toxin. Thus, the extended spacer arm may increase the reactivity of this molecule in some instances.

29.4.2. Conjugation Reaction

Moneric and dimeric forms of sFv5AF were purified and conjugated to the 15 kD protein in separate reactions. sFv5AF was derivatized with sulfo-LC-SMPT (Pierce Chemical Co.). The sulfo-LC-SMPT was reacted with primary amines on sFv5AF, and SATP-thiolated 15 kD protein was added to the derivatized sFv5AF. A chemical bond forms between the pyridyl disulfide group on 5AF-LC-SMPT. This conjugate is designated [sFv5AF]-[sulfo-LC-SMPT]-[SATP]-[15 kD protein].

29.4.3. Purification of [SFv5AF]-[Sulfo-LC-SMPT]-[SATP]-[15 kD Protein] Conjugates

29.4.3.1. Purification of [SFv5AF Monomer]-[LC-SMPT]-[15 kD Protein] Conjugates

The [sFv5AF monomer]-[LC-SMPT]-[15 kD protein] was purified from the conjugation reaction by size exclusion chromatography (SEC) using a 1×30 cm Superdex 75 column with 100 mM phosphate and 1 mM EDTA, pH 7.5, at 0.4 ml/min. Based on the elution profile, sets of fractions were pooled and concentrated in a Microcon YM-10 concentrator, and protein concentrations were determined by the BCA assay.

29.4.3.2. Purification of [SFv5AF Dinomer]-[LC-SMPT]-[15 kD Protein] Conjugate

The [sFv5AF dimer]-[LC-SMPT]-[15 kD protein] conjugate was purified from the conjugation reaction by SEC using a 1×30 cm Superdex 75 column with 100 mM phosphate and 1 mM EDTA, pH 7.5, at 0.4 ml/min. Based on the elution profile, sets of fractions were pooled.

29.4.3.3. Coomassie-Stained Gels and Western Analysis

Fractions and pools of fractions were analyzed using Coomassie-stained gels and Western analysis in order to determine which fractions and pools contained the conjugate, as well as the approximate amount (concentration) of the conjugate therein. Fractions M2, M3, M4 and M5 were used as preparations of [monomer sFv5AF]-[sulfo-LC-SMPT]-[SATP]-[15 kD protein] conjugates and fractions D2 and D3 were used as preparations of [dimer sFv5AF]-[sulfo-LC-SMPT]-[SATP]-[15 kD protein] conjugates.

29.5. Conjugation of SFv5AF to SATP-Thiolated 15 kD Protein Using LC-SMCC

29.5.1. Description of Crosslinker

Succinimidyl 4-(N-maleimidomethyl)cyclohexane-1-carboxylate (SMCC) is a heterobifunctional cross-linker with NHS-ester and maleimide functional groups connected by a non-cleavable bridge. NHS-esters react with primary amines, and maleimides react with sulfhydryls. Typically, the NHS reaction is performed first, then excess crosslinker is removed, followed by addition of the component with the SH groups, and crosslinking is achieved. Crosslinking reaction is favored by using higher protein concentrations. LC-SMCC, Succiminidyl-4-(N-maleimidomethyl)cyclohexane-1-carboxy-(6-amido-caproate), is a sulfhydryl-reactive and amine-reactive heterobifunctional cross-linking agent. Its properties are similar to those of SMCC, but it has an extended aliphatic spacer arm (Yoshitake et. al., Eur. J. Biochem. 101, 395-399, 1979).

29.5.2. Conjugation Reaction

A preparation of sFv5AF was derivatized with either 1.5-fold or 2.5-fold molar excess LC-SMCC (Pierce Chemical Co.). The derivatized sFv5AF was desalted on a 5 ml G25 superfine column, and fractions 10-17 were pooled. The purified, derivatized sFv5AF was quantitated by absorbance at 280 nm, and then used in conjugation reactions.

The 15 kD protein was thiolated using 2.75-fold molar excess SATP, and then de-acetylated with 50 mM hydroxylamine. The activated 15 kD protein was then desalted on a 5 ml G25 superfine column, and fractions 9-16 were pooled. The 15 kD protein was quantiated by absorbance at 280 nm, and the derivatization ratio was determined using DTNB. The purified, derivatized 15 kD protein was then used in conjugation reactions.

Conjugation reactions using either 1.5-fold or 2.5-fold molar excess LC-SMCC to sFv5AF both had 1:1 ratios of sFv5AF-15 kD protein conjugates. Electrophoresesis and Western probing of conjugates detected a sFv5AF band at 28 kD and a conjugate band at 43 kD. Both reactions had nearly equal intensities between the two bands, indicating good conjugation yield.

29.5.3. Purification of [SFv5AF]-[LC-SMCC]-[15 kD Protein] Conjugate

The [sFv5AF]-[LC-SMCC]-[15 kD Protein] protein conjugate was purified on a 1×30 cm Superdex 75 column, with a flow rate of 0.30 ml/min and collection of 0.2 ml fractions. The two conjugations using LC-SMCC as the crosslinker were pooled, concentrated and purified by size exclusion chromatography on Superdex 75 in three separate batches.

Fractions 17-20 were pooled as the target 1:1 conjugate. About 0.5 ml of the pooled peak fractions were desalted into phosphate-buffered saline (PBS) on a 5 ml G25 Superfine column, and concentrated in a Centricon concentrator. The remaining purified conjugate was dialyzed overnight in PBS and concentrated in a Centricon concentrator.

### Example 30

### Liposomal Formulations

30.1. Structure of Liposomes

Liposomes are microscopic spheres having an aqueous core surrounded by one or more outer layer(s) made up of lipids arranged in a bilayer configuration (see, generally, Chonn et al., Current Op. Biotech., 1995, 6, 698). Liposomes may be used as cellular delivery vehicles for bioactive agents in vitro and in vivo (Mannino et al., Biotechniques, 1988, 6, 682; Blume et al., Biochem. et Diophys. Acta, 1990, 1029, 91; Lappalainen et al., Antiviral Res., 1994, 23, 119. For example, it has been shown that large unilamellar vesicles (LUV), which range in size from about 0.2 to about 0.4 microns, can encapsulate a substantial percentage of an aqueous buffer containing large macromolecules. RNA, DNA and intact virions can be encapsulated within the aqueous interior of liposomes and delivered to brain cells in a biologically active form (Fraley et al., Trends Biochem. Sci., 1981, 6, 77). Liposome-based gene therapy is reviewed by Tseng et al., Pharm. Sci. Tech. Today 1:206-213, 1998; and Ropert, Braz. J. Biol. Res. 32:63-169, 1999. U.S. Pat. No. 5,834,441 is stated to describe liposomes for the delivery of AAV-derived nucleic acids.

Liposomes may be unilamellar (single layer) or multilamellar (multilayer, often compared to an onion skin) and they may be loaded with drugs, peptides, proteins, nucleic acids, carbohydrates, plasmids, vitamins, cosmetics, and the like (Bakker-Woudenberg et al., Liposomes as carriers of antimicrobial agents or immunomodulatory agents in the treatment of infections, Eur J Clin Microbiol Infect Dis 1993; 12 Suppl 1:S61-67; Gregoriadis et al., Liposomes in drug delivery. Clinical, diagnostic and ophthalmic potential, Drugs 1993 45:15-28). Examples of techniques for encapsulating molecules into liposomes are described by Mayer et al., Techniques for encapsulating bioactive agents into liposomes, Chem Phys Lipids 40:333-345, 1986.

Liposomes make it possible to encapsulate water soluble and water insoluble substances and avoid the use of other formulations that depend on emulsification and/or surfactants. Liposomes enable the ability to control delivery characteristics of substances with the use of biodegradable and nontoxic materials that comprise the liposome formulation. While substances are contained in the liposome, they are resistant to enzymes and oxidants that exist in the vicinity of the liposome. Liposomes may be injected into a patient; intravenous or subcutaneous injection may be used. In addition, liposomes can be administered to the gastrointestinal tract or the respiratory tract. Liposomes may be encapsulated.

Liposomes are formed from vesicle-forming lipids which generally include one or more neutral or negatively charged phospholipids, typically one or more neutral phospholipids, usually in combination with one or more sterols, particularly cholesterol. Non-limiting examples of lipids useful in liposome production include phosphatidyl compounds, such as phosphatidylglycerol, phosphatidylcholine, phosphatidylserine, sphingolipids, phosphatidylethanolamine, cerebrosides and gangliosides.

Often, the major lipid component of liposomes is a phosphatidylcholine (PC) or PC derivative. PC derivatives with a variety of acyl chain groups of varying chain length and degree of saturation are commercially available or may be synthesized by known techniques. For purposes of filter sterilization, less-saturated PCs are generally more easily sized, particularly when the liposomes must be sized below about 0.3 microns. PCs containing saturated fatty acids with carbon chain lengths in the range of about 14 to about 22 carbon atoms are commonly used particularly diacyl phosphatidylglycerols. Illustrative phospholipids include, for example, dipalmitoylphosphatidylcholine, phosphatidylcholine and distearoylphosphatidylcholine. Phosphatidylcholines with mono- and di-unsaturated fatty acids and mixtures of saturated and unsaturated fatty acids may also be used. Other suitable phospholipids include those with head groups other than choline, such as, for example, ethanolamine, serine, glycerol and inositol. Other suitable lipids include phosphonolipids in which the fatty acids are linked to glycerol via ether linkages rather than ester linkages. In some embodiments, liposomes include a sterol, e.g., cholesterol, at molar ratios of from about 0.1 to about 1.0 (sterol: phospholipid).

30.2. Sterically Stabilized Liposomes

The term “sterically stabilized liposome” refers to a liposome comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome comprises one or more glycolipids, such as monosialoganglioside GM1, or is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (Allen et al., FEBS Letts., 1987, 223, 42; Wu et al., Cancer Res., 1993, 53, 3765; Papahadjopoulos et al., Ann. N.Y. Acad. Sci., 1987, 507, 64; Gabizon et al., Proc. Natl. Acad. Sci. USA, 1988, 85, 6949; U.S. Pat. No. 4,837,028 and published PCT application WO 88/04924, both to Allen et al. U.S. Pat. No. 5,543,152 to Webb et al.; and published PCT application WO 97/13499 to Lim et al.

Many liposomes comprising lipids derivatized with one or more hydrophilic polymers, and methods of preparation thereof, are known in the art. Sunamoto et al. (Bull. Chem. Soc. Jpn., 1980, 53, 2778) describe liposomes comprising a nonionic detergent. Liposomes comprising phosphatidylethanolamine (PE) derivatized with PEG or PEG stearate or other PEG-derivatized phospholipids, e.g., DSPE-PEG, formed from the combination of distearoylphosphatidylethanolamine (DSPE) and PEG have significant increases in blood circulation half-lives. (Blume et al. Biochimica et Biophysica Acta, 1990, 1029, 91; Klibanov et al., FEBS Letts., 1990, 268, 235). Liposomes having covalently bound PEG moieties on their external surface are described in European Patent No. 0,445,131 BE and WO 90/04384 to Fisher. Liposome compositions containing about 1 to about 20 mole percent of PE derivatized with PEG, and methods of use thereof, are described by Woodle et al. (U.S. Pat. Nos. 5,013,556 and 5,356,633) and Martin et al. (U.S. Pat. No. 5,213,804 and European Patent No. EP 0,496,813 B1). Liposomes comprising a number of other lipid-polymer conjugates are disclosed in WO 91/05545 and U.S. Pat. No. 5,225,212 (both to Martin et al.) and in WO 94/20073 (Zalipsky et al.) Liposomes comprising PEG-modified ceramide lipids are described in WO 96/10391 (Choi et al.). U.S. Pat. No. 5,540,935 (Miyazaki et al.) and U.S. Pat. No. 5,556,948 (Tagawa et al.) describe PEG-containing liposomes that can be further derivatized via functional surface moieties.

30.3. Targeting of Liposomes

Liposomes can be either passively or actively targeted. Passive targeting utilizes the natural tendency of liposomes to distribute to cells of the reticuloendothelial system in organs that contain sinusoidal capillaries. Active targeting, by contrast, involves modification of the liposome by coupling thereto a specific ligand such as a viral protein coat (Morishita et al., Proc. Natl. Acad. Sci. USA, 1993, 90, 8474), monoclonal antibody (or a suitable binding portion thereof), sugar, glycolipid or protein (or a suitable oligopeptide fragment thereof), or by changing the composition and/or size of the liposome in order to achieve distribution to organs and cell types other than the naturally occurring sites of localization.

Targeting of liposomes may be achieved in a variety of ways. Various linking groups are used to join lipid chains of the liposome to a targeting element. The targeting element binds a specific cell surface molecule found predominantly on cells to which delivery of the compounds of the invention is desired. Targeting elements include, by way of non-limiting for example, a hormone, growth factor or a suitable oligopeptide fragment thereof which is bound by a specific cellular receptor predominantly displayed on by cells to which delivery is desired, or a polyclonal or monoclonal antibody, or a suitable fragment thereof (e.g., Fab; sFv) that specifically binds an antigenic epitope found predominantly on targeted cells.

The targeting of liposomes may be controlled by coating the outside surface of the liposome with targeting agents such as an antibody, F(ab′)2 or Fab fragment of an antibody, cytokines, enzymes, domains and portions of proteins, peptides, polypeptides, carbohydrates, nucleic acids, oligonucleotides, etc. Such coating substances may be present in various amounts on the surface of the liposomes. In the present invention, fusion proteins that project a pIgR ligand from a bi-layer lipid membrane are used to target liposomes.

Targeting of liposomes to different cell types can also be modulated by manipulating the type and ratio of lipids present therein. See, for example, Duzgune et al., Mechanisms and kinetics of liposome-cell interactions, Adv Drug Deliv Rev 1999 40:3-18; Schreier et al., Targeting of liposomes to cells expressing CD4 using glycosylphosphatidylinositol-anchored gp120. Influence of liposome composition on intracellular trafficking. J Biol Chem 1994 269:9090-9098; and Shi et al., Noninvasive gene targeting to the brain, Proc. Natl. Acad. Sci. USA 97:7567-7572, 2000; Shimizu et al., Formulation of liposomes with a soybean-derived sterylglucoside mixture and cholesterol for liver targeting. Biol Pharm Bull 1997 20:881-886.

30.4. Preparation of Liposomes

Liposomes are prepared by any of a variety of known techniques. For example, liposomes can be formed by any conventional technique for preparing multilamellar lipid vesicles (MLVs), i.e., by depositing one or more selected lipids on the inside wall of a suitable vessel by dissolving the lipid in chloroform, evaporating the chloroform and then adding an aqueous solution which comprises the agent(s) to be encapsulated to the vessel, allowing the aqueous solution to hydrate the lipid, and swirling or vortexing the resulting lipid suspension. This process yields a mixture including the desired liposomes.

As another example, techniques used for producing large unilamellar vesicles (LUVs), such as, e.g., reverse-phase evaporation, infusion procedures and detergent dilution, can be used to produce the liposomes. These and other methods for producing lipid vesicles are described in Liposome Technology, Volume I (Gregoriadis, Ed., CRC Press, Boca Raton, Fla., 1984). The liposomes can be in the form of steroidal lipid vesicles, stable plurilamellar vesicles (SPLVs), monophasic vesicles (MPVs) or lipid matrix carriers (LMCs) of the type disclosed in U.S. Pat. Nos. 4,588,578 and 4,610,868 (both to Fountain et al.), U.S. Pat. No. 4,522,803 (to Lenk et al.), and U.S. Pat. No. 5,008,050 (to Cullis et al.). In the case of MLVs, the liposomes can be subjected to multiple (five or more) freeze-thaw cycles to enhance their trapped volumes and trapping efficiencies and to provide a more uniform interlamellar distribution of solute if desired (Mayer et al., J. Biol. Chem., 1985, 260, 802). Specific methods for making particular oligodeoxynucleotide:liposome compositions are described in U.S. Pat. No. 5,665,710 to Rahman et al.

Following their preparation, liposomes may be sized to achieve a desired size range and relatively narrow distribution of sized particles. In preferred embodiments, the liposomes have a lower range of diameters of from about 50 to about 75 nM, most preferably about 60 nM, and an upper range of diameters from about 75 to about 150 nM, most preferably about 125 nM, where “about” indicates ±10 nM.

Several techniques are available for sizing liposomes to a desired size range. Sonicating a liposome suspension by either bath or probe sonication produces a progressive size reduction down to small unilamellar vesicles (SUVs) less than about 0.05 microns in size. Homogenization, which relies on shearing energy to fragment large liposomes into smaller ones, is another known sizing technique in which MLVs are recirculated through a standard emulsion homogenizer until a selected liposome size range, typically between about 0.1 and about 0.5 microns, is achieved. Extrusion of liposomes through a filter or membrane is another method for producing liposomes having a desired size range (see, for example, U.S. Pat. No. 4,737,323 to Martin et al. and U.S. Pat. No. 5,008,050 to Cullis et al.). Other useful sizing methods are known to those skilled in the art. In most such methods, the particle size distribution can be monitored by conventional laser-beam size determination or other means known in the art.

Liposomes may be dehydrated, preferably under reduced pressure using standard freeze-drying equipment, for extended storage. Whether dehydrated or not, the liposomes and their surrounding media can first be frozen in liquid nitrogen and placed under reduced pressure. Although the addition of the latter freezing step makes for a longer overall dehydration process, there is less damage to the lipid vesicles, and less loss of their internal contents, when the liposomes are frozen before dehydration.

To ensure that a significant portion of the liposomes will endure the dehydration process intact, one or more protective sugars may be made available to interact with the lipid vesicle membranes and keep them intact as water is removed. Appropriate sugars include, but are not limited to, trehalose, maltose, sucrose, lactose, glucose, dextran and the like. In general, disaccharide sugars may work better than monosaccharide sugars, with trehalose and sucrose being particularly effective in most cases, but other, more complicated sugars may alternatively be used. The amount of sugar to be used depends on the type of sugar and the characteristics of the lipid vesicles. Persons skilled in the art can readily test various sugars and concentrations to determine what conditions work best for a particular lipid vesicle preparation (see, generally, Harrigan et al., Chem. Phys. Lipids, 1990, 52, 139, and U.S. Pat. No. 4,880,635 to Janoff et al.). Generally, sugar concentrations of greater than or equal to about 100 mM have been found to result in the desired degree of protection. Once the liposomes have been dehydrated, they can be stored for extended periods of time until they are to be used. The appropriate conditions for storage will depend on the chemical composition of the lipid vesicles and their encapsulated active agent(s). For example, liposomes comprising heat labile agents should be stored under refrigerated conditions so that the potency of the active agent is not lost.

30.6. Pharmaceutical Formulations of Liposomes

Numerous pharmaceutical formulations of liposomes have been developed for delivery to a variety of cell types and tissues have been described. Non-limiting examples include formulations for the intranasal administration of vaccines (U.S. Pat. No. 5,756,104), and aerosol formulations for the delivery of anti-cancer drugs (U.S. Pat. No. 6,090,407). Liposomes may be encapsulated by, and/or incorporated into, formulations such as pills, tablets, capsules, caplets, suppositories, liquids designed for deliver via the alimentary canal, preferably via oral administartion. Pharmaceutical formulations that comprise liposomes and which are used for the delivery of macromolecules, including but not limited to proteins and nucleic acids, are described in, by way of non-limiting example, U.S. Pat. No. 6,132,764, Targeted polymerized liposome diagnostic and treatment agents; U.S. Pat. No. 5,879,713, Targeted delivery via biodegradable polymers; U.S. Pat. No. 5,851,548, Liposomes containing cationic lipids and vitamin D; U.S. Pat. No. 5,759,519, Method for the intracellular delivery of biomolecules using thiocationic lipids; U.S. Pat. No. 5,756,352, Thiocationic lipid-nucleic acid conjugates; U.S. Pat. No. 5,739,271, Thiocationic lipids; U.S. Pat. No. 5,711,964, Method for the intracellular delivery of biomolecules using liposomes containing cationic lipids and vitamin D; and U.S. Pat. No. 5,494,682, Ionically cross-linked polymeric microcapsules.

### Example 31

### Oral Transport

The ability of a composition or compound of the invention to deliver a biologically active complex or compound, or a bioactive portion or metabolite thereof, via the gastrointestinal tract is evaluated as follows.

31.1. Animal Preparation

A cannula is placed in the jejunum, ileum, or colon of a rat. The end of the cannula is threaded under the skin until it exits the skin between the shoulders of the rat; when located in this manner, the rat cannot damage the cannula and the cannula remains patent for long times. A cannula is also placed in the jugular vein and threaded under the skin so it also exits between the shoulders. A harness may be used to further protect the cannula. The intestinal cannula may be used for administering materials directly into the intestine. The jugular cannula may be used to withdraw blood, which can be further analyzed for the quantity and for biological and biophysical characteristics and functions. The test article (0.2 to 1 ml) is administered through the cannula into the intestine and blood is withdrawn at timed intervals. Heparin is used to keep the jugular vein from clotting.

Alternatively, a urethral catheter, such as C.R. Bard, Inc. Covington, Ga., All-Purpose Urethral Catheter with Funnel End, 16 inches length, two eyes, X-ray opaque rubber, is used to administer the test article to the colon. In this case, a rat is anesthetized with Ketamine. The catheter is cut so that about 8 cm of catheter remains. A Luer lock fitting is placed on the cut end of the catheter and a 1 ml syringe containing the test article is attached to the Luer lock fitting. The catheter is filled with the test article. A mark at 7.5 from the tip of the catheter is made with ink and the catheter is inserted into the rectum of the rat until the mark is just visible. The syringe is then used to deliver the required volume of test article, generally about 0.05 to about 5 ml in volume.

31.2. Assays

The test article may be detected by radioiodinating or otherwise radiolabeling it. One or more components of a conjugate protein may be labeled. Blood that is collected is then used to determine the number of cpm in a measured weight or volume of blood. The test article may be detected by an appropriate biochemical or biological assay, including without limitation ELISA, enzyme, receptor binding, etc. A monoclonal antibody directed to an antigen present in the biologically active complex or compound, or a bioactive portion or metabolite thereof, is typically used.

The biophysical features of the test article may be detected by immunoprecipitation, by SDS-PAGE and detection of the radiolabel by various imaging processes including autoradiography, by Western blotting with agents that bind to the test article, by gel sizing on Sephadex or Sepharose resins of an appropriate size, etc.

31.3. Pharmacokinetics

A graph of the amount of test article present in blood as a function of time allows one to observe the amount of transport over time. Dividing the amount of test article in blood by the amount of test article administered to the rat yields the percent absorbed dose. By administering the same amount of test article through the intestine and through an intravenous injection and comparing the area under the curve, the absolute bioavailability is determined. The bioavailabilty relative to other routes of injection, such as subcutaneous, may also be obtained. Those skilled in the art will know how to perform the pharmacokinetic analysis.

The transport of the test article may be compared to controls of the complex or compound that has not been conjugated to a targeting element. Such comparison demonstrates the specificity and selectivity of transport.

### Example 32

### Assays for the In Vivo Delivery of Biologically Active Complexes or Compounds, and/or Bioactive Portions or Metabolites Thereof

A variety of assays are used to determine the extent of delivery of a biologically active complex or compound, or a bioactive portion or metabolite thereof, from the lumen of an organ to the body of an animal. Non-limiting examples of such organs are the gastrointestinal tract and the lung. For example, in order to determine the delivery a biologically active complex or compound, or a bioactive portion or metabolite thereof, from the gastrointestinal tract is determined according to the following procedures.

32.1. Animal Preparation

A cannula is implanted into the jugular vein of a rat for the purpose of collecting blood samples at various times. Another cannula is implanted into a region of the intestine, jejunum, ileum, or colon, for the purpose of administering the therapeutic entity to the intestine. A 350-375 gram Spraque-Dawley rat is suitable for this purpose although other strains of rats may be used. The cannulae are guided under the skin so that they exit the skin directly between the shoulders of the rat. This position prevents the rat from damaging the cannulae. A single rat per cage is required. The fusion protein is administered to the rat 2 to 7 days after the cannulae are implanted. During this time, the rat is observed for its general health and to determine the patency of the cannulae.

32.2. Administration of Test Article and Sample Collection

The test article (i.e., a composition comprising a complex or compound of the invention) is given to the rat through the intestinal cannula. Before administration, a sample of blood (approximately 200 microliters) is withdrawn through the jugular vein cannula. Samples of blood are collected over a 8 to 48 hour period. The jugular cannula is kept patent by using saline with a small amount of heparin to prevent clotting. The blood is collected into a 1.5 ml Eppendorf tube that contains 5 microliters of heparin (about 5 to 50 units/ml) to prevent clotting. The blood is kept on ice for up to 1 hour, but no longer, before it is centrifuged in a table top Eppendorf centrifuge for 30 to 60 seconds. The supernatant is collected (plasma) and stored in a suitable manner, usually by freezing at −80° C. Blood may also be collected and allowed to clot and form clotted material, which is then separated from the serum by centrifugation. The serum is stored in a suitable manner, usually by freezing at −80° C.

32.3. Assays

The presence and amount of a biologically active complex or compound, or a bioactive portion or metabolite thereof, is measured using any appropriate assay. For example, the complex or compound may be radioiodinated with ¹²⁵I using any of the usual methods of radioiodination that are known to those skilled in the art. These methods include using chloramine-T, immobilized chloramine-T, iodine monochloride, lactoperoxidase beads, or Iodogen. Radioiodinated biologically active complexes or compounds are separated from unreacted ¹²⁵I by chromatography including, by way of non-limiting example, size separation on Sephadex or Sepharose, or by dialysis. The weight of the blood is determined by collecting the blood into a preweighed Eppendorf or small glass tube and determining the weight of the blood by subtraction after weighing the tube containing the blood. The entire tube may be counted in a gamma counter and the number of counts per minute divided by the weight of the blood to determine the number of cpm per gram of blood (essentially equivalent to the cpm/ml of blood). A graph of the cpm/ml of blood as a function of time after administration of the radiolabelled therapeutic entity is used to illustrate the transport of the complex or compound, and/or bioactive portions or metabolites thereof, from the intestine into blood.

The test article may be examined to determine if it has the same molecular weight by SDS-PAGE. A sample of the plasma may be compared on SDS-PAGE with a sample of the radiolabelled test article that was administered through the cannula. If the patterns of radioactivity (autoradiography) are the same, then it is concluded that the complex or compound present in blood is not degraded. The blood sample is reacted to immunoprecipitate the therapeutic entity. The immunoprecipitated sample is compared to an immunoprecipitated sample from the stock radiolabelled fusion protein by separation and visualization on SDS-PAGE. A quantitative estimate of the amount of a complex or compound, or a bioactive portion or metabolite thereof, is made by comparing the amount of cpm that was immunoprecipitated from blood samples and from stock radiolabelled fusion protein.

An immunoassay such as, for example, an enzyme linked immunosorbent assay (ELISA) is used to determine the concentration of the test article. In this case, the test article is not radiolabelled. A monoclonal antibody that recognizes an epitope present in the complex, compound, or bioactive portion or metabolite thereof, i.e., the antigen to which the Mab is directed, is coated to the bottom of 96-well plates. After washing, the presence and quantity of bound Mab is determined by adding to the immobilized Mab a second antibody that is directed to the Mab and conjugated to a detectable moeity such as, e.g., horse radish peroxidase or alkaline phosphatase. After washing, a substrate for horse radish peroxidase or alkaline phosphatase is incubated in the well. Substrate is detectable or results in a detectable product. The amount of the product determined by spectrophotometry at an appropriate wavelength. A control curve (using known quantities of the fusion protein) is used to determine the concentration of the biologically active complex or compound, or bioactive portion or metabolite thereof, in the plasma samples.

32.4. Related Protocols

Similar experiments are conducted to examine the ability of compositions and components of the invention for the rectal delivery of complexes or compounds via, e.g., a suppository. In these experiments, a composition or compound of the invention is administered by a rectal tube. A catheter is inserted through the anus of an anesthetized rat. The urinary catheter inserted 7.5 cm through the anus will result in delivery within the colon.

Similarly, the above described procedures can be used to examine the delivery of complex or compound, or a bioactive portion or metabolite thereof, via inhalation. In these experiments, the fusion protein is administered as an aerosol or microparticulate formulation to the nasal or pulmonary cavity.

### Example 33

### In Vivo Testing

Rat cancer models are used to determine the efficacy of compositions and compounds of the invention (for an example of the application of such methods, see Beneditti et al., Cancer Res. 59:645-652, 1999). For example, a pIgR-targeted Mab that reacts with epidermal growth factor receptor (EGFR) is tested for its ability to inhibit the growth of tumors implanted into a rat. If the Mab reacts with rat EGFR, the tumor cells that are implanted are of rat origin and grown in a wild type rat. If the Mab reacts with human EGFR (e.g., Cetuximab, ABX-EGF), the tumors cells that are implanted are of human origin and are grown in a severely immune compromised (SCID) rat.

33.1. Animal Preparation

The rat is prepared for administration of the therapeutic entity by inserting a cannula into a region of the intestine, such as the jejunum, ileum, or colon. After the surgery required to insert the cannula, the rat is optionally allowed to rest for 2 to 7 days to recover. During this time, the rat is observed for its general health and the patency of the cannula. During this time, or shortly before the surgery, tumor cells are injected subcutaneously into the flank of the rat. Depending on the specific tumor cell line used and its ability to form tumors, 10,000 to 5,000,000 cells are injected subcutaneously. The cells are first grown in tissue culture medium and then taken up as a suspension. The cells are injected into the animal subcutaneously.

The tumor cells are allowed to grow for 5 to 14 days before the tumor is treated with the test article administered through an intestinal cannula in a formulation appropriate for the gastrointestinal tract. Alternatively, formulations for the inhalation delivery of proteins are tested via administered through the pulmonary or nasal cavity using an aerosol. The EGFR-expressing cell line TE8, an esophageal squamous cell carcinoma, and the EGFR-deficient cell line H69 may be used to determine the efficacy of the test article. (Suwa et al., International Journal of Cancer. 75:626-634, 1998). The A431 cell line, a human epidermoid carcinoma tumor cell line, may also be used to test the effects of the test article. The A431 cells are grown in athymic rodents, including rats. Athymic nude rats bearing orthotopically implanted LNCAP tumors may be implanted subcutaneously and treated with the test article. (Rubenstein et al., Medical Oncology 14:131-136, 1997).

Tumor cells, such as C6 cells, may also be implanted stereotactically into the right caudate nucleus of Wistar rats. A cannula into the intestine may also be put into these rats for the purpose of administering the fusion protein. Rats with well-established cerebral C6 glioma foci may be given the fusion protein through the intestinal cannula.

33.2. Assays

Measurements of the tumor size are made using calipers to measure the dimensions of the tumor in two directions. The volume of the tumor is determined by multiplying the longest dimension times the square of the shortest dimension and dividing the product by 2. By plotting the tumor volume as a function of time (using the average or mean tumor volume) for a group of rats given the fusion protein, and comparing the same plot for a group of untreated rats bearing a tumor prepared in the same manner, one skilled in the art can determine the ability of the test article to inhibit or slow the growth of the tumor and preferably, to eradicate the tumor.

The mean survival time of tumor bearing rats is about 15-20 days in this model. The efficacy of the test article may be measured by comparing the life span of control rats (i.e, tumor bearing rats given no test article) to rats given the test article (Pu et al., Journal of Neurosurgery 92:132-139, 2000).

### Example 34

### Pharmaceutical Formulations of Compositions, Complexes and Compounds

34.1. Capsules, Tablets and Caplets

A preferred pharmaceutical formulation of a composition or compound of the invention is a pill, e.g., a capsule, tablet, caplet or the like, that is suitable for oral administration. Numerous capsule manufacturing, filling, and sealing systems are well-known in the art. Preferred capsule dosage forms can be prepared from gelatin and starch. Gelatin has been the traditional material, and the dosage forms are generally produced by well known dip molding techniques. After manufacture, gelatin capsules are filled with the desired composition and then sealed. A more recently developed alternative to gelatin dosage forms are capsules produced from starch. Starch capsules (typically made from potato starch) afford several advantages compared to gelatin capsules, including pH-independent dissolution, better suitability for enteric coating, water in the dosage form is tightly bound to the starch (and is thus less likely to migrate into the composition encapsulated in the dosage form), and the absence of animal-derived ingredients (which may be antigenic or contaminated with pathogens). Vilivilam, et al., PSTT 3:64-69, 2000). Starch capsules are odorless and rigid, and exhibit similar dissolution properties as compared to gelatin capsules.

Capsules of any suitable size can be manufactured. Starch capsules are typically made in two pieces, a cap and a body, using injection molding techniques. See Eith et al., Manuf. Chem. 58: 21-25, 1987; Idrissi et al., Pharm. Acta. Helv. 66: 246-252, 1991; Eith et al., Drug Dev. Ind. Pharm. 12: 2113-2126, 1986. The two pieces are then sealed together during filling to prevent separation. Sealing can achieved by applying a hydroalcoholic solution to the inner surface of the cap.

34.2. Enteric Coatings

After making the capsule dosage forms, if desired, they can be coated with one or more suitable materials. For example, when it is desired to deliver the encapsulated composition to the intestines, one or enteric coatings may be applied. Traditionally, enteric coatings were used to prevent gastric irritation, nausea, or to prevent the active ingredient from being destroyed by acid or gastric enzymes. However, these coatings can also be used to deliver agents to particular gastrointestinal regions.

A variety of enteric coatings are known in the art, and any suitable coating, or combinations of coatings, may be employed. Suitable coatings for starch capsules include aqueous dispersions of methacrylic acid copolymers and water-based reconstituted dispersion of cellulose acetate phthalate (CAP). See Brogmann et al., Pharm. Res. 11:S-167; Vilivalam, et al., Pharm. Res. 14:S-659, 1999; Vilivalam et al., Pharm. Res. 15:S-645, 1998; Burns et al., Int. J. Pharm. 134: 223-230, 1996; Davis et al., Eur. J. Nucl. Med., 19: 971-986, 1992. Indeed, a variety of coatings can be used to coat encapsulated dosage forms. These coatings include pH-sensitive materials, redox-sensitive materials, and materials that can be broken down by specific enzymes or microorganisms present in the intestine. Watts et al. (1995), WIPO publication WO35100, reports an enteric-coated starch capsule system for targeting sites in the colon. The pH sensitive enteric coating begins to dissolve when the dosage form enters the small intestine, and coating thickness dictates in which region of the intestine the capsule disintegrates, for example, in the terminal ileum or in the ascending, transverse, or descending colon. Other coatings, or combinations of coatings, can also be used to achieve the same effect.

34.3. Packaging

After a dosage from is prepared, it is typically packaged in a suitable material. For pill or tablet dosage forms, the dosage forms may be packaged individually or bottled en masse. An example of individual packaging PVC-PVdC-Alu, where aluminum blisters are covered with PVC (polyvinyl chloride) coated with PVdC (polyvinylidene chloride) to improve water vapor and oxygen protection. Suitable bottling materials include tinted, transluscent, or opaque high density polyethylene.

Those skilled in the art will be able to use the preceding information to prepare appropriate formulations for the gastrointestinal delivery of the fusion proteins of the invention. Other related information is known in the art and may be utilized to prepare appropriate formulations for gastrointestinal delivery of the fusion proteins.

### Example 35

### Formulations and Medical Devices for Inhalation Therapy

One aspect of the invention relates to an aerosol inhaler, or other medical device, for delivery of a monoclonal antibody. Such devices are useful for inhalation therapies based on the compositions and compounds of the invention. The term “inhalation therapy” refers to the delivery of a therapeutic agent, such as a drug or a fusion protein of the invention, in an aerosol form to the respiratory tract (i.e, pulmonary delivery). For reviews, see Gonda (J. Pharm. 89:940-945, 2000); Byron et al. (J. Aerosol Med. 7:49-75, 1994; and Niven (Crit. Rev. Ther. Drug Carrier Syst. 12:151-231, 1995).

The compositions and compounds of the invention are formulated for pulmonary delivery, and incorporated into medical devices such as inhalers, according to the following considerations and criteria, as well as other considerations and criteria known to those skilled in the art. A practicioner of the art will be able to use the following information to prepare appropriate formulations and medical devices for pulmonary delivery of the compositions and compounds of the invention.

35.1. Inhalation Therapy Using Monoclonal Antibodies

Inhalers comprising bioactive, particulary therapeutic, drugs complexes and compoundds may be used to deliver them quickly, and via self-administration. Such medical devices can be used to treat chronic or acute disorders or disease where it is desired to deliver a drug via an inhalation route and in a short period of time. Chronic attacks of a disorder or disease include, for example, asthma attacks. A non-limiting example of a drug useful for treating asthma is the monoclonal antibody CDP 835. Other Mab's that may desirably be delivered via inhalation include without limitation BEC2, ABX-EGF, E25, Palivixumab, and the like.

35.2. Formulations for Inhalation Therapy

Compositions and compounds that are intended to be used in inhalation therapy must be formulated into a composition that is appropriate for delivery via inhalation. Two formulations of therapeutic agents that are useful for inhalation therapy include those in the form of liquid particles and solid particles. The liquid formulations are generated by nebulizing solutions of the therapeutic agent. Solid particle formulations are either in the form of a powder suspended in a propellant which is administered from a metered dose inhaler, or simply as a powder that is administered from a dry powder inhaler. In the case of polypeptide therapeutic agents, solid particle aerosols can be made by lyophilizing the polypeptide from solution and then milling or grinding the lyophilized drug to the desired particle size for pulmonary administration.

Non-limiting examples of formulations of therapeutic agents, including proteins, for inhalation therapy are described in Bittner et al. (J. Microencapsul. 16:325-341, 1999; Flament et al. (Int. J. Pharm. 178:101-109, 1999); and Langenback et al. (Pediatr. Pulmonol. 27:124-129, 1999), and references cited therein. Non-limiting examples of inhalation formulations of proteins are described in U.S. Pat. Nos. 5,230,884; 5,354,562; 5,457,044; 5,888,477; 5,952,008; 5,970,973; 6,000,574; 6,051,551; 6,060,069; 6,085,753; and 6,121,247.

35.3. Aerosol Inhalers

An “aerosol inhaler” or “inhaler” is a device by which a patient can actively breathe in a given dose of a therapeutic agent. A typical application for such a medical device is for the treatment of an acute asthma attack. Delivery of drugs via inhalation, however, can be used for many other treatments including those described herein. For example, drugs administered by inhalation may be taken up by cells lining the interior of the pulmonary system and be delivered into the body therefrom. In the present invention, fusion proteins that comprise a biologically active polypeptide and an appropriate pIgR targeting polypeptide and, as a result of reverse transcytosis, will be delivered into the circulatory system of a patient.

Inhalers have long been used to deliver drugs into a patient's lungs. Typically, an inhaler provides a mixture of therapeutic agents and air or some other type of propellant gas. The formulation of the therapeutic agent is delivered into the patient when he or she inhales from a mouthpiece on the inhaler. In general aerosol delivery systems rely on a mixture of the therapeutic agent with one or more propellants, and optional inactive ingredients, to increase dispersion and stability of the therapeutic agent. Inhalation of the formulation can be by either the nose or mouth and often is self-administered. Because of the small volume of each dosage, the propellant generally evaporates simultaneously or shortly after delivery of the therapeutic agent.

Correct inhalation of an aerosol formulation may require good hand-breath coordination. In the case of some inhalers, delivery ideally proceeds in such a manner that a patient first exhales and then applies the device to his mouth and as he begins to inhale, triggers the action of the inhaler by activating an actuating element thereof. Upon such activation, the aerosol formulation consisting of a propellant and therapeutic agent present in the said propellant and distributed therein, passes from the inhaler through a nozzle into the respiratory system of the patient. Inhalation of the therapeutic formulation into the respiratory system can be via the nasal cavity, the bucal cavity, or both. As the patient actively inhales gases from these cavities the aerosol formulation is delivered to the lungs. Atomization and dispersion of the therapeutic formulation in an inhaler can be triggered electronically or mechanically.

In general, there are three types of inhalers that are used to deliver therapeutic agents during inhalation therapy: nebulizers, metered dose inhalers (MDIs) and dry powder inhalers (DPIs). Each of these types of inhaler may be used to deliver the fusion proteins of the invention.

Nebulizers are electrical devices that send a therapeutic composition directly into a patient's mouth by tube or, in children, by clear mask. Nebulizers require no hand-breath coordination. The prescribed amount of medicine is placed in the device, a tube in inserted into the mouth (or, in the case of children, a mask is placed the child's nose and mouth), and breathing commences normally until the therapeutic composition is depleted.

Measured-dose inhalers (MDIs, a.k.a. metered dose inhaler) send a measured dose of a therapeutic composition into the mouth using a small amount of pressurized gas. In MDIs, a “spacer” may be placed between the drug reservoir and the mouth to control the amount inhaled in a single application. The therapeutic composition into the spacer, which is then squeezed by the patient as he quickly inhales the composition. MDIs have recently fallen out of favor because the common MDI propellant chlorofluorocarbon (CFC) has been found to deplete the atmosphere's ozone layer, and there are international agreements to phase out the production and use of CFC.

Dry-powder inhalers (DPIs) provide a popular alternative to aerosol-based inhalers. DPIs have the advantage of not requiring a propellant. However, because they have no propellant, PDIs depend on the force of inhalation to get the therapeutic composition into the lungs. Children, people with severe asthma, and people suffering acute attacks may be unable to produce enough airflow to use dry-powder inhalers successfully. Nonetheless, DPIs are used in inhalation therapies involving the fusion proteins of the invention.

Various types of inhalers for delivering therapeutic agents are known. By way of non-limiting examples, see U.S. Pat. Nos. 3,938,516; 4,627,432; 5,941,240; 6,116,239; 6,119,688; and 6,119,684. One example of a dry powder inhaler that is within the scope of the invention is the Diskhaler, which is described in U.S. Pat. No. 4,627,432. The Spiros inhaler, described in U.S. Pat. No. 5,921,237, is another dry powder inhaler that is within the scope of the invention. Other dry powder inhalers that are within the scope of the invention include but are not limited to those described in U.S. Pat. Nos. 6,012,454; 6,045,828; 6,055,980; 6,056,169; 6,116,237; and 6,116,238.

Those skilled in the art will be able to use the preceding information to prepare appropriate formulations and medical devices for pulmonary delivery of the molecules of the invention. Other necessary information is known in the art and may be utilized to prepare appropriate formulations and medical devices.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other documents.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

1-17. (canceled)

18. A complex or compound comprising 2 or more targeting elements directed to one or more ligands that confer transcellular, transcytotic or paracellular transporting properties to an agent specifically bound to said ligand.

19. (canceled)

20. (canceled)

21. The complex or compound of claim 18, wherein said ligand is the pIgR stalk or a domain, conserved sequence or region thereof.

22. (canceled)

23. The complex or compound of claim 18, wherein said ligand is a polypeptide having an amino acid sequence selected from the group consisting of LRKED (SEQ ID NO:16), QLFVNEE (SEQ ID NO:17, LNQLT (SEQ ID NO:18), YWCKW (SEQ ID NO:19), GWYWC (SEQ ID NO:20), STLVPL (SEQ ID NO:21), SYRTD (SEQ ID NO:22), and KRSSK (SEQ ID NO:23).

24. The complex or compound of claim 18, wherein said ligand is in a region of a pIgR, wherein said pIgR can be from any animal, wherein said region is selected from the group consisting of:
R1 From KRSSK (SEQ ID NO:23) to the carboxy terminus of pIgR,
R2a From SYRTD SEQ ID NO:22) to the carboxy terminus of pIgR,
R2b From SYRTD SEQ ID NO:22) to KRSSK (SEQ ID NO:23),
R3a From STLVPL (SEQ ID NO:21) to the carboxy terminus of pIgR,
R3b From STLVPL (SEQ ID NO:21) to KRSSK (SEQ ID NO:23),
R3c From STLVPL (SEQ ID NO:21) to SYRTD SEQ ID NO:22),
R5e From YWCKW (SEQ ID NO:19) to GWYWC (SEQ ID NO:20),
R6e From LNQLT (SEQ ID NO:18) to GWYWC (SEQ ID NO:20),
R6f From LNQLT (SEQ ID NO:18) to YWCKW (SEQ ID NO 19),
R7e From QLFVNEE (SEQ ID NO:17) to GWYWC (SEQ ID NO:20),
R7f From QLFVNEE (SEQ ID NO:17 to YWCKW (SEQ ID NO:19),
R7g From QLFVNEE (SEQ ID NO:17) to LNQLT (SEQ ID NO:18),
R8e From LRKED (SEQ ID NO:16) to GWYWC (SEQ ID NO:20),
R8f From LRKED (SEQ ID NO:16) to YWCKW (SEQ ID NO:19),
R8g From LRKED (SEQ ID NO:16) to LNQLT (SEQ ID NO:18), and
R8h From LRKED (SEQ ID NO:16) to QLFVNEE (SEQ ID NO:17).

25-46. (canceled)

47. A method for transporting a biologically active agent through an epithelial or mucosal barrier, comprising contacting said epithelial or mucosal barrier with the complex or compound of claim 18.

48. A method of treating a disease in an animal, comprising contacting said animal with the complex or compound of claim 18.

49. A medical device or kit comprising a pharmaceutical composition comprising the complex or compound of claim 18.

50-53. (canceled)

54. A method for delivery of a molecule to an animal in need thereof, comprising:
administering via a pulmonary or nasopharyngeal route a composition comprising a multimeric targeting element comprising two to four single chain variable region fragments (sFv), each sFv comprising a heavy chain variable domain covalently linked, directly or through a polypeptide linker, to a light chain variable domain, wherein one or more of said sFvs is covalently or noncovalently associated with said molecule, and wherein said multimeric targeting element confers apical to basolateral transcytosis to said molecule in an in vitro transcytotic assay.

55. A method according to claim 54, wherein said multimeric targeting element comprises two to four sFvs that specifically bind to an epitope on pIgR.

56. A method according to claim 55, wherein said multimeric targeting element comprises two to four sFvs that specifically bind to an epitope on pIgR that is distinct from secretory component.

57. A method according to claim 54, wherein said multimeric targeting element comprises two to four sFvs that specifically bind to one or more epitopes on pIgR stalk.

58. A method according to claim 54, wherein said molecule is a polypeptide molecule.

59. A method according to claim 58, wherein said multimeric targeting element comprises one or more sFvs synthesized as a fusion protein with said polypeptide molecule.

60. A method according to claim 58, wherein said polypeptide molecule is selected from the group consisting of interferon α, interferon β, interferon γ, interleukin 1, interleukin 2, interleukin 3, interleukin 4, interleukin 5, interleukin 6, interleukin 7, interleukin 8, interleukin 9, interleukin 10, interleukin 11, interleukin 12, interleukin 13, interleukin 14, interleukin 15, granulocyte colony stimulating factor (G-CSF), erythropoietin (EPO), granulocyte macrophage colony stimulating factor (GM-CSF), fibroblast growth factor 1, fibroblast growth factor 2, fibroblast growth factor 3, fibroblast growth factor 4, human growth hormone, platelet derived growth factor, epidermal growth factor, factor VIII, insulin, insulin-like growth factor 1 (IGF1), calcitonin, antibody to TNFα, and functional derivatives thereof.

61. A method according to claim 54, wherein said multimeric targeting element comprises two sFvs.

62. A method for delivery of a polypeptide molecule to an animal in need thereof, comprising:
administering via a pulmonary or nasopharyngeal route a composition comprising a multimeric targeting element comprising two to four sFvs that specifically bind to an epitope on pIgR, each sFv comprising a heavy chain variable domain covalently linked, directly or through a polypeptide linker, to a light chain variable domain, wherein one or more of said sFvs is synthesized as a fusion protein with said polypeptide molecule.

63. A method according to claim 62, wherein said multimeric targeting element comprises two sFvs.

64. A method according to claim 62, wherein said multimeric targeting element comprises two to four sFvs that specifically bind to an epitope on pIgR that is distinct from secretory component.

65. A method according to claim 62, wherein said multimeric targeting element comprises two to four sFvs that specifically bind to one or more epitopes on pIgR stalk.

66. A method according to claim 62, wherein said polypeptide molecule is selected from the group consisting of interferon α, interferon β, interferon γ, interleukin 1, interleukin 2, interleukin 3, interleukin 4, interleukin 5, interleukin 6, interleukin 7, interleukin 8, interleukin 9, interleukin 10, interleukin 11, interleukin 12, interleukin 13, interleukin 14, interleukin 15, granulocyte colony stimulating factor (G-CSF), erythropoietin (EPO), granulocyte macrophage colony stimulating factor (GM-SCF), fibroblast growth factor 1, fibroblast growth factor 2, fibroblast growth factor 3, fibroblast growth factor 4, human growth hormone, platelet derived growth factor, epidermal growth factor, factor VIII, insulin, insulin-like growth factor 1 (IGF 1), calcitonin, antibody to TNFα, and functional derivatives thereof.
